(19) United States
(12) Patent Application Publication
(10) Pub. No.: US 2022/0298141 A1
(43) Pub. Date: Sep. 22, 2022

(54) ENZYME INHIBITORS

(71) Applicant: KALVISTA PHARMACEUTICALS LIMITED, Salisbury (GB)

(72) Inventors: Rebecca Louise DAVIE, Salisbury (GB); Hannah Joy EDWARDS, Salisbury (GB); David Michael EVANS, Salisbury (GB); Simon Teanby HODGSON, Ampthill (GB); Andrew Peter CRIDLAND, Harlow Essex (GB); Emanuela GANCIA, Harlow Essex (GB); Erica Lee GOLDSMITH, Saffron Walden Essex (GB); Paul Stuart HINCHLIFFE, Saffron Walden Essex (GB); Karamjit Singh JANDU, Saffron Walden Essex (GB); Alun John SMITH, Nottingham (GB)

(21) Appl. No.: 17/633,973

(22) PCT Filed: Aug. 21, 2019
(86) PCT No.: PCT/GB2019/052357

§ 371 Date: Feb. 9, 2022

(51) Int. Cl. C07D 401/14; C07D 471/04; C07D 401/12; C07D 217/22; A61P 7/02

(52) U.S. Cl. CPC C07D 401/14; C07D 471/04; C07D 401/12; C07D 217/22; A61P 7/02

(57) ABSTRACT

The present invention provides compounds of formula (I) compositions comprising such compounds; the use of such compounds in therapy; and methods of treating patients with such compounds; wherein A, B, and, n, are as defined herein.

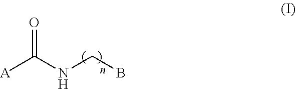

This invention relates to enzyme inhibitors that are inhibitors of Factor XIIa (FXIIa), and to the pharmaceutical compositions, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is (3FXIIa. The separate catalytic activities of αFXIIa and βFXIIα contribute to the activation and biochemical functions of FXIIa. Mutations and polymorphisms in the F12 gene can alter the cleavage of FXII and FXIIa.

FXIIα has a unique and specific structure that is different from many other serine proteases. For instance, the Tyr99 in FXIIα points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIα has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst. 2019, D75, 1-14; “Structures of human plasma β-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIα converts plasma prekallikrein (PK) to plasma kallikrein (PKa), which provides positive feedback activation of FXII to FXIIa. FXII, PK, and high molecular weight kininogen (HK) together represent the contact system. The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIα has additional substrates both directly, and indirectly via PKa, including Proteinase-activated receptors (PARs), plasminogen, and neuropeptide Y (NPY) which can contribute to the biological activity of FXIIa. Inhibition of FXIIα could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Göbel et al., Proc Natl Acad Sci USA. 2019 Jan. 2; 116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 August; 136(2):474-80. doi: 10.1016/j.thromres.2015.06.028). PKa proteolytically cleaves NPY and thereby alters its binding to NPY receptors (Abid et al., J Biol Chem. 2009 Sep. 11; 284(37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-0-12-800100-4.00002-7; and Hopp et al., J Neuroinflammation. 2017 Feb. 20; 14(1):39. doi: 10.1186/s12974-017-0815-8). CSL-312, an antibody inhibitory against FXIIa, is currently in clinical trials for the prophylactic prevention and treatment of both C1 inhibitor deficient and normal C1 inhibitor hereditary angioedema (HAE), which results in intermittent swelling of face, hands, throat, gastro-intestinal tract and genitals (see https://www.clinicaltrials.gov/ct2/show/NCT03712228). Mutations in FXII that facilitate its activation to FXIIa have been identified as a cause of HAE (see Bjorkqvist et al., J Clin Invest. 2015 Aug. 3; 125(8):3132-46. doi: 10.1172/JC177139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20; 129(16):2280-2290. doi: 10.1182/blood-2016-09-740670; Frohlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omy112. doi: 10.1093/omcr/omy112; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 Jan. 28 and Gao et al., J Proteome Res. 2008 June; 7(6):2516-25. doi: 10.1021/pr800112g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol Vis Sci. 2016 May 1; 57(6):2390-9. doi: 10.1167/iovs.15-18272). FXII deficiency is protective against VEGF induced retinal edema in mice (Clermont et al., ARVO talk 2019). Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31; 112(13):4068-73. doi: 10.1073/pnas.1423764112; Simões et al., J Neurochem. 2019 August; 150(3):296-311. doi: 10.1111/jnc.14793; Gobel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renne et al., J Exp Med. 2005 Jul. 18; 202(2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLoS One. 2016 Jan. 27; 11(1):e0146783. doi: 10.1371/journal.pone.0146783; Leung et al., Transl Stroke Res. 2012 September; 3(3):381-9. doi: 10.1007/s12975-012-0186-5; Simão et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

However, there remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis. In particular, there remains a need to develop new FXIIa inhibitors.

### DESCRIPTION OF THE INVENTION

The present invention relates to a series of heterocyclic derivatives that are inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these composition.

In a first aspect, the present invention provides a compound of formula (I)

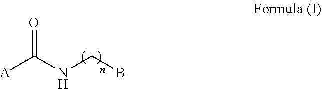

wherein:
n is 0, 1, or 2;
A is a 6-membered heteroaryl of formula (II),

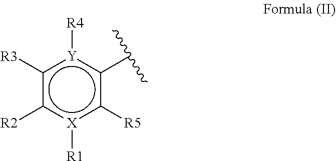

wherein X and Y are independently selected from C and N, wherein at least one of X or Y is N;
wherein R5 is selected from —NR12(CH₂)₀₋₃(heterocyclyl), NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁₋₄NR13R14, and —NR12(CH₂)₀₋₃O(aryl);
wherein R2 and R3 are independently selected from H, halo, alkoxy, alkyl, cycloalkyl, aryl, and heteroaryl;
wherein R1 and R4 are independently absent, or independently selected from H, halo, alkoxy, alkyl, cycloalkyl, aryl, and heteroaryl; or
wherein X and Y are independently selected from C and N, wherein at least one of X or Y is N;
wherein R1, R4, and R5 are independently absent or independently selected from H, halo and alkyl;
wherein one of R2 or R3 is

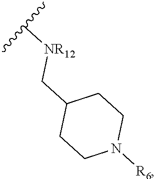

and the other of R2 or R3 is selected from H, halo or alkyl;
wherein R6 is H, alkyl, or heteroaryl^{b}; or
wherein X and Y are independently selected from C and N, wherein at least one of X or Y is N;
wherein R1 and R4 are independently absent or independently selected from H, halo and alkyl;
wherein R3 is halo;
wherein R2 is —(CH₂)₀₋₃NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁₋₄NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) and —O—(CH₂)₀₋₃(heteroaryl); and
wherein R5 is H, alkyl and halo; or
wherein X and Y are C;
wherein R4 is H, halo, alkyl;
wherein R5 is H or alkyl;
wherein R3 is H or halo;
wherein one of R1 and R2 is —(CH₂)(heterocyclyl) or —N(R12)CO(CH₂)₀₋₃(heterocyclyl), and the other of R1 and R2 is selected from H and alkyl;
wherein X is C or N, and Y is C;
R1 is absent, H or alkyl;
R4 is H or alkyl;
R5 is H or alkyl;
wherein either: (a) R2 and R3 together with the carbon atoms to which they are bonded form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}, or (b) R2 and R3 are independently selected from H and halo, wherein at least one of R2 or R3 is halo, or (c) R2 and R3 are independently selected from H, aryl^{b} and heteroaryl^{b}, wherein at least one of R2 or R3 is aryl^{b}, or heteroaryl^{b};
B is one of:
(i) a fused 6,5- or 6,6-heteroaromatic bicyclic ring, containing N and, optionally, one or two additional heteroatoms independently selected from N, O and S;
   wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF3 and —NR13R14;
   wherein the 6,5-heteroaromatic bicyclic ring may be attached via the 6- or 5-membered ring;
(ii) phenyl, which may be optionally substituted with 1, 2 or 3 substituents independently selected from, alkyl, heteroaryl, alkoxy, heterocyclyl, OH, halo, CN, CF₃, and a carbon-containing 4-, 5-, 6- or 7-membered heterocylic ring containing 1, 2 or 3 heteroatoms independently selected from N and N12, which may be saturated or unsaturated with 1 or 2 double bonds and which may be optionally mono- or di-substituted with substituents independently selected from oxo, alkyl, alkoxy, OH, halo, and CF₃; or
(iii) phenyl, wherein two adjacent carbon atoms on the phenyl are either linked together by —N═C—N(R8)-C(═O)— to form a quinazolinone or linked together by —CH₂—N(R8)-C(═O)— to form an isoindolinone; or
(iv) heteroaryl; or
(v) a fused 6,5- or 6,6-bicyclic ring containing N and containing an aromatic ring fused to a non-aromatic ring and, optionally, one or two additional heteroatoms independently selected from N, O and S;
   wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14;
   wherein the 6,5-bicyclic ring may be attached via the 6- or 5-membered ring;
      alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro;
      alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, and heterocyclyl^{b};
      alkyl^{b} is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂, —NHCOCH₃, CF₃, halo, oxo, heterocyclyl^{b}, and cyclopropane;
      alkylene is a bivalent linear saturated hydrocarbon having 1 to 5 carbon atoms (C₁-C₅); alkylene may optionally be substituted with 1 or 2 substituents independently selected from alkyl, (C₁-C₅)alkoxy, OH, CN, CF₃, and halo;
      aryl is phenyl, biphenyl or naphthyl; aryl may be optionally substituted with 1, 2 or 3 substituents independently selected from alkyl, alkoxy, OH, —SO₂CH₃, halo, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR13, —CONR13R14, —(CH₂)₀₋₃—NR13R14, OCF₃ and CF₃; or two adjacent carbon ring atoms on the aryl may be optionally linked by a heteroalkylene to form a non-aromatic ring containing 5, 6, or 7 ring members; or optionally wherein two adjacent ring atoms on aryl are linked to form a 5- or 6-membered aromatic ring containing 1 or 2 heteroatoms that are selected from N, NR8, S, and O, which may be optionally substituted as for heteroaryl^{b};
      aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R12)₂, halo, CN, and CF₃; or two adjacent carbon ring atoms on the aryl may be optionally linked by a heteroalkylene to form a non-aromatic ring containing 5, 6, or 7 ring members;
      cycloalkyl is a monocyclic saturated hydrocarbon ring of between 3 and 6 carbon atoms (C₃-C₆); cycloalkyl may optionally be substituted with 1 or 2 substituents independently selected from alkyl^{b}, (C₁-C₆)alkoxy, OH, CN, CF₃, and halo;
      halo is F, Cl, Br, or I;
      heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₆)alkoxy, OH, CN, CF₃, and halo;
      heteroaryl is a 5- or 6-membered carbon-containing aromatic ring containing 1, 2, 3, or 4 ring members that are selected from N, NR8, S, and O; heteroaryl may be optionally substituted with 1, 2 or 3 substituents independently selected from alkyl, alkoxy, aryl^{b}, OH, OCF₃, halo, heterocyclyl^{b}, CN, and CF₃;
      heteroaryl^{b} is a 5- or 6-membered carbon-containing aromatic ring containing one, two or three ring members that are selected from N, NR8, S, and O; heteroaryl^{b} may be optionally substituted with 1, 2 or 3 substituents independently selected from methyl, ethyl, propyl, isopropyl, alkoxy, CH₂aryl^{b}, OH, OCF₃, halo, CN, and CF₃;
      heterocyclyl is a 4-, 5-, 6-, or 7-membered carbon-containing non-aromatic ring containing one, two or three ring members that are selected from N, NR8, S, SO, SO₂ and O; heterocyclyl may be optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl;
      heterocyclyl^{b} is a 4-, 5-, 6-, or 7-membered carbon-containing non-aromatic ring containing one, two or three ring members that are selected from N, NR12, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃;
      R13 and R14 are independently selected from H, —SO₂CH₃, alkyl^{b}, heteroaryl^{b}, and cycloalkyl; or R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R13 and R14 together with the nitrogen atom to which they are attached form a carbon-containing 5- or 6-membered heterocylic ring, which is fused to an aryl^{b} or a heteroaryl^{b};
      R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, and —(CH₂)₀₋₃heterocyclyl^{b}; or R8 is a carbon-containing 4-, 5-, 6- or 7-membered heterocylic ring containing 1, 2 or 3 heteroatoms independently selected from N, N12, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃;
      R12 is independently selected from H, —SO₂CH₃, methyl, ethyl, propyl, isopropyl, and cycloalkyl; and tautomers, isomers, stereoisomers (including enantiomers, diastereoisomers and racemic and scalemic mixtures thereof), deuterated isotopes, and pharmaceutically acceptable salts and/or solvates thereof.

The invention is also described by the appended numbered embodiments.

The compounds of the present invention have been developed to be inhibitors of FXIIa. As noted above, FXIIa has a unique and specific binding site and there is a need for small molecule FXIIa inhibitors.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that certain compounds of the present invention may exist in solvated, for example hydrated, as well as unsolvated forms. It is to be understood that the present invention encompasses all such solvated forms.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, R2 can be —(CH₂)₀₋₃heterocyclyl, which more specifically can be piperidinyl. In this case, piperidinyl can be optionally substituted in the same manner as “heterocyclyl”.

It will be understood that “alkylene” has two free valencies i.e. it is bivalent, meaning that it is capable of being bonded to twice. For example, when two adjacent ring atoms on A“are linked by an alkylene to form a cyclopentane, the alkylene will be —CH₂CH₂CH₂—.

It will be understood that when any variable (e.g. alkyl) occurs more than once, its definition on each occurrence is independent of every other occurrence.

It will be understood that combinations of substituents and variables are permissible only if such combinations result in stable compounds.

As is clear from the definitions above, and for the avoidance of any doubt, it will be understood that “B” and “Y” define closed groups as defined above, and do not encompass boron and yttrium, respectively.

As noted above, “heteroalkylene” is a bivalent linear saturated hydrocarbon having 2 to 5 carbon atoms (C₂-C₅), wherein at least one of the 2 to 5 carbon atoms is replaced with NR8, S, or O. For example, —CH₂O is a “heteroalkylene” having 2 carbon atoms wherein one of the 2 carbon atoms has been replaced with O.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

As noted above, A can be a 6-membered heteroaryl of formula (II),

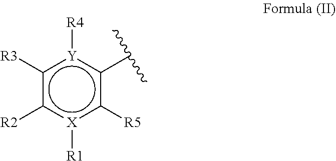

wherein X and Y are independently selected from C and N, wherein at least one of X or Y is N;
wherein R5 is selected from —NR12(CH₂)₀₋₃(heterocyclyl), —NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁₋₄NR13R14, and —NR12(CH₂)₀₋₃O(aryl);
wherein R2 and R3 are independently selected from H, halo, alkoxy, alkyl, cycloalkyl, aryl, and heteroaryl;
wherein R1 and R4 are independently absent, or independently selected from H, halo, alkoxy, alkyl, cycloalkyl, aryl, and heteroaryl.

X can be N. Y can be N. Both X and Y can be N.

When X is N, R1 is absent. When Y is N, R4 is absent.

When X is C, R1 can be H, halo, alkoxy, alkyl, cycloalkyl, aryl, and heteroaryl. More specifically, R1 can be H, halo, alkoxy, alkyl, or cycloalkyl.

When Y is C, R4 can be H, halo, alkoxy, alkyl, cycloalkyl, aryl, and heteroaryl. More specifically, R4 can be H, halo, alkoxy, alkyl, or cycloalkyl.

R2 can be H. R2 can be halo (e.g. chloro). R2 can be alkyl (e.g. methyl). R2 can be alkoxy (e.g. methoxy). R2 can be cycloalkyl (e.g. cyclopropane). R2 can be aryl (e.g. phenyl). R2 can be heteroaryl (e.g. pyridinyl).

R3 can be H. R3 can be halo (e.g. chloro). R3 can be alkyl (e.g. methyl). R3 can be alkoxy (e.g. methoxy). R3 can be cycloalkyl (e.g. cyclopropane). R3 can be aryl (e.g. phenyl). R3 can be heteroaryl (e.g. pyridinyl).

At least one of R2 and R3 can be halo, in particular, chloro.

R5 can be —NR12(CH₂)₀₋₃(heterocyclyl). R5 can be —NR12(heterocyclyl). R5 can be —NR12CH₂(heterocyclyl). R5 can be —NR12(CH₂)₂(heterocyclyl). R5 can be —NR12(CH₂)₃(heterocyclyl).

R5 can be —O(CH₂)₀₋₃(heterocyclyl). R5 can be —O(heterocyclyl). R5 can be —OCH₂(heterocyclyl). R5 can be —O(CH₂)₂(heterocyclyl). R5 can be —O(CH₂)₃(heterocyclyl).

“Heterocyclyl” can be selected from tetrahydropyranyl, piperidinyl, morpholinyl, piperazinyl, pyrrolidinyl, and azetidinyl, which as noted above can all be optionally substituted in the same manner as “heterocyclyl”. For example, two adjacent ring atoms on the heterocyclyl can be linked to form a 5- or 6-membered aromatic ring containing 1 or 2 heteroatoms that are selected from N, NR8, S, and O, such as imidazole. When NR8 is present, R8 can be alkyl (e.g. —CH₂CH₂OCH₃) or cycloalkyl (e.g. cyclopropane).

R5 can be —NR12(CH₂)₀₋₃(heteroaryl). R5 can be —NR12(heteroaryl). R5 can be —NR12CH₂(heteroaryl). R5 can be —NR12(CH₂)₂(heteroaryl). R5 can be —NR12(CH₂)₃(heteroaryl).

“Heteroaryl” can be imidazolyl or pyridinyl, which as noted above can be optionally substituted in the same manner as “heteroaryl”.

R5 can be —NR12(CH₂)₀₋₃(aryl). R5 can be —NR12(aryl). R5 can be —NR12CH₂(aryl). R5 can be —NR12(CH₂)₂(aryl). R5 can be —NR12(CH₂)₃(aryl).

R5 can be —O(CH₂)₀₋₃(aryl). R5 can be —O(aryl). —OCH₂(aryl). R5 can be —O(CH₂)₂(aryl). R5 can be —O(CH₂)₃(aryl).

R5 can be —NR12(CH₂)₀₋₃O(aryl). R5 can be —NR12-O-(aryl). R5 can be —NR12(CH₂)O(aryl). R5 can be —NR12(CH₂)₂O(aryl). R5 can be —NR12(CH₂)₃O(aryl).

“Aryl” can be phenyl, which as noted above can be optionally substituted in the same manner as “aryl”. For example, aryl (e.g. phenyl) can be substituted with heterocyclyl^{b} e.g. piperazine or piperidine. Alternatively, two adjacent carbon ring atoms on the aryl (e.g. phenyl) may be optionally linked by a heteroalkylene to form a non-aromatic ring containing 5, 6, or 7 ring members (e.g. a 6-membered ring such as piperidine).

R5 can be —NR13R14.

R5 can be —O—(CH₂)₁₋₄NR13R14. R5 can be —O—(CH₂)NR13R14. R5 can be —O—(CH₂)₂NR13R14. R5 can be —O—(CH₂)₃NR13R14. R5 can be —O—(CH₂)₄NR13R14.

R13 can be H and R14 can be cycloalkyl (e.g. cyclopentane). R13 can be H and R14 can be alkyl^{b}, e.g. alkyl^{b} substituted with —NHCOCH₃.

Alternatively, R13 and R14, together with the nitrogen atom to which they are attached can form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds. More specifically, R13 and R14, together with the nitrogen atom to which they are attached can form azetidine, pyrrolidine, piperidine, or azetane, which as noted above can be optionally substituted in the same manner as R13 and R14. For instance, the ring formed by R13 and R14 can be substituted with oxo.

X can be N, Y can be C, and R3 can be halo. X can be N, Y can be C, and R3 can be halo and R5 can be —NR12(CH₂)₀₋₃(heterocyclyl). More specifically, X can be N, Y can be C, and R3 can be halo and R5 can be —NR12(CH₂)(heterocyclyl), e.g. —NH(CH₂)(heterocyclyl). More specifically, “heterocyclyl” can be piperidine. The heterocyclyl (e.g. piperidine) can contain an NR8 group. The NR8 group can be N(alkyl^{b}). More specifically, the NR8 group can be NCH₃. Alternatively, the NR8 group can be N(CH₂CH₃). Alternatively, the NR8 group can be N(CH₂CH₂OCH₂).

X can be N, Y can be C, and R3 can be halo. X can be N, Y can be C, and R3 can be halo and R5 can be —NR12(CH₂)₀₋₃(heterocyclyl). More specifically, X can be N, Y can be C, and R3 can be halo and R5 can be —NR12(CH₂)(heterocyclyl), e.g. —NH(CH₂)(heterocyclyl). More specifically, “heterocyclyl” can be piperidine. The heterocyclyl (e.g. piperidine) can contain an NR8 group. The NR8 group can be N(cycloalkyl). More specifically, the NR8 group can be N(cyclopropane).

X can be N, Y can be C, and R3 can be halo. X can be N, Y can be C, and R3 can be halo and R5 can be —NR12(CH₂)₀₋₃(heterocyclyl). More specifically, X can be N, Y can be C, and R3 can be halo and R5 can be —NR12(CH₂)(heterocyclyl), e.g. —NH(CH₂)(heterocyclyl). More specifically, “heterocyclyl” can be piperidine. Two adjacent ring atoms on the heterocyclyl (e.g. piperidine) can be linked to form a 5- or 6-membered aromatic ring containing 1 or 2 heteroatoms that are selected from N, NR8, S, and O. More specifically, the two adjacent ring atoms on the heterocyclyl (e.g. piperidine) can be linked to form imidazole.

X and Y can be N. X and Y can be N, and R2 and R3 can be H. X and Y can be N, R2 and R3 can be H, and R5 can be —NR12(CH₂)₀₋₃(aryl). More specifically, X and Y can be N, R2 and R3 can be H, and R5 can be —NR12(aryl), e.g. —NH(aryl). The “aryl” can be phenyl. More specifically, two adjacent carbon ring atoms on the aryl (e.g. phenyl) can be linked by a heteroalkylene to form a non-aromatic ring containing 5, 6, or 7 ring members. For instance, the two adjacent ring atoms on the aryl (e.g. phenyl) can be linked to form piperidine. The piperidine formed can contain an NR8 group that is NCH₃.

X and Y can be N. X and Y can be N, and R2 and R3 can be H. X and Y can be N, R2 and R3 can be H, and R5 can be —NR12(CH₂)₀₋₃(heterocyclyl). More specifically, X and Y can be N, R2 and R3 can be H, and R5 can be —NR12(CH₂)(heterocyclyl), e.g. —NH(CH₂)(heterocyclyl). The “heterocyclyl” can be piperidine. The piperidine can have an NR8 group that is NCH₃.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF3 and —NR13R14; wherein the 6,5-heteroaromatic bicyclic ring may be attached via the 6- or 5-membered ring.

B can be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

B can be a fused 6,5-heteroaromatic bicyclic ring. The fused 6,5-heteroaromatic bicyclic ring can be attached via the 6-membered ring. The fused 6,5-heteroaromatic bicyclic ring can be attached via the 5-membered ring. Exemplary fused 6,5-heteroaromatic bicyclic rings can be selected from: 5-azathianaphthene, indolizine, indole, isoindole, indazole, benzimidazole, benzoxazole, and benzothiazole, which can all be optionally substituted in the same manner as “a fused 6,5-heteroaromatic bicyclic ring”.

B can also be phenyl, which may be optionally substituted with 1, 2 or 3 substituents independently selected from, alkyl, heteroaryl, alkoxy, heterocyclyl, OH, halo, CN, CF₃, and a carbon-containing 4-, 5-, 6- or 7-membered heterocylic ring containing 1, 2 or 3 heteroatoms independently selected from N and N12, which may be saturated or unsaturated with 1 or 2 double bonds and which may be optionally mono- or di-substituted with substituents independently selected from oxo, alkyl, alkoxy, OH, halo, and CF₃.

More specifically, B can be phenyl substituted with heteroaryl (e.g. tetrazole or triazole), halo (e.g. fluoro), and alkoxy (e.g. methoxy). Alternatively, B can be phenyl substituted with a carbon-containing 5-membered heterocylic ring containing 3 heteroatoms independently selected from N and N12, which is substituted with oxo.

Alternatively, B can be phenyl substituted with —CH₂NH₂ and two methyl groups.

B can also be a phenyl, wherein two adjacent carbon atoms on the phenyl are either linked together by —N═C—N(R8)-C(═O)— to form a quinazolinone or linked together by —CH₂—N(R8)-C(═O)— to form an isoindolinone.

Alternatively, B can be heteroaryl (e.g. imidazolyl).

B can also be a fused 6,5- or 6,6-bicyclic ring containing N and containing an aromatic ring fused to a non-aromatic ring and, optionally, one or two additional heteroatoms independently selected from N, O and S; wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14; wherein the 6,5-bicyclic ring may be attached via the 6- or 5-membered ring; More specifically, B can be a fused 6,5-bicyclic ring. More specifically, B can be a fused 6,5-bicyclic ring that is attached via the 5-membered ring. More specifically, the 5-membered ring can be cyclopropane and the 6-membered ring can be pyridine, (e.g. pyridine substituted with —NH₂).

Alternatively, A can be a 6-membered heteroaryl of formula (II),

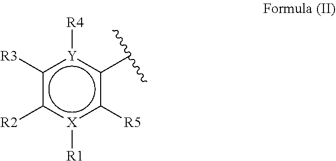

wherein X and Y are independently selected from C and N, wherein at least one of X or Y is N;
wherein R1, R4, and R5 are independently absent or independently selected from H, halo and alkyl;
wherein one of R2 or R3 is

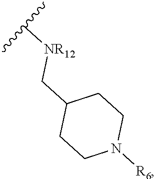

and the other of R2 or R3 is selected from H, halo or alkyl;
wherein R6 is H, alkyl, or heteroaryl^{b}.

X can be N. Y can be N. Both X and Y can be N. When X is N, R1 is absent. When Y is N, R4 is absent.

When X is C, R1 can be H. R1 can be halo (e.g. chloro). R1 can be alkyl (e.g. methyl).

When Y is C, R4 can be H. R4 can be halo (e.g. chloro). R4 can be alkyl (e.g. methyl).

R5 can be H. R5 can be halo (e.g. chloro). R4 can be alkyl (e.g. methyl).

R2 can be

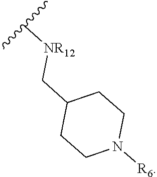

R12 can be H. R12 can be alkyl (e.g. methyl). R6 can be H. R6 can be alkyl (e.g. methyl). R6 can be heteroaryl^{b} (e.g. pyridinyl). R12 can be H and R6 can be alkyl (e.g. methyl). R12 can be alkyl (e.g. methyl) and R6 can be heteroaryl^{b} (e.g. pyridinyl). When R2 is

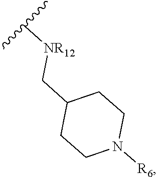

R3 can be H. Alternatively, R3 can be halo (e.g. chloro). Alternatively, R3 can be alkyl (e.g. methyl).

R3 can be

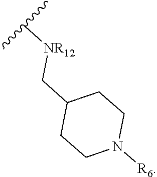

R12 can be H. R12 can be alkyl (e.g. methyl). R6 can be H. R6 can be alkyl (e.g. methyl). R6 can be heteroaryl^{b} (e.g. pyridinyl). R12 can be H and R6 can be alkyl (e.g. methyl). R12 can be alkyl (e.g. methyl) and R6 can be heteroaryl^{b} (e.g. pyridinyl). When R3 is

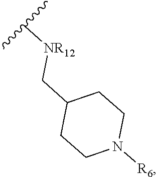

R2 can be H. Alternatively, R2 can be halo (e.g. chloro). Alternatively, R2 can be alkyl (e.g. methyl).

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF3 and —NR13R14; wherein the 6,5-heteroaromatic bicyclic ring may be attached via the 6- or 5-membered ring;

B can preferably be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline.

The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

Alternatively, A can be a 6-membered heteroaryl of formula (II),

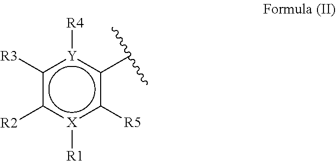

wherein X and Y are independently selected from C and N, wherein at least one of X or Y is N;
wherein R1 and R4 are independently absent or independently selected from H, halo and alkyl;
wherein R3 is halo;
wherein R2 is —(CH₂)₀₋₃NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁₋₄NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) and —O—(CH₂)₀₋₃(heteroaryl); and
wherein R5 is H, alkyl and halo.

X can be N. Y can be N. Both X and Y can be N.

When X is C, R1 can be H. R1 can be halo (e.g. chloro). R1 can be alkyl (e.g. methyl).

When Y is C, R4 can be H. R4 can be halo (e.g. chloro). R4 can be alkyl (e.g. methyl).

R5 can be H. R5 can be halo (e.g. chloro). R5 can be alkyl (e.g. methyl).

R3 is halo. R3 can be fluoro. R3 can be bromo. Preferably, R3 can be chloro.

R2 can be —NR13R14.

R2 can be —NR12(CH₂)₀₋₃NR13R14. R2 can be —NR12(CH₂)₁₋₃NR13R14. R2 can be —NR12(CH₂)NR13R14. R2 can be —NR12(CH₂)₂NR13R14. R2 can be —NR12(CH₂)₃NR13R14.

R2 can be —O—(CH₂)₁₋₄NR13R14. R2 can be —O—(CH₂)NR13R14. R2 can be —O—(CH₂)₂NR13R14. R2 can be —O—(CH₂)₃NR13R14. R2 can be —O—(CH₂)₄NR13R14.

R13 can be H and R14 can be cycloalkyl (e.g. cyclopentane). R13 can be H and R14 can be alkyl^{b}, e.g. alkyl^{b} substituted with —NHCOCH₃. R13 and R14 can both be alkyl^{b} (e.g. methyl, ethyl, or isopropyl).

Alternatively, R13 and R14, together with the nitrogen atom to which they are attached can form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds. More specifically, R13 and R14, together with the nitrogen atom to which they are attached can form azetidine, pyrrolidine, piperidine, or azetane, which as noted above can be optionally substituted in the same manner as R13 and R14. For instance, the ring formed by R13 and R14 can be substituted with, e.g. —OH and oxo.

R2 can be —NR12(CH₂)₀₋₃(aryl). R2 can be —NR12(aryl). R2 can be —NR12(CH₂)(aryl). R2 can be —NR12(CH₂)₂(aryl). R2 can be —NR12(CH₂)₃(aryl). R12 can be e.g. H or alkyl (e.g. methyl).

R2 can be —(CH₂)₀₋₃O(CH₂)₀₋₃(aryl). R2 can be —O(CH₂)₀₋₃(aryl). R2 can be —(CH₂)O(CH₂)₀₋₃(aryl). R2 can be —(CH₂)₂O(CH₂)₀₋₃(aryl). R2 can be —(CH₂)₃O(CH₂)₀₋₃(aryl). R2 can be —(CH₂)₀₋₃O(aryl). R2 can be —(CH₂)₀₋₃O(CH₂)(aryl). R2 can be —(CH₂)₀₋₃O(CH₂)₂(aryl). R2 can be —(CH₂)₀₋₃O(CH₂)₃(aryl). R2 can be —O(aryl). R2 can be —(CH₂)O(CH₂)(aryl). R2 can be —(CH₂)₂O(CH₂)(aryl). R2 can be —(CH₂)O(CH₂)₂(aryl). R2 can be —(CH₂)O(CH₂)₃(aryl). R2 can be —(CH₂)₃O(CH₂)₃(aryl).

“Aryl” can be phenyl, which as noted above, can be substituted in the same manner as “aryl”. For example, the aryl (e.g. phenyl) can be substituted with alkoxy e.g. alkoxy substituted with N(R12)₂. The aryl (e.g. phenyl) can be substituted with halo (e.g. chloro). The aryl (e.g. phenyl) can be substituted with CN. The aryl (e.g. phenyl) can be substituted with heterocyclyl^{b}, which e.g. can be morpholinyl, or piperazinyl. The aryl (e.g. phenyl) can be substituted with —(CH₂)₀₋₃—NR13R14. Alternatively, two adjacent ring atoms on the “aryl” (e.g. phenyl) can be linked to form a 5- or 6-membered aromatic ring containing 1 or 2 heteroatoms that are selected from N, NR8, S, and O, which may be optionally substituted as for heteroaryl^{b}, e.g. the aromatic ring formed can be imidazole.

R2 can be —(CH₂)NR12(CH₂)₀₋₃(heterocyclyl). R2 can be —(CH₂)NR12(heterocyclyl). R2 can be —(CH₂)NR12(CH₂)(heterocyclyl). R2 can be —(CH₂)NR12(CH₂)₁(heterocyclyl). R2 can be —(CH₂)NR12(CH₂)₂(heterocyclyl). R2 can be —(CH₂)NR12(CH₂)₃(heterocyclyl).

R2 can be —O—(CH₂)₀₋₃(heterocyclyl). R2 can be —O-(heterocyclyl). R2 can be —O—(CH₂)₁(heterocyclyl). R2 can be —O—(CH₂)₂(heterocyclyl). R2 can be —O—(CH₂)₃(heterocyclyl).

The “heterocyclyl” can be selected from tetrahydropyranyl, piperidinyl, morpholinyl, piperazinyl, pyrrolidinyl, and azetidinyl, which as noted above can all be optionally substituted in the same manner as “heterocyclyl”. The “heterocyclyl” can be substituted by oxo. When NR8 is present, R8 can be alkyl (e.g. —CH₂CH₂OCH₃) or cycloalkyl (e.g. cyclopropane). R8 can also be heteroaryl^{b} (e.g. piperidinyl or thiazole). R8 can also be —(CH₂)₀₋₃aryl^{b}, e.g. —(CH₂)₀₋₃(phenyl). R8 can also be —SO₂CH₃. R8 can also be —COCH₃.

R2 can be —(CH₂)₀₋₃NR12(CH₂)₀₋₃(heteroaryl). R2 can be —NR12(CH₂)₀₋₃(heteroaryl). R2 can be —(CH₂)NR12(CH₂)₀₋₃(heteroaryl). R2 can be —(CH₂)₂NR12(CH₂)₀₋₃(heteroaryl). R2 can be —(CH₂)₃NR12(CH₂)₀₋₃(heteroaryl). R2 can be —(CH₂)₀₋₃NR12(heteroaryl). R2 can be —(CH₂)₀₋₃NR12(CH₂)(heteroaryl). R2 can be —(CH₂)₀₋₃NR12(CH₂)₂(heteroaryl). R2 can be —(CH₂)₀₋₃NR12(CH₂)₃(heteroaryl).

R2 can be —O—(CH₂)₀₋₃(heteroaryl). R2 can be —O-(heteroaryl). R2 can be O—(CH₂)(heteroaryl). R2 can be —O—(CH₂)₂(heteroaryl). R2 can be —O—(CH₂)₃(heteroaryl).

The “heteroaryl” can be selected from imidazolyl, pyridinyl, triazole, and thiazole, which as noted above can be optionally substituted in the same manner as “heteroaryl”.

X can be N and Y can be C. X can be N, Y can be C, R4 can be H and R3 can be halo (e.g. chloro). X can be N, Y can be C, R4 can be H, R3 can be halo (e.g. chloro), and R2 can be —(CH₂)₀₋₃NR13R14. More specifically, X can be N, Y can be C, R4 can be H, R3 can be halo (e.g. chloro), and R2 can be —(CH₂)₀₋₃NR13R14. More specifically, R2 can be —CH₂NR13R14, wherein R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds. More specifically, X can be N, Y can be C, R4 can be H, R3 can be halo (e.g. chloro), and R2 can be —NR13R14, wherein R13 and R14, together with the N to which they are attached, form piperazine. The piperazine can have an NR8 group. The R8 group can be heteroaryl^{b}. The heteroaryl^{b} can be pyridine.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CFₛ and —NR13R14; wherein the 6,5-heteroaromatic bicyclic ring may be attached via the 6- or 5-membered ring;

B can preferably be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

Alternatively, A can be a 6-membered heteroaryl of formula (II),

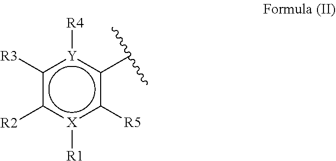

wherein X and Y are C;
wherein R4 is H, halo, alkyl;
wherein R5 is H or alkyl;
wherein R3 is H or halo;
wherein one of R1 and R2 is —(CH₂)(heterocyclyl) or —N(R12)CO(CH₂)₀₋₃(heterocyclyl), and the other of R1 and R2 is selected from H and alkyl.

R4 can be H. R4 can be halo (e.g. chloro). R4 can be alkyl (e.g. methyl).

R5 can be H. R5 can be alkyl (e.g. methyl).

R3 can be H. R3 can be halo (e.g. chloro).

R1 can be —(CH₂)(heterocyclyl) or —N(R12)CO(CH₂)₀₋₃(heterocyclyl). When R1 is —(CH₂)(heterocyclyl) or —N(R12)CO(CH₂)₀₋₃(heterocyclyl), R2 can be H. When R1 is —(CH₂)(heterocyclyl) or —N(R12)CO(CH₂)₀₋₃(heterocyclyl), R2 can be alkyl (e.g. methyl).

R2 can be —(CH₂)(heterocyclyl) or —N(R12)CO(CH₂)₀₋₃(heterocyclyl). When R2 is —(CH₂)(heterocyclyl) or —N(R12)CO(CH₂)₀₋₃(heterocyclyl), R1 can be H. When R1 is —(CH₂)(heterocyclyl) or —N(R12)CO(CH₂)₀₋₃(heterocyclyl), R1 can be alkyl (e.g. methyl).

The “heterocyclyl” can be piperazinyl or piperidinyl. The piperazinyl can contain an NR8 group. R8 can be heteroaryl^{b} (e.g. pyridine) R8 can be alkyl^{b} (e.g. methyl). R8 can be alkyl^{b} substituted with —(CH₂)₀₋₃cycloalkyl e.g. —CH₂(cyclopentane).

R4 can be H and R3 can be halo. R4 can be H and R3 can be halo (e.g. chloro). R4 can be H, R3 can be halo (e.g. chloro), and R1 is H. R4 can be H, R3 can be halo (e.g. chloro), R1 is H, and R2 is —(CH₂)(heterocyclyl). More specifically, R4 can be H, R3 can be halo (e.g. chloro), R1 is H, and R2 is —(CH₂)(piperazinyl). The heterocyclyl (e.g. piperazinyl) can contain a NR8 group. The R8 group can be heteroaryl^{b} (e.g. pyridinyl).

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF3 and —NR13R14; wherein the 6,5-heteroaromatic bicyclic ring may be attached via the 6- or 5-membered ring.

B can preferably be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

Alternatively, A is a 6-membered heteroaryl of formula (II),

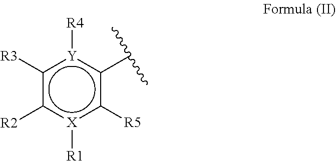

wherein X is C or N, and Y is C;
R1 is absent, H or alkyl;
R4 is H or alkyl;
R5 is H or alkyl;
wherein either: (a) R2 and R3 together with the carbon atoms to which they are bonded form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}, or (b) R2 and R3 are independently selected from H and halo, wherein at least one of R2 or R3 is halo, or (c) R2 and R3 are independently selected from H, aryl^{b} and heteroaryl^{b}, wherein at least one of R2 or R3 is aryl^{b}, or heteroaryl^{b}.

X can be C. X can be N. When X is N, R1 is absent. When X is C and R1 is H. When X is C and R1 is alkyl (e.g. methyl).

R4 can be H. R4 can be alkyl (e.g. methyl).

R5 can be H. R5 can alkyl (e.g. methyl).

R2 and R3 together with the carbon atoms to which they are bonded can form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}. More specifically, R2 and R3 together with the carbon atoms to which they are bonded can form a 5-membered nitrogen-containing heteroaryl, e.g. pyrrole.

At least one of R2 and R3 can be halo. More specifically, at least one of R2 and R3 can be bromo. R2 can be bromo. R3 can be bromo. More specifically, at least one of R2 and R3 can be chloro. R2 can be chloro. R3 can be chloro. More specifically, at least one of R2 and R3 can be fluoro. R2 can be fluoro. R3 can be fluoro. When at least one of R2 and R3 is halo, the other of R2 and R3 can be H.

At least one of R2 or R3 can be aryl^{b}, or heteroaryl^{b}. When at least one of R2 or R3 is aryl^{b}, the aryl^{b} can be phenyl. R2 can be aryl^{b} (phenyl). R3 can be aryl^{b} (phenyl). When at least one of R2 or R3 is heteroaryl^{b}, the heteroaryl^{b} can be pyrazole. When at least one of R2 and R3 is halo, the other of R2 and R3 can be H.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring, and in particular, when one of R2 or R3 is halo. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro). Additionally, or in the alternative, the isoquinoline can also be substituted with alkoxy (e.g. methoxy).

The present invention also encompasses, but is not limited to, the compounds below in Tables 1 to 11, and pharmaceutically acceptable salts and/or solvates thereof.

TABLE 1

| Structure | Example |
|---|---|
| Molecular formula | No. |
| 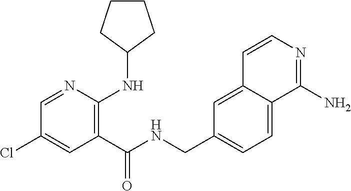 [SMILES: Nc1nccc2cc(CNC(=O)c3cc(Cl)cnc3NC3CCCC3)ccc12] C₂₁H₂₂ClN₅O | 1.01 |
| 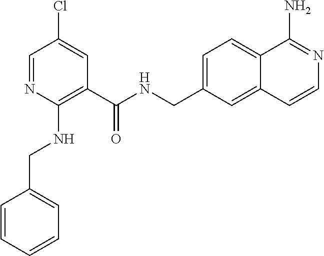 [SMILES: Nc1nccc2cc(CNC(=O)c3cc(Cl)cnc3NCc3ccccc3)ccc12] C₂₃H₂₀ClN₅O | 1.02 |
| 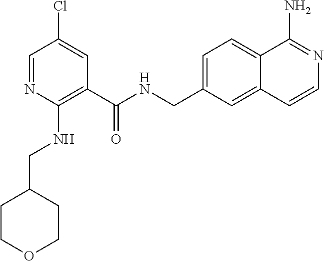 [SMILES: Nc1nccc2cc(CNC(=O)c3cc(Cl)cnc3NCC3CCOCC3)ccc12] C₂₂H₂₄ClN₅O₂ | 1.03 |
| 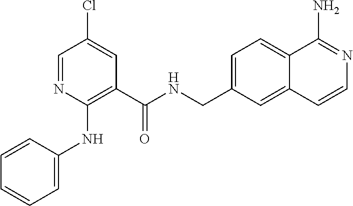 [SMILES: Nc1nccc2cc(CNC(=O)c3cc(Cl)cnc3Nc3ccccc3)ccc12] C₂₂H₁₈ClN₅O | 1.04 |
| 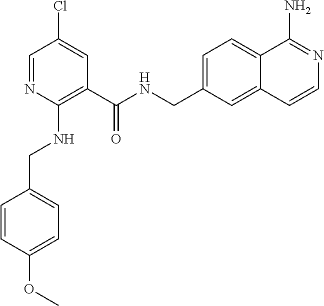 [SMILES: COc1ccc(CNc2ncc(Cl)cc2C(=O)NCc2ccc3c(N)nccc3c2)cc1] C₂₄H₂₂ClN₅O₂ | 1.05 |
| 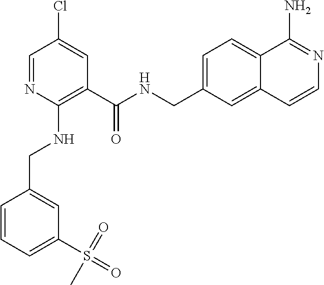 [SMILES: CS(=O)(=O)c1cccc(CNc2ncc(Cl)cc2C(=O)NCc2ccc3c(N)nccc3c2)c1] C₂₄H₂₂ClN₅O₃S | 1.06 |
| 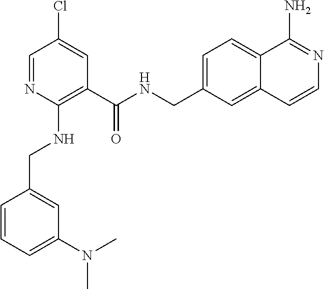 [SMILES: CN(C)c1cccc(CNc2ncc(Cl)cc2C(=O)NCc2ccc3c(N)nccc3c2)c1] C₂₅H₂₅ClN₆O | 1.07 |
| 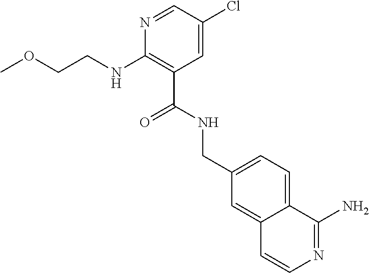 [SMILES: COCCNc1ncc(Cl)cc1C(=O)NCc1ccc2c(N)nccc2c1] C₁₉H₂₀ClN₅O₂ | 1.08 |
| 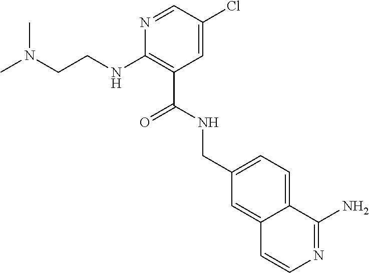 [SMILES: CN(C)CCNc1ncc(Cl)cc1C(=O)NCc1ccc2c(N)nccc2c1] C₂₀H₂₃ClN₆O | 1.09 |
| 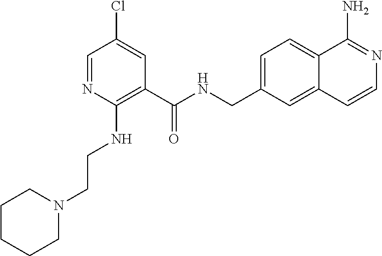 [SMILES: Nc1nccc2cc(CNC(=O)c3cc(Cl)cnc3NCCN3CCCCC3)ccc12] C₂₃H₂₇ClN₆O | 1.10 |
| 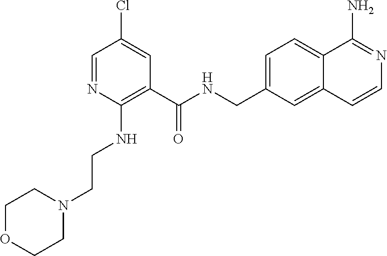 [SMILES: Nc1nccc2cc(CNC(=O)c3cc(Cl)cnc3NCCN3CCOCC3)ccc12] C₂₂H₂₅ClN₆O₂ | 1.11 |
| 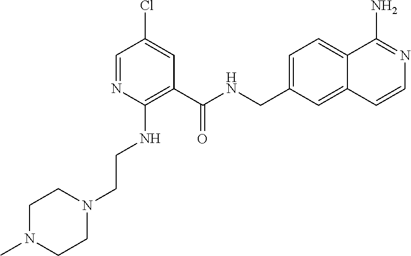 [SMILES: CN1CCN(CCNc2ncc(Cl)cc2C(=O)NCc2ccc3c(N)nccc3c2)CC1] C₂₃H₂₈ClN₇O | 1.12 |
| 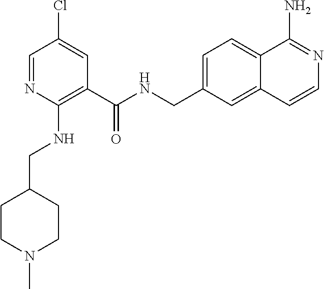 [SMILES: CN1CCC(CNc2ncc(Cl)cc2C(=O)NCc2ccc3c(N)nccc3c2)CC1] C₂₃H₂₇ClN₆O | 1.13 |
| 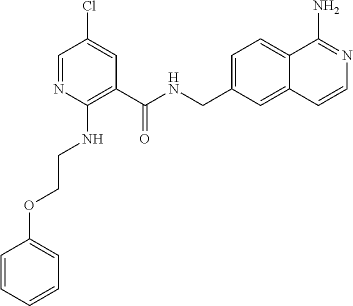 [SMILES: Nc1nccc2cc(CNC(=O)c3cc(Cl)cnc3NCCOc3ccccc3)ccc12] C₂₄H₂₂ClN₅O₂ | 1.14 |
| 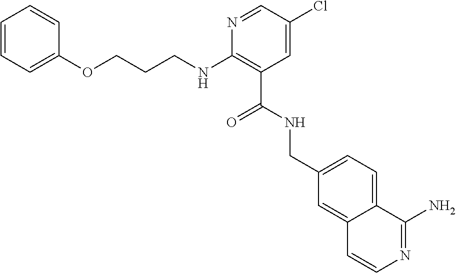 [SMILES: Nc1nccc2cc(CNC(=O)c3cc(Cl)cnc3NCCCOc3ccccc3)ccc12] C₂₅H₂₄ClN₅O₂ | 1.15 |
| 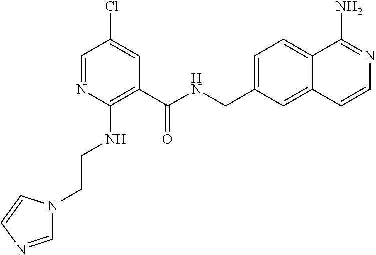 [SMILES: Nc1nccc2cc(CNC(=O)c3cc(Cl)cnc3NCCn3ccnc3)ccc12] C₂₁H₂₀ClN₇O | 1.16 |
| 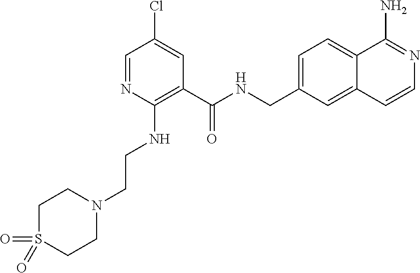 [SMILES: Nc1nccc2cc(CNC(=O)c3cc(Cl)cnc3NCCN3CCS(=O)(=O)CC3)ccc12] C₂₂H₂₅ClN₆O₃S | 1.17 |
| 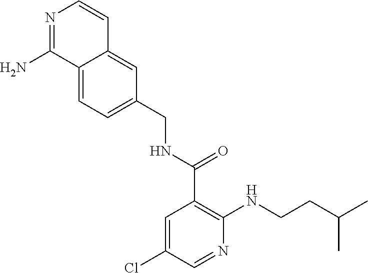 [SMILES: CC(C)CCNc1ncc(Cl)cc1C(=O)NCc1ccc2c(N)nccc2c1] C₂₁H₂₄ClN₅O | 1.18 |
| 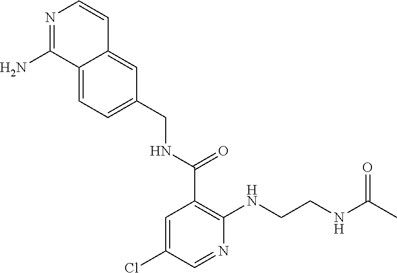 [SMILES: CC(=O)NCCNc1ncc(Cl)cc1C(=O)NCc1ccc2c(N)nccc2c1] C₂₀H₂₁ClN₆O₂ | 1.19 |
| 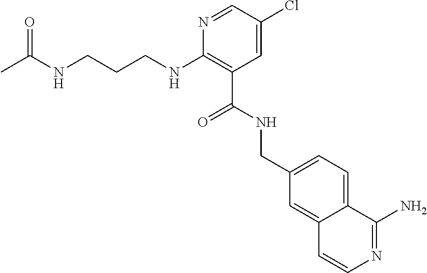 [SMILES: CC(=O)NCCCNc1ncc(Cl)cc1C(=O)NCc1ccc2c(N)nccc2c1] C₂₁H₂₃ClN₆O₂ | 1.20 |
| 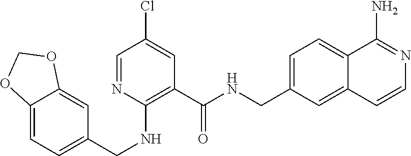 [SMILES: Nc1nccc2cc(CNC(=O)c3cc(Cl)cnc3NCc3ccc4c(c3)OCO4)ccc12] C₂₄H₂₀ClN₅O₃ | 1.21 |
| 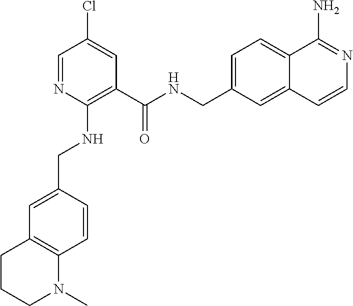 [SMILES: CN1CCCc2cc(CNc3ncc(Cl)cc3C(=O)NCc3ccc4c(N)nccc4c3)ccc21] C₂₇H₂₇ClN₆O | 1.22 |
| 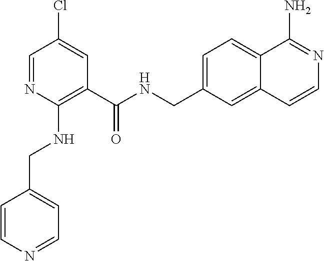 [SMILES: Nc1nccc2cc(CNC(=O)c3cc(Cl)cnc3NCc3ccncc3)ccc12] C₂₂H₁₉ClN₆O | 1.23 |
| 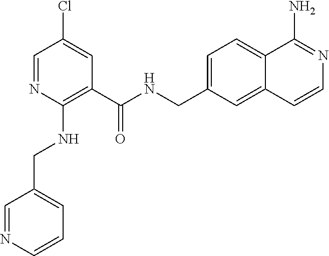 [SMILES: Nc1nccc2cc(CNC(=O)c3cc(Cl)cnc3NCc3cccnc3)ccc12] C₂₂H₁₉ClN₆O | 1.24 |
| 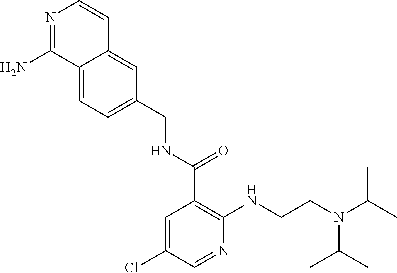 [SMILES: CC(C)N(CCNc1ncc(Cl)cc1C(=O)NCc1ccc2c(N)nccc2c1)C(C)C] C₂₄H₃₁ClN₆O | 1.25 |
| 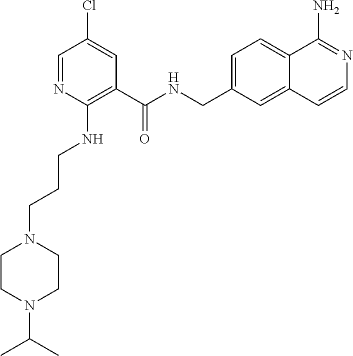 [SMILES: CC(C)N1CCN(CCCNc2ncc(Cl)cc2C(=O)NCc2ccc3c(N)nccc3c2)CC1] C₂₆H₃₄ClN₇O | 1.26 |
| 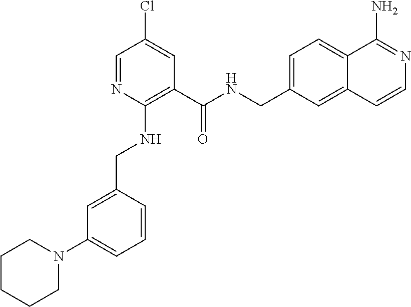 [SMILES: Nc1nccc2cc(CNC(=O)c3cc(Cl)cnc3NCc3cccc(N4CCCCC4)c3)ccc12] C₂₈H₂₉ClN₆O | 1.27 |
| 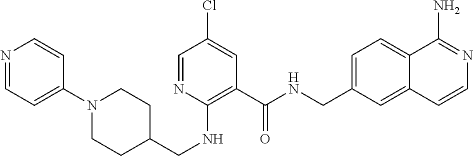 [SMILES: Nc1nccc2cc(CNC(=O)c3cc(Cl)cnc3NCC3CCN(c4ccncc4)CC3)ccc12] C₂₇H₂₈ClN₇O | 1.28 |
| 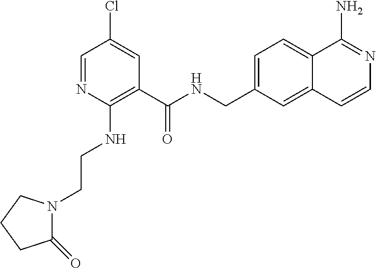 [SMILES: Nc1nccc2cc(CNC(=O)c3cc(Cl)cnc3NCCN3CCCC3=O)ccc12] C₂₂H₂₃ClN₆O₂ | 1.29 |
| 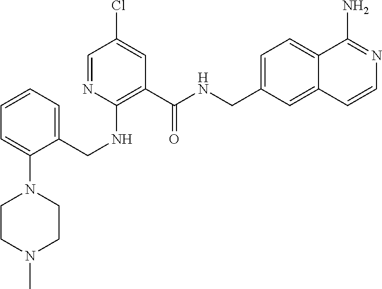 [SMILES: CN1CCN(c2ccccc2CNc2ncc(Cl)cc2C(=O)NCc2ccc3c(N)nccc3c2)CC1] C₂₈H₃₀ClN₇O | 1.30 |
| 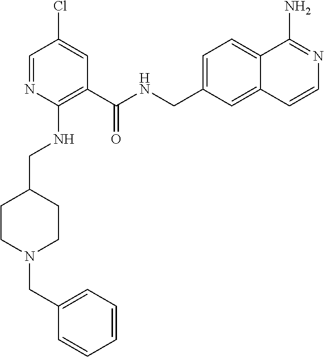 [SMILES: Nc1nccc2cc(CNC(=O)c3cc(Cl)cnc3NCC3CCN(Cc4ccccc4)CC3)ccc12] C₂₉H₃₁ClN₆O | 1.31 |
| 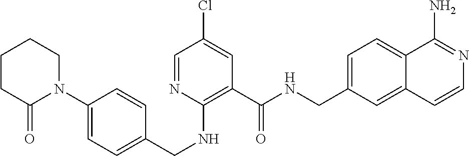 [SMILES: Nc1nccc2cc(CNC(=O)c3cc(Cl)cnc3NCc3ccc(N4CCCCC4=O)cc3)ccc12] C₂₈H₂₇ClN₆O₂ | 1.32 |
| 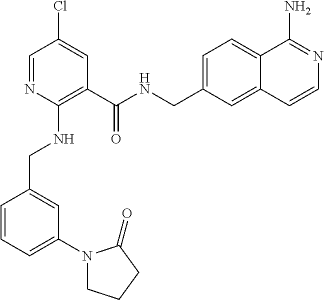 [SMILES: Nc1nccc2cc(CNC(=O)c3cc(Cl)cnc3NCc3cccc(N4CCCC4=O)c3)ccc12] C₂₇H₂₅ClN₆O₂ | 1.33 |
| 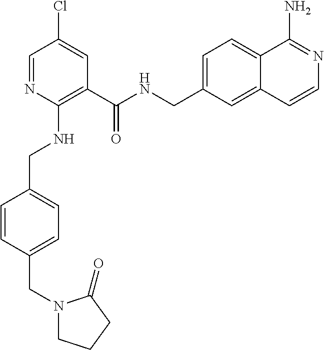 [SMILES: Nc1nccc2cc(CNC(=O)c3cc(Cl)cnc3NCc3ccc(CN4CCCC4=O)cc3)ccc12] C₂₈H₂₇ClN₆O₂ | 1.34 |
| 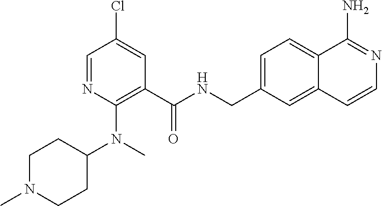 [SMILES: CN1CCC(N(C)c2ncc(Cl)cc2C(=O)NCc2ccc3c(N)nccc3c2)CC1] C₂₃H₂₇ClN₆O | 1.35 |
| 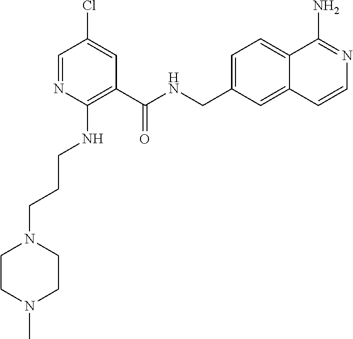 [SMILES: CN1CCN(CCCNc2ncc(Cl)cc2C(=O)NCc2ccc3c(N)nccc3c2)CC1] C₂₄H₃₀ClN₇O | 1.36 |
| 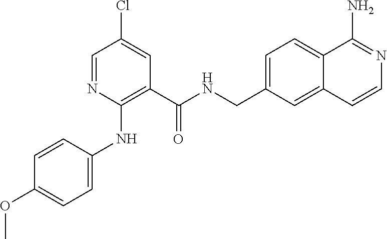 [SMILES: COc1ccc(Nc2ncc(Cl)cc2C(=O)NCc2ccc3c(N)nccc3c2)cc1] C₂₃H₂₀ClN₅O₂ | 1.37 |
| 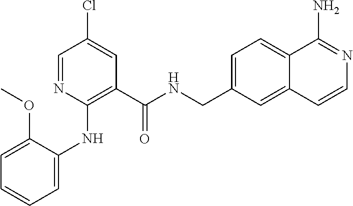 [SMILES: COc1ccccc1Nc1ncc(Cl)cc1C(=O)NCc1ccc2c(N)nccc2c1] C₂₃H₂₀ClN₅O₂ | 1.38 |
| 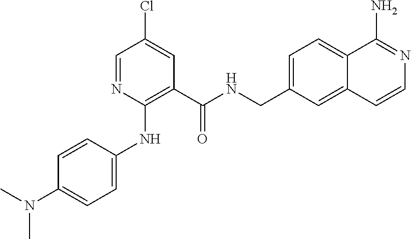 [SMILES: CN(C)c1ccc(Nc2ncc(Cl)cc2C(=O)NCc2ccc3c(N)nccc3c2)cc1] C₂₄H₂₃ClN₆O | 1.39 |
| 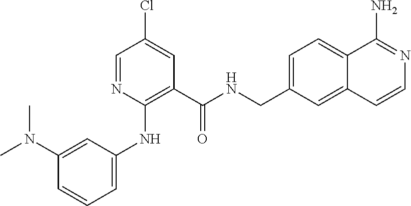 [SMILES: CN(C)c1cccc(Nc2ncc(Cl)cc2C(=O)NCc2ccc3c(N)nccc3c2)c1] C₂₄H₂₃ClN₆O | 1.40 |
| 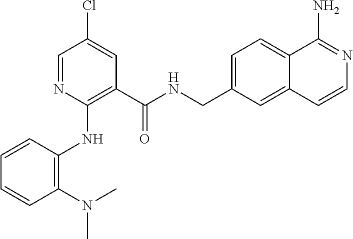 [SMILES: CN(C)c1ccccc1Nc1ncc(Cl)cc1C(=O)NCc1ccc2c(N)nccc2c1] C₂₄H₂₃ClN₆O | 1.41 |
| 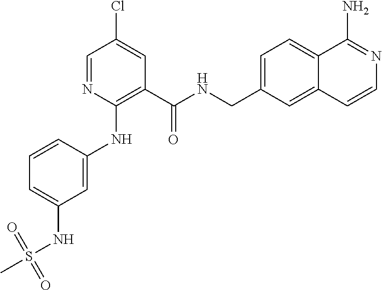 [SMILES: CS(=O)(=O)Nc1cccc(Nc2ncc(Cl)cc2C(=O)NCc2ccc3c(N)nccc3c2)c1] C₂₃H₂₁ClN₆O₃S | 1.42 |
| 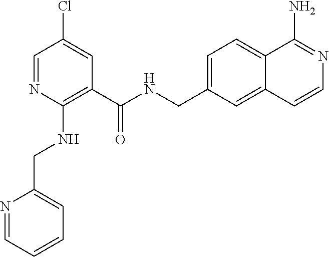 [SMILES: Nc1nccc2cc(CNC(=O)c3cc(Cl)cnc3NCc3ccccn3)ccc12] C₂₂H₁₉ClN₆O | 1.43 |
| 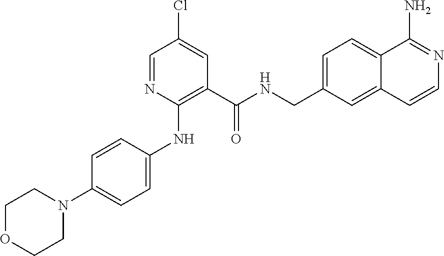 [SMILES: Nc1nccc2cc(CNC(=O)c3cc(Cl)cnc3Nc3ccc(N4CCOCC4)cc3)ccc12] C₂₆H₂₅ClN₆O₂ | 1.44 |
| 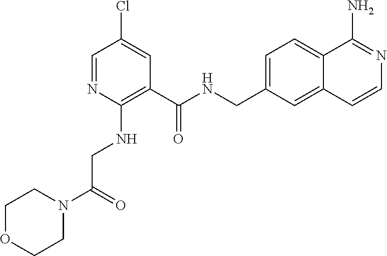 [SMILES: Nc1nccc2cc(CNC(=O)c3cc(Cl)cnc3NCC(=O)N3CCOCC3)ccc12] C₂₂H₂₃ClN₆O₃ | 1.45 |
| 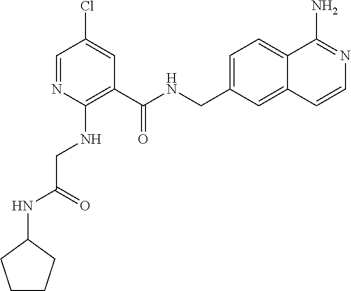 [SMILES: Nc1nccc2cc(CNC(=O)c3cc(Cl)cnc3NCC(=O)NC3CCCC3)ccc12] C₂₃H₂₅ClN₆O₂ | 1.46 |
| 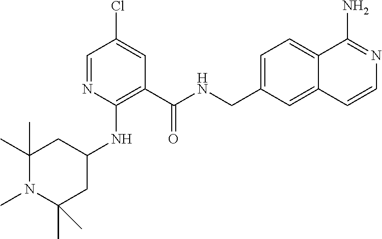 [SMILES: CN1C(C)(C)CC(Nc2ncc(Cl)cc2C(=O)NCc2ccc3c(N)nccc3c2)CC1(C)C] C₂₆H₃₃ClN₆O | 1.47 |
| 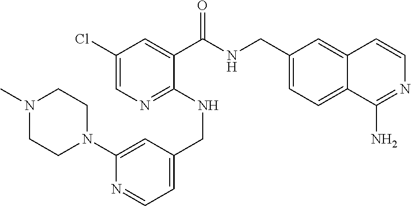 [SMILES: CN1CCN(c2cc(CNc3ncc(Cl)cc3C(=O)NCc3ccc4c(N)nccc4c3)ccn2)CC1] C₂₇H₂₉ClN₈O | 1.48 |
| 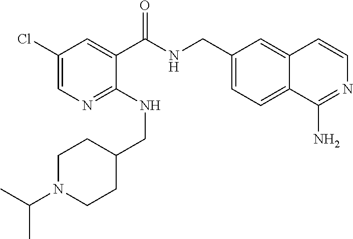 [SMILES: CC(C)N1CCC(CNc2ncc(Cl)cc2C(=O)NCc2ccc3c(N)nccc3c2)CC1] C₂₅H₃₁ClN₆O | 1.49 |
| 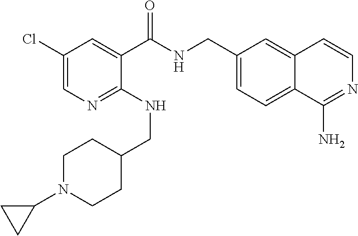 [SMILES: Nc1nccc2cc(CNC(=O)c3cc(Cl)cnc3NCC3CCN(C4CC4)CC3)ccc12] C₂₅H₂₉ClN₆O | 1.50 |
| 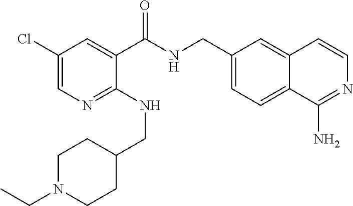 [SMILES: CCN1CCC(CNc2ncc(Cl)cc2C(=O)NCc2ccc3c(N)nccc3c2)CC1] C₂₄H₂₉ClN₆O | 1.51 |
| 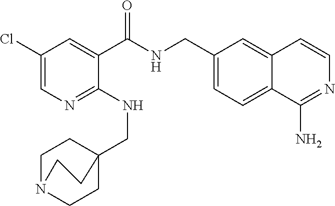 [SMILES: Nc1nccc2cc(CNC(=O)c3cc(Cl)cnc3NCC34CCN(CC3)CC4)ccc12] C₂₄H₂₇ClN₆O | 1.52 |
| 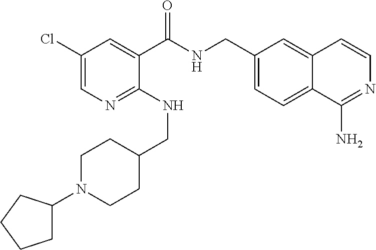 [SMILES: Nc1nccc2cc(CNC(=O)c3cc(Cl)cnc3NCC3CCN(C4CCCC4)CC3)ccc12] C₂₇H₃₃ClN₆O | 1.53 |
| 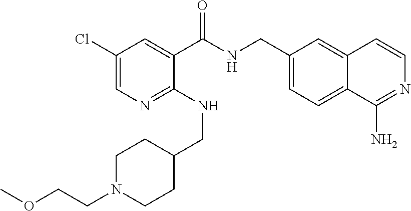 [SMILES: COCCN1CCC(CNc2ncc(Cl)cc2C(=O)NCc2ccc3c(N)nccc3c2)CC1] C₂₅H₃₁ClN₆O₂ | 1.54 |
| 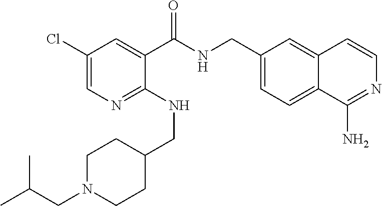 [SMILES: CC(C)CN1CCC(CNc2ncc(Cl)cc2C(=O)NCc2ccc3c(N)nccc3c2)CC1] C₂₆H₃₃ClN₆O | 1.55 |
| 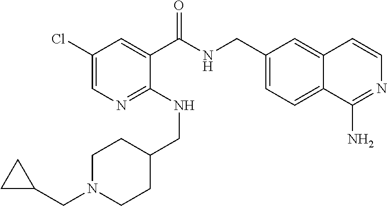 [SMILES: Nc1nccc2cc(CNC(=O)c3cc(Cl)cnc3NCC3CCN(CC4CC4)CC3)ccc12] C₂₆H₃₁ClN₆O | 1.56 |
| 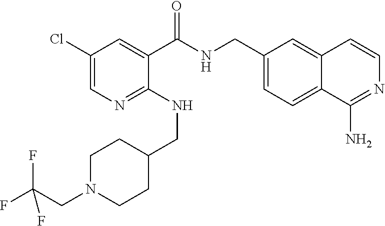 [SMILES: Nc1nccc2cc(CNC(=O)c3cc(Cl)cnc3NCC3CCN(CC(F)(F)F)CC3)ccc12] C₂₄H₂₆ClF₃N₆O | 1.57 |
| 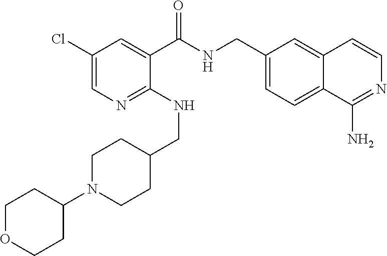 [SMILES: Nc1nccc2cc(CNC(=O)c3cc(Cl)cnc3NCC3CCN(C4CCOCC4)CC3)ccc12] C₂₇H₃₃ClN₆O₂ | 1.58 |
| 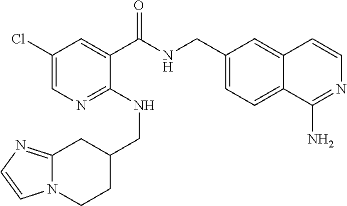 [SMILES: Nc1nccc2cc(CNC(=O)c3cc(Cl)cnc3NCC3CCn4ccnc4C3)ccc12] C₂₄H₂₄ClN₇O | 1.59 |
| 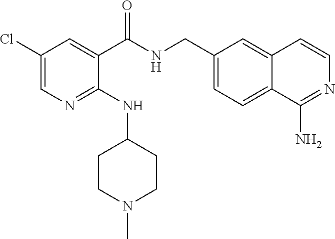 [SMILES: CN1CCC(Nc2ncc(Cl)cc2C(=O)NCc2ccc3c(N)nccc3c2)CC1] C₂₂H₂₅ClN₆O | 1.60 |
| 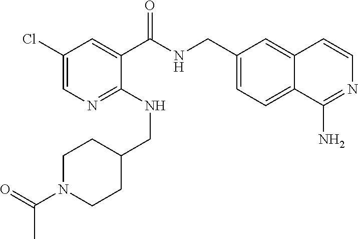 [SMILES: CC(=O)N1CCC(CNc2ncc(Cl)cc2C(=O)NCc2ccc3c(N)nccc3c2)CC1] C₂₄H₂₇ClN₆O₂ | 1.61 |
| 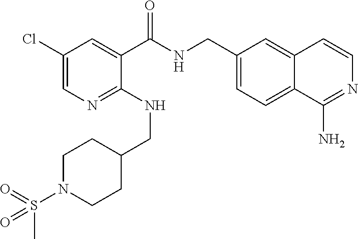 [SMILES: CS(=O)(=O)N1CCC(CNc2ncc(Cl)cc2C(=O)NCc2ccc3c(N)nccc3c2)CC1] C₂₃H₂₇ClN₆O₃S | 1.62 |
| 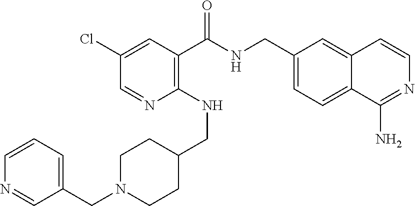 [SMILES: Nc1nccc2cc(CNC(=O)c3cc(Cl)cnc3NCC3CCN(Cc4cccnc4)CC3)ccc12] C₂₈H₃₀ClN₇O | 1.63 |
| 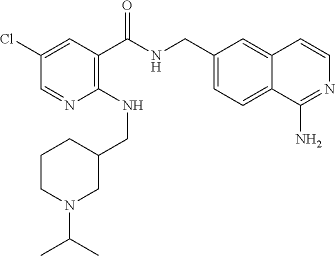 [SMILES: CC(C)N1CCCC(CNc2ncc(Cl)cc2C(=O)NCc2ccc3c(N)nccc3c2)C1] C₂₅H₃₁ClN₆O | 1.64 |
| 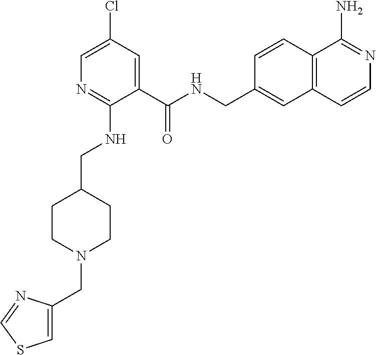 [SMILES: Nc1nccc2cc(CNC(=O)c3cc(Cl)cnc3NCC3CCN(Cc4cscn4)CC3)ccc12] C₂₆H₂₈ClN₇OS | 1.65 |
| 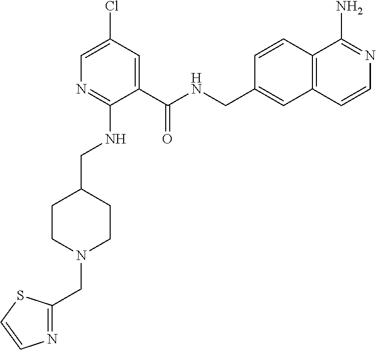 [SMILES: Nc1nccc2cc(CNC(=O)c3cc(Cl)cnc3NCC3CCN(Cc4nccs4)CC3)ccc12] C₂₆H₂₈ClN₇OS | 1.66 |
| 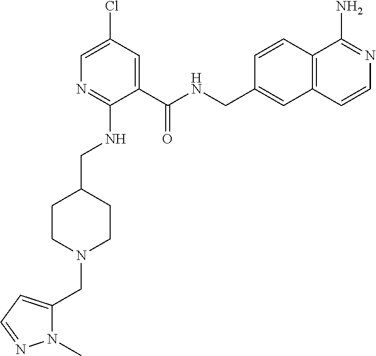 [SMILES: Cn1nccc1CN1CCC(CNc2ncc(Cl)cc2C(=O)NCc2ccc3c(N)nccc3c2)CC1] C₂₇H₃₁ClN₈O | 1.67 |
| 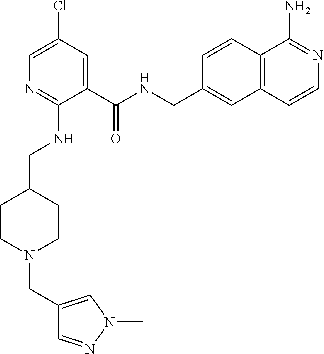 [SMILES: Cn1cc(CN2CCC(CNc3ncc(Cl)cc3C(=O)NCc3ccc4c(N)nccc4c3)CC2)cn1] C₂₇H₃₁ClN₈O | 1.68 |
| 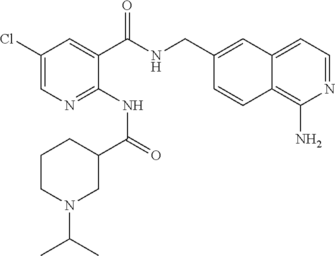 [SMILES: CC(C)N1CCCC(C(=O)Nc2ncc(Cl)cc2C(=O)NCc2ccc3c(N)nccc3c2)C1] C₂₅H₂₉ClN₆O₂ | 1.69 |
| 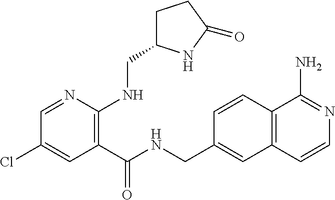 [SMILES: Nc1nccc2cc(CNC(=O)c3cc(Cl)cnc3NC[C@@H]3CCC(=O)N3)ccc12] C₂₁H₂₁ClN₆O₂ | 1.70 |
| 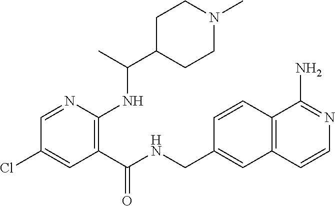 [SMILES: CC(Nc1ncc(Cl)cc1C(=O)NCc1ccc2c(N)nccc2c1)C1CCN(C)CC1] C₂₄H₂₉ClN₆O | 1.71 |
| 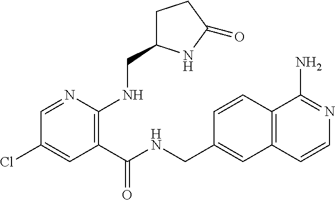 [SMILES: Nc1nccc2cc(CNC(=O)c3cc(Cl)cnc3NC[C@H]3CCC(=O)N3)ccc12] C₂₁H₂₁ClN₆O₂ | 1.72 |

TABLE 2

| Structure | Example |
|---|---|
| Molecular formula | No. |
| 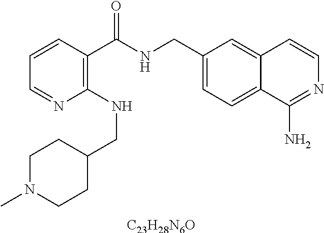 [SMILES: CN1CCC(CNc2ncccc2C(=O)NCc2ccc3c(N)nccc3c2)CC1 ; "C23H28N6O"] | 4.01 |
| 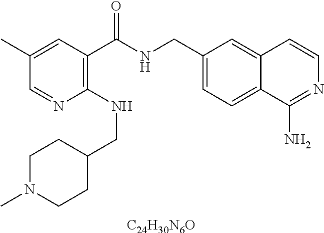 [SMILES: Cc1cnc(NCC2CCN(C)CC2)c(C(=O)NCc2ccc3c(N)nccc3c2)c1 ; "C24H30N6O"] | 4.02 |
| 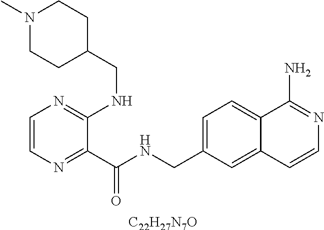 [SMILES: CN1CCC(CNc2nccnc2C(=O)NCc2ccc3c(N)nccc3c2)CC1 ; "C22H27N7O"] | 4.03 |
| 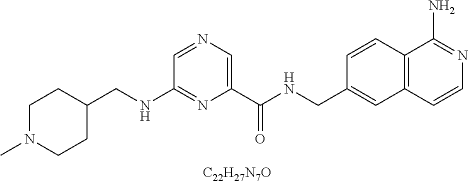 [SMILES: CN1CCC(CNc2cncc(C(=O)NCc3ccc4c(N)nccc4c3)n2)CC1 ; "C22H27N7O"] | 4.04 |
| 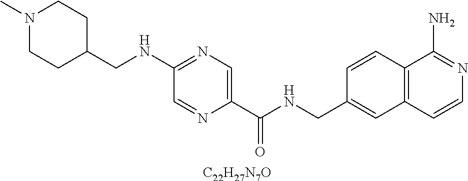 [SMILES: CN1CCC(CNc2cnc(C(=O)NCc3ccc4c(N)nccc4c3)cn2)CC1 ; "C22H27N7O"] | 4.05 |
| 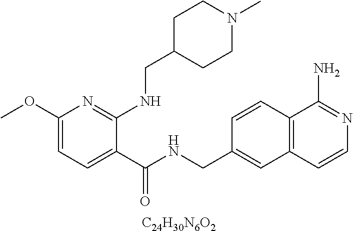 | 4.06 |
| 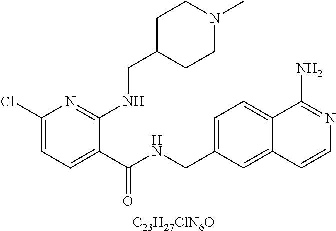 [SMILES: CN1CCC(CNc2nc(Cl)ccc2C(=O)NCc2ccc3c(N)nccc3c2)CC1 ; "C23H27ClN6O"] | 4.07 |
| 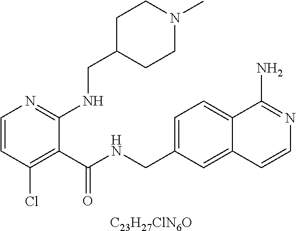 [SMILES: CN1CCC(CNc2nccc(Cl)c2C(=O)NCc2ccc3c(N)nccc3c2)CC1 ; "C23H27ClN6O"] | 4.08 |
| 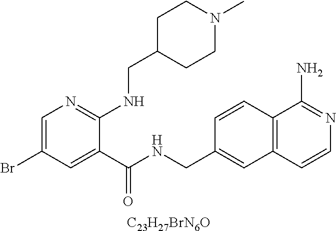 [SMILES: CN1CCC(CNc2ncc(Br)cc2C(=O)NCc2ccc3c(N)nccc3c2)CC1 ; "C23H27BrN6O"] | 4.09 |
| 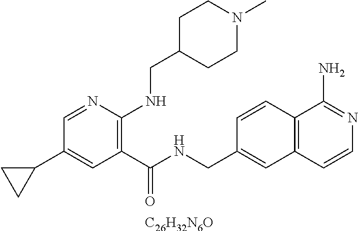 | 4.10 |
| 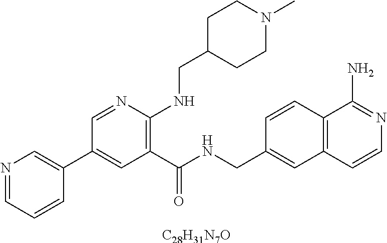 [SMILES: CN1CCC(CNc2ncc(-c3cccnc3)cc2C(=O)NCc2ccc3c(N)nccc3c2)CC1 ; "C28H31N7O"] | 4.11 |
| 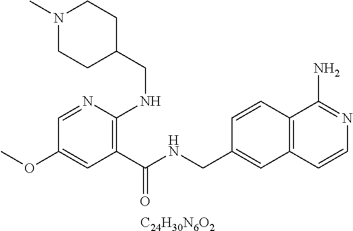 | 4.12 |
| 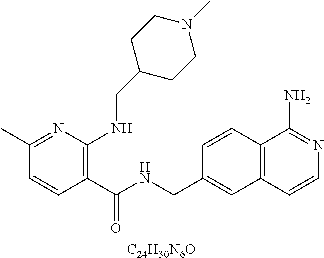 | 4.13 |
| 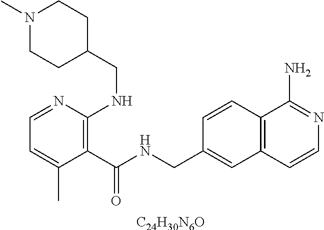 | 4.14 |
| 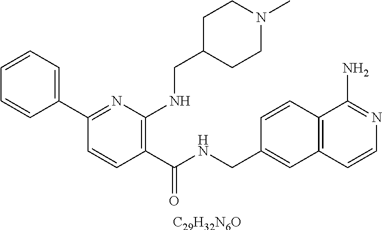 | 4.15 |
| 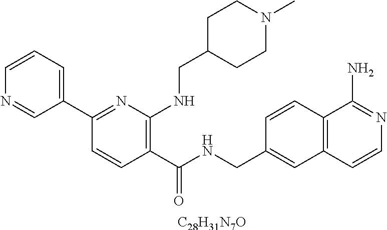 [SMILES: CN1CCC(CNc2nc(-c3cccnc3)ccc2C(=O)NCc2ccc3c(N)nccc3c2)CC1 ; "C28H31N7O"] | 4.16 |
| 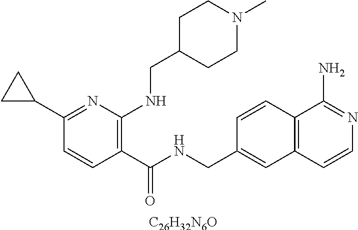 | 4.17 |
| 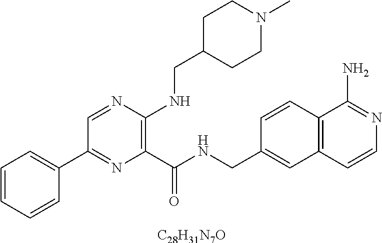 [SMILES: CN1CCC(CNc2ncc(-c3ccccc3)nc2C(=O)NCc2ccc3c(N)nccc3c2)CC1 ; "C28H31N7O"] | 4.18 |
| 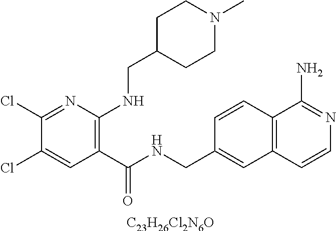 [SMILES: CN1CCC(CNc2nc(Cl)c(Cl)cc2C(=O)NCc2ccc3c(N)nccc3c2)CC1 ; "C23H26Cl2N6O"] | 4.19 |
| 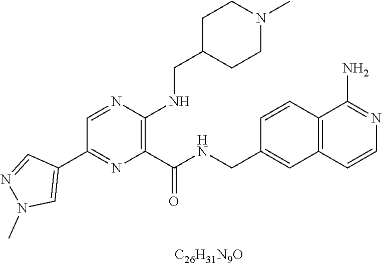 [SMILES: CN1CCC(CNc2ncc(-c3cnn(C)c3)nc2C(=O)NCc2ccc3c(N)nccc3c2)CC1 ; "C26H31N9O"] | 4.20 |
| 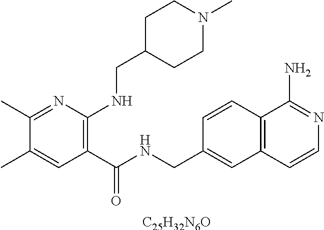 | 4.21 |
| 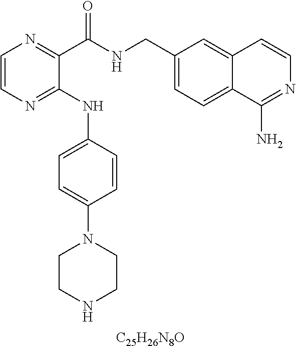 | 4.22 |
| 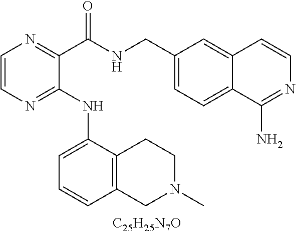 [SMILES: CN1CCc2c(cccc2Nc2nccnc2C(=O)NCc2ccc3c(N)nccc3c2)C1 ; "C25H25N7O"] | 4.23 |
| 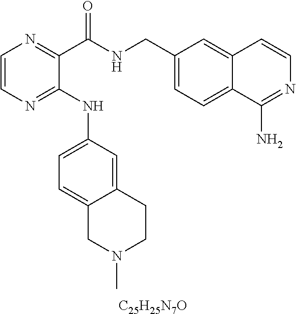 [SMILES: CN1CCc2cc(Nc3nccnc3C(=O)NCc3ccc4c(N)nccc4c3)ccc2C1 ; "C25H25N7O"] | 4.24 |
| 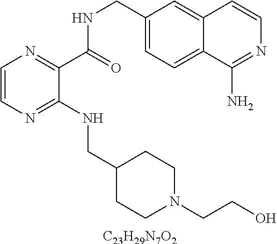 [SMILES: Nc1nccc2cc(CNC(=O)c3nccnc3NCC3CCN(CCO)CC3)ccc12 ; "C23H29N7O2"] | 4.25 |
| 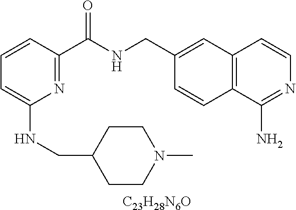 | 4.26 |
| 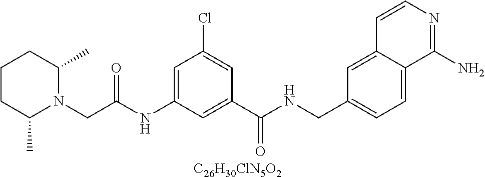 [SMILES: C[C@@H]1CCC[C@H](C)N1CC(=O)Nc1cc(Cl)cc(C(=O)NCc2ccc3c(N)nccc3c2)c1 ; "C26H30ClN5O2"] | 32.05 |

TABLE 3

| Structure | Example |
|---|---|
| Molecular formula | No. |
| 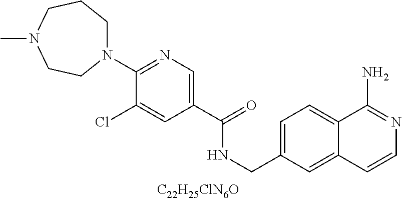 [SMILES: CN1CCCN(c2ncc(C(=O)NCc3ccc4c(N)nccc4c3)cc2Cl)CC1 ; "C22H25ClN6O"] | 8.01 |
| 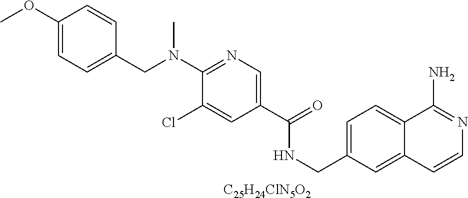 [SMILES: COc1ccc(CN(C)c2ncc(C(=O)NCc3ccc4c(N)nccc4c3)cc2Cl)cc1 ; "C25H24ClN5O2"] | 8.02 |
| 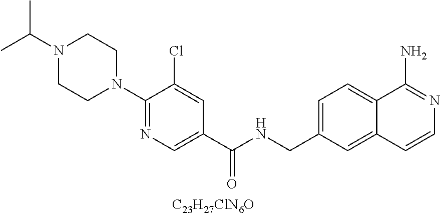 [SMILES: CC(C)N1CCN(c2ncc(C(=O)NCc3ccc4c(N)nccc4c3)cc2Cl)CC1 ; "C23H27ClN6O"] | 8.03 |
| 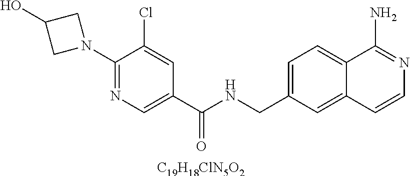 | 8.04 |
| 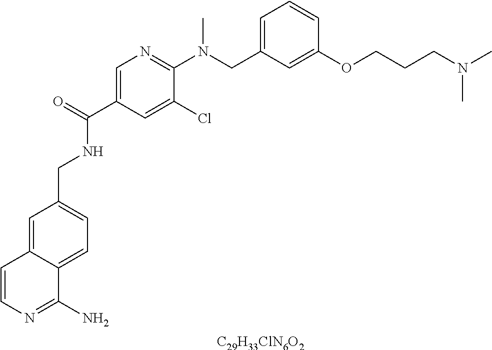 [SMILES: CN(C)CCCOc1cccc(CN(C)c2ncc(C(=O)NCc3ccc4c(N)nccc4c3)cc2Cl)c1 ; "C29H33ClN6O2"] | 8.05 |
| 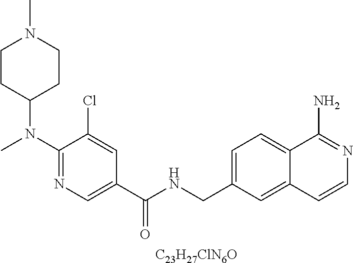 [SMILES: CN1CCC(N(C)c2ncc(C(=O)NCc3ccc4c(N)nccc4c3)cc2Cl)CC1 ; "C23H27ClN6O"] | 8.06 |
| 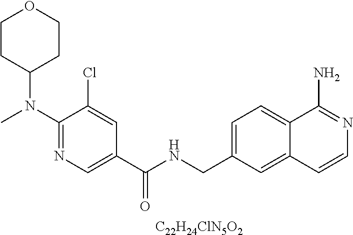 | 8.07 |
| 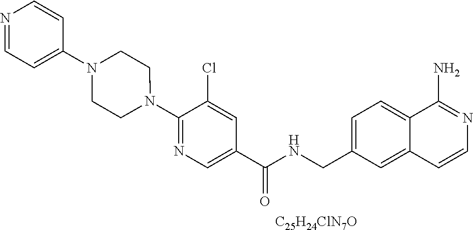 | 8.08 |
| 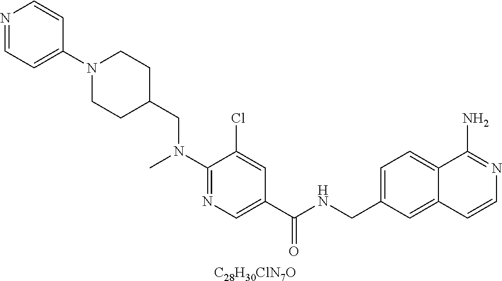 [SMILES: CN(CC1CCN(c2ccncc2)CC1)c1ncc(C(=O)NCc2ccc3c(N)nccc3c2)cc1Cl ; "C28H30ClN7O"] | 8.09 |

TABLE 4

| Structure | Example |
|---|---|
| Molecular formula | No. |
| 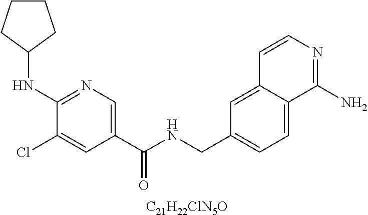 | 11.01 |
| 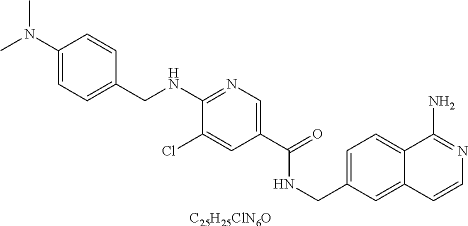 [SMILES: CN(C)c1ccc(CNc2ncc(C(=O)NCc3ccc4c(N)nccc4c3)cc2Cl)cc1 ; "C25H25ClN6O"] | 11.02 |
| 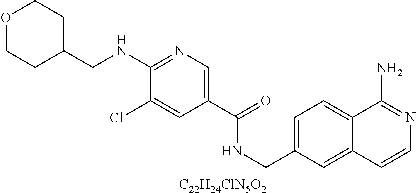 [SMILES: Nc1nccc2cc(CNC(=O)c3cnc(NCC4CCOCC4)c(Cl)c3)ccc12 ; "C22H24ClN5O2"] | 11.03 |
| 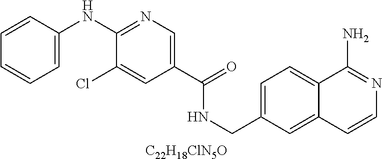 [SMILES: Nc1nccc2cc(CNC(=O)c3cnc(Nc4ccccc4)c(Cl)c3)ccc12 ; "C22H18ClN5O"] | 11.04 |
| 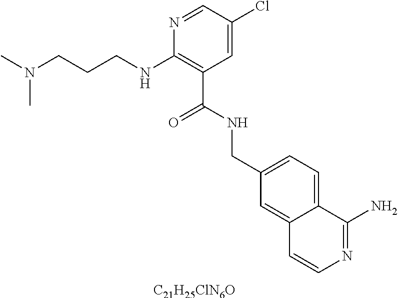 [SMILES: CN(C)CCCNc1ncc(Cl)cc1C(=O)NCc1ccc2c(N)nccc2c1 ; "C21H25ClN6O"] | 11.05 |
| 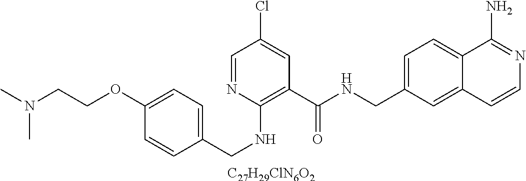 [SMILES: CN(C)CCOc1ccc(CNc2ncc(Cl)cc2C(=O)NCc2ccc3c(N)nccc3c2)cc1 ; "C27H29ClN6O2"] | 11.06 |
| 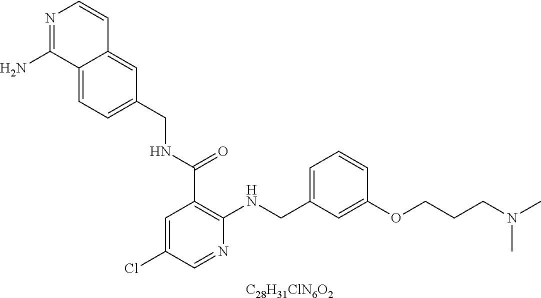 [SMILES: CN(C)CCCOc1cccc(CNc2ncc(Cl)cc2C(=O)NCc2ccc3c(N)nccc3c2)c1 ; "C28H31ClN6O2"] | 11.07 |
| 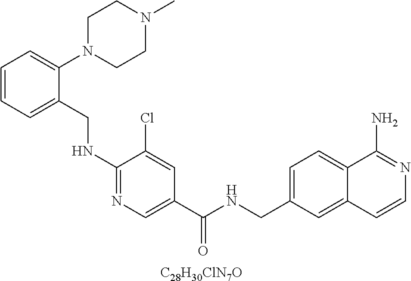 | 11.08 |
| 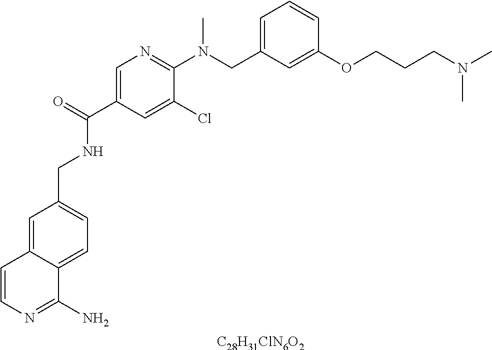 [SMILES: CN(C)CCCOc1cccc(CN(C)c2ncc(C(=O)NCc3ccc4c(N)nccc4c3)cc2Cl)c1 ; "C28H31ClN6O2"] | 11.09 |
| 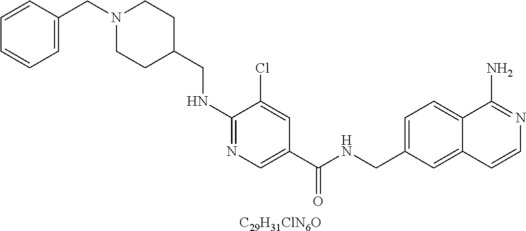 [SMILES: Nc1nccc2cc(CNC(=O)c3cnc(NCC4CCN(Cc5ccccc5)CC4)c(Cl)c3)ccc12 ; "C29H31ClN6O"] | 11.10 |
| 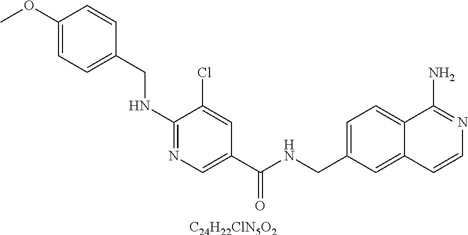 [SMILES: COc1ccc(CNc2ncc(C(=O)NCc3ccc4c(N)nccc4c3)cc2Cl)cc1 ; "C24H22ClN5O2"] | 11.11 |
| 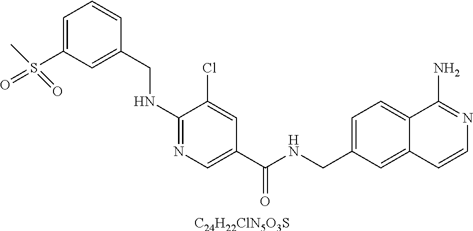 [SMILES: CS(=O)(=O)c1cccc(CNc2ncc(C(=O)NCc3ccc4c(N)nccc4c3)cc2Cl)c1 ; "C24H22ClN5O3S"] | 11.12 |
| 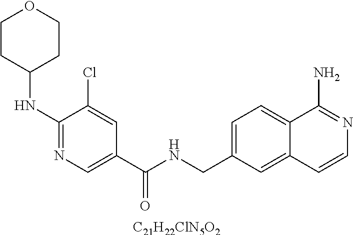 | 11.13 |
| 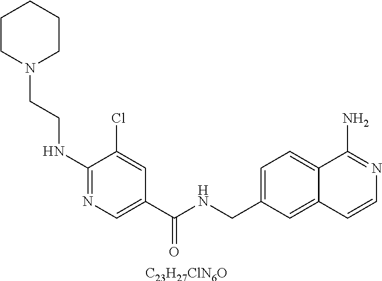 [SMILES: Nc1nccc2cc(CNC(=O)c3cnc(NCCN4CCCCC4)c(Cl)c3)ccc12 ; "C23H27ClN6O"] | 11.14 |
| 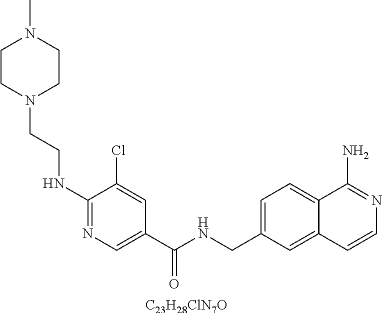 | 11.15 |
| 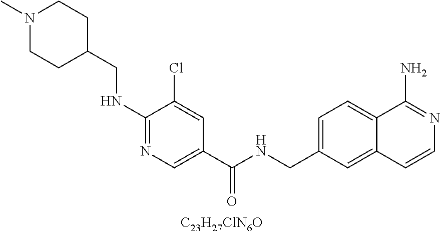 [SMILES: CN1CCC(CNc2ncc(C(=O)NCc3ccc4c(N)nccc4c3)cc2Cl)CC1 ; "C23H27ClN6O"] | 11.16 |
| 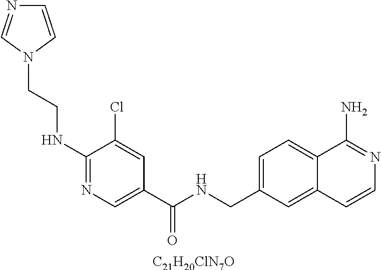 | 11.17 |
| 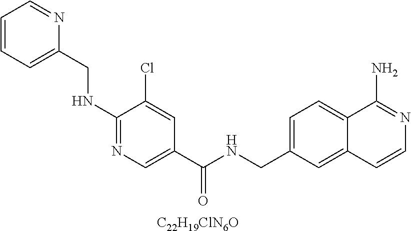 [SMILES: Nc1nccc2cc(CNC(=O)c3cnc(NCc4ccccn4)c(Cl)c3)ccc12 ; "C22H19ClN6O"] | 11.18 |
| 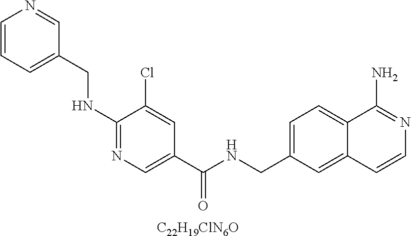 [SMILES: Nc1nccc2cc(CNC(=O)c3cnc(NCc4cccnc4)c(Cl)c3)ccc12 ; "C22H19ClN6O"] | 11.19 |
| 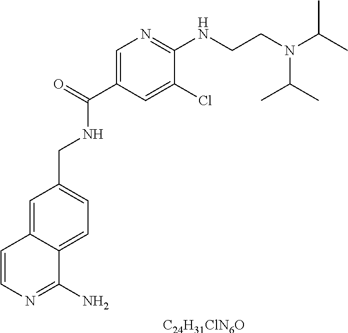 [SMILES: CC(C)N(CCNc1ncc(C(=O)NCc2ccc3c(N)nccc3c2)cc1Cl)C(C)C ; "C24H31ClN6O"] | 11.20 |
| 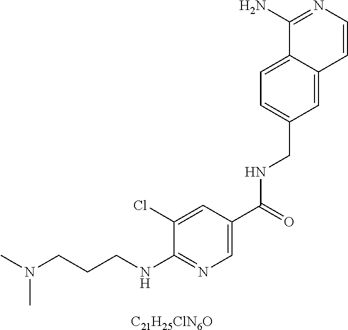 [SMILES: CN(C)CCCNc1ncc(C(=O)NCc2ccc3c(N)nccc3c2)cc1Cl ; "C21H25ClN6O"] | 11.21 |
| 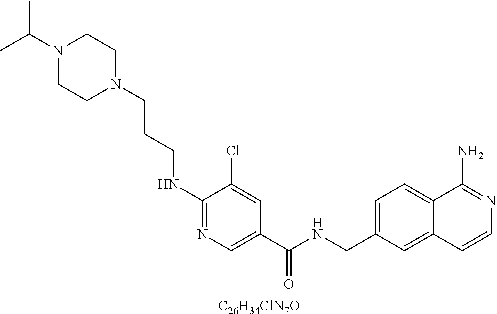 [SMILES: CC(C)N1CCN(CCCNc2ncc(C(=O)NCc3ccc4c(N)nccc4c3)cc2Cl)CC1 ; "C26H34ClN7O"] | 11.22 |
| 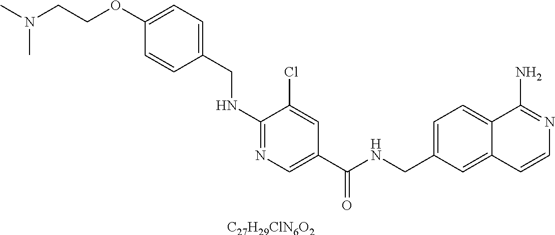 [SMILES: CN(C)CCOc1ccc(CNc2ncc(C(=O)NCc3ccc4c(N)nccc4c3)cc2Cl)cc1 ; "C27H29ClN6O2"] | 11.23 |
| 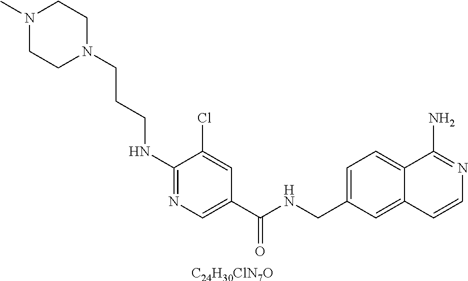 [SMILES: CN1CCN(CCCNc2ncc(C(=O)NCc3ccc4c(N)nccc4c3)cc2Cl)CC1 ; "C24H30ClN7O"] | 11.24 |
| 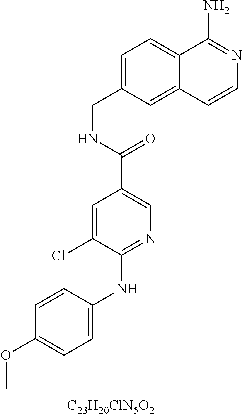 [SMILES: COc1ccc(Nc2ncc(C(=O)NCc3ccc4c(N)nccc4c3)cc2Cl)cc1 ; "C23H20ClN5O2"] | 11.25 |
| 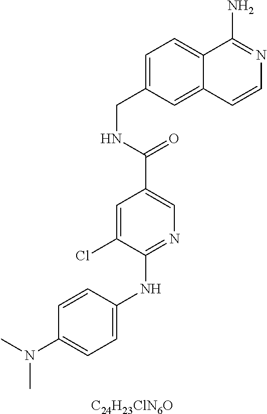 [SMILES: CN(C)c1ccc(Nc2ncc(C(=O)NCc3ccc4c(N)nccc4c3)cc2Cl)cc1 ; "C24H23ClN6O"] | 11.26 |
| 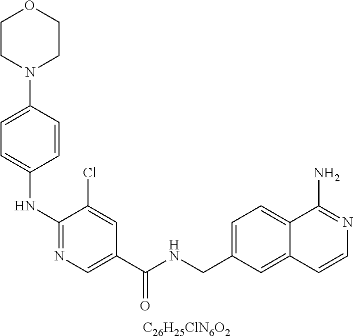 [SMILES: Nc1nccc2cc(CNC(=O)c3cnc(Nc4ccc(N5CCOCC5)cc4)c(Cl)c3)ccc12 ; "C26H25ClN6O2"] | 11.27 |
| 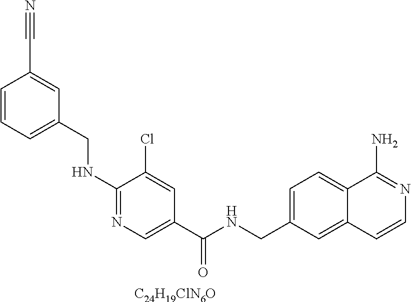 [SMILES: N#Cc1cccc(CNc2ncc(C(=O)NCc3ccc4c(N)nccc4c3)cc2Cl)c1 ; "C24H19ClN6O"] | 11.28 |
| 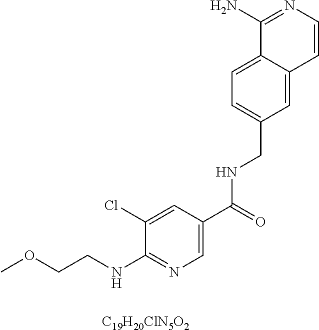 [SMILES: COCCNc1ncc(C(=O)NCc2ccc3c(N)nccc3c2)cc1Cl ; "C19H20ClN5O2"] | 11.29 |
| 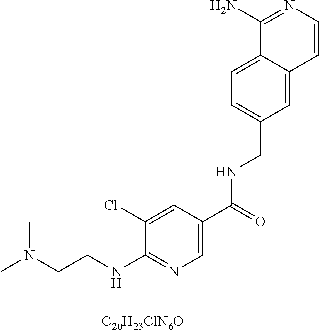 [SMILES: CN(C)CCNc1ncc(C(=O)NCc2ccc3c(N)nccc3c2)cc1Cl ; "C20H23ClN6O"] | 11.30 |
| 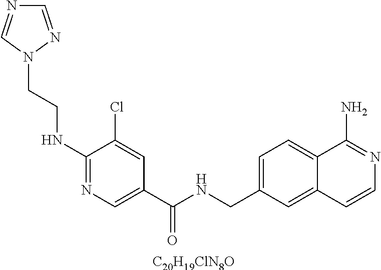 [SMILES: Nc1nccc2cc(CNC(=O)c3cnc(NCCn4cncn4)c(Cl)c3)ccc12 ; "C20H19ClN8O"] | 11.31 |
| 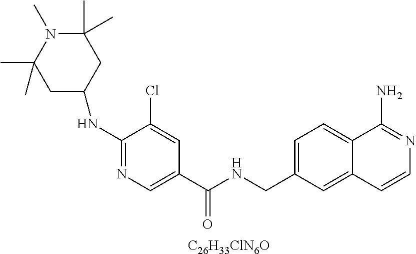 [SMILES: CN1C(C)(C)CC(Nc2ncc(C(=O)NCc3ccc4c(N)nccc4c3)cc2Cl)CC1(C)C ; "C26H33ClN6O"] | 11.32 |
| 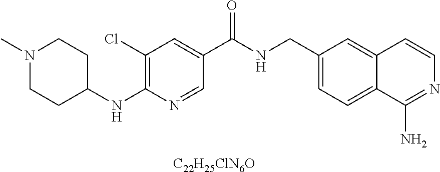 [SMILES: CN1CCC(Nc2ncc(C(=O)NCc3ccc4c(N)nccc4c3)cc2Cl)CC1 ; "C22H25ClN6O"] | 11.33 |
| 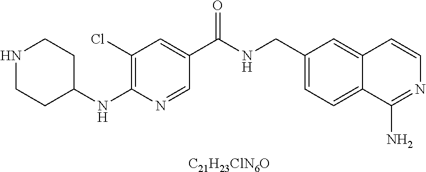 [SMILES: Nc1nccc2cc(CNC(=O)c3cnc(NC4CCNCC4)c(Cl)c3)ccc12 ; "C21H23ClN6O"] | 11.34 |
| 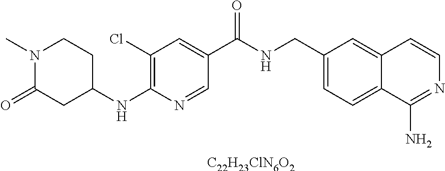 [SMILES: CN1CCC(Nc2ncc(C(=O)NCc3ccc4c(N)nccc4c3)cc2Cl)CC1=O ; "C22H23ClN6O2"] | 11.35 |
| 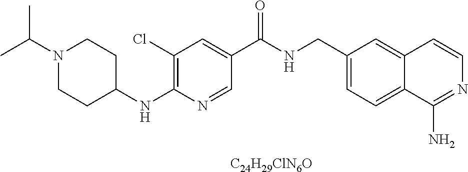 [SMILES: CC(C)N1CCC(Nc2ncc(C(=O)NCc3ccc4c(N)nccc4c3)cc2Cl)CC1 ; "C24H29ClN6O"] | 11.36 |
| 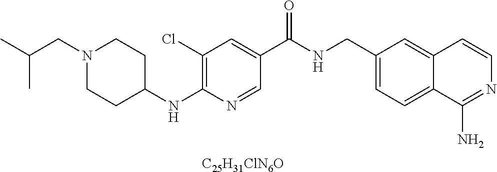 [SMILES: CC(C)CN1CCC(Nc2ncc(C(=O)NCc3ccc4c(N)nccc4c3)cc2Cl)CC1 ; "C25H31ClN6O"] | 11.37 |
| 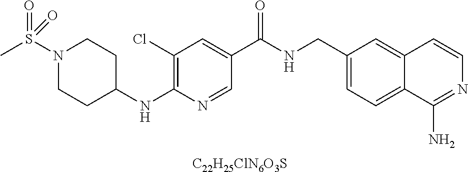 [SMILES: CS(=O)(=O)N1CCC(Nc2ncc(C(=O)NCc3ccc4c(N)nccc4c3)cc2Cl)CC1 ; "C22H25ClN6O3S"] | 11.38 |
| 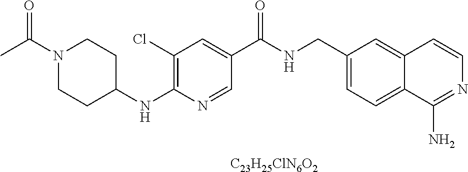 [SMILES: CC(=O)N1CCC(Nc2ncc(C(=O)NCc3ccc4c(N)nccc4c3)cc2Cl)CC1 ; "C23H25ClN6O2"] | 11.39 |
| 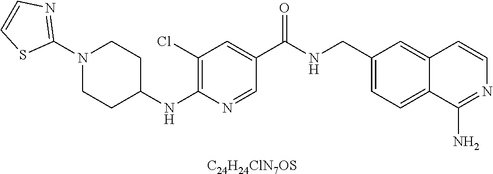 [SMILES: Nc1nccc2cc(CNC(=O)c3cnc(NC4CCN(c5nccs5)CC4)c(Cl)c3)ccc12 ; "C24H24ClN7OS"] | 11.40 |
| 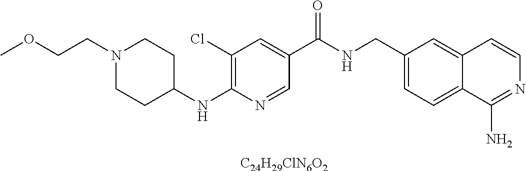 [SMILES: COCCN1CCC(Nc2ncc(C(=O)NCc3ccc4c(N)nccc4c3)cc2Cl)CC1 ; "C24H29ClN6O2"] | 11.41 |
| 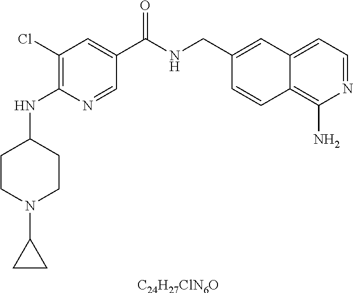 [SMILES: Nc1nccc2cc(CNC(=O)c3cnc(NC4CCN(C5CC5)CC4)c(Cl)c3)ccc12 ; "C24H27ClN6O"] | 11.42 |

TABLE 5

| Structure | Example |
|---|---|
| Molecular formula | No. |
| 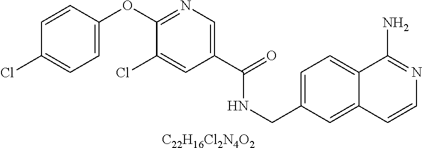 [SMILES: Nc1nccc2cc(CNC(=O)c3cnc(Oc4ccc(Cl)cc4)c(Cl)c3)ccc12 ; "C22H16Cl2N4O2"] | 13.01 |
| 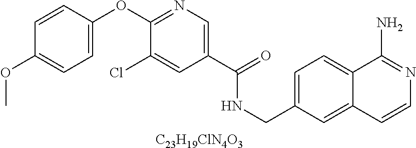 [SMILES: COc1ccc(Oc2ncc(C(=O)NCc3ccc4c(N)nccc4c3)cc2Cl)cc1 ; "C23H19ClN4O3"] | 13.02 |
| 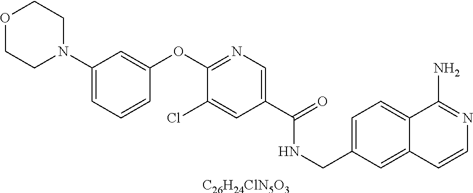 [SMILES: Nc1nccc2cc(CNC(=O)c3cnc(Oc4cccc(N5CCOCC5)c4)c(Cl)c3)ccc12 ; "C26H24ClN5O3"] | 13.03 |
| 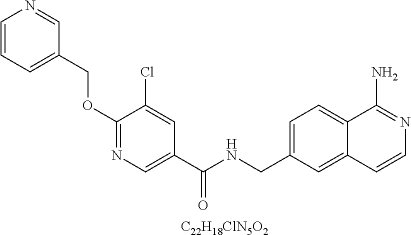 | 13.04 |
| 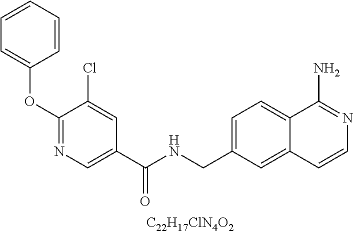 [SMILES: Nc1nccc2cc(CNC(=O)c3cnc(Oc4ccccc4)c(Cl)c3)ccc12 ; "C22H17ClN4O2"] | 13.05 |
| 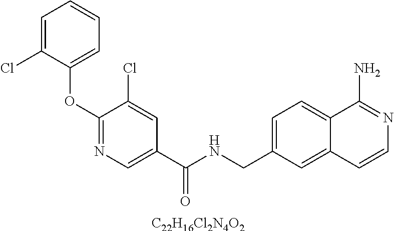 [SMILES: Nc1nccc2cc(CNC(=O)c3cnc(Oc4ccccc4Cl)c(Cl)c3)ccc12 ; "C22H16Cl2N4O2"] | 13.06 |
| 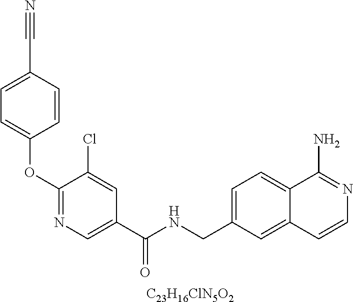 | 13.07 |
| 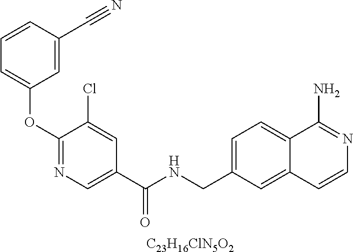 | 13.08 |
| 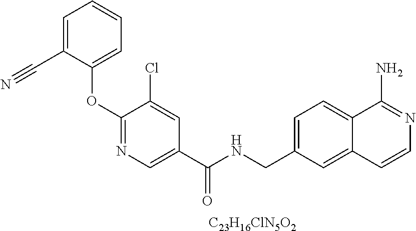 | 13.09 |
| 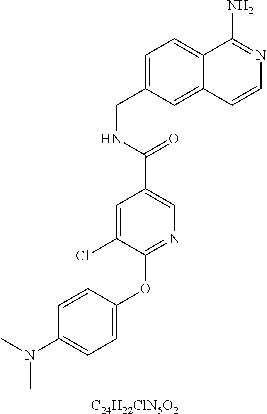 [SMILES: CN(C)c1ccc(Oc2ncc(C(=O)NCc3ccc4c(N)nccc4c3)cc2Cl)cc1 ; "C24H22ClN5O2"] | 13.10 |
| 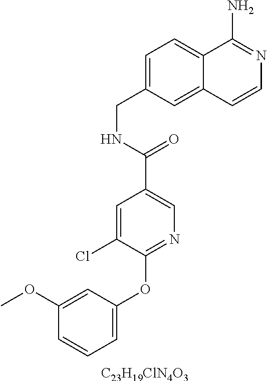 [SMILES: COc1cccc(Oc2ncc(C(=O)NCc3ccc4c(N)nccc4c3)cc2Cl)c1 ; "C23H19ClN4O3"] | 13.11 |
| 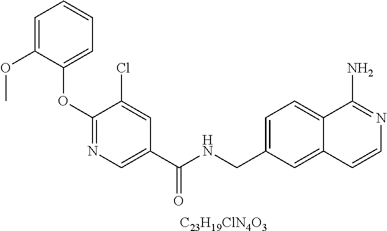 [SMILES: COc1ccccc1Oc1ncc(C(=O)NCc2ccc3c(N)nccc3c2)cc1Cl ; "C23H19ClN4O3"] | 13.12 |
| 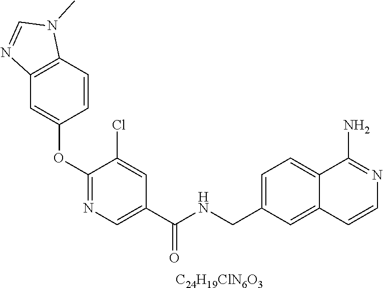 [SMILES: Cn1cnc2cc(Oc3ncc(C(=O)NCc4ccc5c(N)nccc5c4)cc3Cl)ccc21 ; "C24H19ClN6O3"] | 13.13 |
| 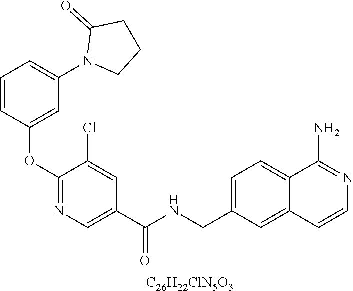 | 13.14 |
| 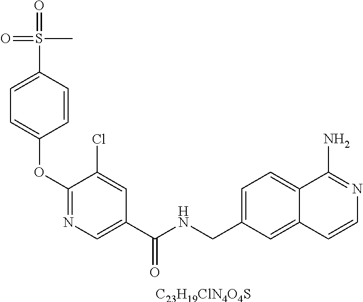 [SMILES: CS(=O)(=O)c1ccc(Oc2ncc(C(=O)NCc3ccc4c(N)nccc4c3)cc2Cl)cc1 ; "C23H19ClN4O4S"] | 13.15 |
| 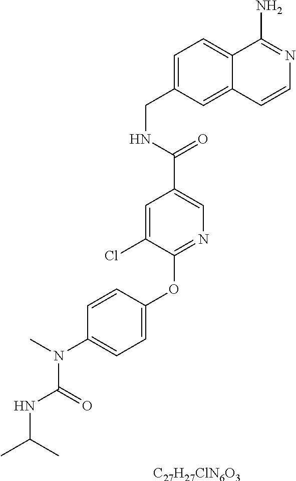 [SMILES: CC(C)NC(=O)N(C)c1ccc(Oc2ncc(C(=O)NCc3ccc4c(N)nccc4c3)cc2Cl)cc1 ; "C27H27ClN6O3"] | 13.16 |
| 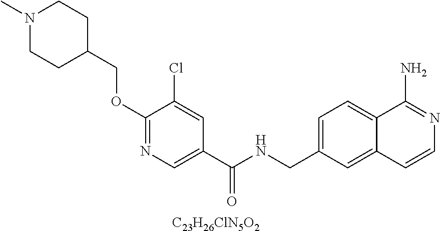 [SMILES: CN1CCC(COc2ncc(C(=O)NCc3ccc4c(N)nccc4c3)cc2Cl)CC1 ; "C23H26ClN5O2"] | 13.17 |
| 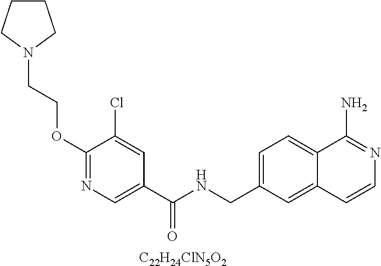 | 13.18 |
| 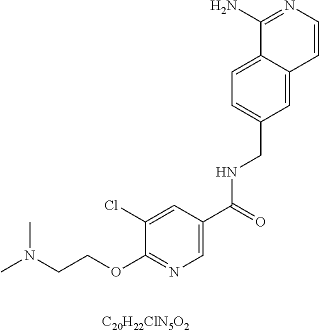 [SMILES: CN(C)CCOc1ncc(C(=O)NCc2ccc3c(N)nccc3c2)cc1Cl ; "C20H22ClN5O2"] | 13.19 |
| 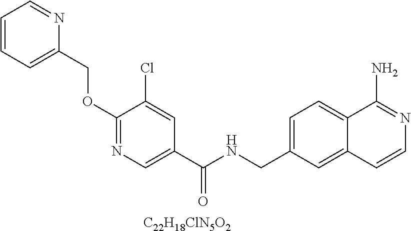 [SMILES: Nc1nccc2cc(CNC(=O)c3cnc(OCc4ccccn4)c(Cl)c3)ccc12 ; "C22H18ClN5O2"] | 13.20 |
| 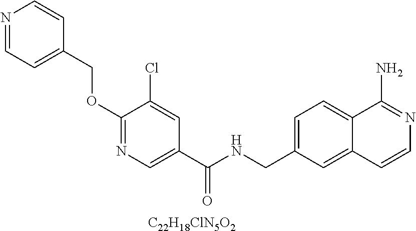 [SMILES: Nc1nccc2cc(CNC(=O)c3cnc(OCc4ccncc4)c(Cl)c3)ccc12 ; "C22H18ClN5O2"] | 13.21 |
| 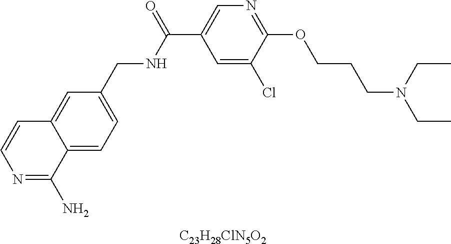 [SMILES: CCN(CC)CCCOc1ncc(C(=O)NCc2ccc3c(N)nccc3c2)cc1Cl ; "C23H28ClN5O2"] | 13.22 |
| 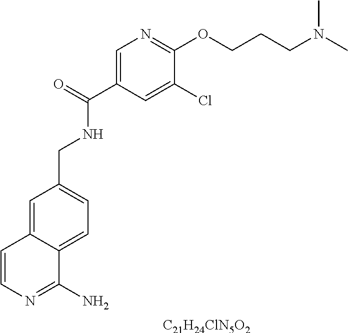 [SMILES: CN(C)CCCOc1ncc(C(=O)NCc2ccc3c(N)nccc3c2)cc1Cl ; "C21H24ClN5O2"] | 13.23 |
| 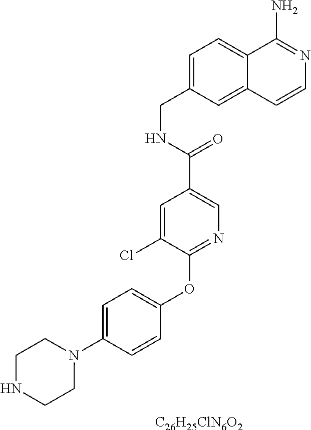 [SMILES: Nc1nccc2cc(CNC(=O)c3cnc(Oc4ccc(N5CCNCC5)cc4)c(Cl)c3)ccc12 ; "C26H25ClN6O2"] | 13.24 |
| 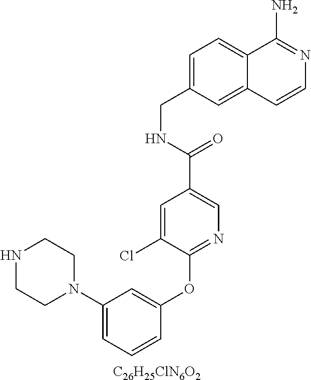 [SMILES: Nc1nccc2cc(CNC(=O)c3cnc(Oc4cccc(N5CCNCC5)c4)c(Cl)c3)ccc12 ; "C26H25ClN6O2"] | 13.25 |
| 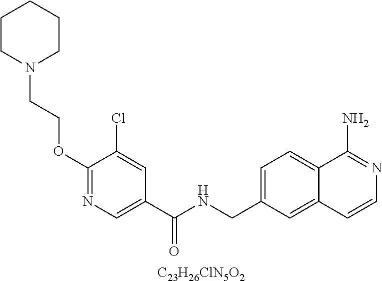 | 13.26 |
| 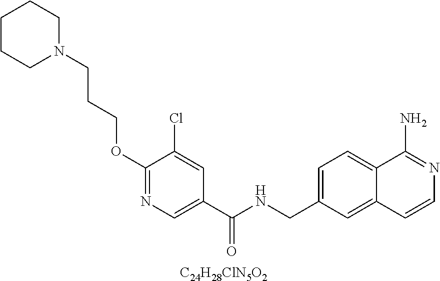 [SMILES: Nc1nccc2cc(CNC(=O)c3cnc(OCCCN4CCCCC4)c(Cl)c3)ccc12 ; "C24H28ClN5O2"] | 13.27 |

TABLE 6

| Structure | Example |
|---|---|
| Molecular formula | No. |
| 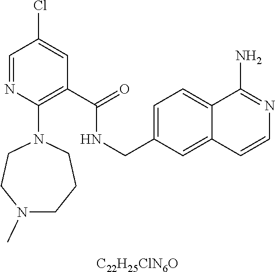 [SMILES: CN1CCCN(c2ncc(Cl)cc2C(=O)NCc2ccc3c(N)nccc3c2)CC1 ; "C22H25ClN6O"] | 16.01 |
| 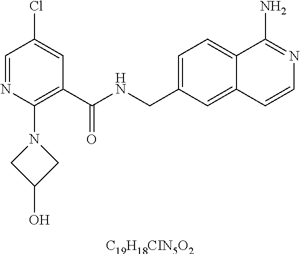 [SMILES: Nc1nccc2cc(CNC(=O)c3cc(Cl)cnc3N3CC(O)C3)ccc12 ; "C19H18CIN5O2"] | 16.02 |
| 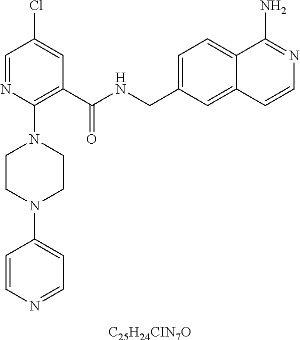 [SMILES: Nc1nccc2cc(CNC(=O)c3cc(Cl)cnc3N3CCN(c4ccncc4)CC3)ccc12 ; "C25H24CIN7O"] | 16.03 |
| 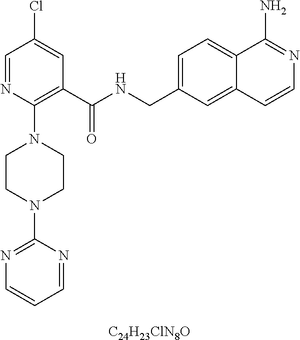 [SMILES: Nc1nccc2cc(CNC(=O)c3cc(Cl)cnc3N3CCN(c4ncccn4)CC3)ccc12 ; "C24H23ClN8O"] | 16.04 |
| 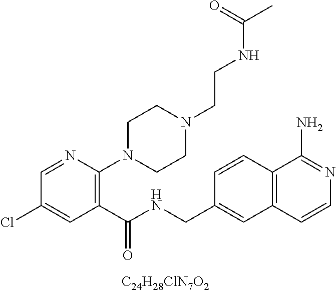 | 16.05 |

TABLE 7

| Structure | Example |
|---|---|
| Molecular formula | No. |
| 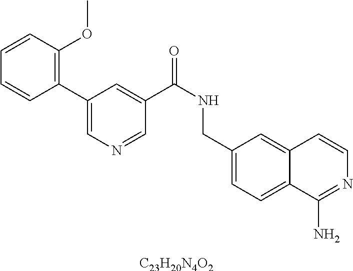 [SMILES: COc1ccccc1-c1cncc(C(=O)NCc2ccc3c(N)nccc3c2)c1 ; "C23H20N4O2"] | 19.01 |
| 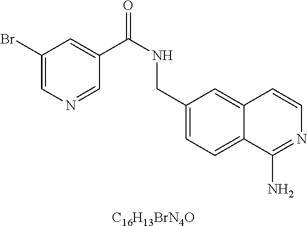 [SMILES: "C16H13BrN4O" ; Nc1nccc2cc(CNC(=O)c3cncc(Br)c3)ccc12] | 19.02 |
| 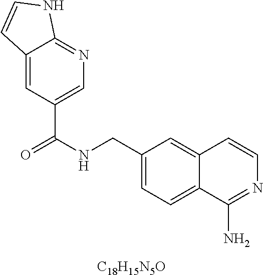 [SMILES: Nc1nccc2cc(CNC(=O)c3cnc4[nH]ccc4c3)ccc12 ; "C18H15N5O"] | 19.03 |
| 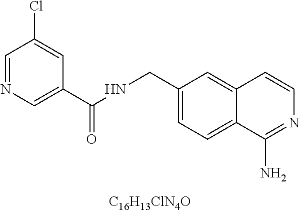 [SMILES: "C16H13ClN4O" ; Nc1nccc2cc(CNC(=O)c3cncc(Cl)c3)ccc12] | 19.04 |
| 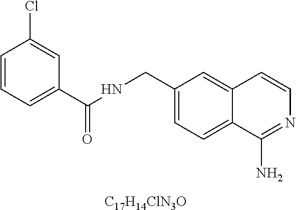 [SMILES: Nc1nccc2cc(CNC(=O)c3cccc(Cl)c3)ccc12 ; "C17H14ClN3O"] | 19.05 |
| 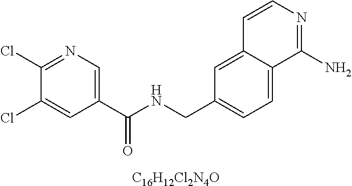 [SMILES: Nc1nccc2cc(CNC(=O)c3cnc(Cl)c(Cl)c3)ccc12 ; "C16H12Cl2N4O"] | 19.06 |

TABLE 8

| Structure | Example |
|---|---|
| Molecular formula | No. |
| 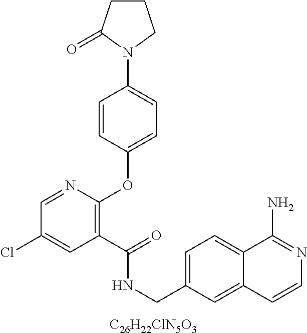 | 22.01 |
| 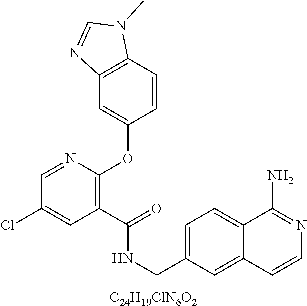 [SMILES: Cn1cnc2cc(Oc3ncc(Cl)cc3C(=O)NCc3ccc4c(N)nccc4c3)ccc21 ; "C24H19ClN6O2"] | 22.02 |
| 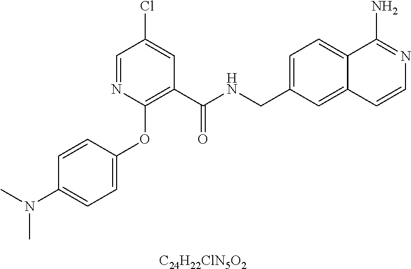 [SMILES: CN(C)c1ccc(Oc2ncc(Cl)cc2C(=O)NCc2ccc3c(N)nccc3c2)cc1 ; "C24H22ClN5O2"] | 22.03 |
| 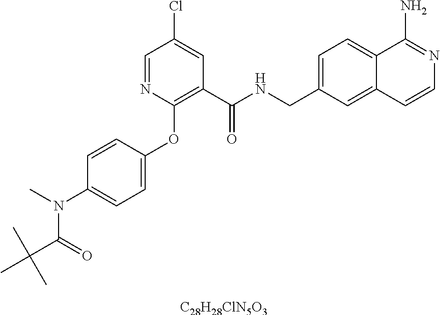 [SMILES: CN(C(=O)C(C)(C)C)c1ccc(Oc2ncc(Cl)cc2C(=O)NCc2ccc3c(N)nccc3c2)cc1 ; "C28H28ClN5O3"] | 22.04 |
| 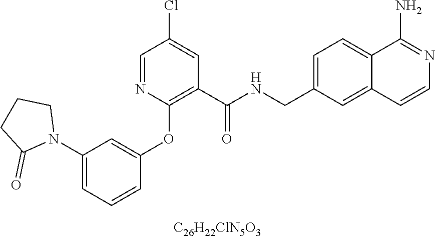 [SMILES: Nc1nccc2cc(CNC(=O)c3cc(Cl)cnc3Oc3cccc(N4CCCC4=O)c3)ccc12 ; "C26H22ClN5O3"] | 22.05 |
| 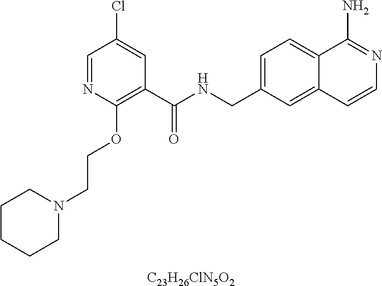 [SMILES: Nc1nccc2cc(CNC(=O)c3cc(Cl)cnc3OCCN3CCCCC3)ccc12 ; "C23H26ClN5O2"] | 22.06 |
| 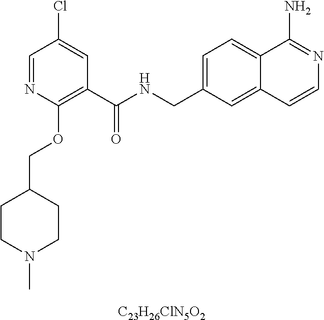 [SMILES: CN1CCC(COc2ncc(Cl)cc2C(=O)NCc2ccc3c(N)nccc3c2)CC1 ; "C23H26ClN5O2"] | 22.07 |
| 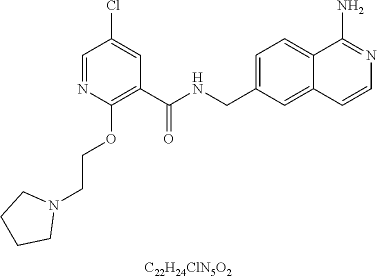 [SMILES: Nc1nccc2cc(CNC(=O)c3cc(Cl)cnc3OCCN3CCCC3)ccc12 ; "C22H24ClN5O2"] | 22.08 |
| 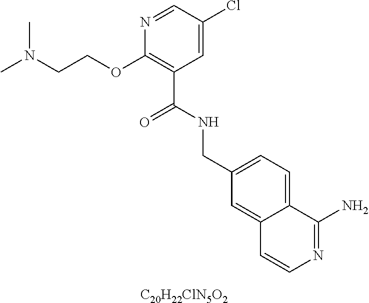 [SMILES: CN(C)CCOc1ncc(Cl)cc1C(=O)NCc1ccc2c(N)nccc2c1 ; "C20H22ClN5O2"] | 22.09 |
| 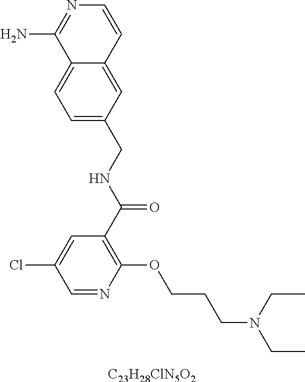 [SMILES: CCN(CC)CCCOc1ncc(Cl)cc1C(=O)NCc1ccc2c(N)nccc2c1 ; "C23H28ClN5O2"] | 22.10 |
| 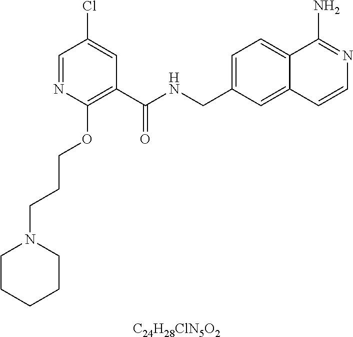 [SMILES: Nc1nccc2cc(CNC(=O)c3cc(Cl)cnc3OCCCN3CCCCC3)ccc12 ; "C24H28ClN5O2"] | 22.11 |
| 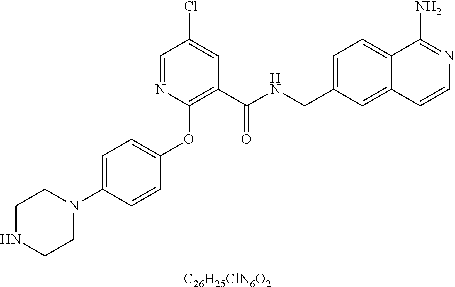 [SMILES: Nc1nccc2cc(CNC(=O)c3cc(Cl)cnc3Oc3ccc(N4CCNCC4)cc3)ccc12 ; "C26H25ClN6O2"] | 22.12 |
| 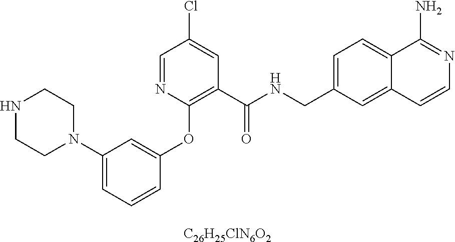 [SMILES: Nc1nccc2cc(CNC(=O)c3cc(Cl)cnc3Oc3cccc(N4CCNCC4)c3)ccc12 ; "C26H25ClN6O2"] | 22.13 |

TABLE 9

| Structure | Example |
|---|---|
| Molecular formula | No. |
| 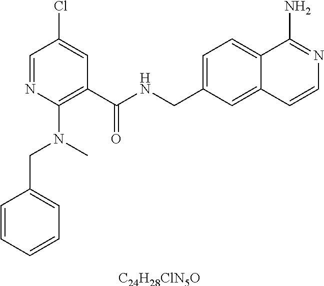 [SMILES: CN(Cc1ccccc1)c1ncc(Cl)cc1C(=O)NCc1ccc2c(N)nccc2c1 ; "C24H28ClN5O"] | 28.01 |
| 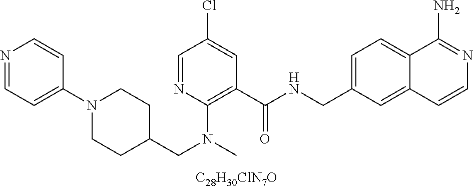 | 28.02 |
| 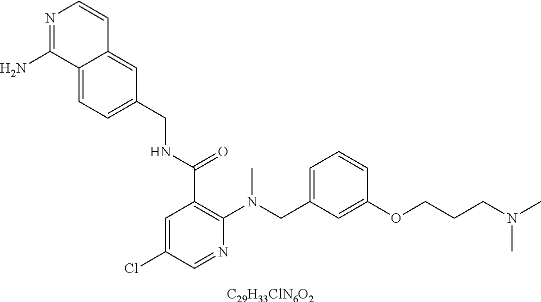 [SMILES: CN(C)CCCOc1cccc(CN(C)c2ncc(Cl)cc2C(=O)NCc2ccc3c(N)nccc3c2)c1 ; "C29H33ClN6O2"] | 28.03 |
| 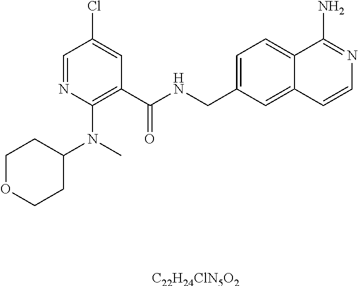 [SMILES: CN(c1ncc(Cl)cc1C(=O)NCc1ccc2c(N)nccc2c1)C1CCOCC1 ; "C22H24ClN5O2"] | 28.04 |
| 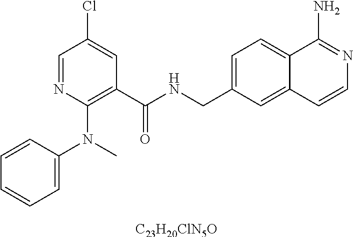 [SMILES: CN(c1ccccc1)c1ncc(Cl)cc1C(=O)NCc1ccc2c(N)nccc2c1 ; "C23H20ClN5O"] | 28.05 |
| 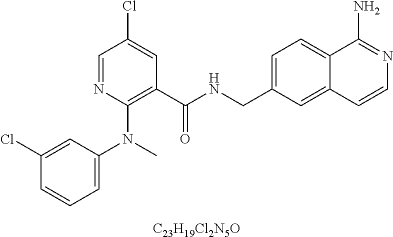 [SMILES: CN(c1cccc(Cl)c1)c1ncc(Cl)cc1C(=O)NCc1ccc2c(N)nccc2c1 ; "C23H19Cl2N5O"] | 28.06 |

TABLE 10

| Structure | Example |
|---|---|
| Molecular formula | No. |
| 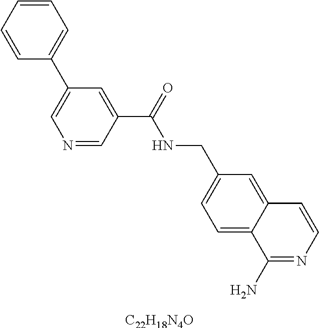 [SMILES: Nc1nccc2cc(CNC(=O)c3cncc(-c4ccccc4)c3)ccc12 ; "C22H18N4O"] | 29.01 |
| 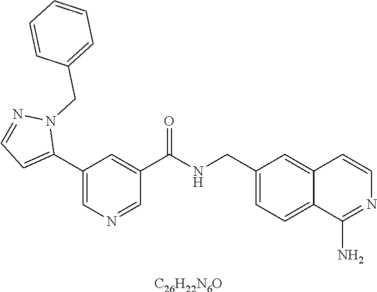 [SMILES: Nc1nccc2cc(CNC(=O)c3cncc(-c4ccnn4Cc4ccccc4)c3)ccc12 ; "C26H22N6O"] | 29.03 |
| 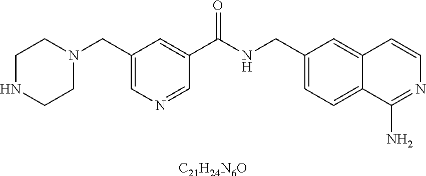 [SMILES: Nc1nccc2cc(CNC(=O)c3cncc(CN4CCNCC4)c3)ccc12 ; "C21H24N6O"] | 29.04 |
| 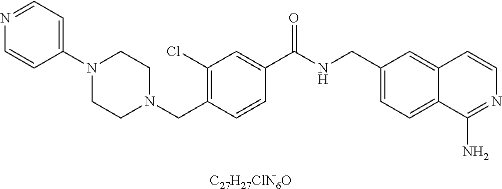 [SMILES: Nc1nccc2cc(CNC(=O)c3ccc(CN4CCN(c5ccncc5)CC4)c(Cl)c3)ccc12 ; "C27H27ClN6O"] | 29.07 |
| 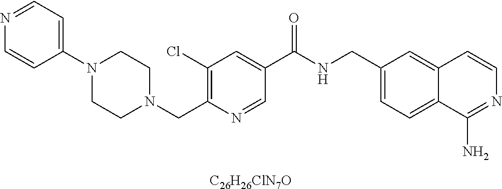 [SMILES: Nc1nccc2cc(CNC(=O)c3cnc(CN4CCN(c5ccncc5)CC4)c(Cl)c3)ccc12 ; "C26H26ClN7O"] | 29.08 |
| 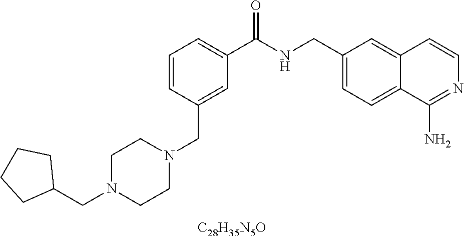 [SMILES: Nc1nccc2cc(CNC(=O)c3cccc(CN4CCN(CC5CCCC5)CC4)c3)ccc12 ; "C28H35N5O"] | 29.09 |
| 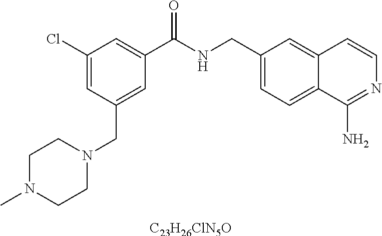 [SMILES: CN1CCN(Cc2cc(Cl)cc(C(=O)NCc3ccc4c(N)nccc4c3)c2)CC1 ; "C23H26ClN5O"] | 29.10 |
| 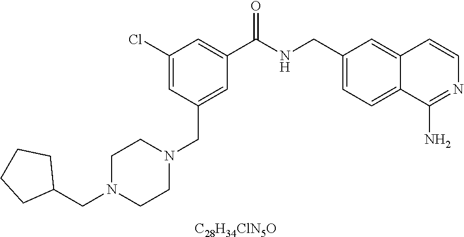 [SMILES: Nc1nccc2cc(CNC(=O)c3cc(Cl)cc(CN4CCN(CC5CCCC5)CC4)c3)ccc12 ; "C28H34ClN5O"] | 29.11 |

TABLE 11

| Structure | Example |
|---|---|
| Molecular formula | No. |
| 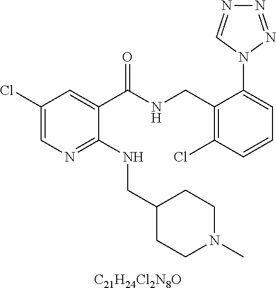 [SMILES: CN1CCC(CNc2ncc(Cl)cc2C(=O)NCc2c(Cl)cccc2-n2cnnn2)CC1 ; "C21H24Cl2N8O"] | 33.01 |
| 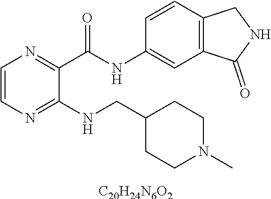 [SMILES: CN1CCC(CNc2nccnc2C(=O)Nc2ccc3c(c2)C(=O)NC3)CC1 ; "C20H24N6O2"] | 33.02 |
| 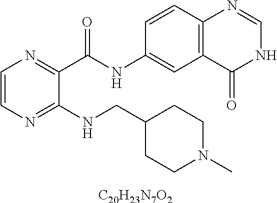 [SMILES: CN1CCC(CNc2nccnc2C(=O)Nc2ccc3nc[nH]c(=O)c3c2)CC1 ; "C20H23N7O2"] | 33.03 |
| 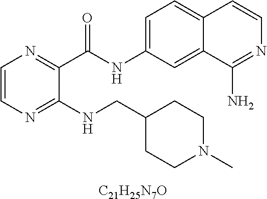 [SMILES: CN1CCC(CNc2nccnc2C(=O)Nc2ccc3ccnc(N)c3c2)CC1 ; "C21H25N7O"] | 33.04 |
| 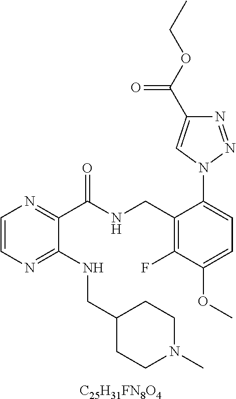 [SMILES: CCOC(=O)c1cn(-c2ccc(OC)c(F)c2CNC(=O)c2nccnc2NCC2CCN(C)CC2)nn1 ; "C25H31FN8O4"] | 33.05 |
| 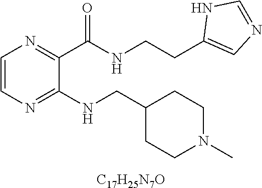 [SMILES: CN1CCC(CNc2nccnc2C(=O)NCCc2cnc[nH]2)CC1 ; "C17H25N7O"] | 33.06 |
| 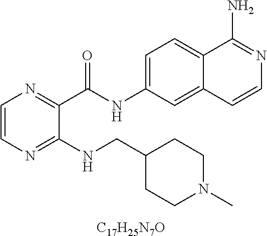 [SMILES: CN1CCC(CNc2nccnc2C(=O)Nc2ccc3c(N)nccc3c2)CC1 ; "C17H25N7O"] | 33.07 |
| 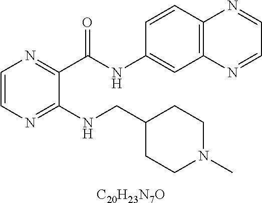 [SMILES: CN1CCC(CNc2nccnc2C(=O)Nc2ccc3nccnc3c2)CC1 ; "C20H23N7O"] | 33.08 |
| 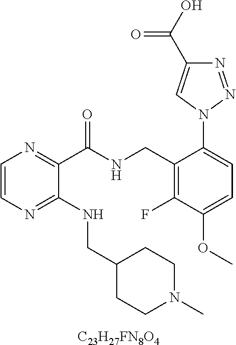 [SMILES: COc1ccc(-n2cc(C(=O)O)nn2)c(CNC(=O)c2nccnc2NCC2CCN(C)CC2)c1F ; "C23H27FN8O4"] | 33.09 |
| 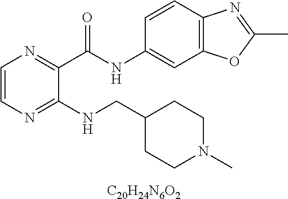 [SMILES: Cc1nc2ccc(NC(=O)c3nccnc3NCC3CCN(C)CC3)cc2o1 ; "C20H24N6O2"] | 33.10 |
| 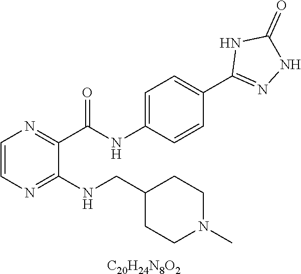 [SMILES: CN1CCC(CNc2nccnc2C(=O)Nc2ccc(-c3n[nH]c(=O)[nH]3)cc2)CC1 ; "C20H24N8O2"] | 33.11 |
| 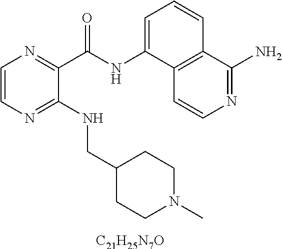 [SMILES: CN1CCC(CNc2nccnc2C(=O)Nc2cccc3c(N)nccc23)CC1 ; "C21H25N7O"] | 33.12 |
| 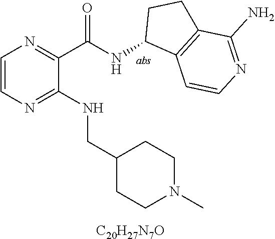 [SMILES: CN1CCC(CNc2nccnc2C(=O)N[C@@H]2CCc3c2ccnc3N)CC1 ; "C20H27N7O"] | 33.13 |
| 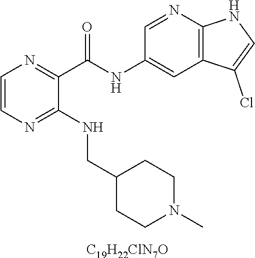 | 33.14 |
| 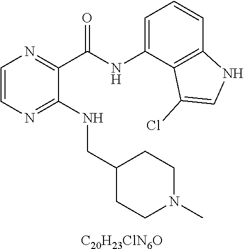 [SMILES: CN1CCC(CNc2nccnc2C(=O)Nc2cccc3[nH]cc(Cl)c23)CC1 ; "C20H23ClN6O"] | 33.15 |
| 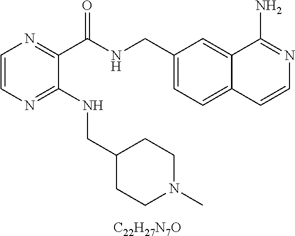 [SMILES: CN1CCC(CNc2nccnc2C(=O)NCc2ccc3ccnc(N)c3c2)CC1 ; "C22H27N7O"] | 33.16 |
| 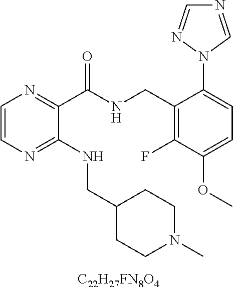 [SMILES: COc1ccc(-n2cncn2)c(CNC(=O)c2nccnc2NCC2CCN(C)CC2)c1F ; "C22H27FN8O4"] | 33.17 |
| 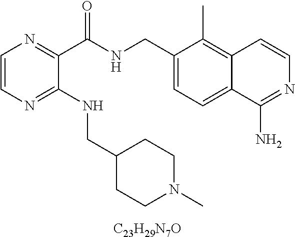 [SMILES: Cc1c(CNC(=O)c2nccnc2NCC2CCN(C)CC2)ccc2c(N)nccc12 ; "C23H29N7O"] | 33.18 |
| 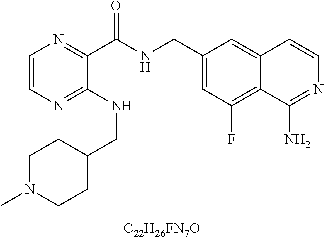 [SMILES: CN1CCC(CNc2nccnc2C(=O)NCc2cc(F)c3c(N)nccc3c2)CC1 ; "C22H26FN7O"] | 33.19 |
| 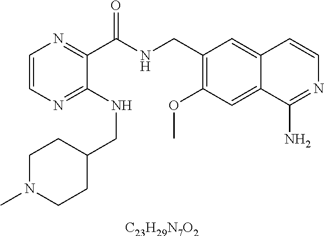 [SMILES: COc1cc2c(N)nccc2cc1CNC(=O)c1nccnc1NCC1CCN(C)CC1 ; "C23H29N7O2"] | 33.20 |
| 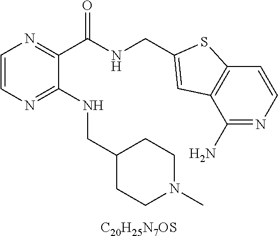 [SMILES: CN1CCC(CNc2nccnc2C(=O)NCc2cc3c(N)nccc3s2)CC1 ; "C20H25N7OS"] | 33.21 |
| 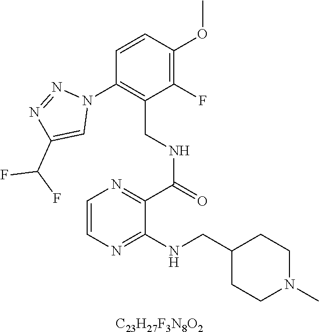 [SMILES: COc1ccc(-n2cc(C(F)F)nn2)c(CNC(=O)c2nccnc2NCC2CCN(C)CC2)c1F ; "C23H27F3N8O2"] | 33.22 |
| 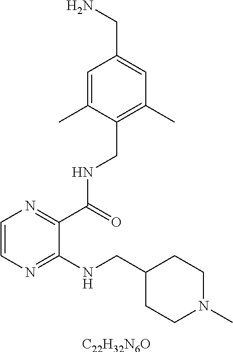 [SMILES: Cc1cc(CN)cc(C)c1CNC(=O)c1nccnc1NCC1CCN(C)CC1 ; "C22H32N6O"] | 33.23 |

The compounds of the invention can be preferably selected from examples: 1.51, 4.09, 4.19, 1.13, 1.25, 1.28, 1.49, 1.5, 1.52, 1.53, 1.54, 1.55, 1.56, 1.59, 1.63, 1.64, 1.68, 1.71, 4.02, 4.03, 4.07, 4.1, 4.11, 4.13, 4.16, 4.18, 4.2, 4.21, 4.23, 4.24, 4.25, 33.18; and pharmaceutically acceptable salts and/or solvates thereof. In particular, the compounds of the invention can be selected from examples: 1.51, 4.09, 4.19; and pharmaceutically acceptable salts and/or solvates thereof.

The compounds of the invention can be selected from Table 1, and pharmaceutically acceptable salts and/or solvates thereof.

The compounds of the invention can be selected from Table 2, and pharmaceutically acceptable salts and/or solvates thereof.

The compounds of the invention can be selected from Table 3, and pharmaceutically acceptable salts and/or solvates thereof.

The compounds of the invention can be selected from Table 4, and pharmaceutically acceptable salts and/or solvates thereof.

The compounds of the invention can be selected from Table 5, and pharmaceutically acceptable salts and/or solvates thereof.

The compounds of the invention can be selected from Table 6, and pharmaceutically acceptable salts and/or solvates thereof.

The compounds of the invention can be selected from Table 7, and pharmaceutically acceptable salts and/or solvates thereof.

The compounds of the invention can be selected from Table 8, and pharmaceutically acceptable salts and/or solvates thereof.

The compounds of the invention can be selected from Table 9, and pharmaceutically acceptable salts and/or solvates thereof.

The compounds of the invention can be selected from Table 10, and pharmaceutically acceptable salts and/or solvates thereof.

The compounds of the invention can be selected from Table 11, and pharmaceutically acceptable salts and/or solvates thereof.

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A and by S. Patel in Retina, 2009 June; 29 (6 Suppl): S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®) and lanadelumab (Takhzyro®); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor VIIa inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, n can be 0, 1, or 2. n is preferable 1.

As noted above, “alkoxy” is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro. Examples of such alkoxy groups include, but are not limited to, C1-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted aboves. More specifically, alkoxy can be linear groups of between 1 and 4 carbon atoms (C₁-C₄), more specifically, between 1 and 3 carbon atoms (C₁-C₃). More specifically, alkoxy can be branched groups of between 3 and 4 carbon atoms (C₃-C₄), optionally substituted as noted above.

As noted above, “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, and heterocyclyl^{b}. As noted above, “alkyl^{b}” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂, —NHCOCH₃, CF₃, halo, oxo, heterocyclyl^{b}, and cyclopropane. Examples of such alkyl or alkyl^{b} groups include, but are not limited, to C₁-methyl, C₂-ethyl, C₃-propyl and C₄-n-butyl, C₃-iso-propyl, C₄-sec-butyl, C₄-iso-butyl, C₄-tert-butyl and C₅-neo-pentyl), optionally substituted as noted above. More specifically, “alkyl” or “alkyl^{b}” can be a linear saturated hydrocarbon having up to 6 carbon atoms (C₁-C₅) or a branched saturated hydrocarbon of between 3 and 6 carbon atoms (C₃-C₅), optionally substituted as noted above. Even more specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b}”, respectively. Preferably, “alkyl” or “alkyl^{b}” can be defined as a “small alkyl” or “small alkyl^{b}”.

As noted above, “alkylene” is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃, and halo. More specifically, alkylene can be a bivalent linear saturated hydrocarbon having 2 to 4 carbon atoms (C₂-C₄), more specifically having 2 to 3 carbon atoms (C₂-C₃), optionally substituted as noted above.

“Aryl” and “aryl^{b}” are defined above. Typically, aryl or aryl^{b} will be optionally substituted with 1, 2 or 3 substituents. Optional substituents are selected from those stated above. Examples of suitable aryl or aryl^{b} groups include phenyl and naphthyl (each optionally substituted as stated above). Preferably aryl is selected from phenyl and substituted phenyl (wherein said substituents are selected from those stated above).

As noted above, “cycloalkyl” is a monocyclic saturated hydrocarbon ring of between 3 and 6 carbon atoms (C₃-C₆); cycloalkyl may optionally be substituted with 1 or 2 substituents independently selected from alkyl^{b}, (C₁-C₅)alkoxy, OH, CN, CF₃, and halo. Examples of suitable monocyclic cycloalkyl groups include cyclopropyl, cyclobutyl, cyclopentyl, and cyclohexyl), optionally substituted as noted above. More specifically, cycloalkyl can be a monocyclic saturated hydrocarbon ring of between 3 and 5 carbon atoms, more specifically, between 3 and 4 carbon atoms), optionally substituted as noted above.

Halo can be selected from Cl, F, Br and I. More specifically, halo can be selected from Cl and F. Preferably, halo is Cl.

As noted above, the term “heteroalkylene” is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₅)alkoxy, OH, CN, CF₃, and halo. More specifically, heteroalkylene can be a valent linear saturated hydrocarbon having 2 to 4 carbon atoms (C₂-C₄), wherein at least one of the 2 to 4 carbon atoms is replaced with NR8, S, or O, or having 2 to 3 carbon atoms (C₂-C₃), wherein at least one of the 2 to 3 carbon atoms is replaced with NR8, S, or O, each optionally substituted as noted above.

“Heteroaryl” and “heteroaryl^{b}” are as defined above. Typically, “heteroaryl” or “heteroaryl^{b}” will be optionally substituted with 1, 2 or 3 substituents. Optional substituents are selected from those stated above. Examples of suitable heteroaryl or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above).

As noted above, “heterocyclyl” is a 4-, 5-, 6-, or 7-membered carbon-containing non-aromatic ring containing one or two ring members that are selected from N, NR8, S, SO, SO₂ and O; heterocyclyl may be optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl. More specifically, “heterocyclyl” can be a 4-, 5-, 6-, or 7-membered carbon-containing non-aromatic ring containing one or two ring members that are selected from N, NR8, and O (optionally substituted in the same manner as “heterocyclyl”).

As noted above, “heterocyclyl^{b}” is a 4-, 5-, 6-, or 7-membered carbon-containing non-aromatic ring containing one or two ring members that are selected from N, NR12, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃. More specifically, “heterocyclyl^{b}” is a 4-, 5-, 6-, or 7-membered carbon-containing non-aromatic ring containing one or two ring members that are selected from N, NR12, and O (optionally substituted in the same manner as “heterocyclyl^{b}”.

The term “O-linked”, such as in “O-linked hydrocarbon residue”, means that the hydrocarbon residue is joined to the remainder of the molecule via an oxygen atom.

The term “N-linked”, such as in “N-linked pyrrolidinyl”, means that the heterocycloalkyl group is joined to the remainder of the molecule via a ring nitrogen atom.

“Triazole” means 1,2,3-triazole and 1,2,4-triazole.

In groups such as —(CH₂)₁₋₃-aryl, “-” denotes the point of attachment of the substituent group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

In the context of the present invention, references herein to “treatment” include references to curative, palliative and prophylactic treatment.

### General Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively where a hydroxy group is protected as a benzyl ether, deprotection conditions comprise hydrogenation with a palladium catalyst under a hydrogen atmosphere.

The compounds according to general formula I can be prepared using conventional synthetic methods for example, but not limited to, the route outlined in Schemes 1-4.

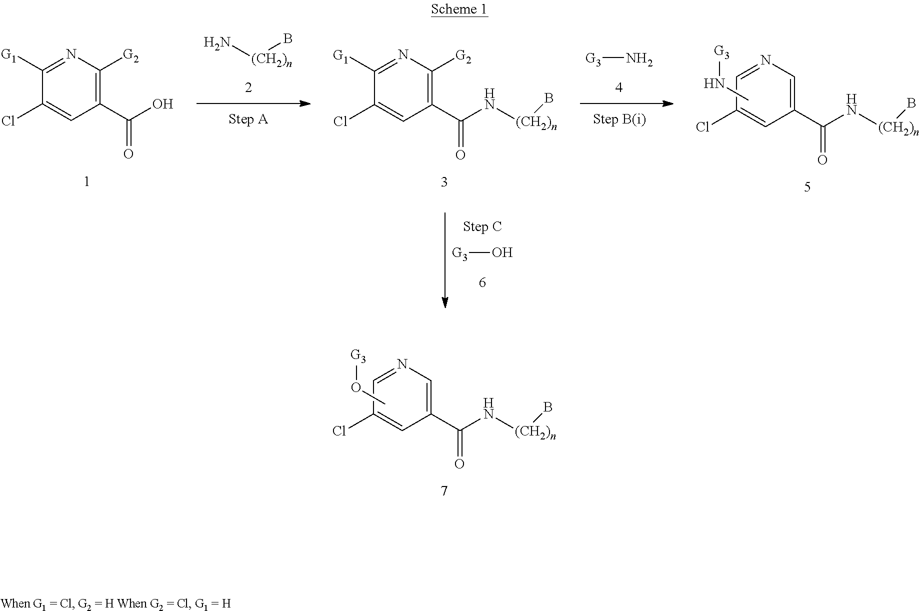

The carboxylic acid 1 is coupled to amine (or salt) 2 (Step A) to give compound 3. This coupling is typically carried out using standard coupling conditions such as hydroxybenzotriazole (HOBt) and carbodiimide such as water soluble carbodiimide in the presence of an organic base. Other standard coupling methods include the reaction of acids with amines in the presence of 2-(1H-benzotriazole-1-yl)-1,1,3,3-tetramethylaminium hexafluorophosphate (HBTU) or benzotriazole-1-yl-oxy-tris-pyrrolidino-phosphonium hexafluorophosphate (PyBOP) or bromo-trispyrolidino-phosphonium hexafluorophosphate (PyBroP) or 2-(3H-[1,2,3]triazolo[4,5-b]pyridin-3-yl)-1,1,3,3-tetramethylisouronium hexafluorophosphate(V) (HATU), or 1-ethyl-3-(3-dimethylaminopropyl)carbodiimide (EDC) or (Propylphosphonic Anhydride (T3P) in the presence of organic bases such as triethylamine, diisopropylethylamine or N-methylmorpholine. Alternatively, the amide formation can take place via an acid chloride in the presence of an organic base. Such acid chlorides can be formed by methods well known in the literature, for example reaction of the acid with oxalyl chloride or thionyl chloride. Alternatively, the carboxylic acid can be activated using 1,1′-carbonyldiimidazole (CDI) and then amine added.

The chloropyridine 3 is reacted with primary or secondary amines 4 in a solvent such as DMSO, typically heating to 100° C. (Step B(i)). Alternatively, the chloropyridine 3 is reacted with an alcohol 6 in the presence of a base such as DBU, or potassium tert-butoxide in a solvent such as DMF or NMP, typically heating to 120° C. (Step C).

Alternatively, the order of steps can be reversed such that the amine substituent can be added earlier in the synthesis prior to the amine coupling as shown in Scheme 2.

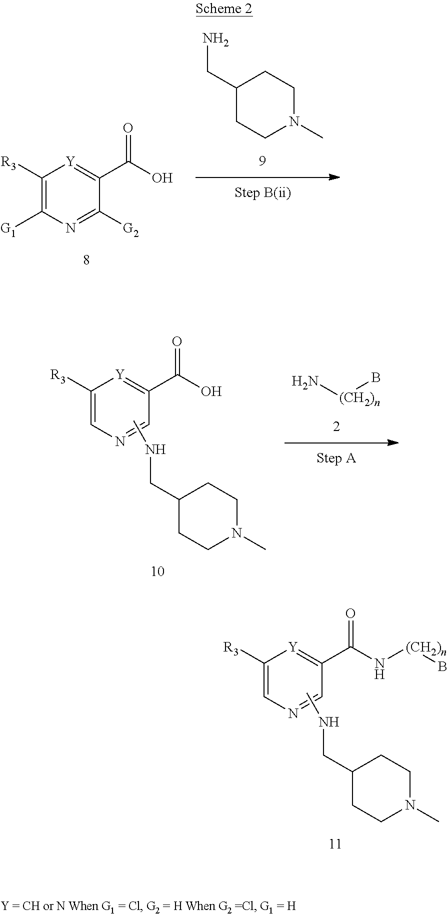

The heteroaryl chloride 8 is reacted with amine 9 under alkylation conditions (Step B(ii)) typically heating to 100° C. in the presence of N,N-diisopropylethylamine, in a solvent such as dioxane. The acid (or salt) 10 is coupled to amine (or salt) 2 (Step A) to give compound 11. This coupling is typically carried out using standard coupling conditions already described.

In both Scheme 1 and 2 the amine 2 may be commercially available or prepared from readily available starting materials using methods known in the art, or as detailed in specific examples herein. Depending on B, the final compound may require removal of protecting groups using methods known in the art.

Examples where the substituent is linked to the central aromatic ring via a carbon can be prepared using conventional synthetic methods for example, but not limited to, the route outlined in Scheme 3.

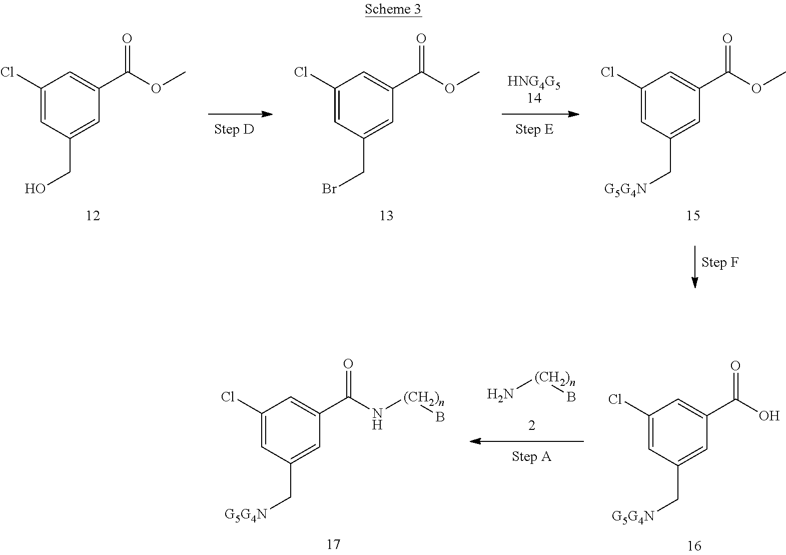

The alcohol 12 is converted to the bromide 13 (Step D). Methods for such transformations are known in the art, for example reaction with N-bromosuccinimide in the presence of triphenylphosphine in a solvent such as tetrahydrofuran. The bromide 13 is reacted with primary or secondary amine 14 (Step E) in the presence of a base such as potassium carbonate in a solvent such as tetrahydrofuran. Alkylation Methods for such transformations are known in the art, for example in the presence of a other bases such as, caesium carbonate, N,N-diisopropylethylamine, triethylamine and in other solvents such as dichloromethane, acetonitrile or dimethylformamide. The ester 15 is hydrolysed (Step F) using standard literature conditions such as NaOH, KOH, or LiOH. This intermediate can be isolated in the form of the salt, such as lithium. The acid (or salt) 16 is coupled to amine (or salt) 2 (Step A) to give compound 17. This coupling is typically carried out using standard coupling conditions already described.

In some circumstances, the substituent can also be added to the heterocyle via a Suzuki reaction as shown in Scheme 4.

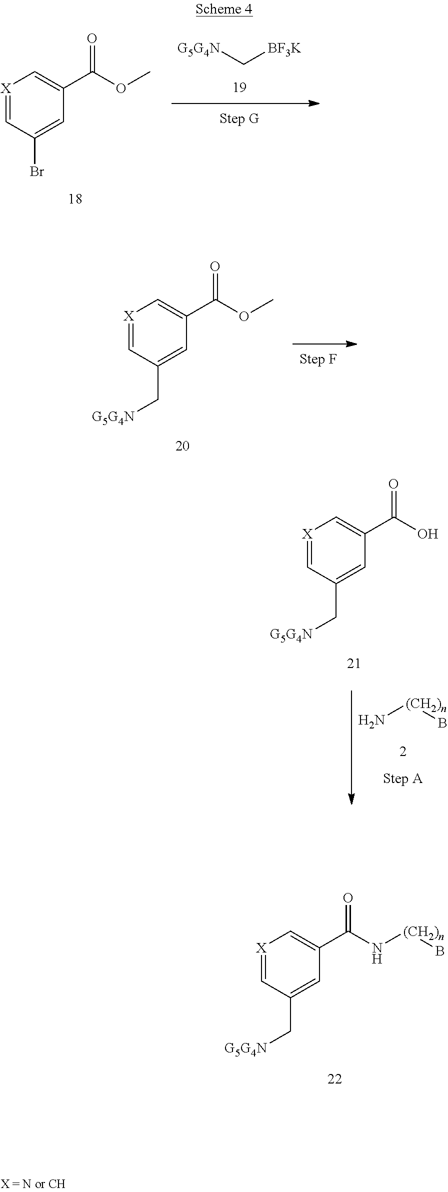

The heteroaryl bromide 18 is reacted with an organoboron compound 19, such as a potassium trifluoroborate or boronic acid under typical Suzuki-Miyaura coupling conditions (Step G). This is a versatile transformation where those skilled in the art would readily be able to select a suitable ligand, catalyst and organoboron reagent with respect to the desired compound. Typically X-Phos, or S-Phos, palladium acetate or tris(dibenzylideneacetone)dipalladum(0) and caesium carbonate are required. The ester can then undergo hydrolysis (Step F) and amide coupling (Step A) under the general conditions.

### EXAMPLES

The invention is illustrated by the following non-limiting examples in which the following abbreviations and definitions are used:

| | |
|---|---|
| Aq | Aqueous solution |
| AIBN | Azobisisobutyronitrile |
| tBu | Tert-Butyl |
| CDI | 1,1′-Carbonyldiimidazole |
| DCM | Dichloromethane |
| DIPEA | N,N-Diisopropylethylamine |
| DMF | N,N-Dimethylformamide |
| DMSO | Dimethyl sulfoxide |
| eq | Equivalent |
| Et₂O | Diethyl ether |
| Et | Ethyl |
| EtOH | Ethanol |
| EtOAc | Ethyl Acetate |
| HATU | 2-(3H-[1,2,3]triazolo[4,5-b]pyridin-3-yl)-1,1,3,3- |
| | tetramethylisouronium hexafluorophosphate(V) |
| hrs | Hours |
| HOBt | Hydroxybenzotriazole |
| IPA | Isopropyl alcohol |
| LCMS | Liquid chromatography mass spectrometry |
| Me | Methyl |
| MeCN | Acetonitrile |
| MsCl | Methanesulfonyl chloride |
| MeOH | Methanol |
| min | Minutes |
| MS | Mass spectrum |
| Ms | Methanesulfonyl |
| NMR | Nuclear magnetic resonance spectrum |
| NMP | N-Methyl-2-pyrrolidone |
| Pet. Ether | Petroleum ether fraction boiling at 60-80° C. |
| Ph | Phenyl |
| iPr | Iso-propyl |
| nPr | n-Propyl |
| SWFI | Sterile water for injection |
| rt | room temperature |
| T3P | Propylphosphonic anhydride |
| TBDMS | tert-Butyldimethylsilyl |
| TBME | tert-Butyl methyl ether |
| THF | Tetrahydrofuran |
| TEA | Triethylamine |
| TFA | Trifluoroacetic acid |

All reactions were carried out under an atmosphere of nitrogen unless specified otherwise.

¹H NMR spectra were recorded on a Bruker (500 MHz or 400 MHz) spectrometer and reported as chemical shift (ppm).

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO2H/MeCN into 0.1% HCO2H/H2O over 3 minutes, flow rate 1 mL/min. Data was collected using a Waters Acquity UPLC mass spectrometer with quadropole dalton, photodiode array and electrospray ionisation detectors.

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as ChemDraw (PerkinElmer) or the Autonom software provided as part of the ISIS Draw package from MDL Information Systems or the Chemaxon software provided as a component of MarvinSketch or as a component of the IDBS E-WorkBook.

### Synthesis of Intermediates

### General Method A: Amide Formation

### (i) Coupling Reagent, Eg HATU

### N-((1-aminoisoquinolin-6-yl)methyl)-2,5-dichloronicotinamide

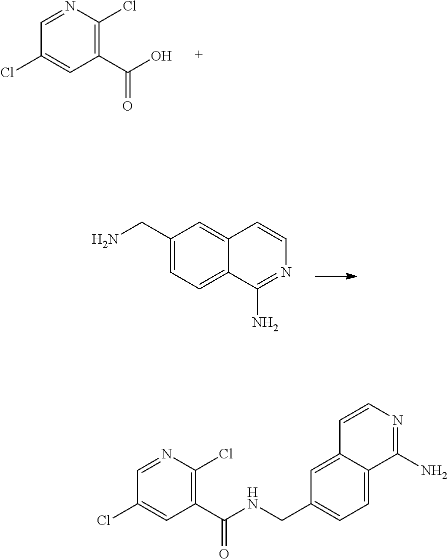 [SMILES: O=C(O)c1cc(Cl)cnc1Cl ; NCc1ccc2c(N)nccc2c1 ; Nc1nccc2cc(CNC(=O)c3cc(Cl)cnc3Cl)ccc12]

To a solution of 6-(aminomethyl)isoquinoline-1-amine dihydrochloride (1.05 g, 4.27 mmol) and 2,5-dichloropyridine-3-carboxylic acid (0.63 g, 3.28 mmol) in dry DMF (10 mL), was added N,N-diisopropylethylamine (2.29 mL, 13.1 mmol). The resulting suspension was stirred at rt for 15 min, then cooled to 0° C. and HATU (1.87 g, 4.92 mmol) added portion-wise over 5 min. The reaction was stirred at rt for 18 hrs. The reaction mixture was partitioned between sat. aq. NaHCO₃ (100 mL) and EtOAc (100 mL). The aqueous layer was extracted with EtOAc (2×50 mL) and the combined organic extracts washed sequentially with water (5×20 mL) and brine (20 mL). The organic layer was and dried (MgSO₄), filtered and concentrated in vacuo. The residue was purified by flash chromatography (4-8% (1% NH₃ in MeOH) in DCM) to afford the title compound (85 mg, 73% yield) as a pale yellow solid. [M+H]⁺=347.3

¹H NMR (DMSO-d6): 4.60 (2H, d, J=5.9 Hz), 6.77 (2H, s), 6.88 (1H, d, J=5.8 Hz), 7.45 (1H, dd, J=1.8, 8.6 Hz), 7.64 (1H, d, J=1.7 Hz), 7.78 (1H, d, J=5.8 Hz); 8.17 (1H, d, J=8.6 Hz); 8.26 (1H, d, J=2.6 Hz); 8.60 (1H, d, J=2.6 Hz); 9.30 (1H, t, J=5.9 Hz).

### General Method A: Amide Formation

### (ii) Coupling Reagent, Eg HOBt

### Example 19.03

### 1H-Pyrrolo[2,3-b]pyridine-5-carboxylic acid (1-amino-isoquinolin-6-ylmethyl)-amide

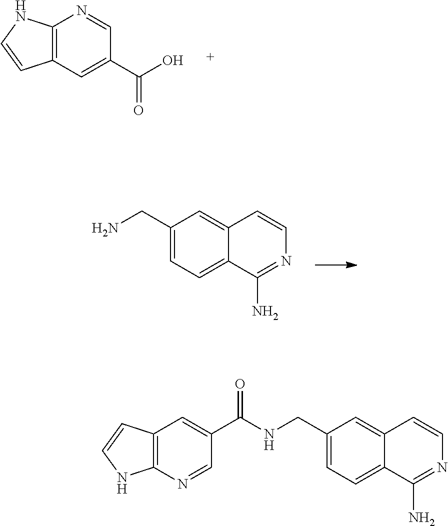 [SMILES: O=C(O)c1cnc2[nH]ccc2c1 ; NCc1ccc2c(N)nccc2c1 ; Nc1nccc2cc(CNC(=O)c3cnc4[nH]ccc4c3)ccc12]

1H-pyrrol[2,3-b]-5-carboxylic acid (50 mg, 0.31 mmol) and 6-(aminomethyl)isoquinoline-1-amine (53 mg, 0.31 mmol) were combined and taken up in DCM at 0° C. and treated with HOBt (50 mg, 0.37 mmol), triethylamine (215 μL, 1.54 mmol) and EDC (83 mg, 0.43 mmol). The reaction was warmed to rt and stirred at rt for 24 hrs. Reaction was diluted with CHCl₃ (50 mL) and washed with minimum sat. aq. NaHCO₃ (10 mL) and concentrated in vacuo. Flash chromatography (0-100% (10% NH₃ in MeOH) in DCM) afforded the title compound (39 mg, 40% yield) as a yellow solid.

[M+H]⁺=317.9

¹H NMR (DMSO): 4.64 (2H, d, J=5.8 Hz), 6.57 (1H, dd, J=3.4, 1.8 Hz), 6.80 (2H, br.s), 6.88 (1H, d, J=5.8 Hz), 7.45 (1H, dd, J=8.6, 1.6 Hz), 7.57 (1H, t, J=2.9 Hz), 7.60 (1H, s), 7.75 (1H, d, J=5.9 Hz), 8.16 (1H, d, J=8.6 Hz), 8.51 (1H, d, J=2.0 Hz), 8.79 (1H, d, J=2.0 Hz), 9.15 (1H, t, J=5.9 Hz), 11.92 (1H, s)

### General Method A: Amide Formation

### (iii) Coupling Reagent, Eg Propylphosphonic Anhydride (T3P)

### tert-Butyl (3,5-dimethyl-4-((3-(((1-methylpiperidin-4-yl)methyl)amino)pyrazine-2-carboxamido)methyl)benzyl)carbamate

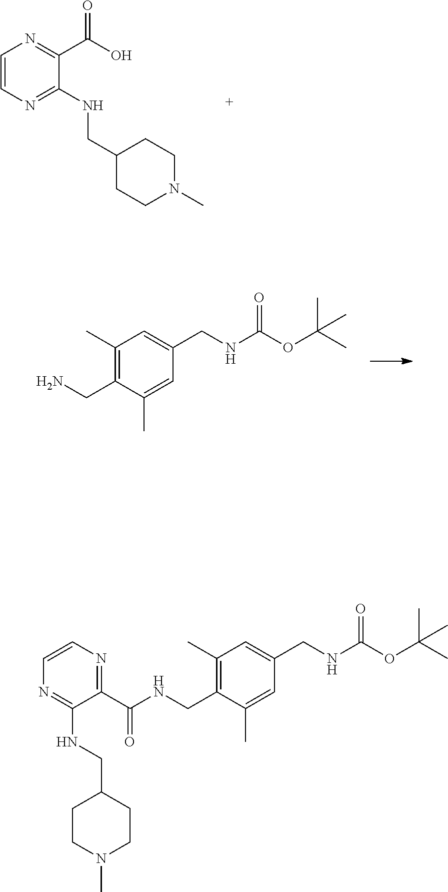 [SMILES: CN1CCC(CNc2nccnc2C(=O)O)CC1 ; Cc1cc(CNC(=O)OC(C)(C)C)cc(C)c1CN ; Cc1cc(CNC(=O)OC(C)(C)C)cc(C)c1CNC(=O)c1nccnc1NCC1CCN(C)CC1]

A solution of 3-(((1-methylpiperidin-4-yl)methyl)amino)pyrazine-2-carboxylic acid hydrochloride salt (100 mg, 0.25 mmol), DIPEA (250 μL, 1.44 mmol) and T3P (50% wt. in DMF) (400 μL, 0.55 mmol) in DMF (0.5 mL) was stirred at rt for 10 min. tert-Butyl (4-(aminomethyl)-3,5-dimethylbenzyl)carbamate (synthesis reported in WO2014108679, CAS 1618647-97-4) (23 mg, 0.09 mmol) was added and the reaction mixture was then stirred at rt for 64 hrs. The reaction mixture was concentrated and product purified by preparative HPLC (20-50% MeCN in water) to afford the title compound (11 mg, 9% yield) as a colourless gum.

### General Method B (i): Aryl CN Formation

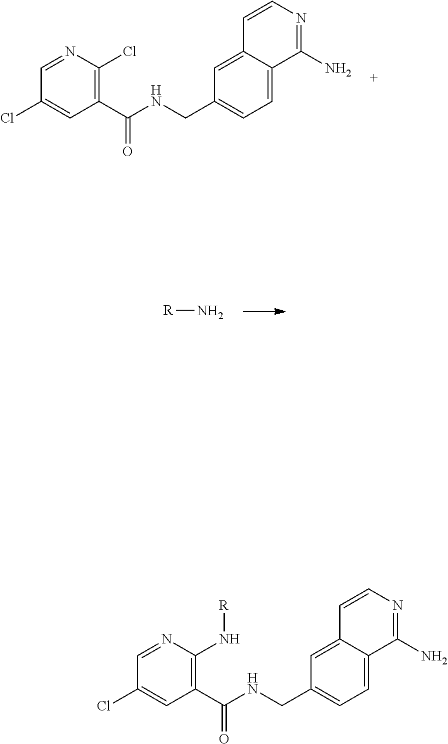

To a solution of N-((1-aminoisoquinolin-6-yl)methyl)-2,5-dichloronicotinamide (35 mg, 0.1 mmol) in DMSO (0.75 mL) was added the required amine (0.3 mmol). The resulting mixture was then heated to 100° C. for 24 hrs. The reaction was cooled to rt and the crude product purified by preparative HPLC. The solvent was removed and the solids lyophilised with MeCN:Water to afford the desired compounds.

### General Method B (ii): Aryl CN Formation

### 3-(((1-Methylpiperidin-4-yl)methyl)amino)pyrazine-2-carboxylic acid

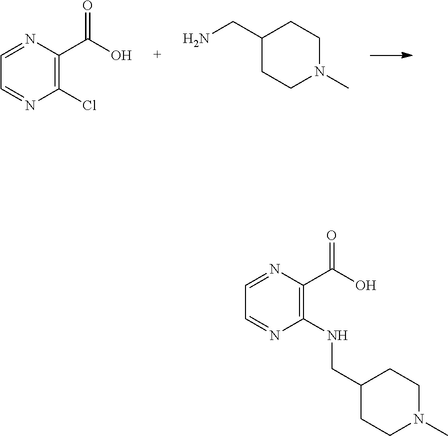

To a solution of 3-chloropyrazine-2-carboxylic acid (5.0 g, 31.4 mmol) and DIPEA (27.5 mL, 157.7 mmol) in dioxane (20 mL) was added (1-methyl-4-piperidinyl)methanamine (4.25 g, 33.1 mmol). The reaction was heated to 100° C. for 18 hrs. The reaction mixture was concentrated in vacuo and purified by reverse phase flash chromatography (5-40% MeCN in (0.1% formic acid in water)). The title compound was isolated (4.06 g, 51% yield) as a white solid.

[M+H]+=251.2

### General Method C: Aryl CO Formation

### (i) For Phenols

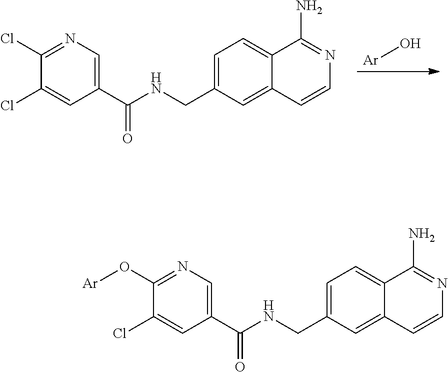

To a generic phenol (0.2 mmol) was added a solution of N-((1-aminoisoquinolin-6-yl)methyl)-5,6-dichloronicotinamide (54.7 mg, 0.1 mmol) in dry DMF (750 μL) followed by DBU (0.033 mL, 0.22 mmol). The reaction was sealed and heated to 120° C. for 18 hrs. The crude product was purified by preparative H PLC.

### General Method C: Aryl CO Formation

### (ii) For Alcohols

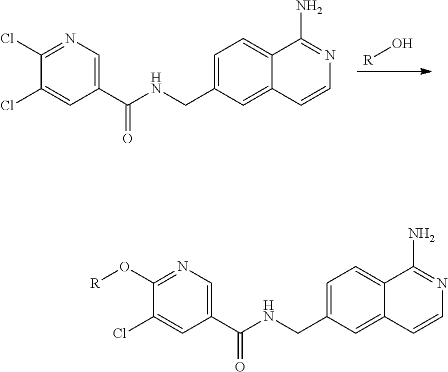

Wells were charged with a generic alcohol (0.2 mmol) and dissolved in NMP (0.5 mL). tert-Butoxypotassium (1.0 M in THF) (0.22 mL, 0.22 mmol) was added to the wells. These were manually mixed then left at rt for 5 min. A solution of N-[(1-amino-6-isoquinolyl)methyl]-2,5-dichloropyridine-3-carboxamide (34.7 mg, 0.1 mmol) in dry NMP (400 μL) was then added to the wells, which were manually mixed then shaken at rt for 2 days. The wells were quenched with acetic acid (0.0172 mL, 0.3 mmol) and the reactions were filtered. The crude products were purified by preparative HPLC.

### General Method D: Bromination

### Methyl 3-(bromomethyl)-5-chlorobenzoate

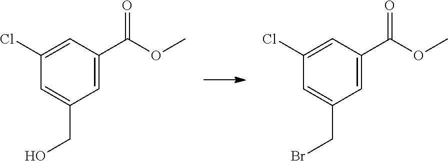

A solution of methyl 3-chloro-5-(hydroxymethyl)benzoate (250 mg, 1.25 mmol) and triphenylphosphine (700 mg, 2.67 mmol) in THE (3 mL) was protected from the light. The reaction mixture was cooled to 0° C. and NBS (450 mg, 2.53 mmol) added in one portion then warmed to rt and stirred for 18 hrs. The reaction mixture was diluted with EtOAc (30 mL) and washed with sat. aq. NaHCO₃ (30 mL) and brine (30 mL) before the organic phase was dried over MgSO₄, filtered and concentrated. The crude product was purified by flash chromatography (0-50% EtOAc in isohexane) to afford the title compound (83 mg, 25% yield) as a colourless glass.

¹H NMR (500 MHz, DMSO-d6) δ 3.89 (s, 3H), 4.80 (s, 2H), 7.85-7.88 (m, 2H), 7.98-8.04 (m, 1H).

### General Method E: N-alkylation (K₂CO₃)

### Methyl 3-(((1-(2-hydroxyethyl)piperidin-4-yl)methyl)amino)pyrazine-2-carboxylate

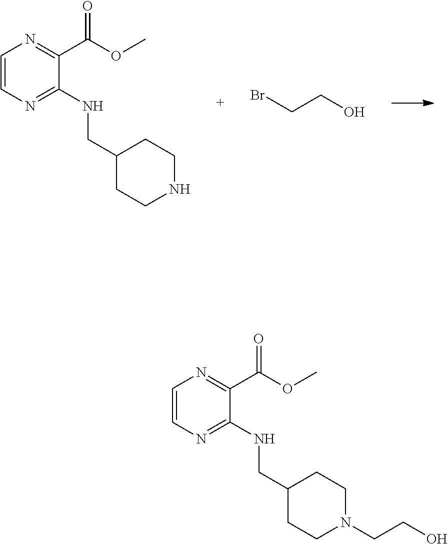

Methyl 3-((piperidin-4-ylmethyl)amino)pyrazine-2-carboxylate (146 mg, 0.58 mmol) was dissolved in dry MeCN (10 mL) then 2-bromoethanol (0.26 mL, 1.75 mmol) and potassium carbonate (161 mg, 1.17 mmol) were added. The reaction mixture was stirred at rt for 18 hrs. The reaction mixture was concentrated in vacuo and the residue partitioned between water (20 mL) and ethyl acetate (25 mL). The organic extract was dried (MgSO₄), filtered and concentrated. The crude was purified by flash chromatography (SCX, 2M NH₃/MeOH) to afford the title compound as a colourless oil (128 mg, 74% yield).

[M]⁺=294.8

### General Method F: Hydrolysis of an ester to a carboxylic acid

### (i) LiOH

### 5-((4-(Cyclopentylmethyl)piperazin-1-yl)methyl)nicotinic acid

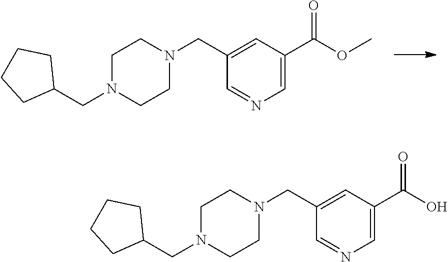 [SMILES: COC(=O)c1cncc(CN2CCN(CC3CCCC3)CC2)c1 ; O=C(O)c1cncc(CN2CCN(CC3CCCC3)CC2)c1]

To a stirred solution of methyl 5-((4-(cyclopentylmethyl)piperazin-1-yl)methyl)nicotinate (171 mg, 0.54 mmol) in THF (4 mL) and water (2 mL) at rt was added lithium hydroxide (64.5 mg, 2.69 mmol). The resulting solution was stirred at rt for 18 hrs. The reaction mixture was purified by flash chromatography (SCX, 1% NH₃ in MecOH). Solvent was evaporated under reduced pressure to afford the title compound as a white powder (145 mg, 80% yield).

[M+H]⁺=304.1

### General Method F

### (ii) NaOH

### 5-((4-Benzylpiperazin-1-yl)methyl)nicotinic acid

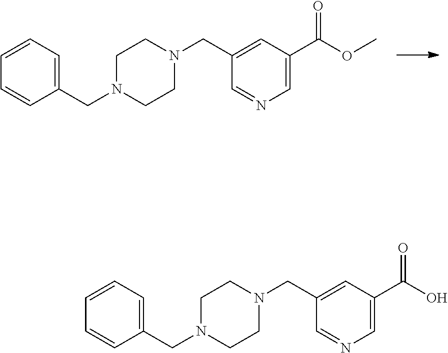 [SMILES: COC(=O)c1cncc(CN2CCN(Cc3ccccc3)CC2)c1 ; O=C(O)c1cncc(CN2CCN(Cc3ccccc3)CC2)c1]

To a stirred solution of methyl 5-((4-benzylpiperazin-1-yl)methyl)nicotinate (130 mg, 0.40 mmol) in THF (3 mL) and MeOH (1 mL) was added 2M NaOH (400 μL, 0.80 mmol). After 90 min at rt the reaction mixture was reduced to half volume under reduced pressure. The crude solution was acidified by addition of acetic acid (0.3 mL) and loaded onto a column of SCX (2 g) in MeCOH. The column was washed with MecOH and then the product was eluted with 0.7M NH₃ in MeOH. The resultant mixture was concentrated in vacuo to afford the title compound as a white powder (122 mg, 95% yield).

[M+H]⁺=312.3

NMR (d6-DMSO) δ: 2.40 (8H, br, m), 3.46 (2H, s), 3.57 (2H, s), 7.21-7.35 (5H, m) 8.12-8.17 (1H, m), 8.64 (1H, d, J=2.1 Hz), 8.94 (1H, d, J=2.0 Hz).

### General Method G: Suzuki

### Tert-butyl 4-((5-(methoxycarbonyl)pyridin-3-yl)methyl)piperazine-1-carboxylate

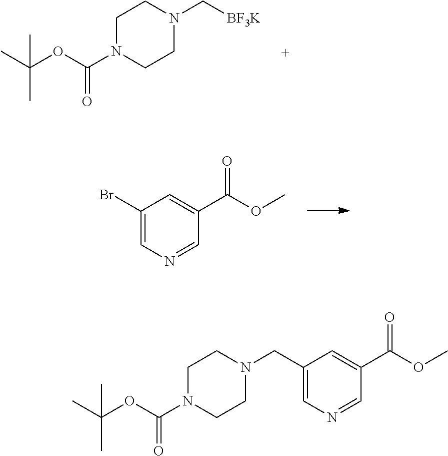

Methyl 5-bromonicotinate (0.588 g, 2.72 mmol), diacetoxypalladium (0.031 g, 0.136 mmol), potassium (4-Boc-piperazin-1-yl)methyltrifluoroborate (1 g, 3.27 mmol), caesium carbonate (2.22 g, 6.80 mmol), and X-Phos (0.130 g, 0.27 mmol) were dissolved in THF (8 mL) and water (2 mL) was added. The resulting mixture was stirred and heated at 70° C. for 18 hrs under a N₂ atmosphere. The mixture was diluted with water (10 mL) and extracted with EtOAc (3×10 mL). The combined organics were dried (MgSO₄), filtered and concentrated. The residue was purified by flash chromatography (0-100% (1% Et₃N/EtOAc) in isohexane) to afford the title compound (834 mg, 82% yield) as a light brown solid.

[M+H]⁺=336.1

### General Method H: Boc Deprotection

### [1-[(2-Methylpyrazol-3-yl)methyl]-4-piperidyl]methanamine

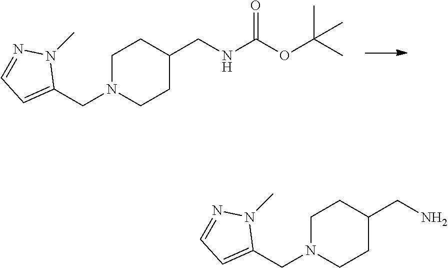 [SMILES: Cn1nccc1CN1CCC(CNC(=O)OC(C)(C)C)CC1 ; Cn1nccc1CN1CCC(CN)CC1]

tert-Butyl N-[[1-[(2-methylpyrazol-3-yl)methyl]-4-piperidyl]methyl]carbamate (95 mg, 0.31 mmol) was dissolved in DCM (1.64 mL) before trifluoroacetic acid (0.024 mL, 0.31 mmol) was added. The reaction was stirred at rt for 3 hrs. The solvent was removed in vacuo. Flash chromatography (SCX, 1.5M NH₃ in MecOH) afforded the title compound (62.5 mg, 97% yield).

### General Method I: Reductive Amination

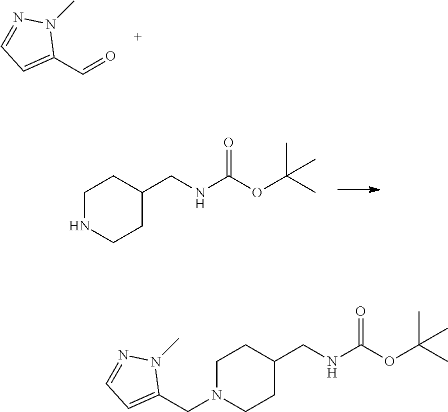

2-Methylpyrazole-3-carbaldehyde (100 mg, 0.91 mmol) and tert-butyl N-(4-piperidylmethyl) carbamate (214 mg, 1.0 mmol) were dissolved in DCE (6.05 mL) under N₂. Acetic acid (327 mg, 5.45 mmol) was added and the reaction stirred at rt for 30 min, sodium triacetoxyborahydride (577 mg, 2.72 mmol) was then added and the reaction stirred at rt for 18 hrs. The reaction was diluted with DCM (3 mL) and then quenched with 1M NaOH (2 mL). The layers were separated and the aqueous phase further extracted with DCM (3×10 mL). The combined organic layers were dried (MgSO₄), filtered and concentrated in vacuo to afford the title compound (95 mg, 28% yield) as a yellow oil.

### INTERMEDIATES

### N-[(1-amino-7-isoquinolyl)methyl]-2,5-dichloro-pyridine-3-carboxamide

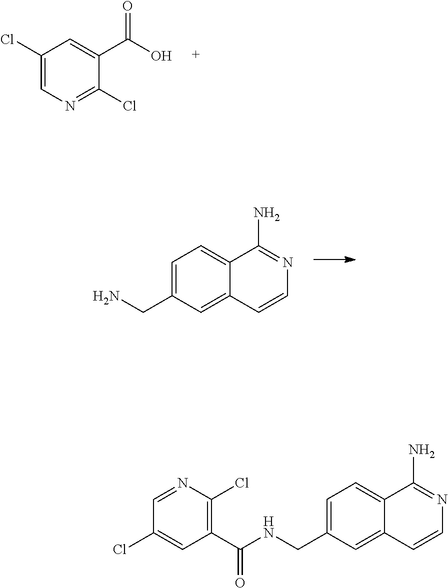 [SMILES: O=C(O)c1cc(Cl)cnc1Cl ; NCc1ccc2c(N)nccc2c1 ; Nc1nccc2cc(CNC(=O)c3cc(Cl)cnc3Cl)ccc12]

Following general conditions A(ii), 6-(aminomethyl)isoquinolin-1-amine dihydrochloride (1.05 g, 4.27 mmol) was reacted with 2,5-dichloropyridine-3-carboxylic acid (0.63 g, 3.28 mmol) to afford the title compound (0.85 g, 73% yield) as a pale yellow solid.

[M+H]⁺=347.3

¹H NMR (DMSO-d6) ppm: 4.60 (2H, d, J=5.9 Hz), 6.77 (2H, s), 6.88 (1H, d, J=5.8 Hz), 7.45 (1H, dd, J=1.8, 8.6 Hz), 7.64 (1H, d, J=1.7 Hz), 7.78 (1H, d, J=5.8 Hz), 8.17 (1H, d, J=8.6 Hz), 8.26 (1H, d, J=2.6 Hz), 8.60 (1H, d, J=2.6 Hz), 9.30 (1H, t, J=5.9 Hz)

### N-((1-aminoisoquinolin-6-yl)methyl)-5,6-dichloronicotinamide

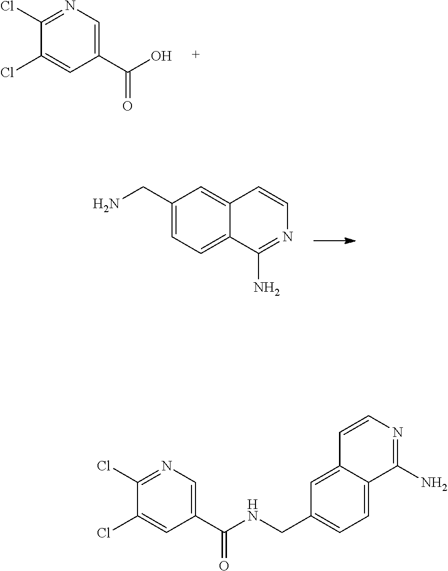 [SMILES: O=C(O)c1cnc(Cl)c(Cl)c1 ; NCc1ccc2c(N)nccc2c1 ; Nc1nccc2cc(CNC(=O)c3cnc(Cl)c(Cl)c3)ccc12]

Following general method A, 6-(aminomethyl)isoquinoline-1-amine dihydrochloride (0.833 g, 3.39 mmol) was reacted with 5,6-dichloropyridine-3-carboxylic acid (0.5 g, 2.6 mmol). The title compound was isolated (0.78 g, 86% yield) as a pale yellow solid.

[M+H]+=347.2

¹H NMR (DMSO, 400 MHz) 54.64 (2H, d, J=5.8 Hz), 6.83 (2H, s), 6.88 (1H, d, J=5.8 Hz), 7.43 (1H, dd, J=8.6, 1.8 Hz), 7.61 (1H, d, J=1.6 Hz), 7.76 (1H, d, J=5.9 Hz), 8.16 (1H, d, J=8.6 Hz), 8.56 (1H, d, J=2.1 Hz), 8.87 (1H, d, J=2.1 Hz), 9.46 (1H, t, J=5.9 Hz)

### tert-Butyl N-[[1-(3-pyridylmethyl)-4-piperidyl]methyl]carbamate

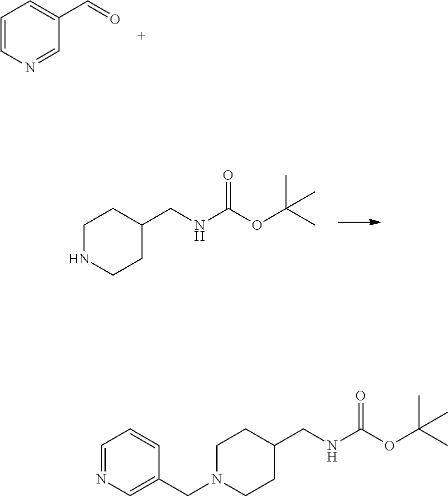 [SMILES: O=Cc1cccnc1 ; CC(C)(C)OC(=O)NCC1CCNCC1 ; CC(C)(C)OC(=O)NCC1CCN(Cc2cccnc2)CC1]

Following general method I, tert-butyl N-(4-piperidylmethyl)carbamate (400 mg, 1.87 mmol) was reacted with pyridine-3-carbaldehyde (200 mg, 1.87 mmol), to afford the title compound (570 mg, 99% yield) as a colourless oil.

[M+H]+=306

¹H NMR (DMSO, 400 MHz) δ 1.09 (2H, qd, J=12.0, 3.8 Hz), 1.37 (10H, s), 1.57 (2H, dd, J=12.9, 3.5 Hz), 1.89 (2H, td, J=11.5, 2.4 Hz), 2.71-2.83 (4H, m), 3.46 (2H, s), 6.81 (1H, t, J=5.9 Hz), 7.34 (1H, dd, J=7.8, 4.7 Hz), 7.68 (1H, dt, J=7.8, 2.0 Hz), 8.40-8.53 (2H, m)

### [1-(3-Pyridylmethyl)-4-piperidyl]methanamine

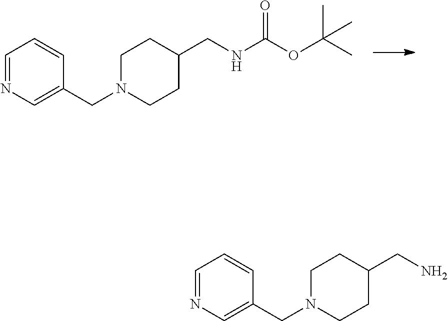 [SMILES: CC(C)(C)OC(=O)NCC1CCN(Cc2cccnc2)CC1 ; NCC1CCN(Cc2cccnc2)CC1]

Following general method H, tert-butyl N-[[1-(3-pyridylmethyl)-4-piperidyl]methyl]carbamate (570 mg, 1.87 mmol) was reacted to afford the title compound (158 mg, 41% yield) as a colourless oil.

[M+H]+=206.1

¹H NMR (DMSO, 400 MHz) δ 1.02-1.25 (3H, m), 1.59-1.69 (2H, m), 1.90 (2H, td, J=11.4, 2.4 Hz), 2.40 (2H, d, J=6.1 Hz), 2.77 (2H, dt, J=11.7, 3.4 Hz), 3.46 (2H, s), 7.34 (1H, dd, J=7.8, 4.7 Hz), 7.69 (1H, dt, J=7.8, 2.0 Hz), 8.37-8.53 (2H, m)

### tert-Butyl N-[[1-(thiazol-4-ylmethyl)-4-piperidyl]methyl]carbamate

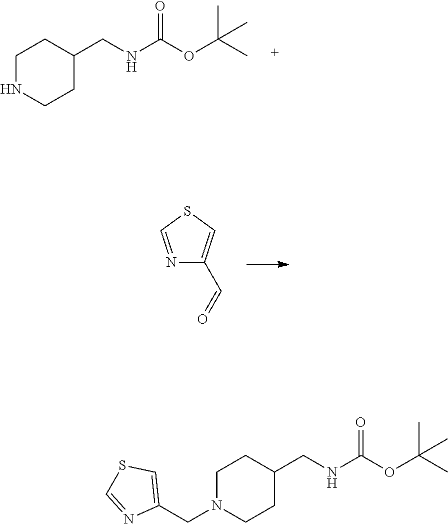 [SMILES: CC(C)(C)OC(=O)NCC1CCNCC1 ; O=Cc1cscn1 ; CC(C)(C)OC(=O)NCC1CCN(Cc2cscn2)CC1]

Following general method I, tert-butyl N-(4-piperidylmethyl)carbamate (104 mg, 0.49 mmol) was reacted with thiazole-4-carbaldehyde (50 mg, 0.44 mmol) to afford the title compound (51 mg, 37% yield).

¹H NMR (400 MHz, Chloroform-d) δ 1.30 (qd, J=12.1, 4.0 Hz, 2H), 1.41 (s, 9H), 1.69-1.60 (m, 2H), 2.03 (td, J=11.6, 2.4 Hz, 2H), 2.94 (dt, J=12.3, 3.9 Hz, 3H), 2.99 (t, J=6.3 Hz, 2H), 3.70 (s, 2H), 4.62 (s, 1H), 7.18 (d, J=2.0 Hz, 1H), 8.75 (d, J=2.0 Hz, 1H)

### [1-(Thiazol-4-ylmethyl)-4-piperidyl]methanamine

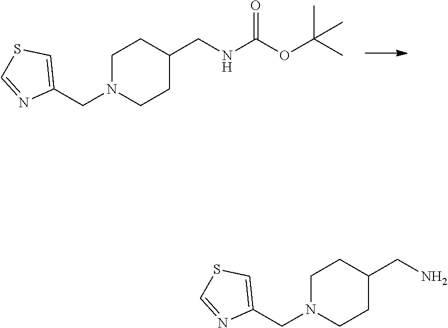 [SMILES: CC(C)(C)OC(=O)NCC1CCN(Cc2cscn2)CC1 ; NCC1CCN(Cc2cscn2)CC1]

tert-Butyl N-[[1-(thiazol-2-ylmethyl)-4-piperidyl]methyl]carbamate (51 mg, 0.16 mmol) was reacted following general method H to afforded the title compound (34 mg, 93% yield) as a white solid.

¹H NMR (400 M Hz, Chloroform-d) δ 8.75 (d, J=2.0 Hz, 1H), 7.16 (d, J=2.1 Hz, 1H), 3.69 (s, 2H), 2.98-2.85 (m, 2H), 2.55 (d, J=5.6 Hz, 2H), 2.06-1.98 (m, 2H), 1.96 (s, 2H), 1.74-1.61 (m, 2H), 1.36-1.19 (m, 3H).

### tert-Butyl N-[[1-(thiazol-2-ylmethyl)-4-piperidyl]methyl]carbamate

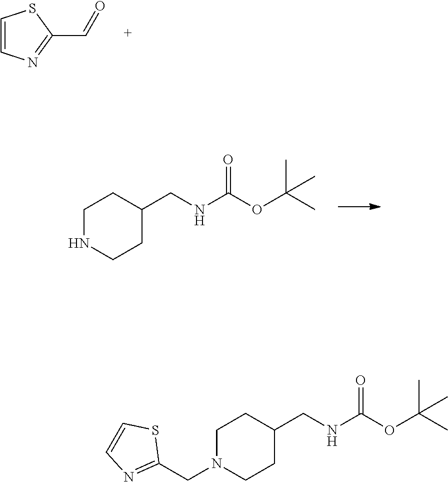 [SMILES: O=Cc1nccs1 ; CC(C)(C)OC(=O)NCC1CCNCC1 ; CC(C)(C)OC(=O)NCC1CCN(Cc2nccs2)CC1]

Following general method I, tert-butyl N-(4-piperidylmethyl)carbamate (104 mg, 0.49 mmol) was reacted with thiazole-2-carbaldehyde (50 mg, 0.44 mmol) to afford the title compound (54 mg, 39% yield) as a yellow oil.

¹H NMR (400 MHz, Chloroform-d) δ 7.64 (d, J=3.3 Hz, 1H), 7.22 (d, J=3.2 Hz, 1H), 4.88 (s, 2H), 3.76 (s, 2H), 2.98-2.83 (m, 4H), 2.06 (td, J=11.6, 2.4 Hz, 2H), 1.65-1.56 (m, 2H), 1.36 (s, 9H), 1.30-1.15 (m, 2H)

### [1-(Thiazol-2-ylmethyl)-4-piperidyl]methanamine

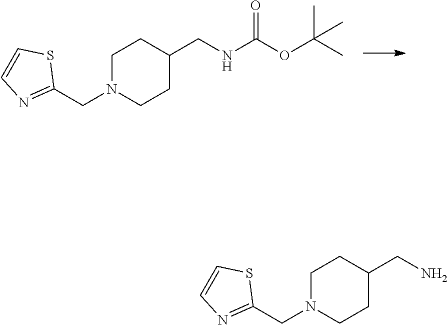 [SMILES: CC(C)(C)OC(=O)NCC1CCN(Cc2nccs2)CC1 ; NCC1CCN(Cc2nccs2)CC1]

Following general method H, tert-butyl N-[[1-(thiazol-2-ylmethyl)-4-piperidyl]methyl]carbamate (51 mg, 0.16 mmol) was reacted to afford the title compound (35 mg, quantitative yield) as a yellow oil.

¹H NMR (400 MHz, Chloroform-d) δ 7.62 (d, J=3.2 Hz, 1H), 7.21 (d, J=3.3 Hz, 1H), 3.77 (s, 2H), 2.94-2.85 (m, 2H), 2.66 (s, 2H), 2.51 (d, J=5.6 Hz, 2H), 2.07 (td, J=11.2, 2.4 Hz, 2H), 1.62 (s, 2H), 1.28-1.14 (m, 3H)

### tert-Butyl N-[[1-(4-pyridylmethyl)-4-piperidyl]methyl]carbamate

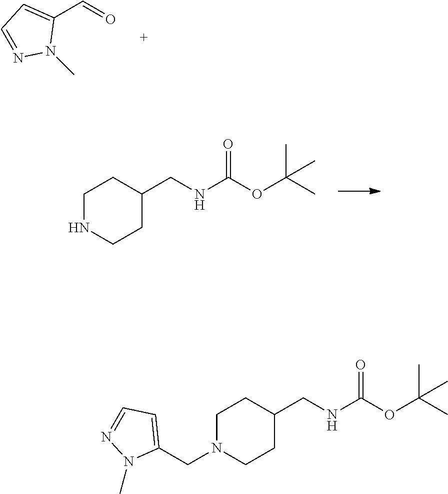

Following general method I, tert-butyl N-(4-piperidylmethyl)carbamate (400 mg, 1.87 mmol) was reacted with pyridine-4-carbaldehyde (200 mg, 1.87 mmol) to afford the title compound (488 mg, 85% yield) as a yellow oil.

[M+H]⁺=306.2

¹H NMR (DMSO, 400 MHz) δ 1.06-1.21 (2H, m), 1.37 (10H, s), 1.58 (2H, dd, J=13.0, 3.3 Hz), 1.91 (2H, tt, J=11.6, 2.3 Hz), 2.72-2.84 (4H, m), 3.47 (2H, s), 6.82 (1H, t, J=5.9 Hz), 7.21-7.37 (2H, m), 8.41-8.57 (2H, m).

### [1-[(2-Methylpyrazol-3-yl)methyl]-4-piperidyl]methanamine

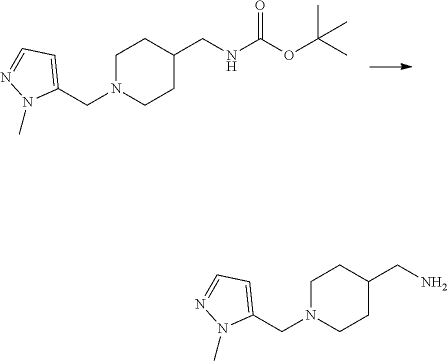 [SMILES: Cn1nccc1CN1CCC(CNC(=O)OC(C)(C)C)CC1 ; Cn1nccc1CN1CCC(CN)CC1]

Following general method H, tert-Butyl N-[[1-[(2-methylpyrazol-3-yl)methyl]-4-piperidyl]methyl]carbamate (95 mg, 0.31 mmol) was reacted to afford the title compound (62.5 mg, 97% yield).

¹H NMR (400 MHz, Chloroform-d) 1.20 (2H, qd, J 12.0, 3.9), 1.30-1.49 (1H, m), 1.71 (2H, d, J 13.1), 1.95 (2H, td, J 11.6, 2.5), 2.52 (2H, br s.), 2.63 (2H, d, J 6.6), 2.78-2.91 (2H, m), 3.47 (2H, s), 3.88 (3H, s), 6.10 (1H, d, J 1.8), 7.37 (1H, d, J 1.8)

### tert-Butyl N-[[1-[(1-methylpyrazol-4-yl)methyl]-4-piperidyl]methyl]carbamate

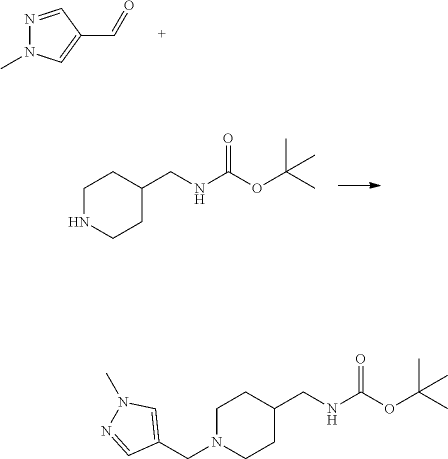

1-Methylpyrazole-4-carbaldehyde (100 mg, 0.91 mmol) was reacted with tert-butyl N-(4-piperidylmethyl)carbamate (195 mg, 0.91 mmol) according to general method I to afford the title compound (298 mg, 97% yield).

### [1-[(1-methylpyrazol-4-yl)methyl]-4-piperidyl]methanamine

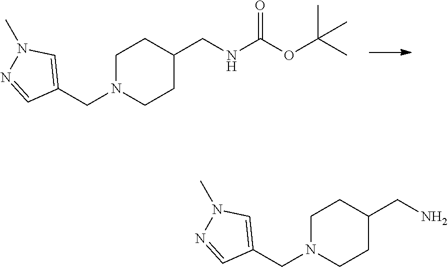 [SMILES: Cn1cc(CN2CCC(CNC(=O)OC(C)(C)C)CC2)cn1 ; Cn1cc(CN2CCC(CN)CC2)cn1]

tert-Butyl N-[[1-[(1-methylpyrazol-4-yl)methyl]-4-piperidyl]methyl]carbamate (298 mg, 0.97 mmol) was reacted following general method H to afford the title compound (178 mg, 82% yield).

¹H NMR (400 MHz, Chloroform-d) δ 7.38 (d, J=0.7 Hz, 1H), 7.33 (s, 1H), 3.87 (s, 3H), 3.44 (s, 2H), 3.02-2.90 (m, 2H), 2.57 (d, J=5.5 Hz, 2H), 1.96 (t, J=11.4 Hz, 2H), 1.79 (d, J=5.8 Hz, 2H), 1.72 (d, J=9.7 Hz, 2H), 1.34-1.24 (m, 3H).

### Potassium(methyl)benzylpiperazinetrifluoroborate hydrobromide

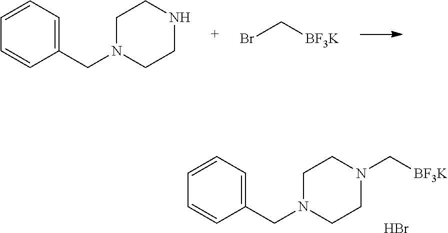

Potassium(bromomethyl)trifluoroborate (692 mg, 3.45 mmol) was added to a solution of 1-benzylpiperazine (638 mg, 3.62 mmol) in anhydrous THF (7 mL) and the resulting suspension heated to 75° C. for 5 hrs. Solvents were removed under vacuum and the residue suspended in a mixture of acetone (150 mL) and potassium carbonate (476 mg, 3.45 mmol). After stirring at ambient temperature for 30 minutes, the mixture was filtered through a pad of Celite and concentrated under vacuum. The residue was dissolved in a minimum quantity of hot acetone (20 mL) and Et₂O added slowly (35 mL), leading to precipitation of product. The product was filtered and dried under vacuum to afford the title compound (553 mg, 42% yield) as a white powder.

[M−H]⁻=257.0

### 1-Isopropyl-3-carbonyl chloride

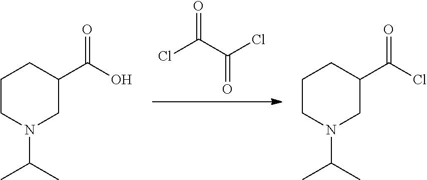

To a stirred suspension of 1-isopropylpiperidine-3-carboxylic acid (100 mg, 0.58 mmol) in DCM (2 mL) was added oxalyl chloride (148 mg, 1.17 mmol). Catalytic DMF (10 μL) was added and the resulting solution stirred for 60 min. The reaction was concentrated in vacuo to afford the title compound (110 mg, quantitative yield).

### Methyl 5-chloro-2-[(1-isopropylpiperidine-3-carbonyl)amino]pyridine-3-carboxylate

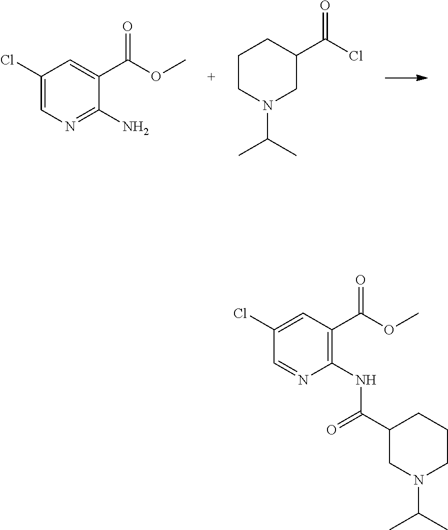

To a solution of 1-isopropyl-3-carbonyl chloride (112 mg, 0.59 mmol) in DCM (2 mL) was added methyl 2-amino-5-chloro-pyridine-3-carboxylate (100 mg, 0.54 mmol) and N,N-diisopropylethylamine (208 mg, 1.61 mmol). The reaction was left stirring at rt for 18 hrs. The reaction was concentrated in vacuo and flash chromatography (0-5% MeOH in DCM) afforded the title compound (200 mg, 82% yield) as a yellow oil.

[M+H]⁺=340.1

### 2-Chloro-6-phenylnicotinic acid

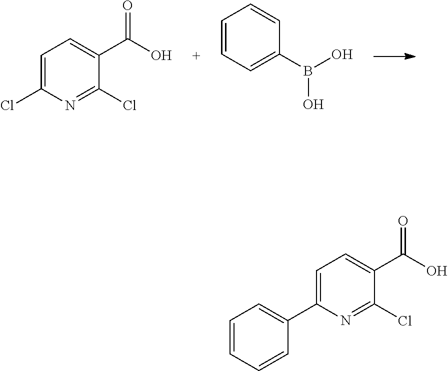

Following a modification to method G, to a microwave vial was added 2,6-dichloronicotinic acid (500 mg, 2.60 mmol), phenylboronic acid (413 mg, 3.39 mmol), potassium carbonate (1.44 g, 10.4 mmol) and bis(triphenylphosphine)palladium (II) dichloride (91 mg, 0.13 mmol) in DME (5 mL), water (5 mL) and EtOH (5 mL). The vial was sealed and heated to 140° C. for 40 min. The crude mixture was diluted with water (20 mL) and extracted with EtOAc (2×5 mL) before acidifying the aqueous to pH 5 with 2M HCl. The acidified aqueous was extracted with further EtOAc (3×50 mL) before combining the organics, drying (MgSO₄) and concentrating in vacuo to afford the title compound (285 mg, 47% yield) as a white solid.

[M+H]⁺=234.1/236.1

### 6-Chloro-[2,3′-bipyridine]-5-carboxylic acid

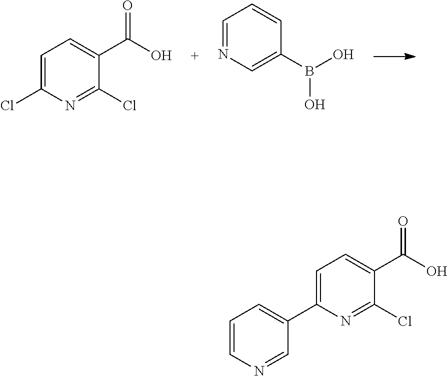

Following a modification to method G, to a microwave vial was added 2,6-dichloronicotinic acid (500 mg, 2.60 mmol), pyridin-3-ylboronic acid (416 mg, 3.39 mmol), potassium carbonate (1.44 g, 10.4 mmol) and bis(triphenylphosphine)palladium (II) dichloride (91 mg, 0.13 mmol) in DME (5 mL), water (5 mL) and EtOH (5 mL). The vial was sealed and heated to 140° C. for 40 min. The crude mixture was diluted with water (20 mL) and extracted with EtOAc (2×5 mL) before acidifying the aqueous to pH 5 with 2M HCl. The acidified aqueous was extracted with further EtOAc (3×20 mL) before combining the organics, drying (MgSO₄) and concentrating in vacuo to afford the title compound (482 mg, 79% yield) as a white solid.

### 2-Chloro-6-cyclopropylnicotinic acid

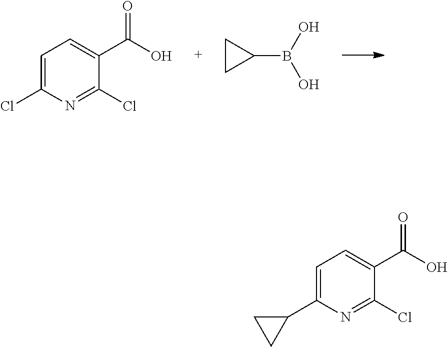

Following a modification to method G, to a microwave vial was added 2,6-Dichloronicotinic acid (500 mg, 2.60 mmol), cyclopropylboronic acid (291 mg, 3.39 mmol), potassium carbonate (1440 mg, 10.4 mmol), bis(triphenylphosphine)palladium (II) dichloride (91 mg, 0.13 mmol) in DME (5 mL), water (5 mL) and EtOH (5 mL). The vessel was sealed and heated to 140° C. for 40 min. The crude mixture was diluted with water (200 mL) and extracted with EtOAc (2×100 mL). The aqueous phase was acidified with 2M HCl and further extracted with EtOAc (3×30 mL). The organics were combined, dried (MgSO₄) and concentrated in vacuo. Reverse phase preparative HPLC afforded the title compound (125 mg, 24% yield).

### Methyl 6-bromo-2-(((1-methylpiperidin-4-yl)methyl)amino)nicotinate

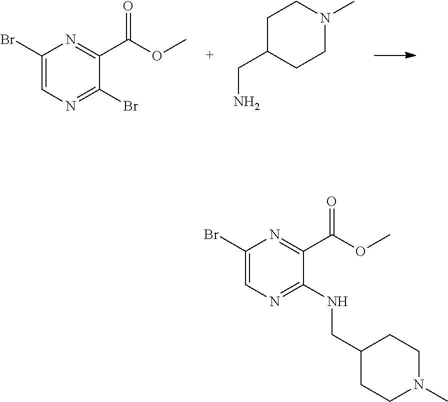

Following general method E, methyl 3,6-dibromopyrazine-2-carboxylate (500 mg, 1.69 mmol) was reacted with (1-methylpiperidin-4-yl)methanamine (433 mg, 3.30 mmol) to afford the title compound (568 mg, 98% yield) as a yellow solid.

### Methyl 3-((4-(4-(tert-butoxycarbonyl)piperazin-1-yl)phenyl)amino)pyrazine-2-carboxylate

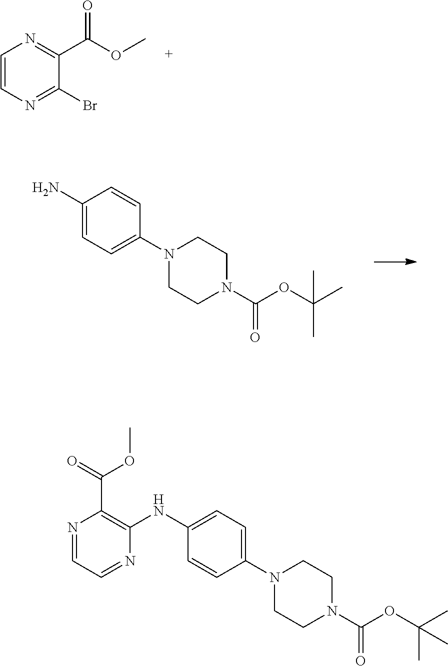

To a solution of methyl-3-bromopyrazine-2-carboxylate (50 mg, 0.23 mmol) and 1-piperazinecarboxylic acid, 4-(4-aminophenyl)-, 1, 1-dimethylethyl ester (67 mg, 0.24 mmol) in 1,4-dioxane (1 mL) was added palladium (II) acetate (5.2 mg, 0.02 mmol), potassium carbonate (96 mg, 0.69 mmol) and xantphos (27 mg, 0.046 mmol) to a sealed vessel. This was purged with N₂ and heated to 90° C. for 60 min. Flash chromatography (0-3% MeOH in DCM) afforded the title compound as a pale brown oil (88 mg, 93% yield.)

### 6-(((1-Methylpiperidin-4-yl)methyl)amino)picolinonitrile

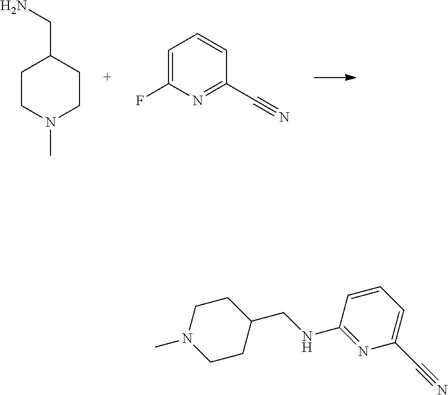

To a solution of (1-methylpiperidin-4-yl)methanamine (315 mg, 2.46 mmol), 6-fluoropicolinonitrile (300 mg, 2.46 mmol) in NMP (3 mL) was added potassium carbonate (679 mg, 4.91 mmol) and stirred in the microwave at 100° C. for 3 hrs. Flash chromatography (SCX, 7M NH₃ in MecOH) afforded the title compound (566 mg, 95% yield) as a yellow solid.

[M+H]⁺=231.1

¹H NMR (500 MHz, DMSO-d6) δ 1.13-1.24 (m, 2H), 1.64-1.74 (m, 2H), 1.76-1.84 (m, 2H), 2.13 (s, 3H), 2.15-2.23 (m, 1H), 2.71-2.79 (m, 2H), 3.07-3.14 (m, 2H), 6.75-6.83 (m, 1H), 6.97-7.05 (m, 1H), 7.17 (t, J=5.6 Hz, 1H), 7.41-7.52 (m, 1H)

### 6-(((1-Methyl piperidin-4-yl)methyl)amino)picolinic acid

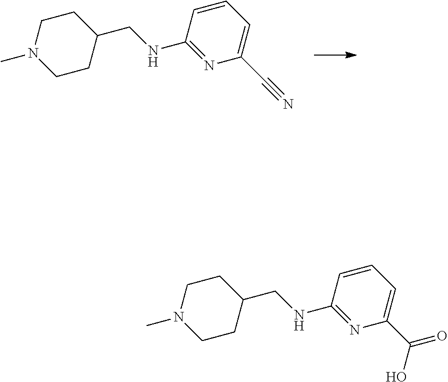 [SMILES: CN1CCC(CNc2cccc(C#N)n2)CC1 ; CN1CCC(CNc2cccc(C(=O)O)n2)CC1]

6-(((1-methylpiperidin-4-yl)methyl)amino)picolinonitrile (614 mg, 2.67 mmol) was dissolved in a mixture of ethanol (5 mL) and potassium hydroxide (4 M) (5 mL, 20.0 mmol) and heated in the microwave at 100° C. for 60 mins. The solution was concentrated in vacuo and taken onto the next step without further purification.

[M+H]⁺=250.1

### Synthesis of (2-chloro-6-(1H-tetrazol-1-yl)phenyl)methanamine

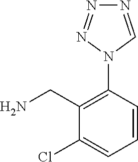 [SMILES: NCc1c(Cl)cccc1-n1cnnn1]

### (2-Chloro-6-tetrazol-1-yl-phenyl)-methanol

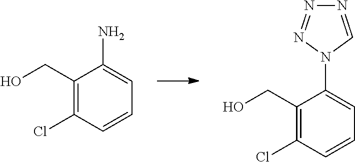 [SMILES: Nc1cccc(Cl)c1CO ; OCc1c(Cl)cccc1-n1cnnn1]

(2-Amino-6-chlorophenyl)methanol (1.0 g, 6.3 mmol) was dissolved in acetic acid (10 mL). Trimethyl orthoformate (2.0 g, 19.0 mmol) and sodium azide (1.23 g, 19.0 mmol) were added. The reaction mixture was stirred at rt for 18 hrs and then heated at 50° C. for 4 hrs. The reaction mixture was cooled to rt and diluted with EtOAc (100 mL), washed with water (30 mL), brine (30 mL), dried (Na₂SO₄), filtered through PS paper and evaporated. The residue was azeotroped with toluene and purified by flash chromatography (0-40% EtOAc in hexane) to afford the title compound as a white solid (760 mg, 57% yield)

[M+MeCN]⁺=252.1

### 1-(2-Bromomethyl-3-chloro-phenyl)-1H-tetrazole

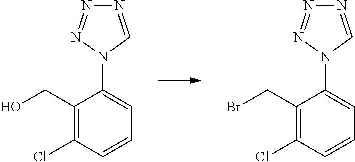 [SMILES: OCc1c(Cl)cccc1-n1cnnn1 ; Clc1cccc(-n2cnnn2)c1CBr]

(2-Chloro-6-tetrazol-1-yl-phenyl)-methanol (760 mg, 3.6 mmol) was dissolved in DCM (40 mL) and phosphorous tribromide (1.95 g, 7.2 mmol) was added. The reaction mixture was stirred at rt for 2 hrs then diluted with CHCl₃ (50 mL) and washed with sat. NaHCO₃ (100 mL), water (10 mL) and brine (10 mL), dried (Na₂SO₄), filtered through PS paper and evaporated. Purification by flash chromatography (0-40% EtOAc in hexane) afforded the title compound as a white solid (900 mg, 91% yield). [M+MeCN]⁺=314.1/316.1

### 1-(2-Azidomethyl-3-chloro-phenyl)-1H-tetrazole

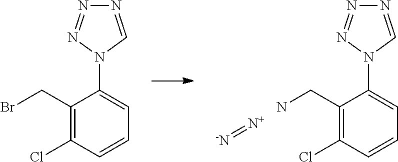

To a solution of 1-(2-bromomethyl-3-chloro-phenyl)-1H-tetrazole (900 mg, 3.3 mmol) in DMF (10 mL) was added sodium azide (428 mg, 6.6 mmol). The reaction mixture was stirred for 18 hrs under a nitrogen atmosphere then diluted with ethyl acetate (60 mL) and washed with water (4×30 mL) followed by brine (20 mL). The organic layer was dried (MgSO₄), filtered and concentrated to low volume. Product was purified by flash chromatography (0-40% EtOAc in hexane) concentrated to low volume then azeotroped with THF (3×50 mL) and used immediately in the next reaction. Yield not obtained as azide was not concentrated to dryness (assume 775 mg, quantitative).

[M+H]⁺=241.9

### [2-chloro-6-(1H-1,2,3,4-tetrazol-1-yl)phenyl]methanamine

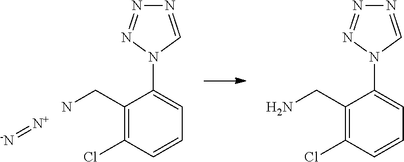

To a solution of 1-(2-Azidomethyl-3-chloro-phenyl)-1H-tetrazole (775 mg, 3.33 mmol) in THF (20 mL) and water (7 mL) was added triphenylphosphine (3.24 g, 12.33 mmol). The reaction mixture was stirred for 18 hrs at rt under a nitrogen atmosphere. The solvent was removed under vacuum and EtOAc (7.5 mL) added followed by 4M HCl in 1,4-dioxane (2 mL) and diethyl ether (6 mL), the liquid was decanted off and the oil triturated with EtOH to give the title compound (585 mg, 85% yield) as a white solid.

[M+H]⁺=206.2/208.0

### Synthesis of isoquinoline-1,6-diamine

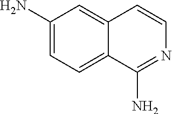 [SMILES: Nc1ccc2c(N)nccc2c1]

### tert-Butyl N-(tert-butoxycarbonyl)-N-(6-nitroisoquinolin-1-yl)carbamate

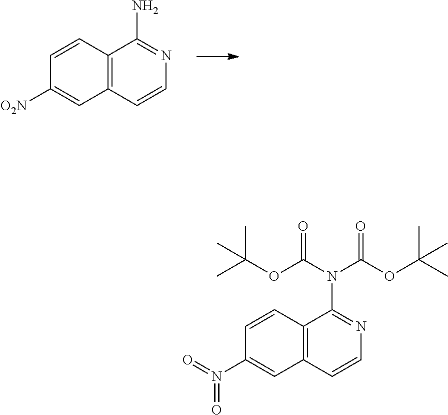 [SMILES: Nc1nccc2cc([N+](=O)[O-])ccc12 ; CC(C)(C)OC(=O)N(C(=O)OC(C)(C)C)c1nccc2cc([N+](=O)[O-])ccc12]

To a solution of 6-nitroisoquinolin-1-amine (1.0 g, 5.286 mmol) in DMPU (5 mL) was added di-tert-butyl dicarbonate (2.538 g, 11.63 mmol) and DMAP (32 mg, 0.26 mmol). The reaction was stirred at 70° C. for 30 min. The reaction was quenched with water (70 mL) diluted with ethyl acetate (75 mL) and separated. The organic layer was washed with water (100 mL) followed by brine (50 mL), dried (MgSO₄), filtered and the solvent removed under vacuum to afford an orange solid. The residue was purified via flash chromatography (0-100% EtOAc in Pet. Ether) to afford the title compound as an orange oil (1.01 g, 49% yield).

[M+H]⁺=390.2

¹H NMR (400 MHz, DMSO): 1.33 (18H, s), 7.86 (1H, d, J=5.3 Hz), 8.15 (1H, d, J=9.2 Hz), 8.39 (1H, dd, J=9.2, 2.2 Hz), 8.6 (1H, d, J=5.7 Hz), 8.82 (1H, d, J=2.2 Hz).

### tert-Butyl N-(6-aminoisoquinolin-1-yl)-N-(tert-butoxycarbonyl)carbamate)

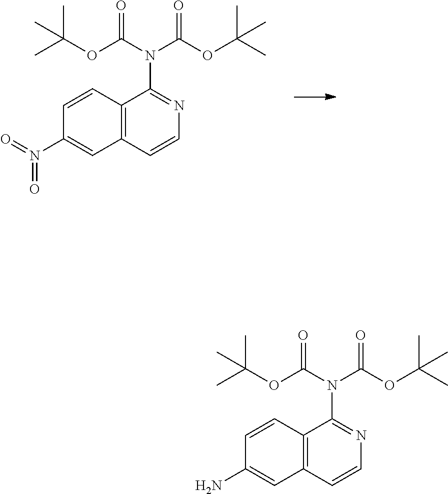 [SMILES: CC(C)(C)OC(=O)N(C(=O)OC(C)(C)C)c1nccc2cc([N+](=O)[O-])ccc12 ; CC(C)(C)OC(=O)N(C(=O)OC(C)(C)C)c1nccc2cc(N)ccc12]

tert-Butyl N-(tert-butoxycarbonyl)-N-(6-nitroisoquinolin-1-yl)carbamate (1.23 g, 3.0 mmol) was dissolved in methanol (75 mL). This solution was hydrogenated over 10% Pd/C (100 mg). After 3.5 hrs, the catalyst was filtered off through Celite and the residue washed with methanol (50 mL). The combined filtrates were evaporated in vacuo and purified by flash chromatography (0-10% MeOH in DCM) to afford the title compound as a yellow green solid (1.0 g, 88% yield).

[M+H]⁺=360.3

¹H NMR (DMSO): 1.31 (18H, s), 6.05 (2H, br.s), 6.78 (1H, d, J=2.1 Hz), 7.05 (1H, dd, J=9.0, 2.1 Hz), 7.38 (1H, d, J=5.8 Hz), 7.49 (1H, d, J=9.0 Hz), 8.03 (1H, d, J=5.7 Hz).

### Isoquinoline-1,6-diamine

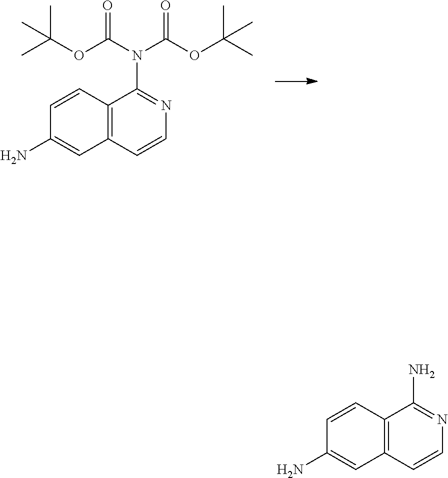 [SMILES: CC(C)(C)OC(=O)N(C(=O)OC(C)(C)C)c1nccc2cc(N)ccc12 ; Nc1ccc2c(N)nccc2c1]

Following a modification to general method H, tert-Butyl N-(6-aminoisoquinolin-1-yl)-N-(tert-butoxycarbonyl)carbamate) (75 mg, 0.14 mmol) was deprotected in 1,4-dioxane (1 mL) using 4N HCl in dioxane (2 mL). Purification by flash chromatography (0-10% (1% NH₃ in MeCOH) in DCM) afforded the title compound as an orange solid (7 mg, 32% yield).

[M+H]⁺=160.2

¹H NMR (DMSO): 6.62 (2H, br.s), 6.71 (1H, d, J=2.1 Hz), 6.83 (1H, d, J=7.1 Hz), 6.95 (1H, dd, J=9.1, 2.2 Hz), 7.39 (1H, d, J=7.1 Hz), 8.16 (1H, d, J=9.1 Hz), 8.27 (21H, br.s), 12.05 (1H, br.s)

### Synthesis of Isoquinoline-1,5-diamine

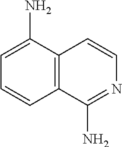 [SMILES: Nc1cccc2c(N)nccc12]

### 1-N,5-N-bis(diphenylmethylidene)isoquinoline-1,5-diamine

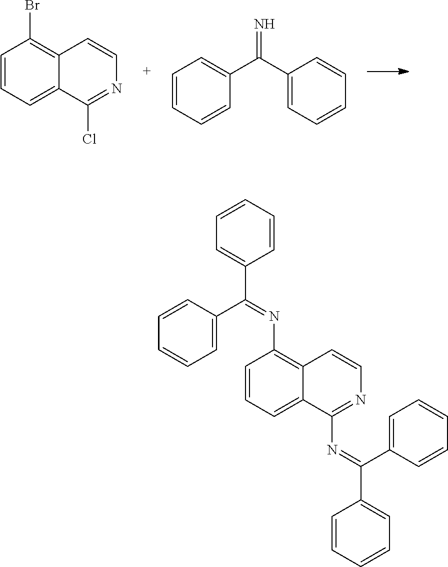 [SMILES: Clc1nccc2c(Br)cccc12 ; N=C(c1ccccc1)c1ccccc1 ; c1ccc(C(=Nc2cccc3c(N=C(c4ccccc4)c4ccccc4)nccc23)c2ccccc2)cc1]

To 1-chloro-5-bromoisoquinoline (83 mg, 0.34 mmol) was added BINAP (64 mg, 0.10 mmol), sodium tert-butoxide (82 mg, 0.86 mmol), benzophenone imine (124 mg, 0.685 mmol) and dry toluene (2 mL). Tris(dibenzylideneacetone)dipalladium(0) (47 mg, 0.051 mmol) was added and the reaction was heated at 50° C. for 18 hrs. Reaction was partitioned between EtOAc (25 mL) and water (10 mL), organic layer was washed with brine (10 mL), dried (MgSO₄) filtered and concentrated in vacuo. Purification by flash chromatography (2-68% EtOAc in Pet. Ether) afforded the title compound (75 mg, 45% yield) as a white solid.

[M+H]⁺=488.3

### Isoquinoline-1,5-diamine

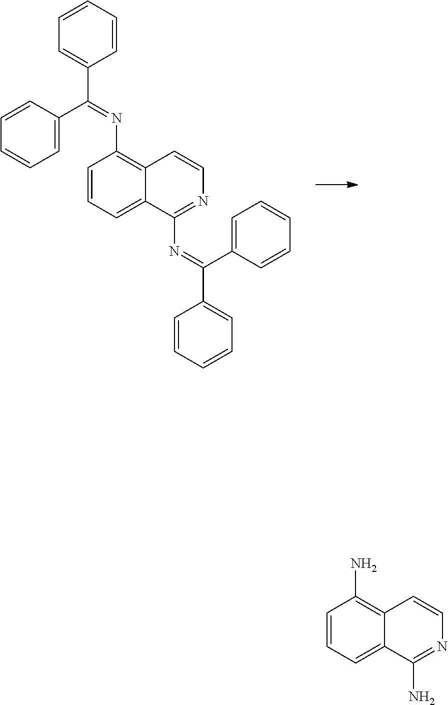 [SMILES: c1ccc(C(=Nc2cccc3c(N=C(c4ccccc4)c4ccccc4)nccc23)c2ccccc2)cc1 ; Nc1cccc2c(N)nccc12]

To 1-N,5-N-bis(diphenylmethylidene)isoquinoline-1,5-diamine (75 mg, 0.154 mmol) in THF (5 mL) was added 12M Hydrochloric acid (1 mL) and the reaction stirred at rt for 18 hrs. The reaction mixture was concentrated in vacuo and diluted with MeCN (20 mL), filtered and solid washed with MeCN (10 mL). The solid was freeze dried to afford the title compound (25 mg, 70% yield) as a white solid.

[M+H]⁺=160.2

¹H NMR: (DMSO) 5.71 (3H, br s), 7.13 (1H, d, J=7.8 Hz), 7.36 (1H, d, J=7.2 Hz), 7.46 (1H, t, J=8.0 Hz), 7.55-7.61 (1H, m), 7.70 (1H, d, J=8.2 Hz), 8.87 (2H, br s), 13.23 (1H, br s).

### Synthesis of 3-Chloro-1H-indol-5-amine

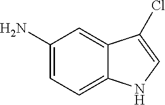 [SMILES: Nc1ccc2[nH]cc(Cl)c2c1]

### tert-Butyl (tert-butoxycarbonyl)(1H-indol-5-yl)carbamate

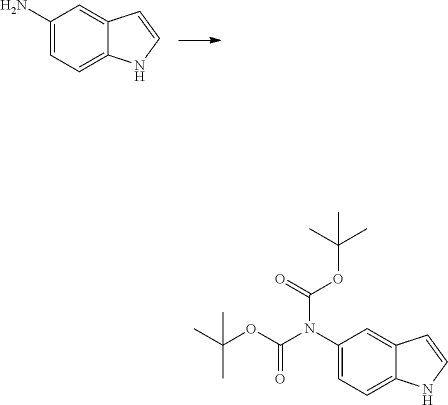 [SMILES: Nc1ccc2[nH]ccc2c1 ; CC(C)(C)OC(=O)N(C(=O)OC(C)(C)C)c1ccc2[nH]ccc2c1]

To a solution of 1H-indol-5-amine (CAS 5192-03-0, 250 mg, 1.88 mmol) in THF (10 mL), was added triethylamine (1.0 mL, 7.51 mmol), di-tert-butyl dicarbonate (0.86 mL, 3.76 mmol) and DMAP (23 mg, 0.19 mmol) before leaving to stir at rt for 18 hrs. The crude mixture was diluted with water (25 mL) and washed with EtOAc (3×25 mL). The organics were combined, dried (MgSO₄) and concentrated in vacuo. Flash chromatography (0-100% EtOAc in cyclohexane) afforded the title compound (427 mg, 68% yield).

[M+H]⁺=334.0

### tert-Butyl (tert-butoxycarbonyl)(3-chloro-1H-indol-5-yl)carbamate

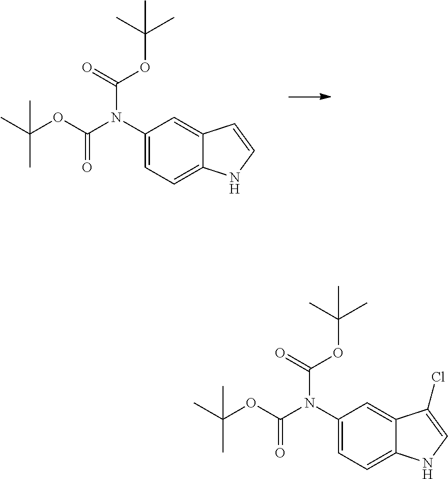 [SMILES: CC(C)(C)OC(=O)N(C(=O)OC(C)(C)C)c1ccc2[nH]ccc2c1 ; CC(C)(C)OC(=O)N(C(=O)OC(C)(C)C)c1ccc2[nH]cc(Cl)c2c1]

tert-Butyl (tert-butoxycarbonyl)(1H-indol-5-yl)carbamate (427 mg, 1.28 mmol) was dissolved in DMF (5 mL) before adding N-Chlorosuccinimide (171 mg, 1.28 mmol) and leaving to stir at 40° C. for 18 hrs. The reaction mixture was diluted with water (35 mL) and extracted with EtOAc (3×25 mL). The organic phases were combined, washed with brine (25 mL), dried (MgSO₄), filtered and concentrated in vacuo. Flash chromatography (0-60% EtOAc in cyclohexane) afforded the title compound (99 mg, 21% yield) [M+H]⁺=368.0

### 3-Chloro-1H-indol-5-amine

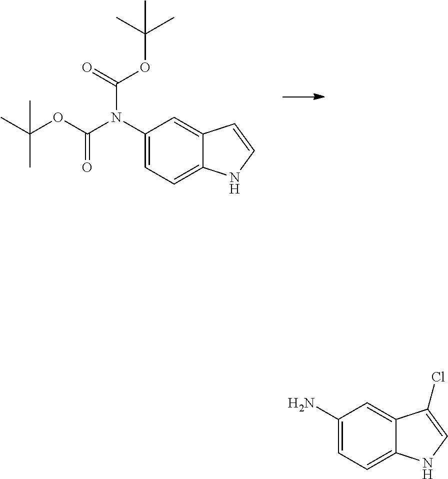 [SMILES: CC(C)(C)OC(=O)N(C(=O)OC(C)(C)C)c1ccc2[nH]ccc2c1 ; Nc1ccc2[nH]cc(Cl)c2c1]

Following a modification to general method H, tert-butyl (tert-butoxycarbonyl)(3-chloro-1H-indol-5-yl)carbamate (99 mg, 0.27 mmol) was deprotected in dioxane (1 mL) and 4M HCl in dioxane (2 mL, 8.07 mmol) to afford the title compound (33 mg, 51% yield).

[M+H]⁺=168.0

### 3-Chloro-1H-indol-4-amine

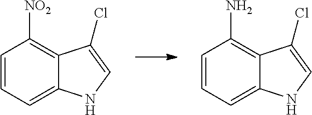 [SMILES: O=[N+]([O-])c1cccc2[nH]cc(Cl)c12 ; Nc1cccc2[nH]cc(Cl)c12]

A solution of Iron (341 mg, 6.10 mmol) in AcOH (2.9 mL, 50.9 mmol) was heated to 65° C. and stirred for 15 min. 3-Chloro-4-nitro-1H-indole (CAS 208511-07-3) (200 mg, 1.02 mmol) in AcOH (7 mL) was then added portionwise over 20 min. The reaction was cooled to rt, filtered over Celite, washed with EtOAc (75 mL) and concentrated in vacuo. The residue was basified with NaHCO₃ (sat. aq.) and extracted with DCM (3×25 mL). The combined organics were washed with brine (20 mL), dried (Na₂SO₄), filtered and concentrated. Flash chromatography (5-95% MeCN in 10 mM NH₄OH) afforded the title compound (9.6 mg, 6% yield).

[M+H]⁻=167.1

### Synthesis of 7-(aminomethyl)isoquinolin-1-amine

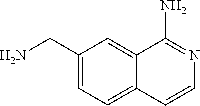 [SMILES: NCc1ccc2ccnc(N)c2c1]

### 1-Aminoisoquinoline-7-carbonitrile

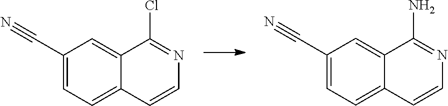 [SMILES: N#Cc1ccc2ccnc(Cl)c2c1 ; N#Cc1ccc2ccnc(N)c2c1]

1-Chloroisoquinoline-2-carbonitrile (250 mg, 1.33 mmol), ammonium acetate (1.53 g, 19.88 mmol) and phenol (1.87 g, 19.88 mmol) were added to a sealed tube and heated to 150° C. for 6 hrs before leaving to cool to rt for 18 hrs. The reaction mixture was diluted in 1M NaOH (25 mL) before extracting with DCM (3×25 mL). The combined organics were dried (MgSO₄), filtered and concentrated in vacuo. Flash chromatography (0-100% EtOAc in cyclohexane) afforded the title compound (194 mg, 88% yield) as an orange solid.

[M+H]⁺=170.0

### tert-Butyl (7-(((tert-butoxycarbonyl)amino)methyl)isoquinolin-1-yl)carbamate

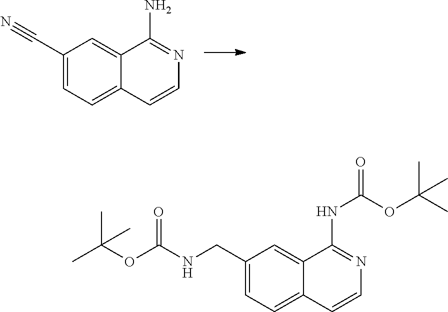 [SMILES: N#Cc1ccc2ccnc(N)c2c1 ; CC(C)(C)OC(=O)NCc1ccc2ccnc(NC(=O)OC(C)(C)C)c2c1]

1-aminoisoquinoline-7-carbonitrile (190 mg, 1.15 mmol) was dissolved in MecOH (5 mL) and cooled to 0° C. before adding nickel (II) chloride hexahydrate (27 mg, 0.12 mmol) and di-tert-butyl decarbonate (750 mg, 3.44 mmol). Sodium borohydride (300 mg, 8.03 mmol) was added portionwise and the reaction stirred at rt for 18 hrs. The reaction mixture was concentrated in vacuo, diluted with NaHCO₃ (20 mL) extracted with EtOAc (3×20 mL). The organic phases were combined, washed with brine (20 mL), dried (MgSO₄), filtered and concentrated in vacuo. Flash chromatography (0-100% EtOAc in cyclohexane) afforded the title compound (155 mg, 36% yield).

### 7-(Aminomethyl)isoquinolin-1-amine

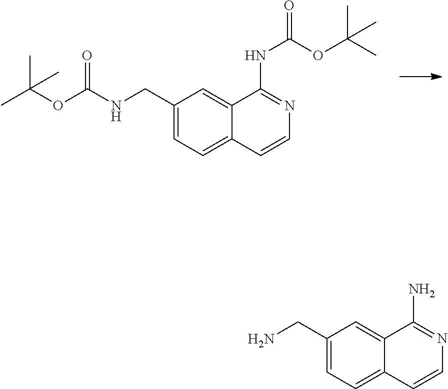 [SMILES: CC(C)(C)OC(=O)NCc1ccc2ccnc(NC(=O)OC(C)(C)C)c2c1 ; NCc1ccc2ccnc(N)c2c1]

Following modified general method H, tert-butyl (7-(((tert-butoxycarbonyl)amino)methyl)isoquinolin-1-yl)carbamate (155 mg, 0.42 mmol) was deprotected in dioxane (1 mL) and 4M HCl in dioxane (3.1 mL, 12.45 mmol). The reaction mixture was triturated (MeOH/EtOAc (1:5)) and filtered before dissolving in MecOH and concentrated in vacuo to afford the title compound (70 mg, 69% yield).

[M+H]⁺=174.1

### Synthesis of 2-(aminomethyl)thieno[3,2-c]pyridin-4-amine

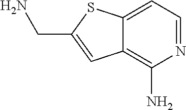 [SMILES: NCc1cc2c(N)nccc2s1]

### 4-Phenoxythieno[3,2-c]pyridine

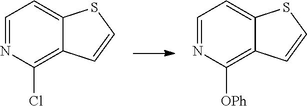 [SMILES: Clc1nccc2sccc12 ; c1ccc(Oc2nccc3sccc23)cc1]

A mixture of 4-chlorothieno[3,2-c]pyridine (10 g, 59.0 mmol) and phenol (36.6 g, 389 mmol) was warmed to 45° C. to form a homogeneous solution. KOH (5.6 g, 100 mmol) was added and the reaction heated to 140° C. for 18 hrs. The reaction mixture was cooled to 50° C. and diluted with 2N NaOH (250 mL), before being further cooled to rt, extracted with DCM (3×400 mL) and washed with brine (100 mL). The combined organic layers were dried (MgSO₄), filtered and concentrated in vacuo to afford 4-phenoxythieno[3,2-c]pyridine (13.25 g, 92% yield) as an dark brown crystalline solid.

[M+H]⁺=228.2

¹H NMR (500 MHz, DMSO-d6) δ 7.21-7.28 (m, 3H), 7.45 (dd, J=8.4, 7.3 Hz, 2H), 7.67 (d, J=5.5 Hz, 1H), 7.80 (d, J=5.6 Hz, 1H), 7.92 (dd, J=5.5, 4.3 Hz, 2H).

### Thieno[3,2-c]pyridin-4-amine

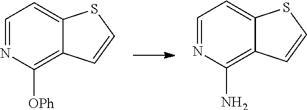 [SMILES: c1ccc(Oc2nccc3sccc23)cc1 ; Nc1nccc2sccc12]

4-phenoxythieno[3,2-c]pyridine (13.2 g, 58.1 mmol) and ammonium acetate (105 g, 1362 mmol) were mixed and heated to 150° C. for 72 hrs. The reaction mixture was cooled to 50° C. and quenched with 2M NaOH (200 mL). The aqueous phase was then cooled to rt and extracted with EtOAc (3×200 mL). The combined organic extracts were washed with brine (200 mL), dried (MgSO₄), filtered and concentrated in vacuo. The crude product was sonicated with 2M NaOH (100 mL). EtOAc (100 mL) was added and the organic layer was separated. The aqueous layer was extracted with EtOAc (3×100 mL). The combined organics were washed with brine (100 mL), dried (MgSO₄), filtered and concentrated in vacuo, to afford the title compound (5.6 g, 63% yield) as a dark brown solid.

¹H NMR (500 MHz, DMSO-d6) δ 6.54 (s, 2H), 7.11-7.14 (m, 1H), 7.56 (d, J=5.5 Hz, 1H), 7.63-7.67 (m, 1H), 7.75 (d, J=5.7 Hz, 1H).

### N-(thieno[3,2-c]pyridin-4-yl)benzamide

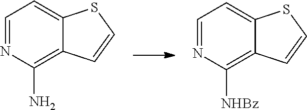

To a solution of thieno[3,2-c]pyridin-4-amine (5.6 g, 37.3 mmol) in pyridine (60 mL) was added benzoic anhydride (9.28 g, 41.0 mmol) at rt. The mixture was heated to 125° C. After 2 hrs the reaction was cooled to rt and the reaction mixture was concentrated in vacuo. The crude mixture was partitioned between water (200 mL) and DCM (200 mL). The organic layer was separated and the aqueous layer was extracted with DCM (2×200 mL). The combined organics were washed with brine (100 mL), dried (MgSO₄), filtered and concentrated in vacuo. The crude product was purified by flash chromatography (5%-100% EtOAc in isohexane) to afford a yellow solid. The product was partitioned in DCM (100 mL) and Na₂CO₃ solution (aq., sat., 100 mL). The mixture was sonicated for 5 min. The organic layer was separated and the aqueous layer was extracted with DCM (2×100 mL). The combined organic extracts were dried (Na₂SO₄), filtered and concentrated in vacuo to afford the title compound (6.62 g, 69% yield) as a foaming yellow solid.

[M+H]⁺=255.2

### N-(2-formylthieno[3,2-c]pyridin-4-yl)benzamide

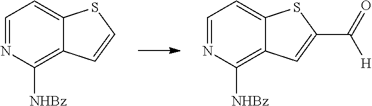

To a solution of N-(thieno[3,2-c]pyridin-4-yl)benzamide (6.6 g, 26.0 mmol) in THF (120 mL) at −78° C., LDA, 2M in THF/heptane/ethylbenzene (28.5 mL, 57.1 mmol) was added dropwise. The reaction mixture was stirred at −78° C. for 45 min then DMF (7 mL, 90 mmol) was added dropwise and the cooling bath was removed. The reaction was stirred at rt for 18 hrs before quenching with NH₄Cl (sat., aq., 100 mL). The aqueous layer was extracted with EtOAc (5×100 mL). The combined organic extracts were dried (Na₂SO₄), filtered and concentrated in vacuo. The crude product was purified by flash chromatography (5-100% THF in isohexane) to afford the title compound (4.62 g, 61% yield) as a pale yellow solid.

[M+H]⁺=283.2

### N-(2-(((2,4-dimethoxybenzyl)amino)methyl)thieno[3,2-c]pyridin-4-yl)benzamide)

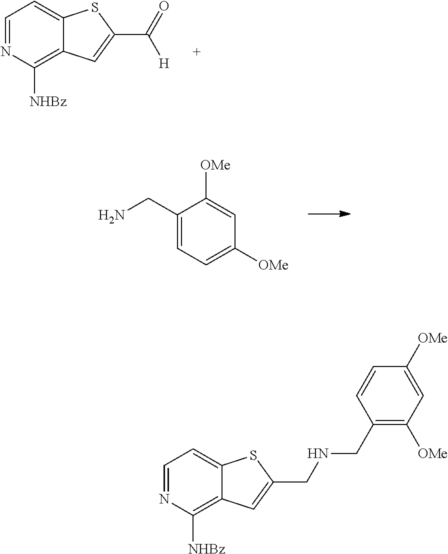

N-(2-formylthieno[3,2-c]pyridin-4-yl)benzamide (4.6 g, 16.29 mmol) and (2,4-dimethoxyphenyl)methanamine (3.27 g, 19.55 mmol) were mixed with AcOH (0.94 mL) and THF (110 mL). After 3 hrs, sodium triacetoxyborohydride (5.18 g, 24.44 mmol) was added. The reaction was stirred at rt for 3 hrs and then heated to 40° C. overnight. The reaction was quenched with NaHCO₃ (sat., aq., 100 mL). The organic layer was separated and the aqueous layer was extracted with EtOAc (3×100 mL). The combined organics were dried (Na₂SO₄), filtered and concentrated in vacuo. The crude product was purified by flash chromatography (0-100% EtOAc in isohexane) to afford the title compound (3.9 g, 49% yield) as a pale yellow solid.

### 2-(Aminomethyl)thieno[3,2-c]pyridin-4-amine

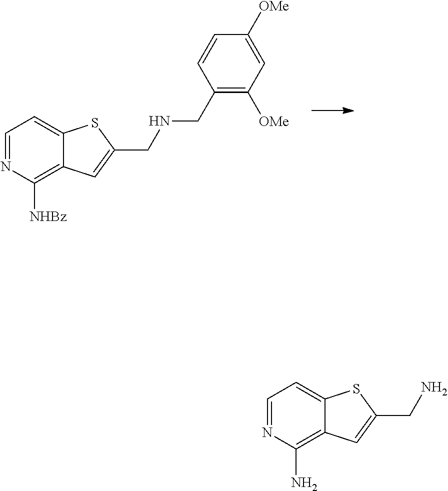

In sealed microwave vial, to a solution of N-(2-(((2,4-dimethoxybenzyl)amino)methyl)thieno[3,2-c]pyridin-4-yl)benzamide (650 mg, 1.5 mmol) in AcOH (6 mL) was added HCl (37 wt %, aq., 9 mL). The solution was heated to 100° C. for 18 hrs. The reaction was cooled to rt and the solvent and excess acid were removed in vacuo. The reaction mixture was partitioned between NaOH solution (aq., 2M, 150 mL) and EtOAc (150 mL). The aqueous phase was extracted with THF (5×200 mL). The combined organic extracts were dried (Na₂SO₄, filtered and concentrated in vacuo to afford a dark red solid. The crude product was purified by reverse phase flash chromatography (0-50% MeCN in 10 mM Ammonium Bicarbonate) to afford the title compound (770 mg, 47% yield) as a pale red solid.

[M+H]⁺=180.2

¹H NMR (500 MHz, DMSO-d6) δ 2.02 (s, 2H), 3.96 (d, J=1.3 Hz, 2H), 6.36 (s, 2H), 7.03 (d, J=5.7 Hz, 1H), 7.38-7.42 (m, 1H), 7.69 (d, J=5.6 Hz, 1H).

### Synthesis of [2-Fluoro-3-methoxy-6-(1,2,4-triazol-1-yl)phenyl]methanamine

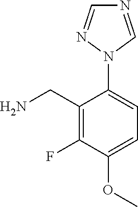 [SMILES: COc1ccc(-n2cncn2)c(CN)c1F]

### (2-Fluoro-6-iodo-3-methoxyphenyl)methanol

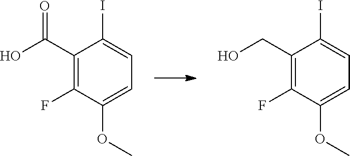

To a solution of 2-fluoro-6-iodo-3-methoxy-benzoic acid (10.0 g, 33.6 mmol) in THF was added 4-methyl morpholine (3.9 mL, 36 mmol) and isobutyl chloroformate (4.4 mL, 34 mmol) dropwise. After 60 min the reaction was filtered and washed with a minimum amount of THF. The filtrate was cooled in an ice-bath and a solution of sodium borohydride (2.0 g, 59 mmol) in cold water (3 mL) was added portion-wise over 20 min. The resulting solution was stirred at rt for 18 hrs. The reaction was acidified with 1M HCl and extracted with TBME (500 mL). The organic layer was washed sequentially with 2M NaOH(aq) (100 mL), 1M HCl (aq) (100 mL) and brine (100 mL), dried over MgSO₄ and concentrated in vacuo. Flash chromatography (0-40% EtOAc in hexane) afforded the title compound (4.9 g, 49% yield).

### [2-Fluoro-3-methoxy-6-(1,2,4-triazol-1-yl)phenyl]methanol

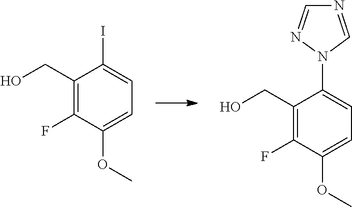 [SMILES: COc1ccc(I)c(CO)c1F ; COc1ccc(-n2cncn2)c(CO)c1F]

A mixture of (2-fluoro-6-iodo-3-methoxy-phenyl)methanol (2.0 g, 7.1 mmol), 1H-1,2,4-triazole (1.0 g, 14 mmol), (1S,2S)—N1,N2-dimethylcyclohexane-1,2-diamine (1.5 g, 11 mmol) and copper(I) iodide (96 mg, 0.50 mmol) was dissolved in DMF (12 mL) then treated with caesium carbonate (3.47 g, 10.7 mmol) and degassed with N₂, then heated at 120° C. for 60 min. The mixture was diluted with DCM (50 mL) and concentrated. Flash chromatography (0 to 50% MeCN in DCM) afforded the title compound (1.2 g, 58% yield).

[M+H]⁺=223.9

### 1-[2-(Chloromethyl)-3-fluoro-4-methoxy-phenyl]-1,2,4-triazole

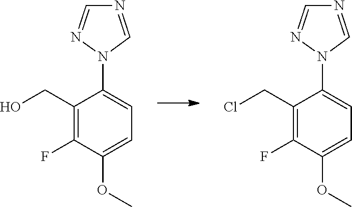 [SMILES: COc1ccc(-n2cncn2)c(CO)c1F ; COc1ccc(-n2cncn2)c(CCl)c1F]

A stirred solution of [2-fluoro-3-methoxy-6-(1,2,4-triazol-1-yl)phenyl]methanol (909 mg, 4.07 mmol) in DCM (25 mL) was treated with TEA (0.91 mL, 6.5 mmol) and cooled in an ice-bath under N₂. Methanesulfonyl chloride (0.45 mL, 5.8 mmol) was added slowly then the ice-bath removed and the mixture warmed to rt and stirred for 2 days. The mixture was diluted with DCM (20 mL) and washed with saturated NaHCO₃(aq) (20 mL). The aqueous layer was extracted with further DCM (2×25 mL). The combined organics were washed with brine (30 mL), dried (Na₂SO₄) and concentrated in vacuo to afford the title compound as a viscous yellow oil (1.0 g, 96% yield).

[M+H]⁺=241.9/243.9

### 2-[[2-Fluoro-3-methoxy-6-(1,2,4-triazol-1-yl)phenyl]methyl]isoindoline-1,3-dione

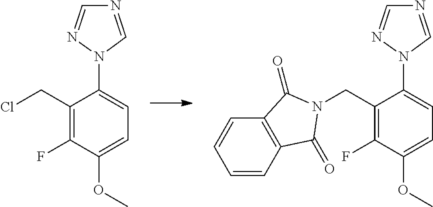 [SMILES: COc1ccc(-n2cncn2)c(CCl)c1F ; COc1ccc(-n2cncn2)c(CN2C(=O)c3ccccc3C2=O)c1F]

Potassium phthalimide (0.868 g, 4.69 mmol) was added to a solution of 1-[2-(chloromethyl)-3-fluoro-4-methoxy-phenyl]-1,2,4-triazole (1.03 g, 4.26 mmol) in DMF (10 mL) and the mixture warmed to 55° C. for 60 min. Water (30 mL) was added to form a thick precipitate which was filtered, washed with water and dried in vacuo in the presence of CaCl₂ to afford the title compound (1.12 g, 74% yield) as a white solid.

[M+H]⁺=352.9

### [2-Fluoro-3-methoxy-6-(1,2,4-triazol-1-yl)phenyl]methanamine

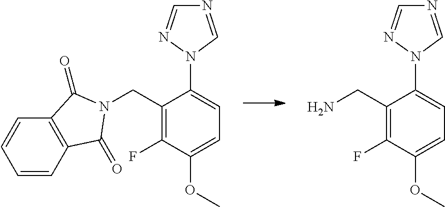 [SMILES: COc1ccc(-n2cncn2)c(CN2C(=O)c3ccccc3C2=O)c1F ; COc1ccc(-n2cncn2)c(CN)c1F]

Hydrazine hydrate (50-60% solution, 0.24 mL) was added to a suspension of 2-[[2-fluoro-3-methoxy-6-(1,2,4-triazol-1-yl)phenyl]methyl]isoindoline-1,3-dione (1.12 g, 3.18 mmol) in MeOH (15 mL) and the reaction mixture heated to 70° C. for 3 hrs. Further hydrazine hydrate (50-60% solution, 0.2 mL) was added and the mixture heated at 70° C. for 60 min, then at rt overnight. The mixture was filtered and the filtrate concentrated in vacuo. The residue was taken up in TBME (40 mL) and sonicated. DCM (10 mL) was added and the mixture worked to a suspension with stirring and sonication. This was filtered and the filtrate concentrated in vacuo then dried in vacuo overnight to afford the title compound (563 mg, 72% yield) as a pale yellow solid.

[M+H]⁺=223.0

### Ethyl 1-(2-(aminomethyl)-3-fluoro-4-methoxyphenyl)-1H-pyrazole-3-carboxylate

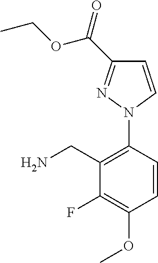 [SMILES: CCOC(=O)c1ccn(-c2ccc(OC)c(F)c2CN)n1]

### Ethyl 6-bromo-2-fluoro-3-methoxy-benzoate

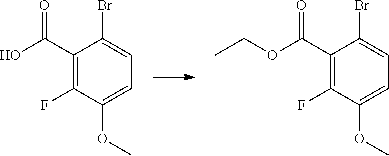

6-Bromo-2-fluoro-3-methoxy-benzoic acid (30.5 g, 123 mmol) was dissolved in MeCN (500 mL). Caesium carbonate (47.9 g, 147 mmol) was added followed by dropwise addition of iodoethane (15.2 mL, 189 mmol). The mixture was stirred at rt for 3 days. The mixture was filtered through Celite, washed with MeCN and concentrated in vacuo. The residue was partitioned between Et₂O (500 mL) and a brine-water mixture (1:2 brine:water, 750 mL). The aqueous phase was extracted with Et₂O (250 mL). The combined organics were dried over Na₂SO₄ and concentrated in vacuo to afford the title compound as an orange oil that solidified on standing (26.8 g, 79% yield).

### Ethyl 6-((tert-butoxycarbonyl)amino)-2-fluoro-3-methoxybenzoate

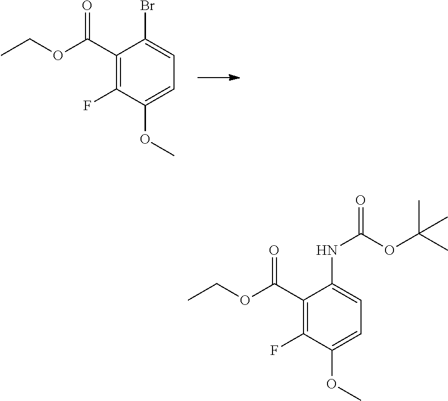 [SMILES: CCOC(=O)c1c(Br)ccc(OC)c1F ; CCOC(=O)c1c(NC(=O)OC(C)(C)C)ccc(OC)c1F]

Ethyl 6-bromo-2-fluoro-3-methoxy-benzoate (10 g, 36 mmol) was dissolved in dioxane (250 mL). tert-Butyl carbamate (4.65 g, 39.7 mmol), 4,5-(bis(diphenylphospheno)-9,9-dimethylxanthene (2.09 g, 3.6 mmol), palladium (II) acetate (810 mg, 3.61 mmol) and caesium carbonate (23.5 g, 72.1 mmol) were added and the mixture was stirred for 18 hrs at 100° C. The mixture was cooled, diluted with EtOAc (250 mL) and filtered through Celite washing with EtOAc (150 mL). The combined filtrates were concentrated in vacuo. Flash chromatography (0-10% EtOAc in Pet. Ether) afforded the title compound as a colourless oil that solidified on standing (8.45 g, 75% yield).

### Ethyl 6-amino-2-fluoro-3-methoxybenzoate

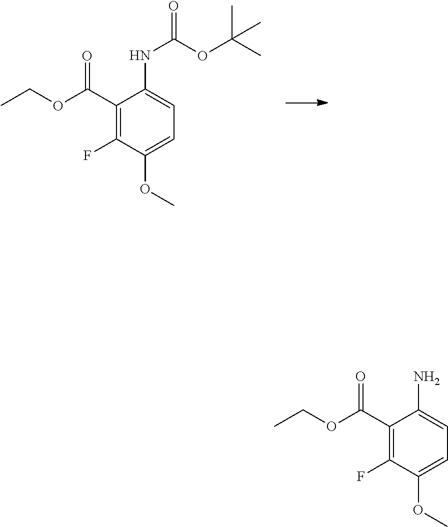 [SMILES: CCOC(=O)c1c(NC(=O)OC(C)(C)C)ccc(OC)c1F ; CCOC(=O)c1c(N)ccc(OC)c1F]

To ethyl 6-((tert-butoxycarbonyl)amino)-2-fluoro-3-methoxybenzoate (3.99 g, 12.7 mmol) was added 4M HCl in 1,4-dioxane (50 mL) and the mixture stirred at rt for 6 hrs. The mixture was concentrated in vacuo to afford the HCl salt of the title compound as a beige solid. (2.83 g, 89% yield).

### (6-Azido-2-fluoro-3-methoxyphenyl)methanol

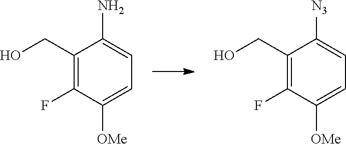 [SMILES: COc1ccc(N)c(CO)c1F ; COc1ccc(N=[N+]=[N-])c(CO)c1F]

A solution of (6-amino-2-fluoro-3-methoxyphenyl)methanol hydrochloride (2.40 g, 11.60 mmol) in methanol (40 mL) was cooled to 0° C. iso-Pentylnitrite (1.60 mL, 11.60 mmol) was added in one portion to the solution, followed by the addition of trimethylsilyl azide (1.60 mL, 11.60 mmol), added slowly over a period of 5 min. After the addition, the mixture was allowed to warm to rt and it was stirred for 3 hrs. The reaction mixture was added to water (100 mL) and methanol was removed in vacuo at 30° C. The mixture was extracted with ethyl acetate (2×100 mL, 1×50 mL), dried over sodium sulfate, filtered and concentrated under reduced pressure at 30° C. The isolated crude material was triturated in the minimum volume of heptane (20 mL). The solid was isolated upon filtration, was washed with heptane and dried to afford the title product (1.85 g, 81% yield).

### Ethyl 1-(3-fluoro-2-(hydroxymethyl)-4-methoxyphenyl)-1H-1,2,3-triazole-4-carboxylate

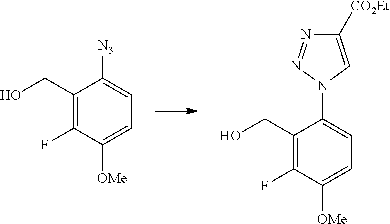 [SMILES: COc1ccc(N=[N+]=[N-])c(CO)c1F ; CCOC(=O)c1cn(-c2ccc(OC)c(F)c2CO)nn1]

Copper(I) iodide (87 mg, 0.457 mmol) and tris[(1-benzyl-1H-1,2,3-triazol-4-yl)methyl]amine (243 mg, 0.457 mmol) were added to a solution of ethyl propiolate (0.55 mL, 5.48 mmol) and (6-azido-2-fluoro-3-methoxyphenyl)methanol (900 mg, 4.57 mmol) in anhydrous acetonitrile (25 mL). The reaction mixture was stirred under nitrogen overnight in darkness. The reaction mixture was concentrated under reduced pressure and then diluted with ethyl acetate (30 mL). The mixture was filtered through a pad of Celite and was washed with ethyl acetate (3×30 mL). The filtrates were washed with conc. ammonium chloride solution (30 mL), water (30 mL) and brine (30 mL). The organic layer was dried over sodium sulfate, filtered and concentrated under reduced pressure in order to give a pale brown solid. The crude material was purified via flash chromatography (0-50% EtOAc in hexane) to afford the title compound (1.10 g, 82% yield).

### Ethyl 1-(2-(chloromethyl)-3-fluoro-4-methoxyphenyl)-1H-1,2,3-triazole-4-carboxylate

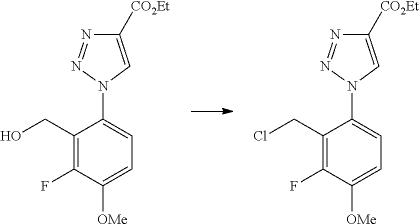 [SMILES: CCOC(=O)c1cn(-c2ccc(OC)c(F)c2CO)nn1 ; CCOC(=O)c1cn(-c2ccc(OC)c(F)c2CCl)nn1]

Triethylamine (0.96 mL, 6.90 mmol) was added to a stirred solution of ethyl 1-(3-fluoro-2-(hydroxymethyl)-4-methoxyphenyl)-1H-1,2,3-triazole-4-carboxylate (1.10 g, 3.73 mmol) in anhydrous dichloromethane (100 mL). The reaction mixture was stirred under nitrogen for 30 min before adding dropwise methane sulfonyl chloride (0.495 mL, 6.40 mmol). The reaction mixture was stirred at room temperature under nitrogen for 3 hrs. The mixture was partitioned between water (20 mL) and dichloromethane (25 mL). The organic layer was washed with water (2×20 mL), an aqueous bicarbonate solution (20 mL) and brine (20 mL), then it was dried over sodium sulfate, filtered and concentrated under reduced pressure to afford the title compound (1.16 g, 83% yield).

### Ethyl 1-(2-(((bis-tert-butoxycarbonyl)amino)methyl)-3-fluoro-4-methoxyphenyl)-1H-1,2,3-triazole-4-carboxylate

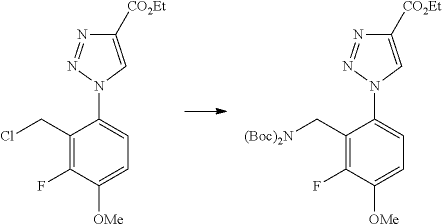

Caesium carbonate (3.04 g, 9.33 mmol) and di-tert-butylaminodicarboxylate (0.679 g, 3.11 mmol) were added to a mixture of ethyl 1-(2-(chloromethyl)-3-fluoro-4-methoxyphenyl)-1H-1,2,3-triazole-4-carboxylate (1.16 g, 3.11 mmol) in dimethylformamide (25 mL). The reaction mixture was stirred at rt for 2 hrs. The mixture was filtered and the filtrates diluted with water. The aqueous layer was extracted with ethyl acetate (3×25 mL). The organic layers were combined, washed with water (20 mL) and brine (20 mL), dried over sodium sulfate, filtered and concentrated under reduced pressure to give the title compound as an orange oil (1.47 g, 96% yield).

### Ethyl 1-(2-(aminomethyl)-3-fluoro-4-methoxyphenyl)-1H-1,2,3-triazole-4-carboxylate hydrochloride

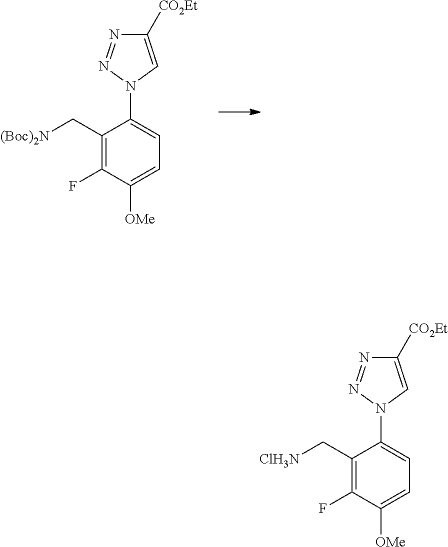

A 4M solution of hydrochloric acid in 1,4-dioxane (15 mL) was added dropwise to a solution of ethyl 1-(2-(((bis-tert-butoxycarbonyl)amino)methyl)-3-fluoro-4-methoxyphenyl)-1H-1,2,3-triazole-4-carboxylate (1.47 g, 2.98 mmol) in 1,4-dioxane (20 mL). The reaction mixture was stirred at rt for 12 hrs then heated to 40° C. for 12 hrs. A beige precipitate was isolated upon filtration and was washed with diethyl ether (2×50 mL) and dried in vacuo in order to give the title compound (875 mg, 89% yield).

[M+H]⁺=295.2

### Synthesis of (6-(4-(Difluoromethyl)-1H-1,2,3-triazol-1-yl)-2-fluoro-3-methoxyphenyl)methanamine

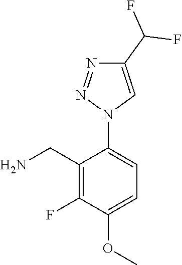 [SMILES: COc1ccc(-n2cc(C(F)F)nn2)c(CN)c1F]

### Ethyl 2-fluoro-6-(4-(hydroxymethyl)-1H-1,2,3-triazol-1-yl)-3-methoxybenzoate

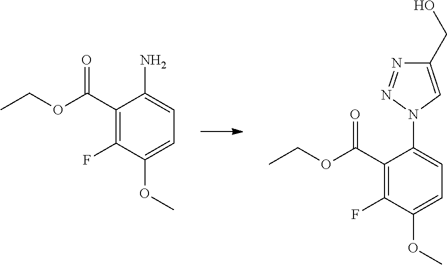 [SMILES: CCOC(=O)c1c(N)ccc(OC)c1F ; CCOC(=O)c1c(-n2cc(CO)nn2)ccc(OC)c1F]

To ethyl 6-amino-2-fluoro-3-methoxybenzoate (500 mg, 2.35 mmol) in MeCN (50 mL) was added 3-methylbutyl nitrite (472 μL, 3.52 mmol) whilst cooling in an ice/water bath. Trimethylsilyl azide (467 μL, 3.52 mmol) was added dropwise. After 10 min the ice/water bath was removed and the mixture allowed to warm to rt and stirred for 3 hrs. The mixture was cooled in an ice/water bath and a further charge of 3-methylbutyl nitrite (100 μL, 0.74 mmol) and trimethylsilyl azide (100 μL, 0.75 mmol) added. The mixture was stirred at rt for 60 min. The mixture was concentrated in vacuo and the residue taken up in EtOAc (50 mL), washed with water (30 mL) and brine (30 mL), dried over MgSO₄ and concentrated in vacuo to afford the intermediate azide.

1,4-Dioxane (50 mL) was added to the reaction flask containing the intermediate azide which was wrapped in foil to protect the reaction from light exposure. Propargyl alcohol (410 μL, 7.04 mmol), CuI (22 mg, 0.12 mmol) and sodium ascorbate (92 mg, 0.47 mmol) were added and the reaction heated at 80° C. overnight. A further charge of CuI (22 mg, 0.12 mmol) and sodium ascorbate (92 mg, 0.47 mmol) were added and heating continued at 80° C. for 24 hrs. The mixture was partitioned between EtOAc (50 mL) and saturated NH₄Cl (aq) (25 mL) and the layers separated. The organic layer was washed with brine (25 mL), dried over Na₂SO₄ and concentrated. Flash chromatography (0-100% EtOAc in Pet. Ether) afforded the title compound as a beige solid (280 mg, 40% yield).

[M+H]⁺=318.2

### Ethyl 2-fluoro-6-(4-formyl-1H-1,2,3-triazol-1-yl)-3-methoxybenzoate

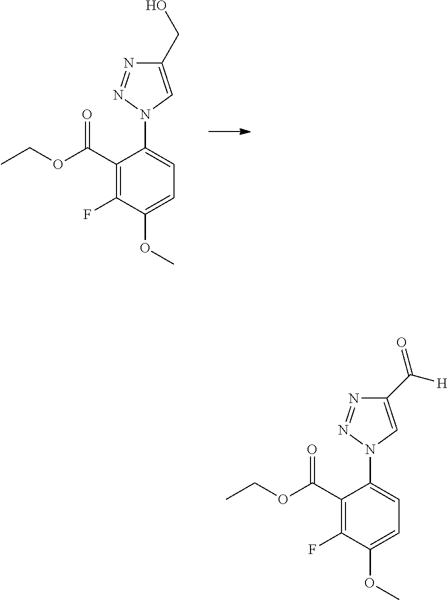 [SMILES: CCOC(=O)c1c(-n2cc(CO)nn2)ccc(OC)c1F ; CCOC(=O)c1c(-n2cc(C=O)nn2)ccc(OC)c1F]

A solution of ethyl 2-fluoro-6-(4-(hydroxymethyl)-1H-1,2,3-triazol-1-yl)-3-methoxybenzoate (225 mg, 0.76 mmol) in EtOAc (75 mL) was treated with 2-iodoxybenzoic acid (1.42 g, 2.29 mmol) and stirred at vigorous reflux for 4 hrs. Additional 2-iodoxybenzoic acid (50 mg) was added and heating continued for a further 60 min. The mixture was cooled to rt and filtered through Celite, washing with EtOAc. The filtrates were concentrated in vacuo and flash chromatography (0-100% EtOAc in Pet. Ether) afforded the title compound as an off white solid (223 mg, 100% yield).

[2M+H]⁺=587.1

### Ethyl 6-(4-(difluoromethyl)-1H-1,2,3-triazol-1-yl)-2-fluoro-3-methoxybenzoate

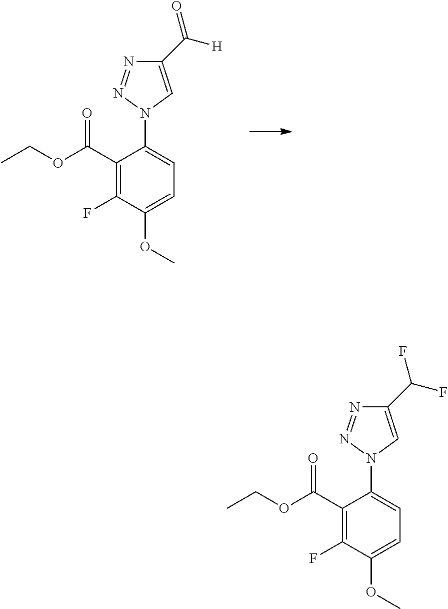 [SMILES: CCOC(=O)c1c(-n2cc(C=O)nn2)ccc(OC)c1F ; CCOC(=O)c1c(-n2cc(C(F)F)nn2)ccc(OC)c1F]

To ethyl 2-fluoro-6-(4-formyl-1H-1,2,3-triazol-1-yl)-3-methoxybenzoate (238 mg, 0.81 mmol) in DCM (5 mL) was added diethylaminosulfur trifluoride (161 μL, 1.22 mmol) and the mixture stirred at rt for 18 hrs. The mixture was poured into 20 mL of an iced solution of NaHCO₃(aq) and extracted with DCM (3×20 mL). The organic phases were combined and washed with water (20 mL) followed by brine (20 mL), dried over MgSO₄ and concentrated in vacuo. Flash chromatography (0-100% EtOAc in Pet. Ether) afforded the title compound as a pale yellow oil which solidified on standing (178 mg, 70% yield).

### (6-(4-(Difluoromethyl)-1H-1,2,3-triazol-1-yl)-2-fluoro-3-methoxyphenyl)methanol

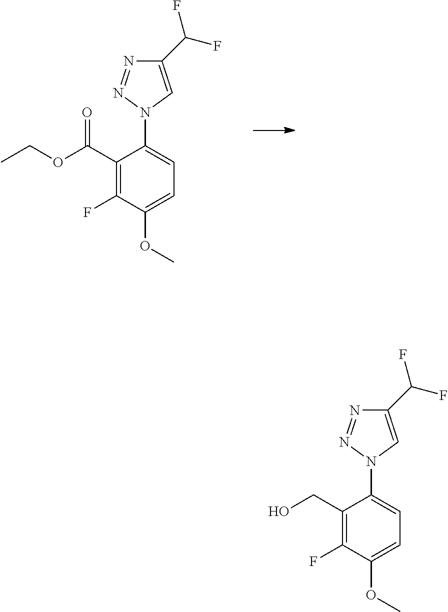 [SMILES: CCOC(=O)c1c(-n2cc(C(F)F)nn2)ccc(OC)c1F ; COc1ccc(-n2cc(C(F)F)nn2)c(CO)c1F]

To a stirred solution of ethyl 6-(4-(difluoromethyl)-1H-1,2,3-triazol-1-yl)-2-fluoro-3-methoxybenzoate (228 mg, 0.72 mmol) in THF (15 mL) at 0° C. was added LiBH₄ (32 mg, 1.4 mmol). The reaction was warmed to rt and stirred for 18 hrs, diluted with water (75 mL) and extracted with EtOAc (3×25 mL). The combined organic phases were dried over MgSO₄ and concentrated in vacuo. Flash chromatography (0-100% EtOAc in Pet. Ether) afforded the title compound as a white crystalline solid (182 mg, 92% yield).

[M+H]⁺=274.1

### 1-(2-(Bromomethyl)-3-fluoro-4-methoxyphenyl)-4-(difluoromethyl)-1H-1,2,3-triazole

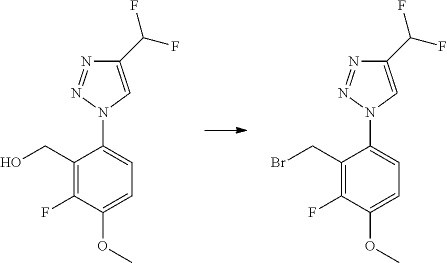 [SMILES: COc1ccc(-n2cc(C(F)F)nn2)c(CO)c1F ; COc1ccc(-n2cc(C(F)F)nn2)c(CBr)c1F]

To a solution of (6-(4-(difluoromethyl)-1H-1,2,3-triazol-1-yl)-2-fluoro-3-methoxyphenyl)methanol (182 mg, 0.67 mmol) in dry THF (10 mL) was added phosphorous tribromide (75 μL, 0.80 mmol) and the reaction stirred at rt for 2 hrs. The reaction mixture was quenched with dilute NaHCO₃ (sat. diluted to 10%) (10 mL) and extracted with DCM (3×25 mL). The combined organics were washed with water (10 mL) and brine (10 mL), dried (MgSO₄), filtered and concentrated to afford the title compound (223 mg, 100% yield) as a colourless oil that solidified on standing.

[M+H]⁺=337.9

### 2-(6-(4-(Difluoromethyl)-1H-1,2,3-triazol-1-yl)-2-fluoro-3-methoxybenzyl)isoindoline-1,3-dione

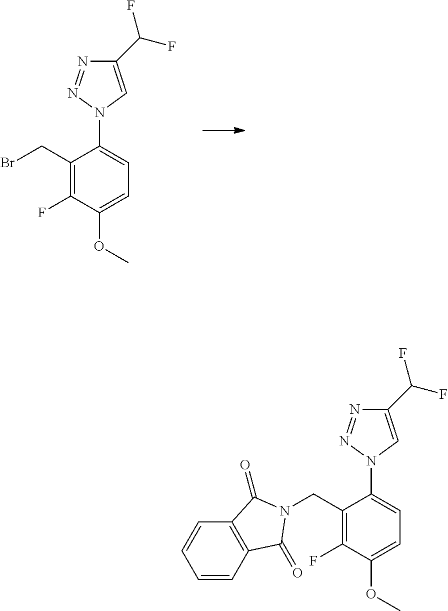 [SMILES: COc1ccc(-n2cc(C(F)F)nn2)c(CBr)c1F ; COc1ccc(-n2cc(C(F)F)nn2)c(CN2C(=O)c3ccccc3C2=O)c1F]

To 1-(2-(bromomethyl)-3-fluoro-4-methoxyphenyl)-4-(difluoromethyl)-1H-1,2,3-triazole (223 mg, 0.66 mmol) in dry DMF (2 mL) was added potassium phthalimde (117 mg, 0.63 mmol) and stirred at rt. After 5 mins potassium carbonate (175 mg, 1.26 mmol) was added and the reaction heated to 85° C. for 60 min. The reaction mixture was concentrated, azeotroping with toluene (4×40 mL). The product was purified by flash chromatography (0-11% (10% NH₃ in MeOH) in DCM) to afford the title compound (238 mg, 94% yield) as a white solid.

[M+H]⁺=403.0

### (6-(4-(Difluoromethyl)-1H-1,2,3-triazol-1-yl)-2-fluoro-3-methoxyphenyl)methanamine

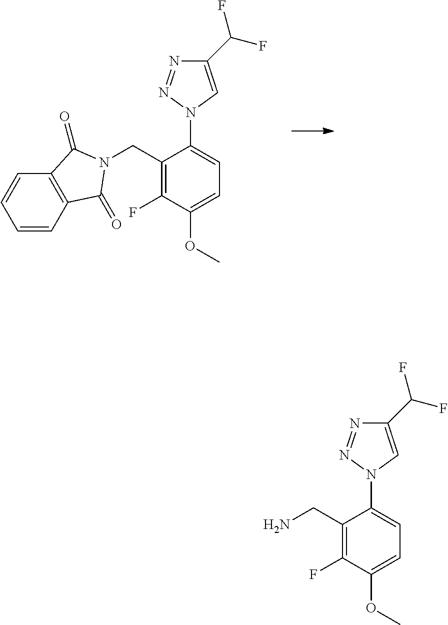 [SMILES: COc1ccc(-n2cc(C(F)F)nn2)c(CN2C(=O)c3ccccc3C2=O)c1F ; COc1ccc(-n2cc(C(F)F)nn2)c(CN)c1F]

To a solution of 2-(6-(4-(difluoromethyl)-1H-1,2,3-triazol-1-yl)-2-fluoro-3-methoxybenzyl)isoindoline-1,3-dione (238 mg, 0.592 mmol) in MeOH (5 mL) was added hydrazine hydrate (345 μL, 0.60 mmol) and the reaction stirred at 80° C. for 2 hrs. The solution was concentrated and azeotroped with toluene (4×40 mL). The residue was purified by flash chromatography (0-10% methanol in DCM) to afford the title compound (81 mg, 50% yield) as a white solid.

[M+H]⁺=273.1 @ 0.92 mins

¹H NMR (MeOD): 3.45 (2H, d, J=2.1 Hz), 3.83 (3H, s), 6.95 (1H, t, J=54.2 Hz), 7.07-7.19 (2H, m), 8.52 (1H, t, J=1.4 Hz)

### 6-(Aminomethyl)isoquinolin-1-amine (CAS 215454-95-8)

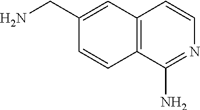 [SMILES: NCc1ccc2c(N)nccc2c1]

The title compound was synthesised according to WO2016083816

### 6-(Aminomethyl)-8-fluoroisoquinolin-1-amine

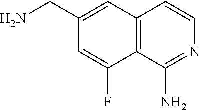 [SMILES: NCc1cc(F)c2c(N)nccc2c1]

The title compound was synthesised according to Xiaojun Zhang et al. J. Med. Chem. 2016, 59 (15), 7125-7137

### 6-(Aminomethyl)-7-methoxyisoquinolin-1-amine (CAS 1938129-46-4)

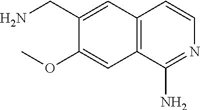

The title compound was synthesised according to Patent WO 2016083816

The following intermediates are widely commercially available:
6-Amino-2,3-dihydro-1H-isoindol-1-one: CAS 675109-45-2
6-Amino-3H-quinazolin-4-one: CAS 17329-31-6
Isoquinoline-1,7-diamine: CAS 244219-96-3
2-(1H-Imidazol-4-yl)ethylamine dihydrochloride: CAS 56-92-8
6-Aminoquinoxaline: CAS 6298-37-9
2-Methylbenzo[d]oxazol-6-amine: CAS 5676-60-8
3-(4-aminophenyl)-4,5-dihydro-1H-1,2,4-triazol-5-one: CAS 62036-31-1
(5R)-5H,6H,7H-cyclopenta[c]pyridine-1,5-diamine dihydrochloride: CAS 2096419-45-1

### SPECIFIC EXAMPLES OF THE PRESENT INVENTION

### Example 1.13

### N-[(1-Amino-6-isoquinolyl)methyl]-5-chloro-2-[(1-methyl-4-piperidyl)methylamino]pyridine-3-carboxamide

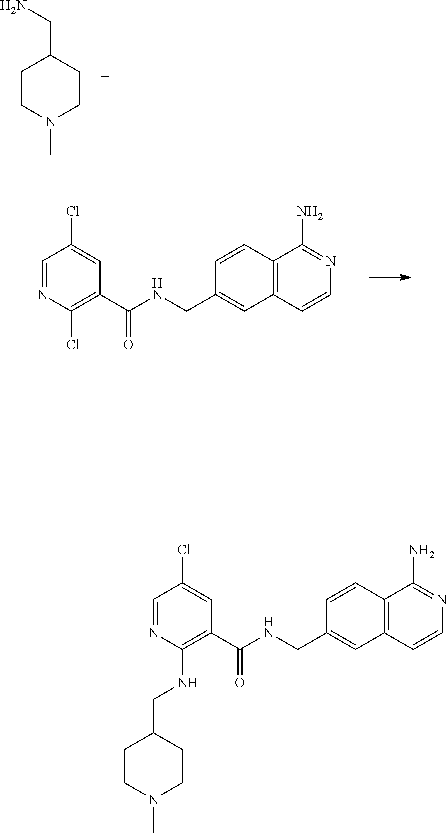

Following general method B(i), (1-methyl-4-piperidyl)methanamine (231 mg, 1.8 mmol) was reacted with N-[(1-amino-6-isoquinolyl)methyl]-2,5-dichloro-pyridine-3-carboxamide (250 mg, 0.72 mmol) to afford the title compound (256 mg, 79% yield) as a yellow powder.

[M+H]⁺=439.5

¹H NMR (DMSO-d6, 400 MHz) δ 1.18 (2H, qd, J=12.0, 3.8 Hz), 1.43-1.52 (1H, m), 1.55-1.66 (2H, m), 1.77 (2H, td, J=11.7, 2.4 Hz), 2.11 (3H, s), 2.72 (2H, dt, J=11.6, 3.2 Hz), 3.23-3.30 (2H, m), 4.56 (2H, d, J=5.7 Hz), 6.73 (2H, s), 6.87 (1H, dd, J=5.9, 0.8 Hz), 7.40 (1H, dd, J=8.6, 1.8 Hz), 7.56 (1H, s), 7.76 (1H, d, J=5.8 Hz), 8.10-8.17 (2H, m), 8.19 (1H, d, J=2.5 Hz), 8.53 (1H, t, J=5.7 Hz), 9.25 (1H, t, J=5.8 Hz)

### Example 1.50

### N-((1-aminoisoquinolin-6-yl)methyl)-5-chloro-2-(((1-cyclopropylpiperidin-4-yl)methyl)amino)nicotinamide

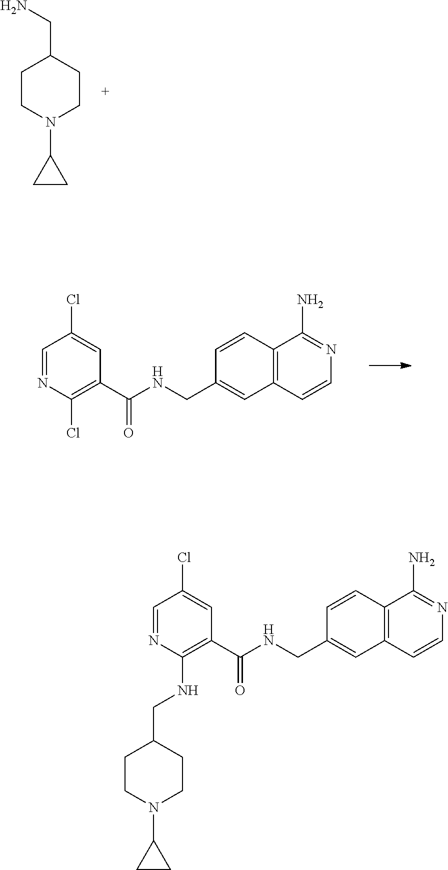 [SMILES: NCC1CCN(C2CC2)CC1 ; Nc1nccc2cc(CNC(=O)c3cc(Cl)cnc3Cl)ccc12 ; Nc1nccc2cc(CNC(=O)c3cc(Cl)cnc3NCC3CCN(C4CC4)CC3)ccc12]

Following general method B(i), N-[(1-amino-6-isoquinolyl)methyl]-2,5-dichloro-pyridine-3-carboxamide (35 mg, 0.1 mmol) was reacted with (1-cyclopropylpiperidin-4-yl)methanamine (31 mg, 0.2 mmol) to afford the title compound (19 mg, 27% yield).

[M+H]⁺=465.6

### Example 1.51

### N-((1-aminoisoquinolin-6-yl)methyl)-5-chloro-2-(((1-ethylpiperidin-4-yl)methyl)amino)nicotinamide

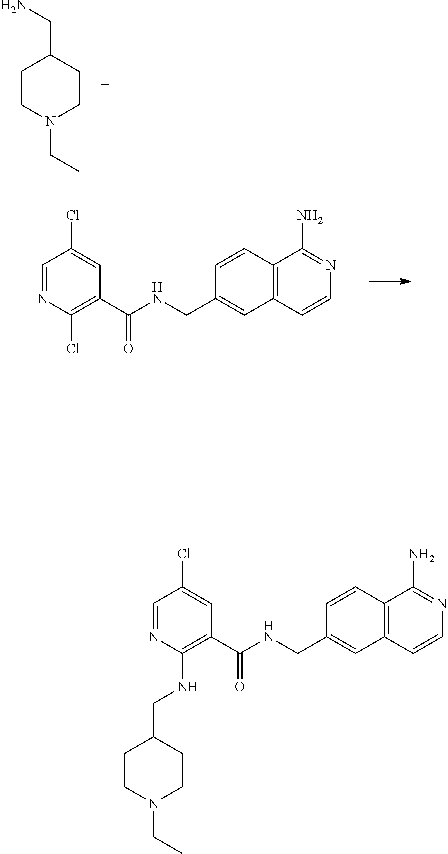

Following general method B(i), N-[(1-amino-6-isoquinolyl)methyl]-2,5-dichloro-pyridine-3-carboxamide (35 mg, 0.1 mmol) was reacted with (1-ethylpiperidin-4-yl)methanamine (28 mg, 0.2 mmol) to afford the title compound (21 mg, 30% yield).

[M+H]⁺=453.5

### Example 1.54

### N-((1-aminoisoquinolin-6-yl)methyl)-5-chloro-2-(((1-(2-methoxyethyl)piperidin-4-yl)methyl)amino)nicotinamide

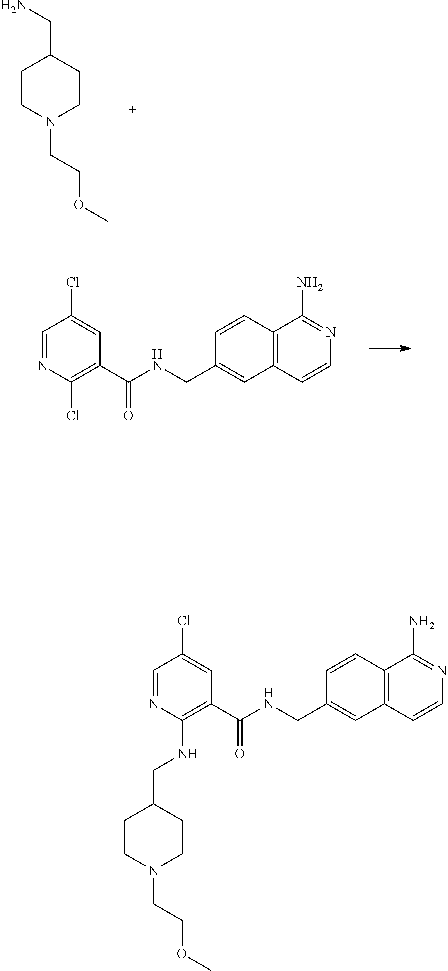 [SMILES: COCCN1CCC(CN)CC1 ; Nc1nccc2cc(CNC(=O)c3cc(Cl)cnc3Cl)ccc12 ; COCCN1CCC(CNc2ncc(Cl)cc2C(=O)NCc2ccc3c(N)nccc3c2)CC1]

Following general method B(i), N-[(1-amino-6-isoquinolyl)methyl]-2,5-dichloro-pyridine-3-carboxamide (35 mg, 0.1 mmol) was reacted with (1-(2-methoxyethyl)piperidin-4-yl)methanamine (34 mg, 0.2 mmol) to afford the title compound (23 mg, 28% yield).

[M+H]⁺=483.6

### Example 1.59

### N-((1-aminoisoquinolin-6-yl)methyl)-5-chloro-2-(((5,6,7,8-tetrahydroimidazo[1,2-a]pyridin-7-yl)methyl)amino)nicotinamide

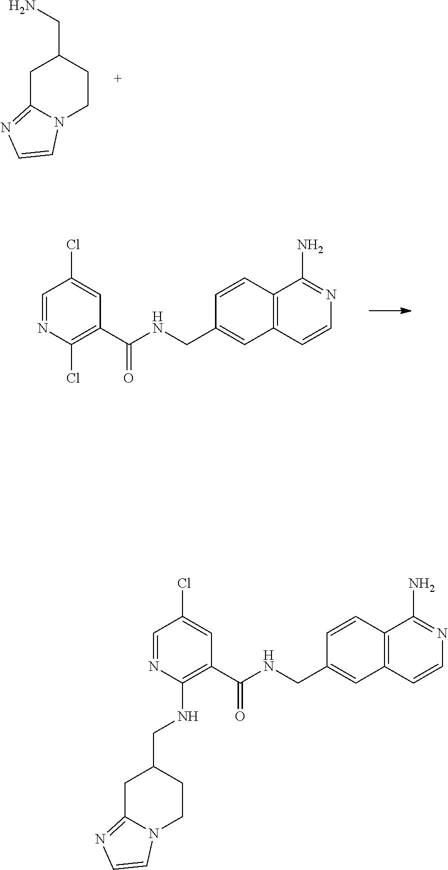 [SMILES: NCC1CCn2ccnc2C1 ; Nc1nccc2cc(CNC(=O)c3cc(Cl)cnc3Cl)ccc12 ; Nc1nccc2cc(CNC(=O)c3cc(Cl)cnc3NCC3CCn4ccnc4C3)ccc12]

Following general method B(i), N-[(1-amino-6-isoquinolyl)methyl]-2,5-dichloro-pyridine-3-carboxamide (35 mg, 0.1 mmol) was reacted with (5,6,7,8-tetrahydroimidazo[1,2-a]pyridin-7-yl)methanamine (30 mg, 0.2 mmol) to afford the title compound (15 mg, 21% yield).

[M+H]⁺=462.5

### Example 1.63

### N-[(1-amino-6-isoquinolyl)methyl]-5-chloro-2-[[1-(3-pyridylmethyl)-4-piperidyl]methylamino]pyridine-3-carboxamide

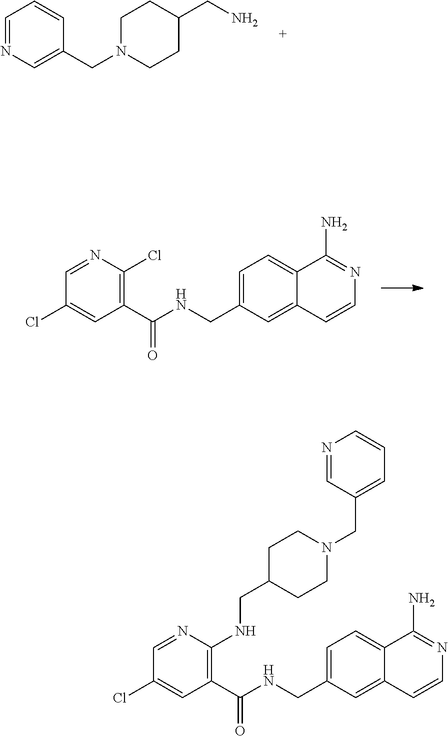 [SMILES: NCC1CCN(Cc2cccnc2)CC1 ; Nc1nccc2cc(CNC(=O)c3cc(Cl)cnc3Cl)ccc12 ; Nc1nccc2cc(CNC(=O)c3cc(Cl)cnc3NCC3CCN(Cc4cccnc4)CC3)ccc12]

[1-(3-pyridylmethyl)4-piperidyl]methanamine (148 mg, 0.72 mmol) and N-[(1-amino-6-isoquinolyl)methyl]-2,5-dichloro-pyridine-3-carboxamide (100 mg, 0.29 mmol) were reacted using general conditions B(i) to afford the title compound (75 mg, 50% yield) as a pale yellow solid.

[M+H]⁺=516.2

¹H NMR (DMSO, 400 MHz) δ 1.20 (2H, qd, J=12.1, 3.9 Hz), 1.59 (3H, t, J=19.8 Hz), 1.85-1.98 (2H, m), 2.78 (2H, d, J=11.1 Hz), 3.29 (2H, t, J=6.0 Hz), 3.46 (2H, s), 4.57 (2H, d, J=5.6 Hz), 6.72 (2H, s), 6.87 (1H, d, J=5.8 Hz), 7.34 (1H, dd, J=7.8, 4.7 Hz), 7.40 (1H, dd, J=8.7, 1.7 Hz), 7.57 (1H, d, J=1.6 Hz), 7.68 (1H, dt, J=7.8, 2.0 Hz), 7.76 (1H, d, J=5.8 Hz), 8.10-8.17 (2H, m), 8.19 (1H, d, J=2.4 Hz), 8.46 (2H, dt, J=6.5, 1.8 Hz), 8.52 (1H, t, J=5.7 Hz), 9.24 (1H, t, J=5.8 Hz).

### Example 1.64

### N-[(1-amino-6-isoquinolyl)methyl]5-chloro-2-[(1-isopropyl-3-piperidyl)methylamino]pyridine-3-carboxamide

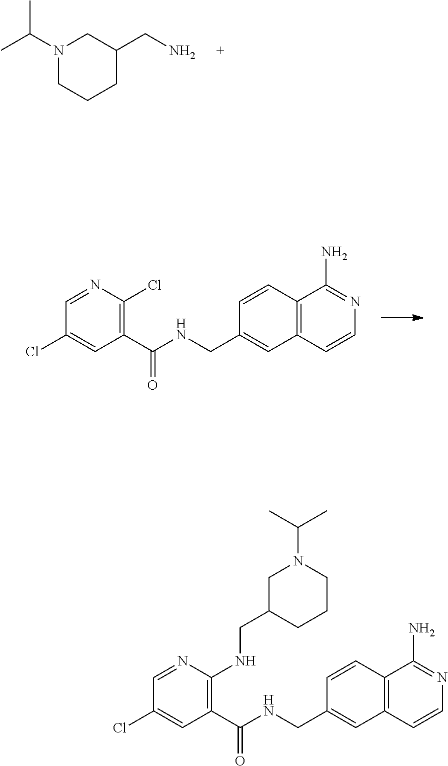 [SMILES: CC(C)N1CCCC(CN)C1 ; Nc1nccc2cc(CNC(=O)c3cc(Cl)cnc3Cl)ccc12 ; CC(C)N1CCCC(CNc2ncc(Cl)cc2C(=O)NCc2ccc3c(N)nccc3c2)C1]

Following general method B(i), (1-isopropyl-3-piperidyl)methanamine (113 mg, 0.72 mmol) was reacted with N-[(1-amino-6-isoquinolyl)methyl]-2,5-dichloro-pyridine-3-carboxamide (100 mg, 0.29 mmol) to afford the title compound (49 mg, 36% yield) as a pale yellow solid.

[M+H]⁺=467.3

¹H NMR (DMSO, 400 MHz) δ 0.91 (7H, t, J=7.0 Hz), 1.29-1.44 (1H, m), 1.55-1.70 (2H, m), 1.75 (1H, d, J=6.1 Hz), 1.90 (1H, t, J=10.1 Hz), 2.07 (1H, t, J=10.7 Hz), 2.56-2.74 (3H, m), 3.25-3.31 (2H, m), 4.57 (2H, d, J=5.7 Hz), 6.72 (2H, s), 6.87 (1H, d, J=5.8 Hz), 7.41 (1H, dd, J=8.6, 1.7 Hz), 7.57 (1H, s), 7.77 (1H, d, J=5.8 Hz), 8.08-8.17 (2H, m), 8.20 (1H, d, J=2.5 Hz), 8.49 (1H, t, J=5.7 Hz), 9.24 (1H, t, J=5.9 Hz).

### Example 1.72

### R—N-[(1-aminoisoquinoline-6-yl)methyl)-5-chloro-2-(((5-oxopyrrolidin-2-yl)methyl)amino)nicotinamide

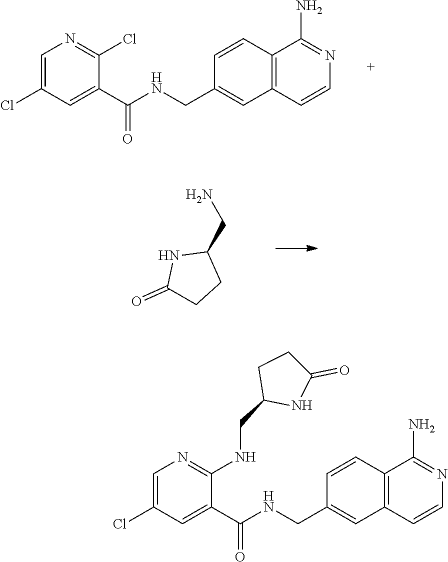

Following general conditions B(ii), N-((1-aminoisoquinoline-6-yl)methyl)-2,5-dichloronicotinamide (100 mg, 0.29 mmol) and (R)-5-(aminomethyl)pyrrolidin-2-one (132 mg, 1.15 mmol) were reacted to afford the title compound as an off white solid (10.1 mg, 2% yield).

[M+H]⁺=425.3

(DMSO): 1.77-1.69 (1H, m), 2.20-2.03 (4H, m), 3.67-3.59 (1H, m), 3.80-3.74 (1H, m), 4.59 (2H, d, J=5.6 Hz), 6.75-6.72 (2H, m), 6.90 (1H, d, J=5.5 Hz), 7.43 (1H, dd, J=1.7, 8.6 Hz), 7.59 (1H, s), 7.79-7.74 (2H, m), 8.21-8.14 (4H, m), 8.58 (1H, t, J=5.9 Hz), 9.25 (1H, t, J=5.8 Hz).

### Example 4.01

### N-((1-aminoisoquinolin-6-yl)methyl)-2-(((1-methylpiperidin-4-yl)methyl)amino)nicotinamide

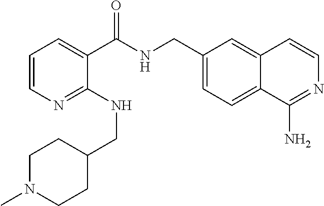 [SMILES: CN1CCC(CNc2ncccc2C(=O)NCc2ccc3c(N)nccc3c2)CC1]

### N-((1-aminoisoquinolin-6-yl)methyl)-2-chloronicotinamide

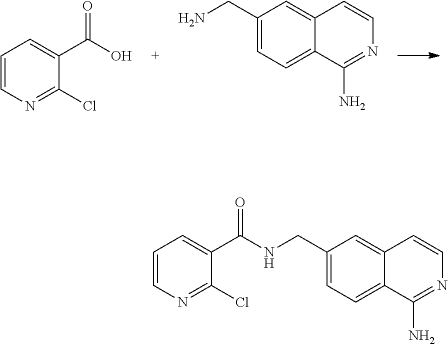

Following general method A, 2-chloro-nicotinic acid (80 mg, 0.51 mmol) and 1-amino-6-aminomethyl-isoquinoline (88 mg, 0.51 mmol) were reacted to afford the title compound (50 mg, 31% yield) as an off white solid.

[M+H]⁺=313.2

¹H NMR (DMSO-d6): 4.65 (2H, d, J=5.9 Hz), 7.12 (1H, d, J=6.6 Hz), 7.54 (1H, dd, J=4.9, 7.6 Hz), 7.67 (1H, d, J=8.5 Hz), 7.71 (1H, d, J=6.6 Hz), 7.81 (1H, s), 8.03 (1H, dd, J=2.0, 7.5 Hz), 8.43 (1H, d, J=8.6 Hz), 8.50 (1H, dd, J=1.8, 4.7 Hz), 9.32 (1H, t, J=5.8 Hz)

### N-((1-aminoisoquinolin-6-yl)methyl)-2-(((1-methylpiperidin-4-yl)methyl)amino)nicotinamide

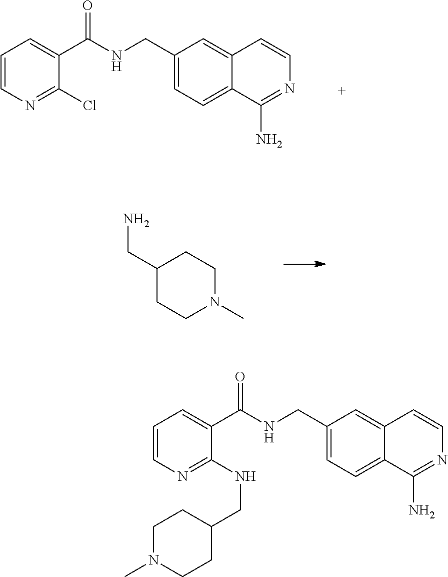

In a modification to general method B(ii), N-((1-aminoisoquinolin-6-yl)methyl)-2-chloronicotinamide (25 mg, 0.08 mmol), N-(1-methyl-piperidin-4-yl)methylamine (66 μL, 0.48 mmol) and triethylamine (22 μL, 0.16 mmol) in n-butanol (0.35 mL) were added to a sealed microwave tube and heated to 120° C. for 3 hrs. The crude reaction mixture was dissolved in DCM (50 mL) and washed with water (3×100 mL), the organic was dried (MgSO₄) and concentrated in vacuo. Flash chromatography (0-10% (1% NH₃ in MecOH) in DCM) afforded the title compound (17 mg, 53% yield) as an off white solid.

[M+H]⁺=405.3

¹H NMR (Methanol-d4): 1.39 (2H, qd, J=3.4, 12.2 Hz), 1.69 (1H, m), 1.86 (2H, d, J=13.5 Hz), 2.09 (2H, t, J 10=11.7 Hz), 2.33 (3H, s), 2.95 (2H, d, J=11.8 Hz), 3.40 (2H, d, J=6.9 Hz), 4.74 (2H, s), 6.65 (1H, dd, J=5.0, 7.6 Hz), 7.01 (1H, d, J=6.0 Hz), 7.55 (1H, dd, J=1.7, 8.6 Hz), 7.69 (1H, s), 7.77 (1H, d, J=6.0 Hz), 7.99 (1H, dd, J=1.6, 7.6 Hz), 8.14 (1H, dd, J=8.6 Hz), 8.17 (1H, dd, J=1.8, 4.9 Hz)

### Example 4.03

### N-((1-aminoisoquinolin-6-yl)methyl)-3-(((1-methylpiperidin-4-yl)methyl)amino)pyrazine-2-carboxamide

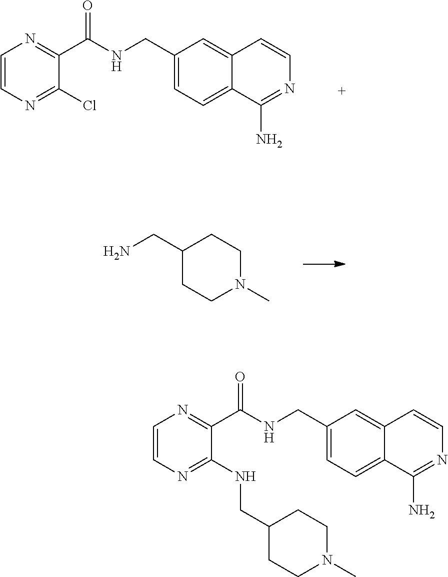

Following general method B(i), N-((1-aminoisoquinolin-6-yl)methyl)-3-chloropyrazine-2-carboxamide (75 mg, 0.24 mmol) and (1-methylpiperidin-4-yl)methanamine (123 mg, 0.96 mmol) were heated to 140° C. for 30 min. The crude mixture was diluted with water (10 mL) and the aqueous was decanted. The product was purified by preparative HPLC, to afford the title compound (34 mg, 35% yield) as a yellow gum.

[M+H]⁺=406.3

¹H NMR (DMSO) 1.35-1.24 (2H, m), 1.71-1.60 (31H, m), 2.15 (2H, dd, J=9.9, 11.8 Hz), 2.32-2.31 (3H, m), 2.94 (2H, d, J=11.7 Hz), 4.61-4.57 (2H, m), 6.80 (2H, s), 6.87 (1H, d, J=5.6 Hz), 7.45-7.42 (1H, m), 7.56 (1H, s), 7.83-7.75 (21H, m), 8.16-8.13 (1H, m), 8.22 (2H, s), 8.29-8.28 (1H, m), 8.84-8.79 (1H, m), 9.50 (1H, t, J=6.3 Hz).

### Example 4.11

### N-((1-aminoisoquinolin-6-yl)methyl)-6-(((1-methylpiperidin-4-yl)methyl)amino)-[3,3′-bipyridine]-5-carboxamide

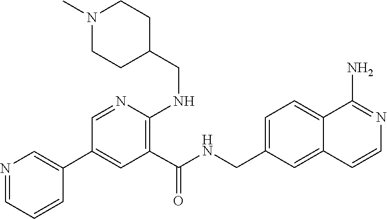 [SMILES: CN1CCC(CNc2ncc(-c3cccnc3)cc2C(=O)NCc2ccc3c(N)nccc3c2)CC1]

### N-((1-Aminoisoquinolin-6-yl)methyl)-2,5-dibromonicotinamide

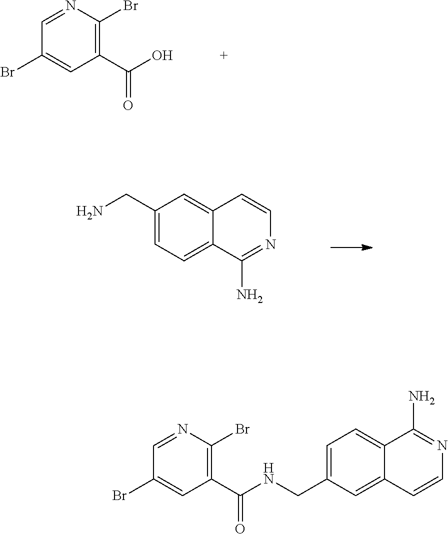 [SMILES: O=C(O)c1cc(Br)cnc1Br ; "+" ; NCc1ccc2c(N)nccc2c1 ; Nc1nccc2cc(CNC(=O)c3cc(Br)cnc3Br)ccc12]

Following general method A(i), 2,5-dibromonicotinic acid (200 mg, 0.71 mmol) was reacted with 6-(aminomethyl)isoquinolin-1-amine hydrochloride (149 mg, 0.71 mmol) to afford the title compound (310 mg, 99% yield).

### N-((1-Aminoisoquinolin-6-yl)methyl)-5-bromo-2-(((1-methylpiperidin-4-yl)methyl)amino)nicotinamide

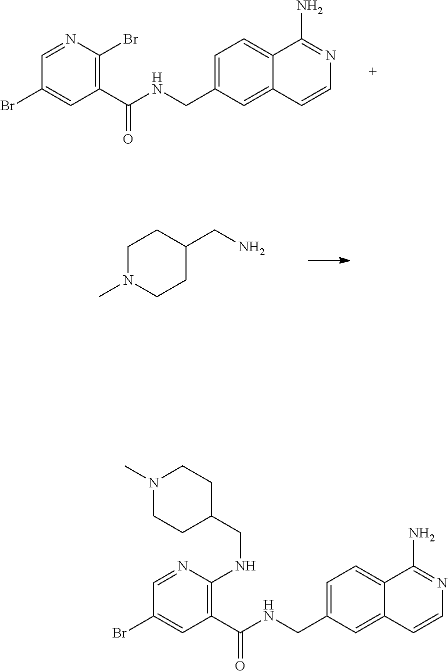

Following general method B(ii), N-((1-Aminoisoquinolin-6-yl)methyl)-2,5-dibromonicotinamide (310 mg, 0.71 mmol) was reacted with (1-methylpiperidin-4-yl)methanamine (456 mg, 3.55 mmol) to afford the title compound (310 mg, 90% yield).

### N-((1-aminoisoquinolin-6-yl)methyl)-6-(((1-methylpiperidin-4-yl)methyl)amino)-[3,3′-bipyridine]-5-carboxamide

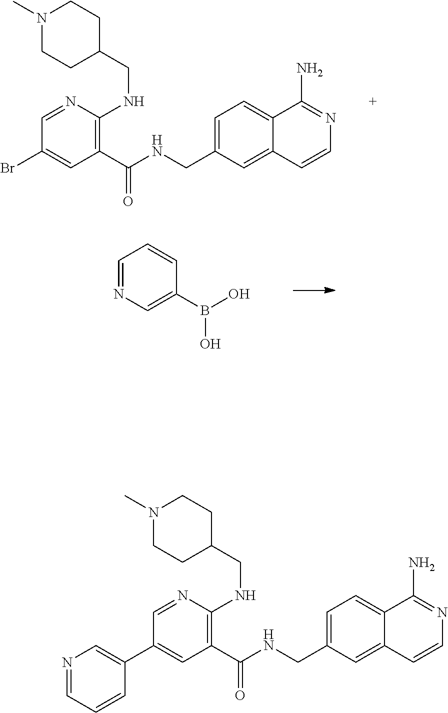

N-((1-aminoisoquinolin-6-yl)methyl)-5-bromo-2-(((1-methylpiperidin-4-yl)methyl)amino) nictoinamide (150 mg, 0.31 mmol), pyridin-3-ylboronic acid (50 mg, 0.40 mmol), palladium (II) acetate (3.5 mg, 0.016 mmol), SPhos (13 mg, 0.03 mmol) and potassium phosphate tribasic (231 mg, 1.09 mmol) was added to 1,4-dioxane (6 mL) and water (1 mL) and stirred at 100° C. for 5 hrs. EtOAc (25 mL) and water (5 mL) were added and the crude mixture was stirred vigorously for 10 min. The organic layer was extracted and concentrated in vacuo. Reverse phase preparative HPLC (0-100% MeCN in (0.1% formic acid in water)) afforded the title compound (20 mg, 13% yield).

[M+H]⁺=482.0

¹H NMR (DMSO) 1.32-1.21 (2H, m), 1.70-1.56 (3H, m), 1.96 (2H, dd, J=10.6, 11.5 Hz), 2.23-2.21 (3H, 10 m), 2.85 (2H, d, J=11.3 Hz), 3.39 (2H, t, J=7.3 Hz), 4.65 (2H, d, J=5.6 Hz), 6.75 (2H, s), 6.89 (1H, d, J=5.6 Hz), 7.50-7.43 (2H, m), 7.61 (1H, s), 7.78 (1H, d, J=5.9 Hz), 8.21-8.20 (3H, m), 8.45 (1H, d, J=2.4 Hz), 8.53 (1H, dd, J=1.5, 4.8 Hz), 8.62 (1H, d, J=2.4 Hz), 8.70 (1H, t, J=5.7 Hz), 8.97 (1H, d, J=1.6 Hz), 9.33 (1H, t, J=5.8 Hz).

### Example 8.06

### N-((1-aminoisoquinolin-6-yl)methyl)-5-chloro-6-(methyl(1-methylpiperidin-4-yl)amino)nicotinamide

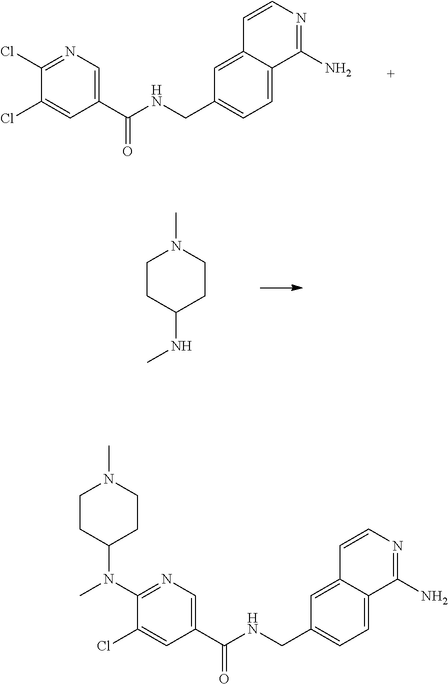

Following general method B(i), N-((1-aminoisoquinolin-6-yl)methyl)-5,6-dichloronicotinamide (35 mg, 0.1 mmol) was reacted with N,1-dimethylpiperidin-4-amine (38 mg, 0.3 mmol). The title compound was isolated (13 mg, 23% yield) as an off white solid.

[M+H]⁺=436.4

### Example 8.09

### N-[(1-amino-6-isoquinolyl)methyl]-5-chloro-6-[methyl-[[1-(4-pyridyl)-4-piperidyl]methyl]amino]pyridine-3-carboxamide

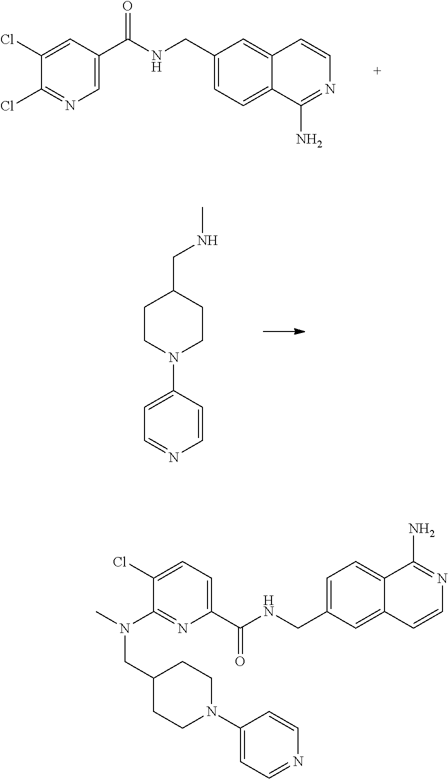 [SMILES: Nc1nccc2cc(CNC(=O)c3cnc(Cl)c(Cl)c3)ccc12 ; "+" ; CNCC1CCN(c2ccncc2)CC1 ; CN(CC1CCN(c2ccncc2)CC1)c1nc(C(=O)NCc2ccc3c(N)nccc3c2)ccc1Cl]

Following general method B(i), N-[(1-amino-6-isoquinolyl)methyl]-5,6-dichloro-pyridine-3-carboxamide (130 mg, 0.37 mmol) was reacted with N-methyl-1-[1-(4-pyridyl)-4-piperidyl]methanamine (200 mg, 0.97 mmol) to afford the title compound (82 mg, 37% yield) as a pale yellow powder.

[M+H]⁺=515.8

¹H NMR (DMSO, 400 MHz): 1.08-1.23 (2H, m), 1.74 (2H, d, J=11.8 Hz), 2.11-2.24 (1H, m), 3.07-3.19 (5H, m), 3.52 (2H, d, J=7.3 Hz), 4.23 (2H, d, J=13.4 Hz), 4.66 (2H, d, J=5.8 Hz), 7.12-7.20 (2H, m), 7.22 (1H, d, J=7.0 Hz), 7.64-7.71 (1H, m), 7.73 (1H, dd, J=8.6, 1.7 Hz), 7.84 (1H, s), 8.14-8.26 (3H, m), 8.59 (1H, d, J=8.6 Hz), 8.70 (1H, d, J=2.1 Hz), 9.20 (2H, br.s), 9.36 (1H, t, J=5.9, 5.9 Hz), 13.41 (1H, s), 13.55 (1H, s).

### Example 11.01

### N-((1-aminoisoquinolin-6-yl)methyl)-5-chloro-6-(cyclopentylamino)nicotinamide

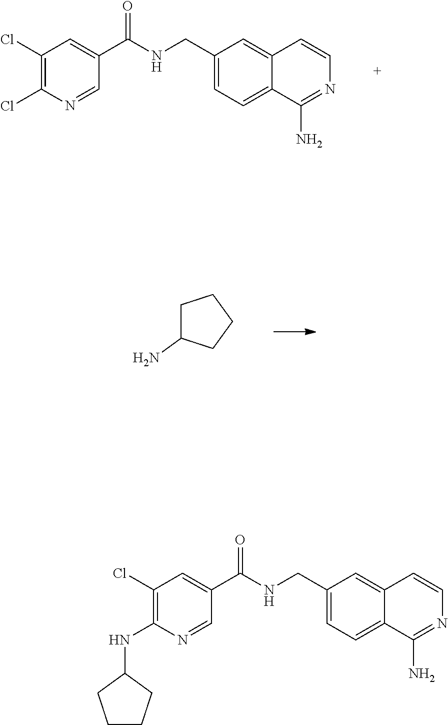 [SMILES: Nc1nccc2cc(CNC(=O)c3cnc(Cl)c(Cl)c3)ccc12 ; "+" ; NC1CCCC1 ; Nc1nccc2cc(CNC(=O)c3cnc(NC4CCCC4)c(Cl)c3)ccc12]

Following a modification to general method E, N-((1-aminoisoquinolin-6-yl)methyl)-5,6-dichloronicotinamide (55 mg, 0.16 mmol) and cyclopentylamine (135 mg, 1.6 mmol) were reacted in DMF at 120° C. for 18 hrs. The reaction was cooled to rt and partitioned between 20% IPA-chloroform (25 mL) and water (20 mL). The aqueous layer was extracted with further 20% IPA-chloroform (2×15 mL). Combined organics were dried (Na₂SO₄), filtered and concentrated and then purified by flash chromatography (0-10% (1% NH₃ in MecOH) in DCM). The product was freeze dried to afford the title compound (11 mg, 18% yield) as an off white solid.

[M+H]⁺=396.1

¹H NMR (DMSO): 1.50-1.60 (4H, m), 1.67-1.78 (2H, m), 1.91-1.98 (2H, m), 4.35-4.42 (1H, m), 4.58 (2H, d, J=5.8 Hz), 6.64 (1H, d, J=7.3 Hz), 6.84 (2H, s), 6.88 (1H, d, J=5.9 Hz), 7.41 (1H, dd, J=8.6, 1.5 Hz), 7.56 (1H, s), 7.75 (1H, d, J=5.9 Hz), 8.06 (1H, d, J=2.1 Hz), 8.15 (1H, d, J=8.6 Hz), 8.58 (1H, d, J=2.0 Hz), 8.93 (1H, t, J=5.9 Hz)

### Example 11.23

### N-((1-aminoisoquinolin-6-yl)methyl)-5-chloro-6-((4-(2-(dimethylamino)ethoxy)benzyl)amino)nicotinamide

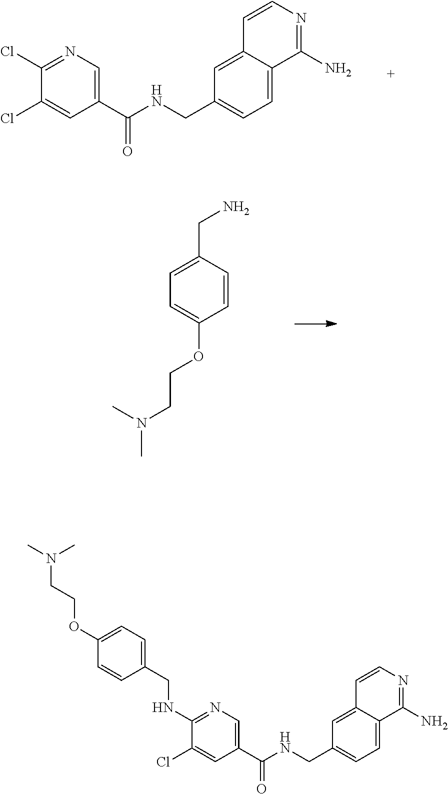 [SMILES: Nc1nccc2cc(CNC(=O)c3cnc(Cl)c(Cl)c3)ccc12 ; "+" ; CN(C)CCOc1ccc(CN)cc1 ; CN(C)CCOc1ccc(CNc2ncc(C(=O)NCc3ccc4c(N)nccc4c3)cc2Cl)cc1]

Following general method B(i), N-((1-aminoisoquinolin-6-yl)methyl)-5,6-dichloronicotinamide (36 mg, 0.1 mmol) was reacted with 2-(4-(aminomethyl)phenoxy)-N,N-dimethylethan-1-amine (24 mg, 0.122 mmol). The title compound was isolated (7 mg, 15% yield) as an off white solid. [M+H]⁺=505.4

### Example 11.20

### N-((1-aminoisoquinolin-6-yl)methyl)-5-chloro-6-((2-(diisopropylamino)ethyl)amino)nicotinamide

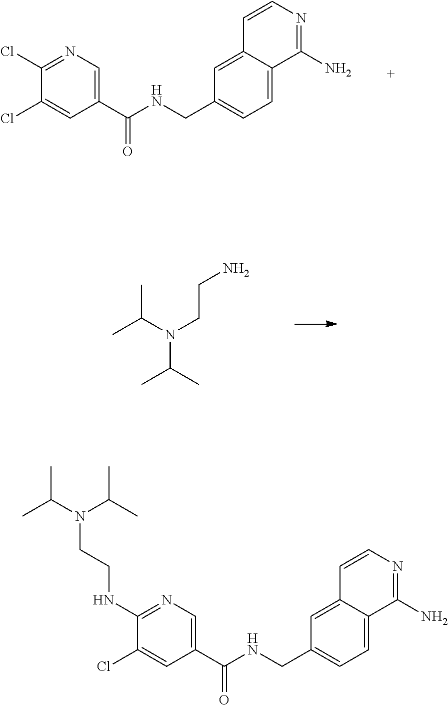

Following general method B(i), N-((1-aminoisoquinolin-6-yl)methyl)-5,6-dichloronicotinamide (35 mg, 0.1 mmol) was reacted with N,N-diisopropylethane-1,2-diamine (18 mg, 0.122 mmol). The title compound was isolated (19 mg, 26% yield) as an off white solid.

[M+H]⁺=455.5

### Example 13.09

### N-((1-aminoisoquinolin-6-yl)methyl)-5-chloro-6-(2-cyanophenoxy)nicotinamide

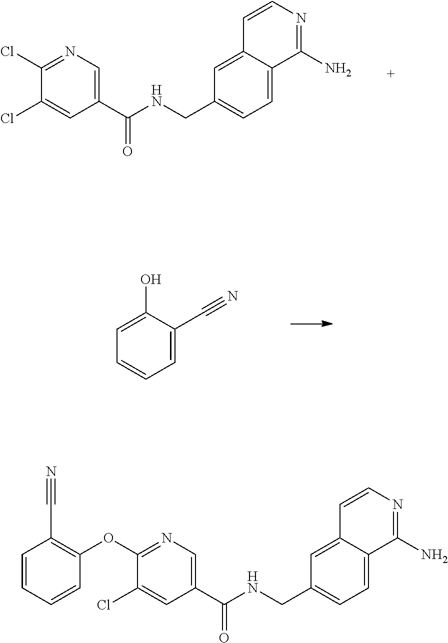 [SMILES: Nc1nccc2cc(CNC(=O)c3cnc(Cl)c(Cl)c3)ccc12 ; "+" ; N#Cc1ccccc1O ; N#Cc1ccccc1Oc1ncc(C(=O)NCc2ccc3c(N)nccc3c2)cc1Cl]

Following general method C for phenols, N-((1-aminoisoquinolin-6-yl)methyl)-5,6-dichloronicotinamide (36 mg, 0.1 mmol) was reacted with 2-hydroxybenzonitrile (24 mg, 0.122 mmol). The title compound was isolated (10 mg, 13% yield) as an off white solid.

[M+H]⁺=430.4

### Example 13.24

### N-((1-aminoisoquinolin-6-yl)methyl)-5-chloro-6-(4-(piperazin-1-yl)phenoxy)nicotinamide

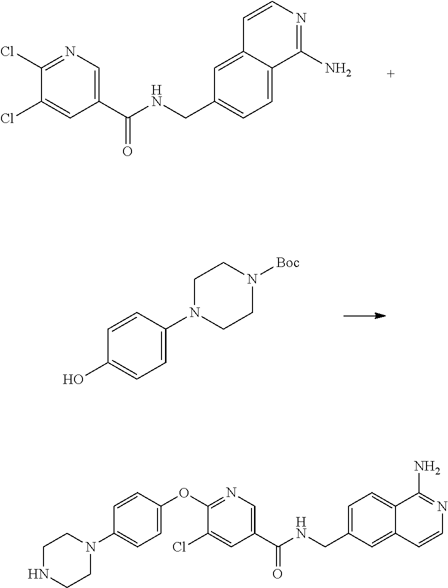 [SMILES: Nc1nccc2cc(CNC(=O)c3cnc(Cl)c(Cl)c3)ccc12 ; "+" ; CC(C)(C)OC(=O)N1CCN(c2ccc(O)cc2)CC1 ; Nc1nccc2cc(CNC(=O)c3cnc(Oc4ccc(N5CCNCC5)cc4)c(Cl)c3)ccc12]

Following general method C for phenols, N-((1-aminoisoquinolin-6-yl)methyl)-5,6-dichloronicotinamide (22 mg, 0.1 mmol) was reacted with tert-butyl 4-(4-hydroxyphenyl)piperazine-1-carboxylate (34 mg, 0.122 mmol). The isolated product was then dissolved in DCM (0.6 mL) and TFA (0.6 mL) added. This was stirred at rt for 3 hrs. The solvent was evaporated and the residue lyophylised with MeCN:water (7:3; 650 μL) to afford the title compound (25 mg, 21% yield) as an off white solid.

[M+H]⁺=489.5

### Example 13.26

### N-((1-aminoisoquinolin-6-yl)methyl)-5-chloro-6-(2-(piperidin-1-yl)ethoxy)nicotinamide

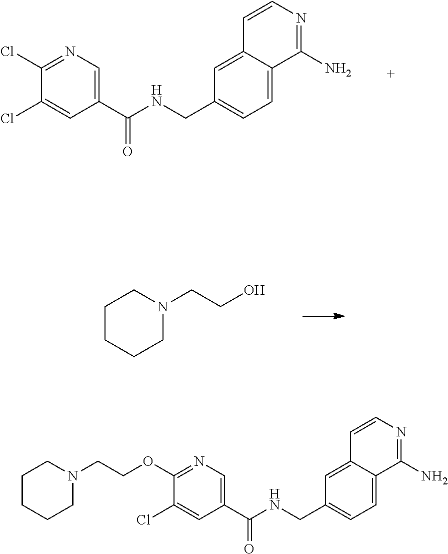 [SMILES: Nc1nccc2cc(CNC(=O)c3cnc(Cl)c(Cl)c3)ccc12 ; "+" ; OCCN1CCCCC1 ; Nc1nccc2cc(CNC(=O)c3cnc(OCCN4CCCCC4)c(Cl)c3)ccc12]

Following general method C for alcohols, N-((1-aminoisoquinolin-6-yl)methyl)-5,6-dichloronicotinamide (35 mg, 0.1 mmol) was reacted with 2-(piperidin-1-yl)ethan-1-ol (26 mg, 0.2 mmol). The title compound was isolated (29 mg, 54% yield) as an off white solid.

[M+H]⁺=440.5

### Example 19.01

### N-((1-aminoisoquinolin-6-yl)methyl)-5-(2-methoxyphenyl)nicotinamide

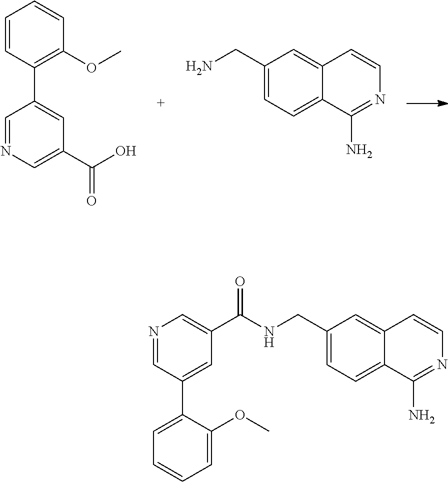 [SMILES: COc1ccccc1-c1cncc(C(=O)O)c1 ; NCc1ccc2c(N)nccc2c1 ; COc1ccccc1-c1cncc(C(=O)NCc2ccc3c(N)nccc3c2)c1]

Following general method A(i), 5-(2-methoxyphenyl)nicotinic acid (109 mg, 0.43 mmol) was reacted with 6-(aminomethyl)isoquinolin-1-amine dihydrochloride (105 mg, 0.43 mmol) to afford the title compound (20.1 mg, 12% yield) as a white solid.

[M+H]⁺=385.2

¹H NMR (d6-DMSO) δ: 3.81 (3H, s), 4.66 (2H, d, J=5.8 Hz), 6.75 (2H, s), 6.89 (1H, d, J=5.6 Hz), 7.06-7.14 (1H, m), 7.16-7.23 (1H, m), 7.40-7.48 (3H, m), 7.61 (1H, s), 7.77 (1H, d, J=5.8 Hz), 8.16 (1H, d, J=8.6 Hz), 8.35 (1H, t, J=2.1 Hz), 8.84 (1H, d, J=2.1 Hz), 9.03 (1H, d, J=2.1 Hz), 9.36 (1H, t, J=5.9 Hz)

### Example 19.02

### N-((1-aminoisoquinolin-6-yl)methyl)-5-bromonicotinamide

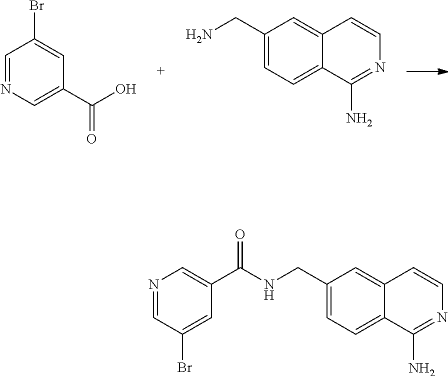 [SMILES: O=C(O)c1cncc(Br)c1 ; NCc1ccc2c(N)nccc2c1 ; Nc1nccc2cc(CNC(=O)c3cncc(Br)c3)ccc12]

Following general method A(i), 5-bromonicotinic acid (86 mg, 0.43 mmol)) was reacted with 6-(aminomethyl)isoquinolin-1-amine dihydrochloride (105 mg, 0.43 mmol) to afford the title compound (15 mg, 10% yield) as a white solid.

[M+H]⁺=357.1

¹H NMR (d6-DMSO) δ: 4.64 (2H, d, J=5.8 Hz), 6.74 (2H, s), 6.89 (1H, d, J=5.6 Hz), 7.43 (1H, dd, J=1.7, 8.6 Hz), 7.61 (1H, s), 7.77 (1H, d, J=5.8 Hz), 8.16 (1H, d, J=8.6 Hz), 8.48-8.52 (1H, m), 8.89 (1H, d, J=2.0 Hz), 9.05 (1H, d, J=1.8 Hz), 9.40 (1H, t, J=5.8 Hz)

### Example 19.04

### N-((1-aminoisoquinolin-6-yl)methyl)-5-chloronicotinamide

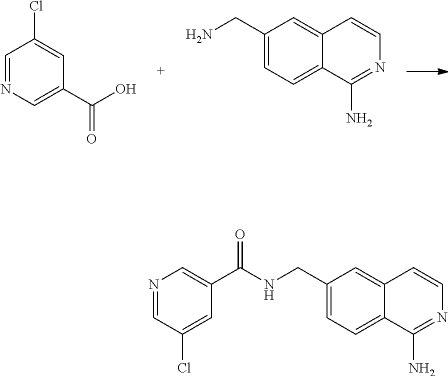 [SMILES: O=C(O)c1cncc(Cl)c1 ; NCc1ccc2c(N)nccc2c1 ; Nc1nccc2cc(CNC(=O)c3cncc(Cl)c3)ccc12]

Following general method A(ii), 5-chloro-3-pyridine carboxylic acid (68 mg, 0.43 mmol) was reacted with 6-(aminomethyl)isoquinoline-1-amine (75 mg, 0.43 mmol) to afford the title compound (48 mg, 36%) as a yellow solid.

[M+H]⁺=312.8

¹H NMR (DMSO): 4.65 (2H, d, J=5.8 Hz), 6.92 (1H, d, J=5.9 Hz), 6.96 (2H, s), 7.47 (1H, dd, J=1.5, 8.6 Hz), 7.63 (1H, s), 7.77 (1H, d, J=5.8 Hz), 8.20 (1H, d, J=8.6 Hz), 8.39 (1H, t, J=2.0 Hz), 8.81 (1H, d, J=2.3 Hz), 9.04 (1H, d, J=1.8 Hz), 9.47 (1H, t, J=5.8 Hz)

### Example 19.05

### N-((1-aminoisoquinolin-6-yl)methyl)-3-chlorobenzamide

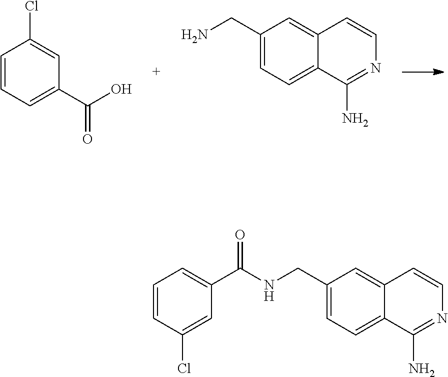 [SMILES: O=C(O)c1cccc(Cl)c1 ; NCc1ccc2c(N)nccc2c1 ; Nc1nccc2cc(CNC(=O)c3cccc(Cl)c3)ccc12]

Following general method A(ii), 3-chlorobenzoic acid (68 mg, 0.43 mmol) was reacted with 6-(aminomethyl)isoquinoline-1-amine (75 mg, 0.43 mmol) to afforded the title compound (50 mg, 37% yield) as an off white solid.

[M+]⁺=311.8

¹H NMR (DMSO): 4.62 (2H, d, J=5.8 Hz), 6.75 (2H, s), 6.88 (1H, d, J=5.8 Hz), 7.42 (1H, dd, J=1.4, 8.6 Hz), 7.52-7.58 (2H, m), 7.62-7.64 (1H, m), 7.77 (1H, d, J=5.8 Hz), 7.89 (1H, d, J=7.8 Hz), 7.97-7.98 (1H, m), 8.16 (1H, d, J=8.6 Hz), 9.27 (1H, t, J=5.8 Hz)

### Example 19.06

### N-(1-Amino-isoquinolin-6-ylmethyl)-5,6-dichloro-nicotinamide

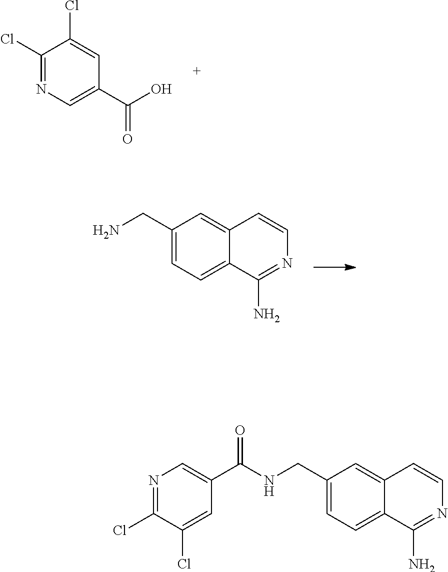 [SMILES: O=C(O)c1cnc(Cl)c(Cl)c1 ; NCc1ccc2c(N)nccc2c1 ; Nc1nccc2cc(CNC(=O)c3cnc(Cl)c(Cl)c3)ccc12]

Following general method A(ii), 5,6-dichloronicotinic acid (222 mg, 1.16 mmol) was reacted with 6-(aminomethyl)isoquinoline-1-amine (200 mg, 1.16 mmol) to afford the title compound (185 mg, 46% yield) as an off white solid.

[M+H]⁺=347.2

¹H NMR (DMSO): 4.64 (2H, d, J=5.8 Hz), 6.75 (2H, s), 6.88 (1H, d, J=5.8 Hz), 7.43 (1H, dd, J=8.6, 1.6 Hz), 7.60 (1H, s), 7.77 (1H, d, J=5.8 Hz), 8.18 (1H, d, J=8.6 Hz), 8.56 (1H, d, J=2.2 Hz), 8.67 (1H, d, J=2.0 Hz), 9.44 (1H, t, J=5.8 Hz)

### Example 22.01

### N-((1-aminoisoquinolin-6-yl)methyl)-5-chloro-2-(4-(2-oxopyrrolidin-1-yl)phenoxy)nicotinamide

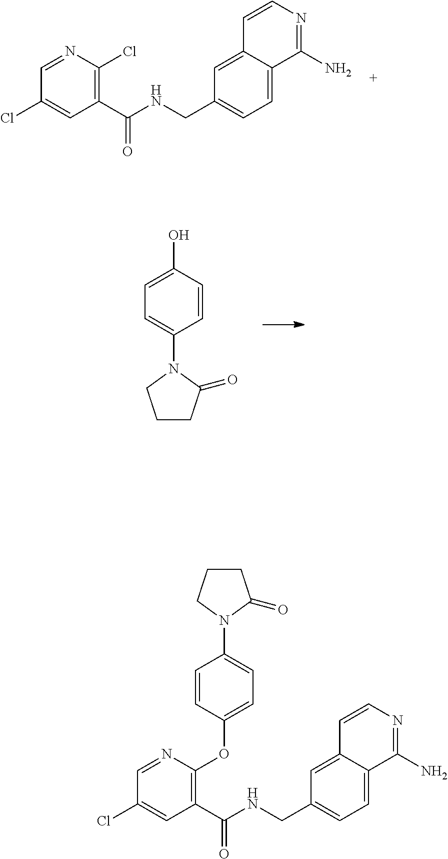 [SMILES: Nc1nccc2cc(CNC(=O)c3cc(Cl)cnc3Cl)ccc12 ; O=C1CCCN1c1ccc(O)cc1 ; Nc1nccc2cc(CNC(=O)c3cc(Cl)cnc3Oc3ccc(N4CCCC4=O)cc3)ccc12]

Following general method C for phenols, N-((1-aminoisoquinolin-6-yl)methyl)-5,6-dichloronicotinamide (36 mg, 0.1 mmol) was reacted with 1-(4-hydroxyphenyl)pyrrolidin-2-one (35 mg, 0.19 mmol). The title compound was isolated (7 mg, 14% yield) as an off white solid.

[M+H]⁺=488.4

### Example 28.01

### N-((1-aminoisoquinolin-6-yl)methyl)-2-(benzyl(methyl)amino)-5-chloronicotinamide

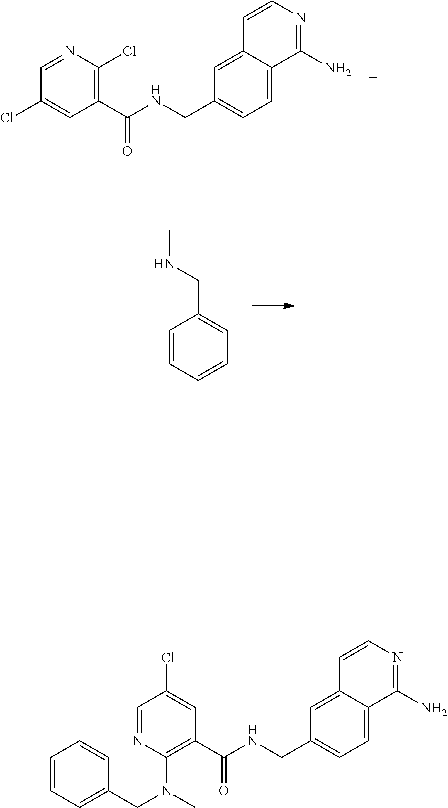

Following general method B(i), N-((1-aminoisoquinolin-6-yl)methyl)-5,6-dichloronicotinamide (35 mg, 0.1 mmol) was reacted with N-methylbenzylamine (36 mg, 0.3 mmol) to afford the title compound (19 mg, 44% yield).

[M+H]⁺=432.4

### Example 28.02

### N-((1-aminoisoquinolin-6-yl)methyl)-5-chloro-2-(methyl((1-(pyridin-4-yl)piperidin-4-yl)methyl)amino)nicotinamide

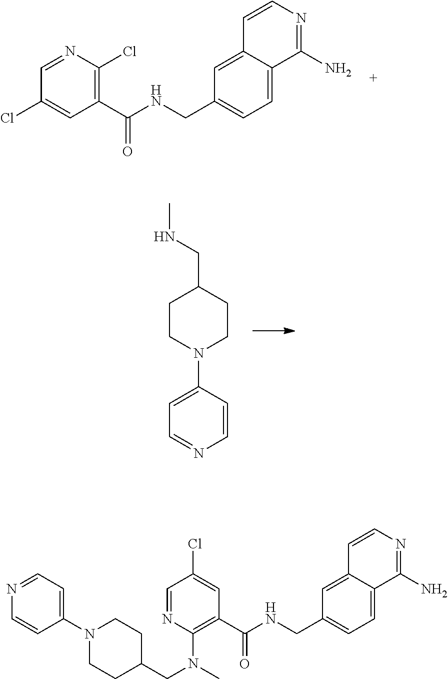 [SMILES: Nc1nccc2cc(CNC(=O)c3cc(Cl)cnc3Cl)ccc12 ; CNCC1CCN(c2ccncc2)CC1 ; CN(CC1CCN(c2ccncc2)CC1)c1ncc(Cl)cc1C(=O)NCc1ccc2c(N)nccc2c1]

Following general method B(i), N-((1-aminoisoquinolin-6-yl)methyl)-5,6-dichloronicotinamide (35 mg, 0.1 mmol) was reacted with N-Methyl-1-(4-pyridinyl)-4-piperidinemethanamine (62 mg, 0.3 mmol) to afford the title compound (21 mg, 41% yield).

[M+H]⁺=516.3

### Example 28.03

### N-((1-aminoisoquinolin-6-yl)methyl)-5-chloro-2-((3-(3-(dimethylamino)propoxy)benzyl)(methyl)amino)nicotinamide

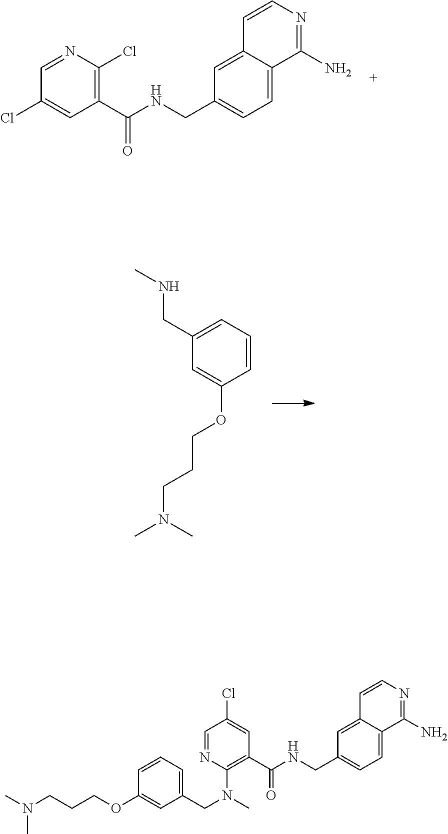 [SMILES: Nc1nccc2cc(CNC(=O)c3cc(Cl)cnc3Cl)ccc12 ; CNCc1cccc(OCCCN(C)C)c1 ; CN(C)CCCOc1cccc(CN(C)c2ncc(Cl)cc2C(=O)NCc2ccc3c(N)nccc3c2)c1]

Following general method B(i), N-((1-aminoisoquinolin-6-yl)methyl)-5,6-dichloronicotinamide (35 mg, 0.1 mmol) was reacted with 3-[3-(Dimethylamino)propoxy]-N-methylbenzylamine (67 mg, 0.3 mmol) to afford the title compound (28 mg, 53% yield).

[M+H]⁺=533.3

### Example 28.04

### N-((1-aminoisoquinolin-6-yl)methyl)-5-chloro-2-(methyl(tetra hydro-2H-pyran-4-yl)amino)nicotinamide

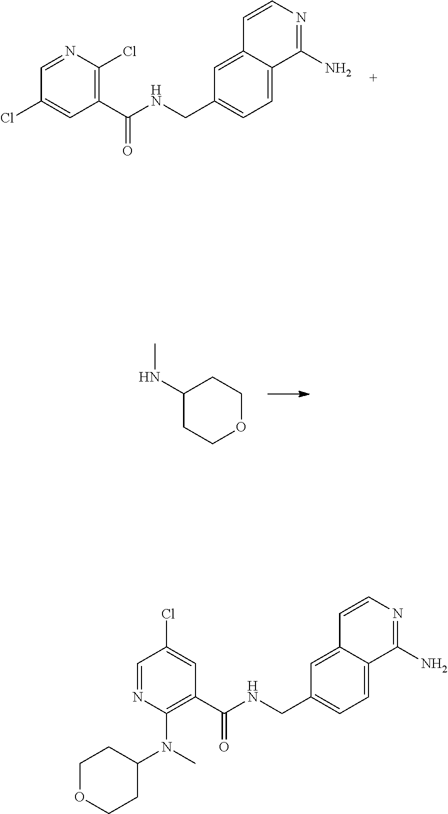

Following general method B(i), N-((1-aminoisoquinolin-6-yl)methyl)-5,6-dichloronicotinamide (35 mg, 0.1 mmol) was reacted with N-Methyl-N-tetrahydro-2H-pyran-4-ylamine (35 mg, 0.3 mmol) to afford the title compound (12 mg, 28% yield).

[M+H]⁺=426.4

### Example 28.05

### N-((1-aminoisoquinolin-6-yl)methyl)-5-chloro-2-(methyl(phenyl)amino)nicotinamide

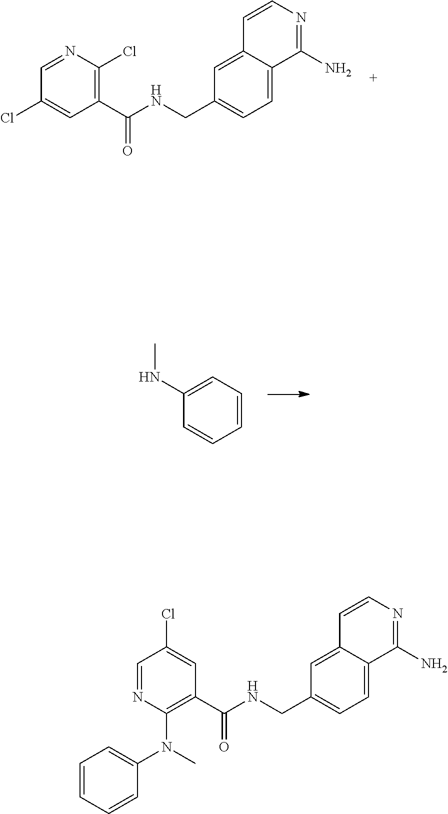

Following general method B(i), N-((1-aminoisoquinolin-6-yl)methyl)-5,6-dichloronicotinamide (35 mg, 0.1 mmol) was reacted with N-methylaniline (32 mg, 0.3 mmol) to afford the title compound (3 mg, 7% yield).

[M+H1]=418.5

### Example 29.03

### N-((1-aminoisoquinolin-6-yl)methyl)-5-(1-benzyl-1H-pyrazol-5-yl)nicotinamide

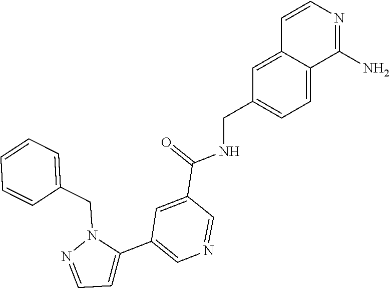 [SMILES: Nc1nccc2cc(CNC(=O)c3cncc(-c4ccnn4Cc4ccccc4)c3)ccc12]

### Methyl 5-(1-benzyl-1H-pyrazol-5-yl)nicotinate

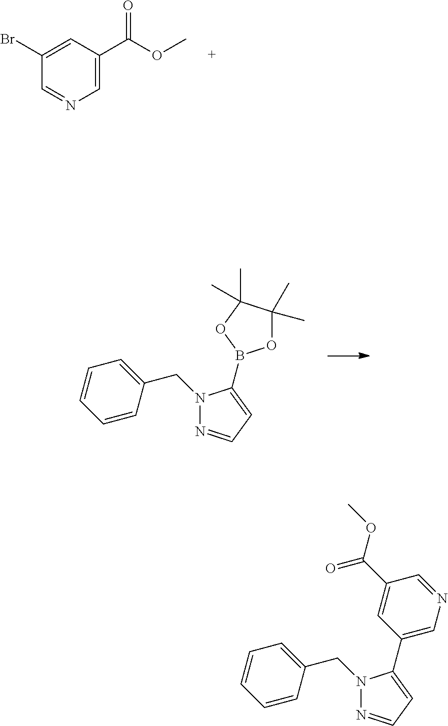 [SMILES: COC(=O)c1cncc(Br)c1 ; CC1(C)OB(c2ccnn2Cc2ccccc2)OC1(C)C ; COC(=O)c1cncc(-c2ccnn2Cc2ccccc2)c1]

A stirred solution of methyl 5-bromonicotinate (250 mg, 1.16 mmol), 1-benzyl-5-(4,4,5,5-tetramethyl-1,3,2-dioxaborolan-2-yl)-1H-pyrazole (460 mg, 1.62 mmol) and potassium carbonate (320 mg, 2.31 mmol) in THF (5 mL) and water (100 μL) was degassed with N₂ and then Pd(PPh₃)₄ (134 mg, 0.12 mmol) was charged and degassed again with N₂. Reaction mixture was heated in the microwave at 80° C. for 30 min. Purification was performed by flash chromatography (0-100% EtOAc in isohexane) to afford the title compound (265 mg, 55% yield) as a pale yellow-green viscous oil.

[M+H]⁺=294.0

### 5-(1-Benzyl-1H-pyrazol-5-yl)nicotinic acid

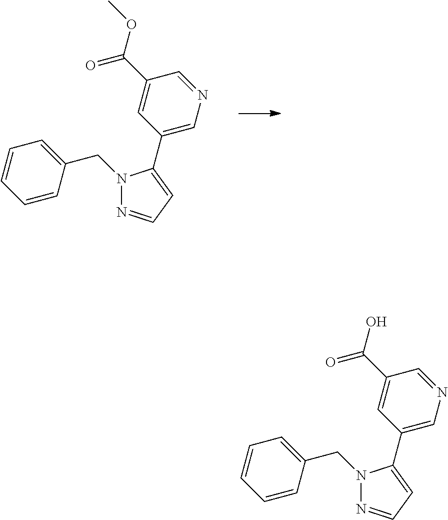 [SMILES: COC(=O)c1cncc(-c2ccnn2Cc2ccccc2)c1 ; O=C(O)c1cncc(-c2ccnn2Cc2ccccc2)c1]

Following general procedure F, methyl 5-(1-benzyl-1H-pyrazol-5-yl)nicotinate (265 mg, 0.63 mmol) was hydrolysed and the crude residue acidified with 2N HCl to pH4 and the resultant cream coloured solid precipitate was filtered, dried under reduced pressure and then placed in the dessicator at 40° C. for 18 hrs to afford the title compound as the hydrochloride salt (186 mg, 91% yield).

[M+H]⁺=280.0

¹H NMR (d6-DMSO) δ 5.44 (2H, s), 6.68 (1H, d, J=1.9 Hz), 6.96-7.01 (2H, m), 7.21-7.31 (3H, m), 7.67 (1H, d, J=1.9 Hz), 8.18 (1H, t, J=2.1 Hz), 8.82 (1H, d, J=2.2 Hz), 9.05 (1H, d, J=2.0 Hz), 13.57 (1H, s).

### N-((1-aminoisoquinolin-6-yl)methyl)-5-(1-benzyl-1H-pyrazol-5-yl)nicotinamide

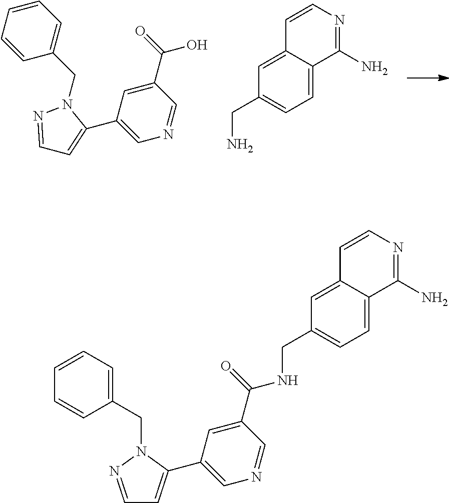 [SMILES: O=C(O)c1cncc(-c2ccnn2Cc2ccccc2)c1 ; NCc1ccc2c(N)nccc2c1 ; Nc1nccc2cc(CNC(=O)c3cncc(-c4ccnn4Cc4ccccc4)c3)ccc12]

Following general procedure A, 5-(1-benzyl-1H-pyrazol-5-yl)nicotinic acid hydrochloride (148 mg, 0.47 mmol) was reacted with 6-(aminomethyl)isoquinolin-1-amine dihydrochloride (115 mg, 0.47 mmol) to afford the title compound (160 mg, 77% yield).

[M+H]⁺=435.1

¹H NMR (d6-DMSO) δ: 4.68 (2H, d, J=5.7 Hz), 5.47 (2H, s), 6.67 (1H, d, J=1.9 Hz), 6.94-7.00 (2H, m), 7.07 (1H, d, J=6.4 Hz), 7.19-7.30 (3H, m), 7.60 (1H, dd, J=1.7, 8.6 Hz), 7.67 (1H, d, J=1.9 Hz), 7.71 (1H, d, J=6.4 Hz), 7.74 (1H, d, J=1.7 Hz), 7.87 (2H, s), 8.29 (1H, t, J=2.1 Hz), 8.35 (1H, d, J=8.6 Hz), 8.76 (1H, d, J=2.1 Hz), 9.07 (1H, d, J=2.1 Hz), 9.41 (1H, t, J=5.9 Hz)

### Example 29.07

### N-((1-aminoisoquinolin-6-yl)methyl)-3-chloro-4-((4-(pyridin-4-yl)piperazin-1-yl)methyl)benzamide

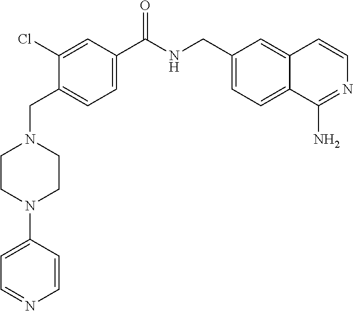 [SMILES: Nc1nccc2cc(CNC(=O)c3ccc(CN4CCN(c5ccncc5)CC4)c(Cl)c3)ccc12]

### Methyl 3-chloro-4-((4-(pyridin-4-yl)piperazin-1-yl)methyl)benzoate

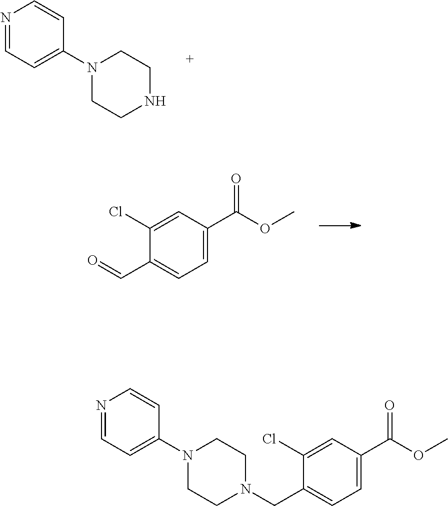 [SMILES: c1cc(N2CCNCC2)ccn1 ; COC(=O)c1ccc(C=O)c(Cl)c1 ; COC(=O)c1ccc(CN2CCN(c3ccncc3)CC2)c(Cl)c1]

Following general method I, methyl 3-chloro-4-formylbenzoate (362 mg, 1.82 mmol) was reacted with 1-(pyridin-4-yl)piperazine (298 mg, 1.83 mmol) in DCM (6 mL) to afford the title compound (200 mg, 31% yield) as a colourless gum.

[M+H]⁺=346.1/348.5

¹H NMR (DMSO-d6, 500 MHz) δ 2.56 (4H, t, J=5.1 Hz), 3.34 (4H, t, J=5.1 Hz), 3.69 (2H, s), 3.87 (3H, s), 6.78-6.84 (2H, m), 7.72 (1H, d, J=7.9 Hz), 7.90-7.96 (2H, m), 8.13-8.19 (2H, m).

### Lithium 3-chloro-4-((4-(pyridin-4-yl)piperazin-1-yl)methyl)benzoate

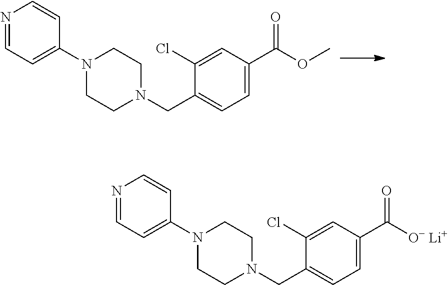

Following general method F(i), methyl 3-chloro-4-((4-(pyridin-4-yl)piperazin-1-yl)methyl)benzoate (200 mg, 0.578 mmol) was reacted with lithium hydroxide (17 mg, 0.71 mmol) to afford the title compound (220 mg, 96% yield) as an off-white solid.

[M+H]⁺=332.2/334.2

¹H NMR (DMSO-d6, 500 MHz) δ 2.54 (4H, t, J=5.1 Hz), 3.32 (4H, t, J=5.1 Hz), 3.61 (2H, s), 6.78-6.83 (2H, m), 7.40 (1H, d, J=7.8 Hz), 7.75 (1H, dd, J=7.8, 1.5 Hz), 7.83 (1H, d, J=1.5 Hz), 8.11-8.18 (2H, m).

### N-((1-aminoisoquinolin-6-yl)methyl)-3-chloro-4-((4-(pyridin-4-yl)piperazin-1-yl)methyl)benzamide

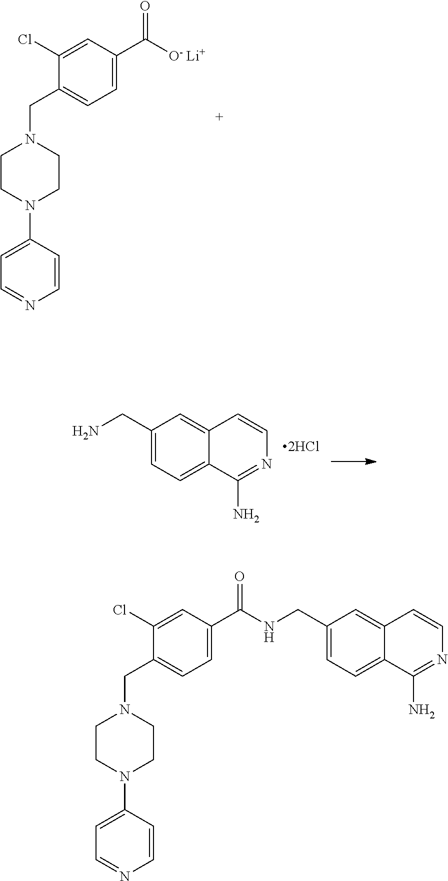

Following general method A(i), lithium 3-chloro-4-((4-(pyridin-4-yl)piperazin-1-yl)methyl)benzoate (115 mg, 0.289 mmol) was reacted with 6-(aminomethyl)isoquinolin-1-amine dihydrochloride (79 mg, 0.329 mmol) in NMP (2 mL) to afford the title compound (75 mg, 51% yield) as cream solid.

[M+H]⁺=487.3/489.3

¹H NMR (DMSO-d6, 500 MHz) δ: 2.56 (4H, t, J=5.1 Hz), 3.32 (4H, s), 3.68 (2H, s), 4.62 (2H, d, J=5.8 Hz), 6.72 (2H, s), 6.79-6.84 (2H, m), 6.87 (1H, d, J=5.8 Hz), 7.42 (1H, dd, J=8.6, 1.8 Hz), 7.57 (1H, d, J=1.7 Hz), 7.66 (1H, d, J=8.0 Hz), 7.77 (1H, d, J=5.8 Hz), 7.90 (1H, dd, J=8.0, 1.8 Hz), 8.00 (1H, d, J=1.8 Hz), 8.12-8.18 (3H, m), 9.24 (1H, t, J=6.0 Hz).

### Example 29.08

### N-[(1-amino-6-isoquinolyl)methyl]-5-chloro-6-[[4-(4-pyridyl)piperazin-1-yl]methyl]pyridine-3-carboxamide

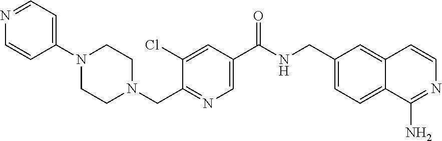 [SMILES: Nc1nccc2cc(CNC(=O)c3cnc(CN4CCN(c5ccncc5)CC4)c(Cl)c3)ccc12]

### Methyl 5-chloro-6-(hydroxymethyl)pyridine-3-carboxylate

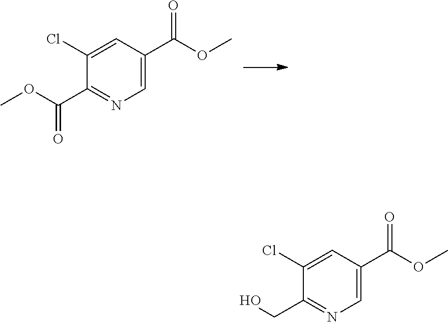

To a solution of dimethyl 3-chloropyridine-2,5-dicarboxylate (CAS 106014-21-5) (2.5 g, 10.9 mmol) in methanol (50 mL) and THF (25 mL) was added powdered calcium chloride (10 g, 90.1 mmol). The mixture was cooled to 0° C., sodium borohydride (1 g, 26.4 mmol) was added portionwise and the reaction stirred at 0° C. for 3 hrs. The reaction was quenched with ice/water (30 mL), concentrated in vacuo to low volume. Extracted with DCM (3×50 mL). The combined extracts were washed with brine (20 mL), dried (MgSO₄), filtered and concentrated in vacuo. Purification by flash chromatography (30-100% EtOAc in isohexane) afforded the title compound (570 mg, 26% yield) as a cream solid.

[M+H]⁺=202.1

¹H NMR (DMSO-d6, 500 MHz) δ 3.91 (3H, s), 4.71 (2H, d, J=6.1 Hz), 5.42 (1H, t, J=6.0 Hz), 8.29 (1H, d, J=1.8 Hz), 9.00 (1H, d, J=1.8 Hz).

### Methyl 5-chloro-6-formyl-pyridine-3-carboxylate

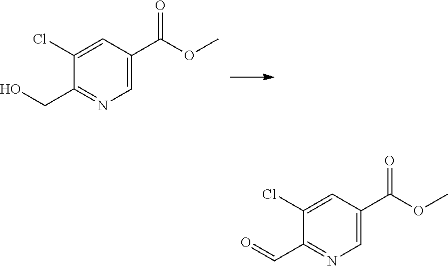

Dess-Martin periodinane (1.77 g, 4.17 mmol) was added to a solution of methyl 5-chloro-6-(hydroxymethyl)pyridine-3-carboxylate (560 mg, 2.78 mmol) in DCM (5 mL) and stirred for 60 min. The reaction mixture was concentrated and the product purified by flash chromatography (0-40% EtOAc in isohexane) to afford the title compound (510 mg, 90% yield) as a white solid.

[M+H]⁺=200.0

¹H NMR (DMSO-d6, 500 MHz) δ 3.95 (3H, s), 8.49 (1H, d, J=1.8 Hz), 9.19 (1H, d, J=1.7 Hz), 10.17 (1H, s).

### Methyl 5-chloro-6-[[4-(4-pyridyl)piperazin-1-yl]methyl]pyridine-3-carboxylate

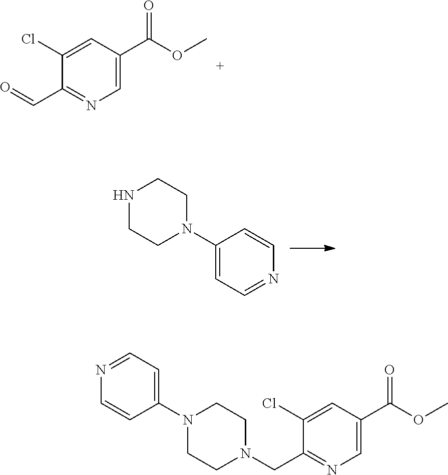 [SMILES: COC(=O)c1cnc(C=O)c(Cl)c1 ; c1cc(N2CCNCC2)ccn1 ; COC(=O)c1cnc(CN2CCN(c3ccncc3)CC2)c(Cl)c1]

1-(4-pyridyl)piperazine (197 mg, 1.21 mmol) and methyl 5-chloro-6-formyl-pyridine-3-carboxylate (240 mg, 1.2 mmol) were dissolved in THF (5 mL) and stirred for 15 min at rt. Acetic acid (0.21 mL, 3.67 mmol) and sodium triacetoxyborohydride (637 mg, 3.01 mmol) were added and the reaction stirred for 20 hrs at rt. The reaction was quenched with sat. NaHCO₃ (20 mL), extracted with DCM (2×20 mL) and the combined extracts washed with brine (10 mL), filtered through a phase separation cartridge and concentrated in vacuo. The product was purified by flash chromatography (0 to 10% (1% NH₃ in MecOH) in DCM) to afford the title compound (185 mg, 44% yield) as a colourless gum.

[M+H]⁺=347.2/349.2

¹H NMR (DMSO-d6, 500 MHz) δ 2.62 (4H, t, J=5.1 Hz), 3.29 (4H, t, J=5.1 Hz), 3.82 (2H, s), 3.91 (3H, s), 6.77-6.82 (2H, m), 8.12-8.17 (2H, m), 8.32 (1H, d, J=1.9 Hz), 8.99 (1H, d, J=1.9 Hz).

### [5-Chloro-6-[[4-(4-pyridyl)piperazin-1-yl]methyl]pyridine-3-carbonyl]oxylithium

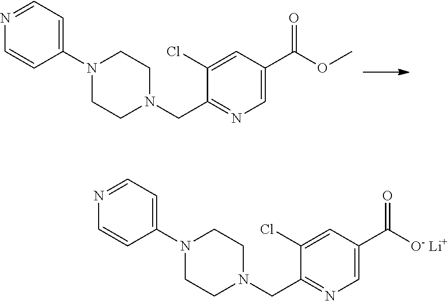

A solution of lithium hydroxide (15 mg, 0.63 mmol) in water (2 mL) was added to a solution of methyl 5-chloro-6-[[4-(4-pyridyl)piperazin-1-yl]methyl]pyridine-3-carboxylate (180 mg, 0.52 mmol) in THF (2 mL) and methanol (4 mL) and stirred at rt for 20 hrs. Reaction was concentrated in vacuo and the residue treated with 1,4-dioxane (15 mL). The resulting solid was filtered off, washed with 1,4-dioxane (10 mL) and Et₂O (10 mL), to afford the title compound (175 mg, 91% yield), as an off-white solid.

[M+H]⁺=333.2/335.2

¹H NMR (DMSO-d6, 500 MHz) δ 2.54-2.62 (4H, m), 3.24-3.32 (4H, m), 3.74 (2H, s), 6.76-6.81 (2H, m), 8.11-8.16 (3H, m), 8.85 (1H, d, J=1.7 Hz).

### N-[(1-amino-6-isoquinolyl)methyl]-5-chloro-6-[[4-(4-pyridyl)piperazin-1-yl]methyl]pyridine-3-carboxamide

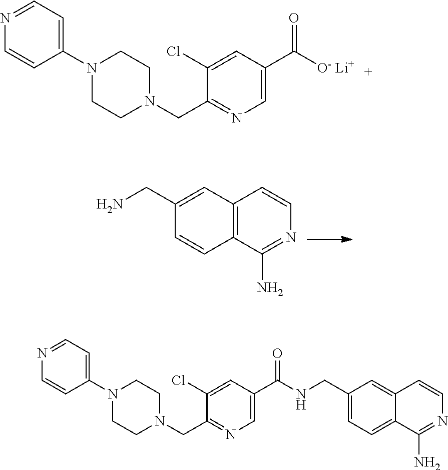

DIPEA (0.42 mL, 2.4 mmol) was added to a solution of [5-chloro-6-[[4-(4-pyridyl)piperazin-1-yl]methyl]pyridine-3-carbonyl]oxylithium (160 mg, 0.47 mmol), 6-(aminomethyl)isoquinolin-1-amine dihydrochloride (129 mg, 0.52 mmol) and HATU (216 mg, 0.57 mmol) in NMP (2 mL) and stirred for 20 hrs. The reaction was diluted with MeOH (20 mL), absorbed onto SCX, washed with MeOH (20 mL) and the product eluted with 0.7M NH₃/MeOH and concentrated in vacuo. The crude product was purified by flash chromatography (0 to 30% (1% NH₃ in MeCOH) in EtOAc). Triturated with MeCOH (3 mL) for 2 hrs, solid filtered off, washed with Et₂O (10 mL) to afford the title compound (28 mg, 12% yield), as an off-white solid.

[M+H]⁺=488.3/490.3

¹H NMR (DMSO-d6, 500 MHz) δ 2.62 (4H, t, J=5.1 Hz), 3.29 (4H, t, J=5.1 Hz), 3.81 (2H, s), 4.64 (2H, d, J=5.7 Hz), 6.73 (2H, s), 6.77-6.82 (2H, m), 6.88 (1H, d, J=5.8 Hz), 7.43 (1H, dd, J=8.6, 1.8 Hz), 7.61 (1H, d, J=1.7 Hz), 7.77 (1H, d, J=5.8 Hz), 8.12-8.18 (3H, m), 8.37 (1H, d, J=1.9 Hz), 8.99 (1H, d, J=1.9 Hz), 9.39 (1H, t, J=5.9 Hz).

### Example 29.11

### N-((1-aminoisoquinolin-6-yl)methyl)-3-chloro-5-((4-(cyclopentylmethyl)piperazin-1-yl)methyl)benzamide

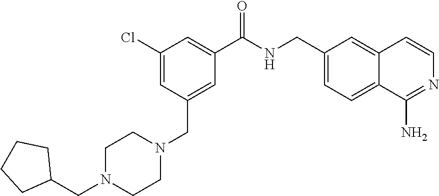 [SMILES: Nc1nccc2cc(CNC(=O)c3cc(Cl)cc(CN4CCN(CC5CCCC5)CC4)c3)ccc12]

### Methyl 3-chloro-5-((4-methylpiperazin-1-yl)methyl)benzoate

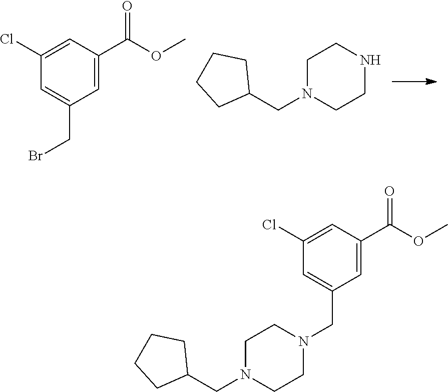

In a modification to general method E, methyl 3-(bromomethyl)-5-chlorobenzoate (30 mg, 0.08 mmol), was reacted with 1-(cyclopentylmethyl)piperazine) (13 mg, 0.08 mmol), in the presence of triethylamine (7.9 mg, 0.08 mmol) in THF (2 mL). The reaction mixture was heated at 60° C. for 18 hrs. The crude reaction mixture was dissolved in DCM (30 mL) and washed with water (3×30 mL), the organic was dried (MgSO₄) and concentrated in vacuo. The crude product was purified was purified by flash chromatography (0-10% (0.7M NH₃ in MecOH) in DCM) to afford the title compound as an off white solid (32 mg, 54% yield).

[M+H]⁺=283.1

### 3-Chloro-5-((4-(cyclopentylmethyl)piperazin-1-yl)methyl)benzoic acid

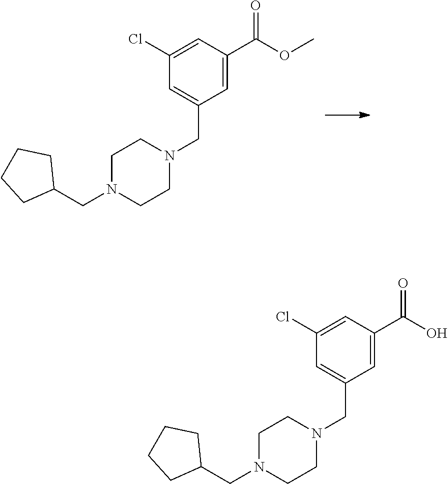 [SMILES: COC(=O)c1cc(Cl)cc(CN2CCN(CC3CCCC3)CC2)c1 ; O=C(O)c1cc(Cl)cc(CN2CCN(CC3CCCC3)CC2)c1]

In a modification to general method F(i) methyl 3-chloro-5-((4-(cyclopentylmethyl)piperazin-1-yl)methyl)benzoate (32 mg, 0.09 mmol) was reacted with 2M LiOH (80 IL, 0.160 mmol) in THF (0.5 mL), MeOH (0.1 mL) at 40° C. to afford the title compound as an off white solid (31 mg, 100% yield).

### N-((1-aminoisoquinolin-6-yl)methyl)-3-chloro-5-((4-(cyclopentylmethyl)piperazin-1-yl)methyl)benzamide

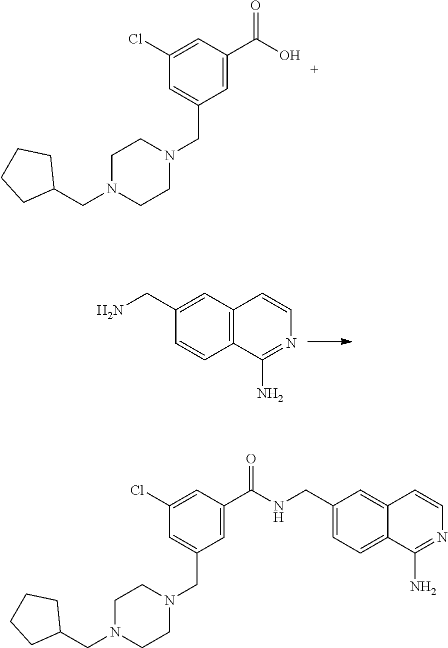 [SMILES: O=C(O)c1cc(Cl)cc(CN2CCN(CC3CCCC3)CC2)c1 ; NCc1ccc2c(N)nccc2c1 ; Nc1nccc2cc(CNC(=O)c3cc(Cl)cc(CN4CCN(CC5CCCC5)CC4)c3)ccc12]

In a modification to general method A(i) 3-Chloro-5-((4-(cyclopentylmethyl)piperazin-1-yl)methyl)benzoic acid (31 mg, 0.09 mmol) was reacted with 6-(aminomethyl)isoquinolin-1-amine dihydrochloride (24 mg, 0.10 mmol), DIPEA (80 μL, 0.46 mmol) and HATU (36 mg, 0.10 mmol). The crude reaction mixture was purified by reverse phase flash chromatography (10-60% MeCN in 10 mM Ammonium Bicarbonate) to afford the title compound as an off white solid (15 mg, 33% yield).

[M+H]⁺=492.0

¹H NMR (500 MHz, DMSO-d6) δ: 1.10-1.21 (2H, m), 1.41-1.57 (4H, m), 1.58-1.70 (2H, m), 1.96-2.08 (1H, m), 2.11-2.20 (2H, m), 2.30-2.45 (8H, m), 3.52 (2H, s), 4.61 (2H, d, J=5.6 Hz), 6.72 (2H, s, 6.87 (1H, d, J=5.8 Hz), 7.41 (1H, dd, J=8.6, 1.8 Hz), 7.51-7.53 (1H, m), 7.57 (1H, s), 7.77 (1H, d, J=5.8 Hz), 7.79-7.81 (1H, m), 7.85-7.87 (1H, m), 8.15 (1H, d, J=8.6 Hz), 9.24 (1H, t, J=5.9 Hz).

### Example 33.01

### 5-Chloro-N-{[2-chloro-6-(1,2,3,4-tetrazol-1-yl)phenyl]methyl}-2-{[(1-methylpiperidin-4-yl)methyl]amino}pyridine-3-carboxamide

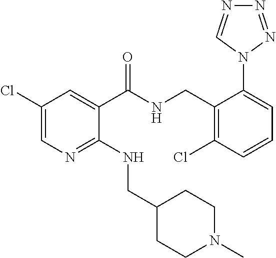 [SMILES: CN1CCC(CNc2ncc(Cl)cc2C(=O)NCc2c(Cl)cccc2-n2cnnn2)CC1]

### 2,5-Dichloro-N-{[2-chloro-6-(1,2,3,4-tetrazol-1-yl)phenyl]methyl}pyridine-3-carboxamide

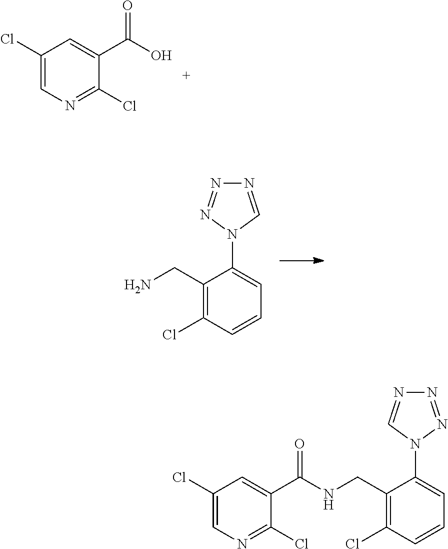 [SMILES: O=C(O)c1cc(Cl)cnc1Cl ; NCc1c(Cl)cccc1-n1cnnn1 ; O=C(NCc1c(Cl)cccc1-n1cnnn1)c1cc(Cl)cnc1Cl]

Following the general method A(ii) [2-chloro-6-(1H-1,2,3,4-tetrazol-1-yl)phenyl]methanamine (100 mg, 0.48 mmol) was reacted with 2,5-dichloro-nicotinic acid (91.6 mg, 0.48 mmol). The crude product was purified by flash chromatography eluting with a (0-6% (10% NH₃ in MeOH) in DCM) to afford the title product as a white solid (109 mg, 60% yield).

[M+H]⁺=424.1

¹H NMR (DMSO-d6): 4.37 (2H, d, J=4.9 Hz), 7.60 (1H, dd, J=1.6, 7.9 Hz), 7.64 (1H, t, J=7.9 Hz), 7.85 (1H, dd, J=1.5, 7.6 Hz), 7.95 (1H, d, J=2.6 Hz), 8.55 (1H, d, J=2.6 Hz), 9.02 (1H, s, J=4.7 Hz), 9.83 (1H, s)

### 5-Chloro-N-{[2-chloro-6-(1,2,3,4-tetrazol-1-yl)phenyl]methyl}-2-{[(1-methylpiperidin-4-yl)methyl]amino}pyridine-3-carboxamide

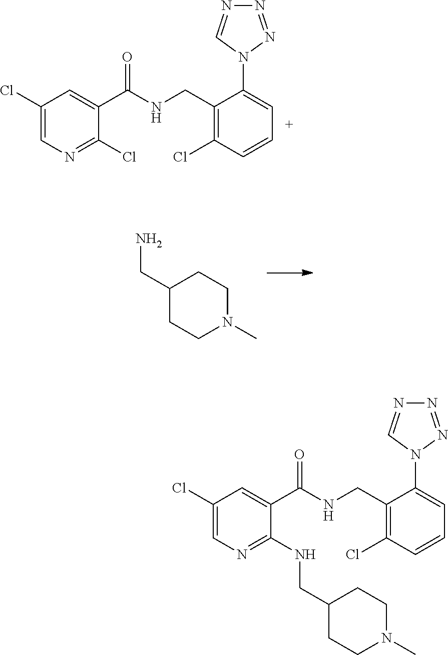

In a modification to general method B(ii), 2,5-dichloro-N-{[2-chloro-6-(1,2,3,4-tetrazol-1-yl)phenyl]methyl}pyridine-3-carboxamide (30 mg, 0.08 mmol), C-(1-methyl-piperidin-4-yl)methylamine (10 mg, 0.08 mmol) and triethylamine (7.9 mg, 0.08 mmol) in n-butanol (0.35 mL) were added to a sealed microwave tube and heated to 120° C. for 3 hrs. The crude reaction mixture was dissolved in DCM (50 mL) and washed with water (3×100 mL), the organic was dried (MgSO₄) and concentrated in vacuo. The crude product was purified by flash chromatography (0-10% (10% NH₃ in MeOH) in DCM) to afford the title compound as an off white solid (3 mg, 8% yield).

[M+H]⁺=475.3

¹H NMR (Methanol-d4): 1.44 (2H, qd, J=3.8, 13.3 Hz), 1.80 (1H, m), 1.92 (2H, d, J=13.5 Hz), 2.44 (2H, t, J 5=14.0 Hz), 2.55 (3H, s), 3.19 (2H, d, J=11.0 Hz), 3.40 (2H, d, J=6.8 Hz), 4.51 (2H, s), 7.52 (1H, dd, J=0.9, 8.0 Hz), 7.63 (1H, t, J=8.0 Hz), 7.81 (1H, d, J=2.5 Hz), 7.84 (1H, dd, J=1.1, 8.1 Hz), 8.10 (1H, d, J=2.5 Hz), 9.64 (1H, s)

### Example 33.09

### 1-(3-Fluoro-4-methoxy-2-{[(3-{[(1-methylpiperidin-4-yl)methyl]amino}pyrazin-2-yl)formamido]methyl}phenyl)-1,2,3-triazole-4-carboxylic acid

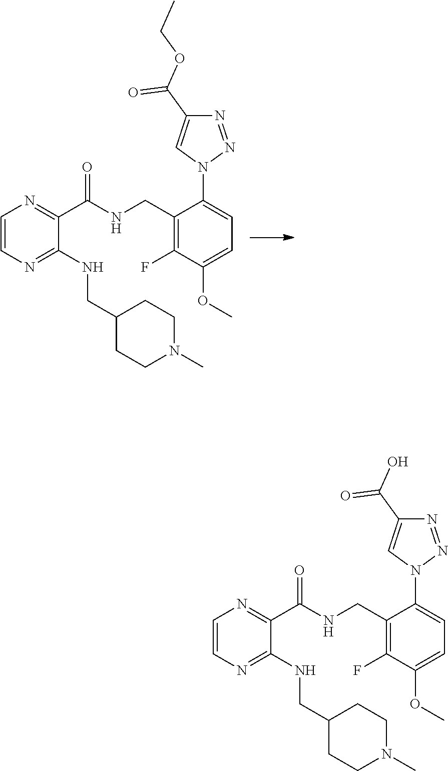 [SMILES: CCOC(=O)c1cn(-c2ccc(OC)c(F)c2CNC(=O)c2nccnc2NCC2CCN(C)CC2)nn1 ; COc1ccc(-n2cc(C(=O)O)nn2)c(CNC(=O)c2nccnc2NCC2CCN(C)CC2)c1F]

A suspension of ethyl 1-(3-fluoro-4-methoxy-2-{[(3-{[(1-methylpiperidin-4-yl)methyl]amino}pyrazin-2-yl)formamido]methyl}phenyl)-1,2,3-triazole-4-carboxylate (25 mg, 0.05 mmol) in a mixture of MecOH (0.2 mL) and water (1 mL) was treated with lithium hydroxide (10 mg, 0.24 mmol). The reaction was stirred at rt for 18 hrs and then concentrated in vacuo. Purified by reverse phase flash chromatography (0-95% MeCN in Water (0.1% formic acid)) to afford the title compound (13 mg, 54% yield) as a white solid.

[M+H]⁺=499.5

¹H NMR (400 MHz, DMSO): 1.66-1.62 (4H, m), 1.79-1.77 (1H, m), 2.60-2.58 (3H, m), 2.69-2.64 (2H, m), 3.30-3.25 (2H, m), 3.64-3.60 (2H, m), 3.97-3.96 (3H, m), 4.40-4.36 (2H, m), 7.37-7.33 (2H, m), 7.69 (1H, d, J=2.4 Hz), 8.20-8.16 (2H, m), 8.78-8.67 (2H, m).

### Example 33.12

### N-(1-aminoisoquinolin-5-yl)-3-(((1-methylpiperidin-4-yl)methyl)amino)pyrazine-2-carboxamide

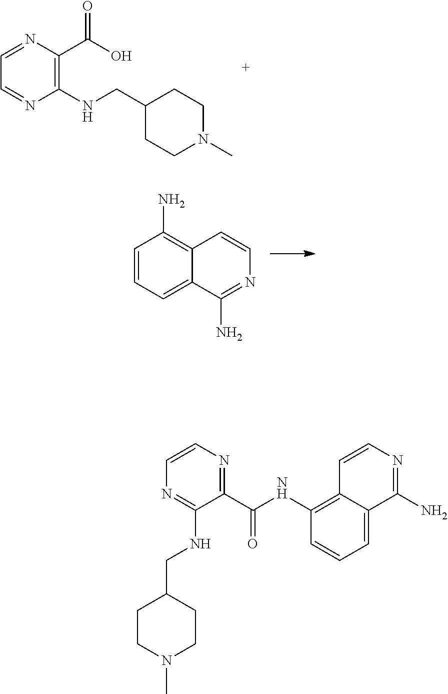

Following method A, 3-(((1-methylpiperidin-4-yl)methyl)amino)pyrazine-2-carboxylic acid (50 mg, 0.20 mmol) and isoquinoline-1,5-diamine (76 mg, 0.20 mmol) were reacted to afford the title compound (6.6 mg, 9% yield).

[M+H]⁺=392.4

¹H NMR (400 MHz, DMSO): 1.31-1.19 (2H, m), 1.62-1.51 (1H, m), 1.66 (2H, d, J=12.9 Hz), 1.84-1.77 (2H, m), 2.15-2.13 (3H, m), 2.76 (2H, d, J=11.4 Hz), 3.39 (2H, t, J=6.0 Hz), 6.92-6.87 (3H, m), 7.51 (1H, t, J=8.0 Hz), 7.87 (1H, d, J=6.0 Hz), 8.00-7.93 (2H, m), 8.14-8.09 (1H, m), 8.39 (1H, d, J=2.3 Hz), 8.73-8.68 (1H, m), 10.65 (1H, s).

### Example 33.18

### N-((1-amino-5-methylisoquinolin-6-yl)methyl)-3-(((1-methylpiperidin-4-yl)methyl)amino)pyrazine-2-carboxamide

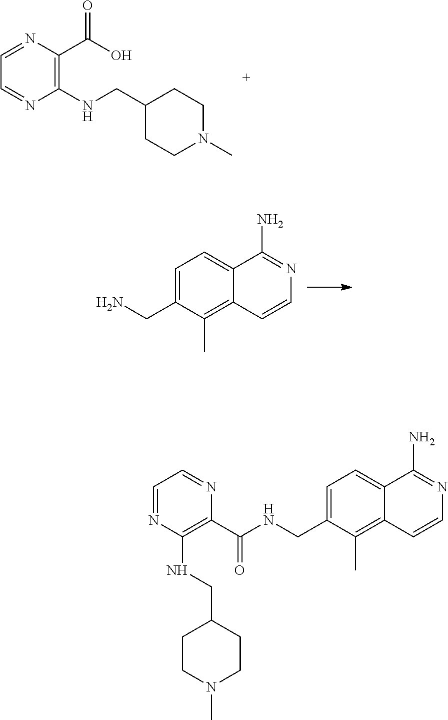 [SMILES: CN1CCC(CNc2nccnc2C(=O)O)CC1 ; Cc1c(CN)ccc2c(N)nccc12 ; Cc1c(CNC(=O)c2nccnc2NCC2CCN(C)CC2)ccc2c(N)nccc12]

Following method A, 3-(((1-methylpiperidin-4-yl)methyl)amino)pyrazine-2-carboxylic acid (30 mg, 0.12 mmol) and 6-(aminomethyl)-5-methylisoquinolin-1-amine (24 mg, 0.13 mmol) (synthesis reported in a previous patent WO2016083816) were reacted to afford the title compound (9.0 mg, 15% yield).

[M+H]⁺=420.3

¹H NMR (400 MHz, DMSO): 1.31-1.16 (3H, m), 1.39 (1H, s), 1.67-1.52 (4H, m), 1.93-1.86 (2H, m), 2.18 (3H, s), 2.38-2.34 (1H, m), 2.80 (2H, d, J=11.5 Hz), 3.87 (1H, s), 4.65-4.61 (2H, m), 6.72-6.68 (2H, m), 7.04 (1H, d, J=6.1 Hz), 7.40-7.36 (1H, m), 7.84-7.79 (2H, m), 8.02-7.98 (1H, m), 8.29-8.24 (3H, m), 8.80 (1H, t, J=5.8 Hz), 9.35 (1H, t, J=6.1 Hz).

TABLE 12
¹H NMR data of examples (solvent d6 DMSO unless otherwise indicated)

| Example. No. | Chemical shift |
|---|---|
| 1.01 | 1.33-1.41 (2H, m), 1.52-1.68 (4H, m), 1.91-1.99 (2H, m), 4.22-4.30 (1H, m), 4.63 |
| | (2H, d, J = 5.6 Hz), 7.26 (1H, d, J = 7.0 Hz), 7.66 (1H, d, J = 7.0 Hz), 7.74 (1H, dd, J = 8.6, 1.4 |
| | Hz), 7.85 (1H, s), 8.18 (1H, d, J = 2.5 Hz), 8.23 (1H, d, J = 2.5 Hz), 8.47 (1H, d, J = 6.8 Hz), |
| | 8.51 (1H, d, J = 8.7 Hz), 8.97 (2H, s), 9.35 (1H, t, J = 5.7 Hz), 12.95 (1H, s). |
| 1.13 | 1.18 (2H, qd, J = 12.0, 3.8 Hz), 1.43-1.52 (1H, m), 1.55-1.66 (2H, m), 1.77 (2H, td, J = |
| | 11.7, 2.4 Hz), 2.11 (3H, s), 2.72 (2H, dt, J = 11.6, 3.2 Hz), 3.23-3.30 (2H, m), 4.56 (2H, |
| | d, J = 5.7 Hz), 6.73 (2H, s), 6.87 (1H, dd, J = 5.9, 0.8 Hz), 7.40 (1H, dd, J = 8.6, 1.8 Hz), |
| | 7.56 (1H, s), 7.76 (1H, d, J = 5.8 Hz), 8.10-8.17 (2H, m), 8.19 (1H, d, J = 2.5 Hz), 8.53 |
| | (1H, t, J = 5.7 Hz), 9.25 (1H, t, J = 5.8 Hz) |
| 1.63 | 1.20 (2H, qd, J = 12.1, 3.9 Hz), 1.59 (3H, t, J = 19.8 Hz), 1.85-1.98 (2H, m), 2.78 (2H, d, |
| | J = 11.1 Hz), 3.29 (2H, t, J = 6.0 Hz), 3.46 (2H, s), 4.57 (2H, d, J = 5.6 Hz), 6.72 (2H, s), |
| | 6.87 (1H, d, J = 5.8 Hz), 7.34 (1H, dd, J = 7.8, 4.7 Hz), 7.40 (1H, dd, J = 8.7, 1.7 Hz), 7.57 |
| | (1H, d, J = 1.6 Hz), 7.68 (1H, dt, J = 7.8, 2.0 Hz), 7.76 (1H, d, J = 5.8 Hz), 8.10-8.17 (2H, |
| | m), 8.19 (1H, d, J = 2.4 Hz), 8.46 (2H, dt, J = 6.5, 1.8 Hz), 8.52 (1H, t, J = 5.7 Hz), 9.24 (1H, |
| | t, J = 5.8 Hz). |
| 1.64 | 0.91 (7H, t, J = 7.0 Hz), 1.29-1.44 (1H, m), 1.55-1.70 (2H, m), 1.75 (1H, d, J = 6.1 Hz), |
| | 1.90 (1H, t, J = 10.1 Hz), 2.07 (1H, t, J = 10.7 Hz), 2.56-2.74 (3H, m), 3.25-3.31 (2H, m), |
| | 4.57 (2H, d, J = 5.7 Hz), 6.72 (2H, s), 6.87 (1H, d, J = 5.8 Hz), 7.41 (1H, dd, J = 8.6, 1.7 Hz), |
| | 7.57 (1H, s), 7.77 (1H, d, J = 5.8 Hz), 8.08-8.17 (2H, m), 8.20 (1H, d, J = 2.5 Hz), 8.49 |
| | (1H, t, J = 5.7 Hz), 9.24 (1H, t, J = 5.9 Hz). |
| 1.65 | (MeOD) 1.28-1.42 (3H, m), 1.56-1.69 (1H, m), 1.72-1.80 (2H, m), 2.10 (2H, td, J = |
| | 11.9, 2.5 Hz), 2.96 (2H, dt, J = 12.0, 3.3 Hz), 3.36 (1H, s), 3.74 (2H, s), 4.68 (2H, s), 6.97 |
| | (1H, dd, J = 6.1, 0.9 Hz), 7.48 (1H, d, J = 2.0 Hz), 7.50 (1H, dd, J = 8.6, 1.8 Hz), 7.64 (1H, |
| | d, J = 1.6 Hz), 7.72 (1H, d, J = 6.0 Hz), 7.96 (1H, d, J = 2.5 Hz), 8.07-8.12 (2H, m), 8.97 |
| | (1H, d, J = 2.0 Hz) |
| 1.66 | (MeOD) 1.19 (1H, d, J = 3.6 Hz), 1.25 (2H, td, J = 12.4, 4.0 Hz), 1.52 (1H, dtq, J = 14.5, |
| | 6.9, 3.4 Hz), 1.60-1.69 (2H, m), 2.03 (2H, td, J = 11.7, 2.5 Hz), 2.84 (2H, dt, J = 11.8, 3.4 |
| | Hz), 3.24 (2H, d, J = 6.8 Hz), 3.74 (2H, s), 4.56 (2H, s), 6.84 (1H, dd, J = 6.1, 0.9 Hz), 7.38 |
| | (1H, dd, J = 8.6, 1.8 Hz), 7.43 (1H, d, J = 3.4 Hz), 7.52 (1H, d, J = 1.5 Hz), 7.56-7.63 (2H, |
| | m), 7.84 (1H, d, J = 2.5 Hz), 7.96 (1H, dd, J = 8.6, 0.8 Hz), 7.99 (1H, d, J = 2.5 Hz) |
| 1.67 | 1.17 (3H, d, J = 12.4 Hz), 1.54 (1H, s), 1.63 (2H, d, J = 12.3 Hz), 1.85-1.95 (2H, m), 2.79 |
| | (2H, d, J = 10.9 Hz), 3.25-3.30 (3H, m), 3.46 (2H, s), 3.77 (3H, s), 4.57 (2H, d, J = 5.7 Hz), |
| | 6.10 (1H, d, J = 1.8 Hz), 6.74 (2H, s), 6.87 (1H, d, J = 5.9 Hz), 7.29 (1H, d, J = 1.8 Hz), 7.41 |
| | (1H, dd, J = 8.5, 1.8 Hz), 7.57 (1H, s), 7.76 (1H, d, J = 5.8 Hz), 8.14 (2H, d, J = 2.5 Hz), 8.19 |
| | (1H, d, J = 2.5 Hz), 8.32 (1H, s), 8.52 (1H, t, J = 5.6 Hz), 9.24 (1H, t, J = 5.8 Hz) |
| 1.68 | 1.10-1.27 (3H, m), 1.47-1.55 (1H, m), 1.61 (2H, d, J = 12.6 Hz), 1.79-1.84 (1H, m), 2.80 |
| | (2H, d, J = 10.9 Hz), 3.23-3.31 (4H, m), 3.78 (3H, s), 4.57 (2H, d, J = 5.7 Hz), 6.72 (2H, s), |
| | 6.87 (1H, d, J = 5.9 Hz), 7.26 (1H, s), 7.41 (1H, dd, J = 8.6, 1.8 Hz), 7.52 (1H, s), 7.57 (1H, |
| | s), 7.77 (1H, d, J = 5.8 Hz), 8.12-8.16 (2H, m), 8.19 (1H, d, J = 2.5 Hz), 8.51 (1H, t, J = 5.6 |
| | Hz), 9.25 (1H, t, J = 5.8 Hz) |
| 1.69 | 0.91 (6H, dd, J = 6.6, 2.4 Hz), 1.39 (2H, d, J = 7.8 Hz), 1.63 (1H, d, J = 10.9 Hz), 1.71-1.76 |
| | (1H, m), 2.15 (1H, s), 2.29 (1H, t, J = 10.0 Hz), 2.53 (2H, s), 2.61-2.76 (2H, m), 4.53 (2H, |
| | t, J = 5.8 Hz), 6.70 (2H, s), 6.88 (1H, d, J = 5.8 Hz), 7.46 (1H, dd, J = 8.6, 1.7 Hz), 7.69 (1H, |
| | d, J = 1.6 Hz), 7.77 (1H, d, J = 5.8 Hz), 8.09 (1H, d, J = 2.6 Hz), 8.14 (1H, d, J = 8.6 Hz), |
| | 8.53 (1H, d, J = 2.5 Hz), 9.07 (1H, t, J = 5.9 Hz), 10.85 (1H, s) |
| 1.70 | 1.67-1.77 (1H, m), 2.01-2.21 (3H, m), 3.48-3.53 (1H, m), 3.59-3.67 (1H, m), 3.73- |
| | 3.80 (1H, m), 4.57-4.61 (2H, m), 6.83-6.93 (3H, m), 7.42-7.47 (1H, m), 7.61 (1H, s), |
| | 7.74-7.79 (2H, m), 8.15-8.22 (3H, m), 8.56-8.61 (1H, m), 9.26 (1H, t, J = 5.8 Hz). |
| 1.71 | 1.11-1.08 (3H, m), 1.38-1.17 (3H, m), 1.55 (1H, d, J = 11.8 Hz), 1.64 (1H, d, J = 12.3 |
| | Hz), 1.81-1.71 (2H, m), 2.12 (3H, s), 2.76 (2H, d, J = 11.2 Hz), 4.11-4.04 (1H, m), 4.58 |
| | (2H, d, J = 5.6 Hz), 6.75-6.72 (2H, m), 6.89 (1H, d, J = 5.6 Hz), 7.42 (1H, dd, J = 1.7, 8.6 |
| | Hz), 7.58 (1H, s), 7.78 (1H, d, J = 5.8 Hz), 8.20-8.15 (3H, m), 8.52-8.48 (1H, m), 9.25 |
| | (1H, t, J = 5.8 Hz). |
| 1.72 | 1.77-1.69 (1H, m), 2.20-2.03 (4H, m), 3.67-3.59 (1H, m), 3.80-3.74 (1H, m), 4.59 |
| | (2H, d, J = 5.6 Hz), 6.75-6.72 (2H, m), 6.90 (1H, d, J = 5.5 Hz), 7.43 (1H, dd, J = 1.7, 8.6 |
| | Hz), 7.59 (1H, s), 7.79-7.74 (2H, m), 8.21-8.14 (4H, m), 8.58 (1H, t, J = 5.9 Hz), 9.25 |
| | (1H, t, J = 5.8 Hz). |
| 4.01 | (Methanol-d4): 1.39 (2H, qd, J = 3.4, 12.2 Hz), 1.69 (1H, m), 1.86 (2H, d, J = 13.5 Hz), |
| | 2.09 (2H, t, J = 11.7 Hz), 2.33 (3H, s), 2.95 (2H, d, J = 11.8 Hz), 3.40 (2H, d, J = 6.9 Hz), |
| | 4.74 (2H, s), 6.65 (1H, dd, J = 5.0, 7.6 Hz), 7.01 (1H, d, J = 6.0 Hz), 7.55 (1H, dd, J = 1.7, |
| | 8.6 Hz), 7.69 (1H, s), 7.77 (1H, d, J = 6.0 Hz), 7.99 (1H, dd, J = 1.6, 7.6 Hz), 8.14 (1H, dd, |
| | J = 8.6 Hz), 8.17 (1H, dd, J = 1.8, 4.9 Hz) |
| 4.02 | (Methanol-d4): 1.39 (2H, qd, J = 3.7, 12.8 Hz), 1.70 (1H, m), 1.87 (2H, d, J = 13.3 Hz), |
| | 2.13 (2H, t, J = 11.8 Hz), 2.27 (3H, s), 2.36 (3H, s), 2.97 (2H, d, J = 11.8 Hz), 4.73 (2H, s), |
| | 4.02 (2H, s), 7.02 (1H, d, J = 6.1 Hz), 7.56 (1H, dd, J = 1.7, 8.6 Hz), 7.69 (1H, s), 7.77 (1H, |
| | d, J = 6.0 Hz), 7.86 (1H, d, J = 2.1 Hz), 8.02 (1H, d, J = 1.7 Hz), 8.14 (1H, d, J = 8.6 Hz) |
| 4.03 | 1.35-1.24 (2H, m), 1.71-1.60 (3H, m), 2.15 (2H, dd, J = 9.9, 11.8 Hz), 2.32-2.31 (3H, |
| | m), 2.94 (2H, d, J = 11.7 Hz), 4.61-4.57 (2H, m), 6.80 (2H, s), 6.87 (1H, d, J = 5.6 Hz), |
| | 7.45-7.42 (1H, m), 7.56 (1H, s), 7.83-7.75 (2H, m), 8.16-8.13 (1H, m), 8.22 (2H, s), |
| | 8.29-8.28 (1H, m), 8.84-8.79 (1H, m), 9.50 (1H, t, J = 6.3 Hz). |
| 4.04 | 9.08 (1H, t, J = 6.4 Hz), 8.25-8.11 (5H, m), 7.77 (1H, d, J = 5.8 Hz), 7.55 (1H, s), 7.44- |
| | 7.38 (2H, m), 6.87 (1H, d, J = 5.9 Hz), 6.78-6.74 (2H, m), 4.65 (2H, d, J = 6.3 Hz), 3.34 |
| | (2H, t, J = 5.5 Hz), 2.83 (2H, d, J = 11.4 Hz), 2.21-2.20 (3H, m), 1.95 (2H, dd, J = 9.5, 12.0 |
| | Hz), 1.74-1.68 (2H, m), 1.57-1.47 (1H, m), 1.35-1.23 (2H, m) |
| 4.05 | 1.44-1.32 (2H, m), 1.94-1.78 (3H, m), 2.95-2.74 (6H, m), 3.32-3.25 (2H, m), 4.68- |
| | 4.64 (2H, m), 7.28-7.21 (1H, m), 7.74-7.65 (2H, m), 7.80 (1H, s), 7.97-7.92 (2H, m), |
| | 8.56-8.49 (2H, m), 9.14-9.05 (3H, m), 9.38-9.33 (1H, m), 13.29-13.26 (1H, m). |
| 4.06 | 1.36-1.24 (2H, m), 1.72-1.59 (3H, m), 2.18-2.08 (2H, m), 2.30 (3H, s), 2.93 (2H, d, J = |
| | 11.7 Hz), 3.36 (2H, s), 3.84 (3H, s), 4.56 (2H, d, J = 5.6 Hz), 5.97 (1H, d, J = 8.5 Hz), 6.79 |
| | (2H, s), 6.87 (1H, d, J = 5.6 Hz), 7.41 (1H, dd, J = 1.6, 8.5 Hz), 7.55 (1H, s), 7.77 (1H, d, |
| | J = 5.9 Hz), 8.04 (1H, d, J = 8.5 Hz), 8.15 (1H, d, J = 8.5 Hz), 8.23 (2H, s), 8.85 (1H, dd, J = |
| | 5.9, 5.9 Hz), 8.97 (1H, dd, J = 5.8, 5.8 Hz). |
| 4.07 | 1.28-1.16 (2H, m), 1.54-1.43 (1H, m), 1.61 (2H, d, J = 12.2 Hz), 1.84-1.76 (2H, m), |
| | 2.13 (3H, s), 2.74 (2H, d, J = 11.4 Hz), 3.27 (2H, t, J = 5.3 Hz), 4.60-4.56 (2H, m), 6.66- |
| | 6.63 (1H, m), 6.73 (2H, s), 6.87 (1H, d, J = 5.6 Hz), 7.40 (1H, dd, J = 1.7, 8.6 Hz), 7.56 (1H, |
| | s), 7.78 (1H, d, J = 5.8 Hz), 8.11 (2H, dd, J = 8.4, 30.0 Hz), 8.79 (1H, t, J = 5.5 Hz), 9.22- |
| | 9.17 (1H, m). |
| 4.08 | 1.33 (2H, q, J = 12.9 Hz), 1.90-1.80 (3H, m), 2.89-2.73 (5H, m), 3.14-3.09 (2H, m), 3.43 |
| | (2H, d, J = 12.3 Hz), 3.85 (1H, s), 4.69-4.64 (2H, m), 6.55-6.52 (1H, m), 6.77-6.73 (1H, |
| | m), 7.18 (1H, d, J = 6.8 Hz), 7.69 (1H, d, J = 6.9 Hz), 7.82-7.78 (1H, m), 7.96-7.91 (2H, m), |
| | 8.57-8.52 (1H, m), 9.07 (2H, s), 9.28 (2H, t, J = 5.6 Hz) |
| 4.09 | 1.31-1.19 (2H, m), 1.67-1.54 (3H, m), 2.06-1.99 (2H, m), 2.26-2.25 (3H, m), 2.90- |
| | 2.84 (2H, m), 3.32 (2H, s), 4.60-4.56 (2H, m), 6.79-6.75 (2H, m), 6.89 (1H, d, J = 5.5 |
| | Hz), 7.42 (1H, dd, J = 1.8, 8.7 Hz), 7.58 (1H, s), 7.78 (1H, d, J = 5.8 Hz), 8.26-8.21 (6H, |
| | m), 8.57 (1H, t, J = 6.5 Hz), 9.27 (1H, d, J = 11.1 Hz). |
| 4.10 | 0.69-0.64 (2H, m), 0.90-0.84 (2H, m), 1.25-1.13 (2H, m), 1.63-1.44 (3H, m), 1.86- |
| | 1.74 (3H, m), 2.13-2.12 (3H, m), 2.77-2.68 (2H, m), 3.27 (2H, t, J = 6.6 Hz), 4.61-4.57 |
| | (2H, m), 6.73 (2H, s), 6.88 (1H, d, J = 5.6 Hz), 7.41 (1H, dd, J = 1.7, 8.6 Hz), 7.55 (1H, s), |
| | 7.67 (1H, d, J = 2.3 Hz), 7.78 (1H, d, J = 5.8 Hz), 8.07-8.05 (1H, m), 8.17-8.14 (1H, m), |
| | 8.30 (1H, t, J = 5.3 Hz), 9.15-9.10 (1H, m). |
| 4.11 | 1.32-1.21 (2H, m), 1.70-1.56 (3H, m), 1.96 (2H, dd, J = 10.6, 11.5 Hz), 2.23-2.21 (3H, |
| | m), 2.85 (2H, d, J = 11.3 Hz), 3.39 (2H, t, J = 7.3 Hz), 4.65 (2H, d, J = 5.6 Hz), 6.75 (2H, s), |
| | 6.89 (1H, d, J = 5.6 Hz), 7.50-7.43 (2H, m), 7.61 (1H, s), 7.78 (1H, d, J = 5.9 Hz), 8.21- |
| | 8.20 (3H, m), 8.45 (1H, d, J = 2.4 Hz), 8.53 (1H, dd, J = 1.5, 4.8 Hz), 8.62 (1H, d, J = 2.4 |
| | Hz), 8.70 (1H, t, J = 5.7 Hz), 8.97 (1H, d, J = 1.6 Hz), 9.33 (1H, t, J = 5.8 Hz). |
| 4.12 | 1.29-1.16 (2H, m), 1.58-1.48 (1H, m), 1.65 (2H, d, J = 12.7 Hz), 1.99 (2H, dd, J = 9.6, |
| | 12.0 Hz), 2.24-2.23 (3H, m), 2.85 (2H, d, J = 11.5 Hz), 3.26 (2H, t, J = 6.2 Hz), 3.78 (3H, |
| | s), 4.62-4.58 (2H, m), 6.77 (2H, s), 6.88 (1H, d, J = 5.6 Hz), 7.42 (1H, dd, J = 1.7, 8.6 Hz), |
| | 7.57 (1H, s), 7.79-7.77 (2H, m), 8.01 (1H, d, J = 3.0 Hz), 8.18-8.07 (2H, m), 8.24 (2H, |
| | s), 9.18 (1H, t, J = 5.8 Hz). |
| 4.13 | 1.28-1.16 (2H, m), 1.52-1.38 (1H, m), 1.65-1.60 (2H, m), 1.84-1.76 (2H, m), 2.13 |
| | (3H, s), 2.33-2.32 (3H, m), 2.77-2.69 (2H, m), 3.32-3.27 (2H, m), 4.57 (2H, d, J = 5.8 |
| | Hz), 6.46-6.43 (1H, m), 6.73-6.70 (2H, m), 6.87 (1H, d, J = 5.5 Hz), 7.40 (1H, dd, J = 1.6, |
| | 8.7 Hz), 7.54 (1H, s), 7.77 (1H, d, J = 5.8 Hz), 7.96-7.93 (1H, m), 8.16-8.13 (1H, m), 8.55 |
| | (1H, t, J = 5.5 Hz), 9.05-9.00 (1H, m). |
| 4.14 | 1.16-1.04 (2H, m), 1.57-1.39 (3H, m), 1.74-1.65 (2H, m), 2.11-2.10 (3H, m), 2.17 |
| | (3H, s), 2.69-2.64 (2H, m), 3.18 (2H, t, J = 6.3 Hz), 4.60-4.57 (2H, m), 5.73 (1H, t, J = |
| | 5.7 Hz), 6.42 (1H, d, J = 5.5 Hz), 6.76-6.73 (2H, m), 6.86 (1H, d, J = 5.6 Hz), 7.47-7.44 |
| | (1H, m), 7.61-7.60 (1H, m), 7.79 (1H, d, J = 5.8 Hz), 7.91-7.89 (1H, m), 8.18-8.15 (1H, |
| | m), 9.02 (1H, t, J = 6.0 Hz). |
| 4.15 | 1.38-1.26 (2H, m), 1.76-1.61 (3H, m), 2.02 (2H, t, J = 11.2 Hz), 2.25-2.23 (3H, m), |
| | 2.92-2.85 (2H, m), 3.48 (2H, t, J = 5.6 Hz), 4.63-4.59 (2H, m), 6.77 (2H, s), 6.89 (1H, d, |
| | J = 5.8 Hz), 7.24-7.21 (1H, m), 7.53-7.42 (4H, m), 7.58 (1H, s), 7.78 (1H, d, J = 5.8 Hz), |
| | 8.18-8.10 (4H, m), 8.23 (1H, s), 8.64 (1H, t, J = 5.5 Hz), 9.18 (1H, t, J = 6.0 Hz). |
| 4.16 | 1.39-1.26 (2H, m), 1.76-1.63 (3H, m), 2.08-2.01 (2H, m), 2.26 (3H, s), 2.90 (2H, d, J = |
| | 11.0 Hz), 3.49-3.44 (2H, m), 4.64-4.60 (2H, m), 6.79 (2H, s), 6.90-6.88 (1H, m), 7.33- |
| | 7.30 (1H, m), 7.44 (1H, dd, J = 1.7, 8.6 Hz), 7.59-7.52 (2H, m), 7.78 (1H, d, J = 5.8 Hz), |
| | 8.25-8.15 (4H, m), 8.48-8.44 (1H, m), 8.70-8.64 (2H, m), 9.23 (1H, t, J = 5.9 Hz), 9.31 |
| | (1H, d, J = 1.5 Hz). |
| 4.17 | 0.97-0.87 (4H, m), 1.30-1.18 (2H, m), 1.68-1.48 (3H, m), 2.06-1.92 (3H, m), 2.25 |
| | (3H, s), 2.90-2.84 (2H, m), 3.29-3.23 (2H, m), 4.58-4.54 (2H, m), 6.54-6.52 (1H, m), |
| | 6.80-6.76 (2H, m), 6.87 (1H, d, J = 5.6 Hz), 7.42-7.38 (1H, m), 7.54 (1H, s), 7.77 (1H, d, |
| | J = 5.8 Hz), 7.93-7.90 (1H, m), 8.17-8.13 (1H, m), 8.25 (2H, s), 8.55 (1H, t, J = 5.8 Hz), |
| | 9.02-8.97 (1H, m). |
| 4.18 | 1.35-1.23 (2H, m), 1.73-1.60 (3H, m), 2.07-1.98 (2H, m), 2.25 (3H, s), 2.88 (2H, d, |
| | J = 11.7 Hz), 3.42 (2H, t, J = 6.0 Hz), 4.68 (2H, d, J = 6.4 Hz), 6.79-6.75 (2H, m), 6.88 (1H, |
| | d, J = 5.6 Hz), 7.40-7.36 (1H, m), 7.50-7.45 (3H, m), 7.60 (1H, s), 7.77 (1H, d, J = 5.8 |
| | Hz), 8.24-8.19 (5H, m), 8.95-8.90 (2H, m), 9.63-9.58 (1H, m). |
| 4.19 | 1.32-1.21 (2H, m), 1.68-1.54 (3H, m), 2.06 (2H, t, J = 11.2 Hz), 2.27-2.26 (3H, m), 2.92- |
| | 2.85 (2H, m), 3.29 (2H, t, J = 6.4 Hz), 4.60-4.56 (2H, m), 6.76 (2H, s), 6.89 (1H, d, J = |
| | 5.5 Hz), 7.44-7.40 (1H, m), 7.58 (1H, s), 7.78 (1H, d, J = 5.8 Hz), 8.19-8.19 (2H, m), 8.33- |
| | 8.33 (1H, m), 8.79 (1H, t, J = 5.7 Hz), 9.31 (1H, t, J = 5.8 Hz). |
| 4.20 | 1.32-1.21 (2H, m), 1.68-1.54 (3H, m), 2.06 (2H, t, J = 11.2 Hz), 2.27-2.26 (3H, m), 2.92- |
| | 2.85 (2H, m), 3.29 (2H, t, J = 6.4 Hz), 4.60-4.56 (2H, m), 6.76 (2H, s), 6.89 (1H, d, J = |
| | 5.5 Hz), 7.44-7.40 (1H, m), 7.58 (1H, s), 7.78 (1H, d, J = 5.8 Hz), 8.19-8.19 (2H, m), 8.33- |
| | 8.33 (1H, m), 8.79 (1H, t, J = 5.7 Hz), 9.31 (1H, t, J = 5.8 Hz). |
| 4.21 | 1.27-1.15 (2H, m), 1.51-1.41 (1H, m), 1.64-1.61 (2H, m), 1.83-1.75 (2H, m), 2.14- |
| | 2.12 (6H, m), 2.31-2.29 (3H, m), 2.76-2.68 (2H, m), 3.29 (2H, t, J = 5.9 Hz), 4.56 (2H, |
| | d, J = 5.8 Hz), 6.72 (2H, s), 6.87 (1H, d, J = 5.5 Hz), 7.40 (1H, dd, J = 1.7, 8.6 Hz), 7.54 (1H, |
| | s), 7.83-7.76 (2H, m), 8.17-8.13 (1H, m), 8.32 (1H, t, J = 5.5 Hz), 8.97 (1H, t, J = 6.0 Hz). |
| 4.22 | 3.07-2.85 (9H, m), 4.69 (2H, d, J = 6.1 Hz), 6.76 (2H, s), 6.98-6.90 (3H, m), 7.57-7.47 |
| | (3H, m), 7.63 (1H, s), 7.81 (1H, d, J = 5.8 Hz), 8.04 (1H, d, J = 1.8 Hz), 8.21-8.17 (1H, m), |
| | 8.41 (1H, d, J = 1.8 Hz), 9.75 (1H, t, J = 6.2 Hz), 10.98 (1H, s). |
| 4.23 | 1.29-1.22 (1H, m), 2.35-2.33 (3H, m), 2.75-2.63 (4H, m), 3.51-3.48 (2H, m), 4.70- |
| | 4.66 (2H, m), 6.72 (2H, s), 6.90-6.79 (2H, m), 7.15 (1H, t, J = 7.8 Hz), 7.48-7.44 (1H, |
| | m), 7.61-7.59 (1H, m), 7.79-7.76 (1H, m), 8.18-8.05 (3H, m), 8.42 (1H, d, J = 2.4 Hz), |
| | 9.77-9.73 (1H, m), 10.99 (1H, s). |
| 4.24 | 2.33-2.31 (3H, m), 2.59-2.55 (2H, m), 2.83-2.77 (2H, m), 3.43 (2H, s), 4.66-4.62 (2H, |
| | m), 6.73-6.69 (2H, m), 6.86 (1H, d, J = 5.7 Hz), 7.02-6.98 (1H, m), 7.47-7.41 (3H, m), |
| | 7.59-7.57 (1H, m), 7.76 (1H, d, J = 5.7 Hz), 8.05 (1H, d, J = 2.4 Hz), 8.15-8.12 (1H, m), |
| | 8.43-8.41 (1H, m), 9.74 (1H, t, J = 6.4 Hz), 11.15-11.13 (1H, m). |
| 4.25 | 1.26-1.15 (2H, m), 1.63-1.51 (3H, m), 1.91-1.84 (2H, m), 2.33 (2H, t, J = 6.4 Hz), 2.84 |
| | (2H, d, J = 11.5 Hz), 3.32-3.35 (2H, m), 3.46 (2H, t, J = 6.1 Hz), 4.36-4.31 (1H, m), 4.58 |
| | (2H, d, J = 6.3 Hz), 6.70 (2H, s), 6.85 (1H, d, J = 5.6 Hz), 7.43-7.39 (1H, m), 7.53 (1H, s), |
| | 7.80-7.74 (2H, m), 8.14-8.10 (1H, m), 8.28-8.26 (1H, m), 8.79 (1H, t, J = 5.7 Hz), 9.48 |
| | (1H, t, J = 6.4 Hz). |
| 4.26 | 1.14-1.28 (2H, m), 1.61-1.68 (2H, m), 1.68-1.78 (2H, m), 2.09 (3H, s), 2.52-2.54 (1H, |
| | m), 2.67-2.77 (2H, m), 3.22-3.28 (2H, m), 4.54-4.71 (2H, m), 6.61-6.69 (1H, m), 6.69- |
| | 6.75 (2H, m), 6.80 (1H, t, J = 5.7 Hz), 6.83-6.88 (1H, m), 7.13-7.20 (1H, m), 7.35-7.44 |
| | (1H, m), 7.46-7.56 (2H, m), 7.69-7.81 (1H, m), 8.09-8.19 (1H, m), 8.89 (1H, t, J = 6.4 |
| | Hz). |
| 32.05 | 1.02 (6H, d, J = 6.3 Hz), 1.24-1.36 (3H, m), 1.50-1.52 (2H, m), 1.62-1.74 (1H, m), 2.62- |
| | 2.67 (2H, m), 3.25 (2H, s), 4.60 (2H, d, J = 5.8 Hz), 6.76 (2H, s), 6.87 (1H, d, J = 5.8 Hz), |
| | 7.41 (1H, dd, J = 1.5, 8.6 Hz), 7.57 (1H, s), 7.67 (1H, t, J = 1.7 Hz), 7.76 (1H, d, J = 5.8 Hz), |
| | 8.07 (2H, d, J = 1.6 Hz), 8.15 (1H, d, J = 8.6 H), 9.22 (1H, t, J = 5.9 Hz), 10.19 (1H, s). |
| 8.09 | 1.08-1.23 (2H, m), 1.74 (2H, d, J = 11.8 Hz), 2.11-2.24 (1H, m), 3.07-3.19 (5H, m), 3.52 |
| | (2H, d, J = 7.3 Hz), 4.23 (2H, d, J = 13.4 Hz), 4.66 (2H, d, J = 5.8 Hz), 7.12-7.20 (2H, m), |
| | 7.22 (1H, d, J = 7.0 Hz), 7.64-7.71 (1H, m), 7.73 (1H, dd, J = 8.6, 1.7 Hz), 7.84 (1H, s), 8.14- |
| | 8.26 (3H, m), 8.59 (1H, d, J = 8.6 Hz), 8.70 (1H, d, J = 2.1 Hz), 9.20 (2H, br.s), 9.36 (1H, t, |
| | J = 5.9, 5.9 Hz), 13.41 (1H, s), 13.55 (1H, s). |
| 11.01 | 1.50-1.60 (4H, m), 1.67-1.78 (2H, m), 1.91-1.98 (2H, m), 4.35-4.42 (1H, m), 4.58 |
| | (2H, d, J = 5.8 Hz), 6.64 (1H, d, J = 7.3 Hz), 6.84 (2H, s), 6.88 (1H, d, J = 5.9 Hz), 7.41 (1H, |
| | dd, J = 8.6, 1.5 Hz), 7.56 (1H, s), 7.75 (1H, d, J = 5.9 Hz), 8.06 (1H, d, J = 2.1 Hz), 8.15 (1H, |
| | d, J = 8.6 Hz), 8.58 (1H, d, J = 2.0 Hz), 8.93 (1H, t, J = 5.9 Hz) |
| 11.42 | 0.25-0.35 (2H, m), 0.35-0.44 (2H, m), 1.46-1.65 (3H, m), 1.72-1.84 (2H, m), 2.18- |
| | 2.30 (2H, m), 2.86-3.00 (2H, m), 3.93-4.06 (1H, m), 4.58 (2H, d, J = 5.8 Hz), 6.55 (1H, |
| | d, J = 7.9 Hz), 6.70 (2H, s), 6.86 (1H, d, J = 5.9 Hz), 7.39 (1H, dd, J = 8.6, 1.8 Hz), 7.54 (1H, |
| | s), 7.76 (1H, d, J = 5.8 Hz), 8.07 (1H, d, J = 2.1 Hz), 8.13 (1H, d, J = 8.6 Hz), 8.57 (1H, d, J = |
| | 2.1 Hz), 8.92 (1H, t, J = 5.9 Hz) |
| 19.01 | 3.81 (3H, s), 4.66 (2H, d, J = 5.8 Hz), 6.75 (2H, s), 6.89 (1H, d, J = 5.6 Hz), 7.06-7.14 |
| | (1H, m), 7.16-7.23 (1H, m), 7.40-7.48 (3H, m), 7.61 (1H, s), 7.77 (1H, d, J = 5.8 Hz), |
| | 8.16 (1H, d, J = 8.6 Hz), 8.35 (1H, t, J = 2.1 Hz), 8.84 (1H, d, J = 2.1 Hz), 9.03 (1H, d, J = |
| | 2.1 Hz), 9.36 (1H, t, J = 5.9 Hz) |
| 19.02 | 4.64 (2H, d, J = 5.8 Hz), 6.74 (2H, s), 6.89 (1H, d, J = 5.6 Hz), 7.43 (1H, dd, J = 1.7, 8.6 Hz), |
| | 7.61 (1H, s), 7.77 (1H, d, J = 5.8 Hz), 8.16 (1H, d, J = 8.6 Hz), 8.48-8.52 (1H, m), 8.89 |
| | (1H, d, J = 2.0 Hz), 9.05 (1H, d, J = 1.8 Hz), 9.40 (1H, t, J = 5.8 Hz) |
| 19.03 | 4.64 (2H, d, J = 5.8 Hz), 6.57 (1H, dd, J = 3.4, 1.8 Hz), 6.80 (2H, br.s), 6.88 (1H, d, J = 5.8 |
| | Hz), 7.45 (1H, dd, J = 8.6, 1.6 Hz), 7.57 (1H, t, J = 2.9 Hz), 7.60 (1H, s), 7.75 (1H, d, J = 5.9 |
| | Hz), 8.16 (1H, d, J = 8.6 Hz), 8.51 (1H, d, J = 2.0 Hz), 8.79 (1H, d, J = 2.0 Hz), 9.15 (1H, t, |
| | J = 5.9 Hz), 11.92 (1H, s) |
| 19.04 | 4.65 (2H, d, J = 5.8 Hz), 6.92 (1H, d, J = 5.9 Hz), 6.96 (2H, s), 7.47 (1H, dd, J = 1.5, 8.6 Hz), |
| | 7.63 (1H, s), 7.77 (1H, d, J = 5.8 Hz), 8.20 (1H, d, J = 8.6 Hz), 8.39 (1H, t, J = 2.0 Hz), 8.81 |
| | (1H, d, J = 2.3 Hz), 9.04 (1H, d, J = 1.8 Hz), 9.47 (1H, t, J = 5.8 Hz) |
| 19.05 | 4.62 (2H, d, J = 5.8 Hz), 6.75 (2H, s), 6.88 (1H, d, J = 5.8 Hz), 7.42 (1H, dd, J = 1.4, 8.6 Hz), |
| | 7.52-7.58 (2H, m), 7.62-7.64 (1H, m), 7.77 (1H, d, J = 5.8 Hz), 7.89 (1H, d, J = 7.8 Hz), |
| | 7.97-7.98 (1H, m), 8.16 (1H, d, J = 8.6 Hz), 9.27 (1H, t, J = 5.8 Hz) |
| 19.06 | 4.64 (2H, d, J = 5.8 Hz), 6.75 (2H, s), 6.88 (1H, d, J = 5.8 Hz), 7.43 (1H, dd, J = 8.6, 1.6 Hz), |
| | 7.60 (1H, s), 7.77 (1H, d, J = 5.8 Hz), 8.18 (1H, d, J = 8.6 Hz), 8.56 (1H, d, J = 2.2 Hz), 8.67 |
| | (1H, d, J = 2.0 Hz), 9.44 (1H, t, J = 5.8 Hz). |
| 29.01 | 4.68 (2H, d, J = 5.8 Hz), 6.73 (2H, s), 6.89 (1H, d, J = 5.6 Hz), 7.43-7.52 (2H, m), 7.52- |
| | 7.60 (2H, m), 7.63 (1H, s), 7.77 (1H, d, J = 5.8 Hz), 7.81-7.88 (2H, m), 8.16 (1H, d, J = 8.6 |
| | Hz), 8.55 (1H, t, J = 2.2 Hz), 9.04-9.11 (2H, m), 9.42 (1H, t, J = 5.8 Hz). |
| 29.03 | 4.68 (2H, d, J = 5.7 Hz), 5.47 (2H, s), 6.67 (1H, d, J = 1.9 Hz), 6.94-7.00 (2H, m), 7.07 |
| | (1H, d, J = 6.4 Hz), 7.19-7.30 (3H, m), 7.60 (1H, dd, J = 1.7, 8.6 Hz), 7.67 (1H, d, J = 1.9 |
| | Hz), 7.71 (1H, d, J = 6.4 Hz), 7.74 (1H, d, J = 1.7 Hz), 7.87 (2H, s), 8.29 (1H, t, J = 2.1 Hz), |
| | 8.35 (1H, d, J = 8.6 Hz), 8.76 (1H, d, J = 2.1 Hz), 9.07 (1H, d, J = 2.1 Hz), 9.41 (1H, t, J = |
| | 5.9 Hz) |
| 29.04 | 2.26-2.42 (4H, m), 2.73 (4H, t, J = 4.8 Hz), 3.33 (1H, br.s), 3.54 (2H, s), 4.64 (2H, d, J = |
| | 5.8 Hz), 6.73 (2H, s), 6.88 (1H, dd, J = 0.8, 5.9 Hz), 7.43 (1H, dd, J = 1.7, 8.7 Hz), 7.59 (1H, |
| | d, J = 1.7 Hz), 7.77 (1H, d, J = 5.8 Hz), 8.11-8.20 (2H, m), 8.63 (1H, d, J = 2.0 Hz), 8.99 |
| | (1H, d, J = 2.1 Hz), 9.34 (1H, t, J = 5.9 Hz). |
| 29.07 | 2.56 (4H, t, J = 5.1 Hz), 3.32 (4H, s), 3.68 (2H, s), 4.62 (2H, d, J = 5.8 Hz), 6.72 (2H, s), |
| | 6.79-6.84 (2H, m), 6.87 (1H, d, J = 5.8 Hz), 7.42 (1H, dd, J = 8.6, 1.8 Hz), 7.57 (1H, d, J = |
| | 1.7 Hz), 7.66 (1H, d, J = 8.0 Hz), 7.77 (1H, d, J = 5.8 Hz), 7.90 (1H, dd, J = 8.0, 1.8 Hz), |
| | 8.00 (1H, d, J = 1.8 Hz), 8.12-8.18 (3H, m), 9.24 (1H, t, J = 6.0 Hz). |
| 29.08 | 2.62 (4H, t, J = 5.1 Hz), 3.29 (4H, t, J = 5.1 Hz), 3.81 (2H, s), 4.64 (2H, d, J = 5.7 Hz), 6.73 |
| | (2H, s), 6.77-6.82 (2H, m), 6.88 (1H, d, J = 5.8 Hz), 7.43 (1H, dd, J = 8.6, 1.8 Hz), 7.61 |
| | (1H, d, J = 1.7 Hz), 7.77 (1H, d, J = 5.8 Hz), 8.12-8.18 (3H, m), 8.37 (1H, d, J = 1.9 Hz), |
| | 8.99 (1H, d, J = 1.9 Hz), 9.39 (1H, t, J = 5.9 Hz). |
| 29.09 | 1.09-1.21 (2H, m), 1.42-1.59 (4H, m), 1.60-1.70 (2H, m), 1.97-2.09 (1H, m), 2.15 |
| | (2H, d, J = 7.6 Hz), 2.25-2.45 (8H, m), 3.50 (2H, s), 4.61 (2H, d, J = 5.9 Hz), 6.71 (2H, s), |
| | 6.86 (1H, d, J = 5.8 Hz), 7.40-7.48 (3H, m), 7.55-7.57 (1H, m), 7.76 (1H, d, J = 5.8 Hz), |
| | 7.79-7.82 (1H, m), 7.83 (1H, s), 8.14 (1H, d, J = 8.6 Hz), 9.12 (1H, t, J = 6.0 Hz). |
| 29.10 | 2.31 (3H, s), 2.38-2.54 (8H, m), 3.55 (2H, s), 4.61 (2H, d, J = 5.9 Hz), 6.80 (2H, s), 6.89 |
| | (1H, d, J = 5.9 Hz), 7.43 (1H, dd, J = 8.6, 1.8 Hz), 7.54-7.56 (1H, m), 7.57-7.59 (1H, m), |
| | 7.77 (1H, d, J = 5.8 Hz), 7.80-7.82 (1H, m), 7.86-7.88 (1H, m), 8.16 (1H, d, J = 8.6 Hz), |
| | 9.25 (1H, t, J = 6.0 Hz). |
| 29.11 | 1.10-1.21 (2H, m), 1.41-1.57 (4H, m), 1.58-1.70 (2H, m), 1.96-2.08 (1H, m), 2.11- |
| | 2.20 (2H, m), 2.30-2.45 (8H, m), 3.52 (2H, s), 4.61 (2H, d, J = 5.6 Hz), 6.72 (2H, s, 6.87 |
| | (1H, d, J = 5.8 Hz), 7.41 (1H, dd, J = 8.6, 1.8 Hz), 7.51-7.53 (1H, m), 7.57 (1H, s), 7.77 |
| | (1H, d, J = 5.8 Hz), 7.79-7.81 (1H, m), 7.85-7.87 (1H, m), 8.15 (1H, d, J = 8.6 Hz), 9.24 |
| | (1H, t, J = 5.9 Hz). |
| 33.01 | (Methanol-d4) 1.44 (2H, qd, J = 3.8, 13.3 Hz), 1.80 (1H, m), 1.92 (2H, d, J = 13.5 Hz), 2.44 |
| | (2H, t, J = 14.0 Hz), 2.55 (3H, s), 3.19 (2H, d, J = 11.0 Hz), 3.40 (2H, d, J = 6.8 Hz), 4.51 |
| | (2H, s), 7.52 (1H, dd, J = 0.9, 8.0 Hz), 7.63 (1H, t, J = 8.0 Hz), 7.81 (1H, d, J = 2.5 Hz), 7.84 |
| | (1H, dd, J = 1.1, 8.1 Hz), 8.10 (1H, d, J = 2.5 Hz), 9.64 (1H, s) |
| 33.02 | 1.33-1.24 (2H, m), 1.74-1.59 (3H, m), 1.89 (2H, t, J = 11.0 Hz), 2.20-2.16 (3H, m), 2.86- |
| | 2.80 (2H, m), 3.41 (2H, t, J = 7.1 Hz), 4.36 (2H, s), 7.58-7.53 (1H, m), 7.99-7.88 (2H, |
| | m), 8.29 (1H, s), 8.36 (1H, d, J = 1.8 Hz), 8.60-8.55 (1H, m), 8.72 (1H, t, J = 4.6 Hz), 10.81 |
| | (1H, s). |
| 33.03 | 1.37-1.26 (2H, m), 1.75-1.61 (3H, m), 2.01 (2H, t, J = 11.4 Hz), 2.24 (3H, s), 2.91-2.84 |
| | (2H, m), 3.43 (2H, t, J = 5.9 Hz), 7.69-7.66 (1H, m), 7.92 (1H, d, J = 2.3 Hz), 8.05 (1H, s), |
| | 8.17 (1H, dd, J = 2.4, 8.8 Hz), 8.22 (1H, s), 8.37 (1H, d, J = 2.3 Hz), 8.73 (1H, t, J = 5.8 Hz), |
| | 8.81 (1H, d, J = 2.5 Hz), 10.95-10.93 (1H, m), 12.23 (1H, s). |
| 33.04 | 1.42-1.31 (2H, m), 1.76-1.69 (3H, m), 2.24-2.16 (2H, m), 2.34 (3H, s), 2.99 (2H, d, J = |
| | 11.4 Hz), 3.44 (2H, t, J = 5.8 Hz), 6.69 (2H, s), 6.93 (1H, d, J = 5.8 Hz), 7.78-7.71 (2H, m), |
| | 7.92 (1H, d, J = 2.0 Hz), 8.10-8.05 (1H, m), 8.25 (2H, s), 8.38 (1H, d, J = 2.0 Hz), 8.46 |
| | (1H, s), 8.78 (1H, t, J = 5.6 Hz), 10.67 (1H, s). |
| 33.05 | 1.17-1.22 (2H, m), 1.31 (3H, t, J = 7.1 Hz), 1.51-1.58 (1H, m), 1.65 (2H, d, J = 14.0 Hz), |
| | 2.02 (2H, t, J = 11.3 Hz), 2.25 (3H, s), 2.87 (2H, d, J = 11.4 Hz), 3.28-3.32 (2H, m), 3.93 |
| | (3H, s), 4.33 (2H, q, J = 7.1 Hz), 4.40 (2H, d, J = 5.6 Hz), 7.31-7.39 (2H, m), 7.70 (1H, d, |
| | J = 2.4 Hz), 8.16 (1H, s), 8.23 (1H, d, J = 2.4 Hz), 8.59 (1H, t, J = 5.8 Hz), 8.78 (1H, t, J = |
| | 5.8 Hz), 9.08 (1H, s) |
| 33.06 | 1.35-1.23 (2H, m), 1.73-1.54 (3H, m), 1.92-1.83 (2H, m), 2.20 (3H, s), 2.81 (4H, t, J = |
| | 7.2 Hz), 3.39 (2H, t, J = 5.9 Hz), 3.57 (2H, dd, J = 6.3, 14.6 Hz), 6.90-6.87 (1H, m), 7.59 |
| | (1H, d, J = 1.1 Hz), 7.81 (1H, d, J = 2.4 Hz), 8.29 (1H, d, J = 2.4 Hz), 8.99-8.90 (2H, m). |
| 33.07 | 1.61-1.49 (2H, m), 1.93-1.84 (3H, m), 2.71-2.68 (3H, m), 2.96-2.84 (2H, m), 3.51- |
| | 3.36 (4H, m), 7.16 (1H, d, J = 7.0 Hz), 7.68 (1H, dd, J = 4.9, 6.5 Hz), 7.97 (1H, d, J = 2.4 |
| | Hz), 8.17-8.13 (1H, m), 8.42 (1H, d, J = 2.3 Hz), 8.69-8.55 (3H, m), 9.12 (2H, s), 10.37 |
| | (1H, s), 11.15 (1H, s), 13.26 (1H, s). |
| 33.08 | 1.34-1.22 (2H, m), 1.73-1.57 (3H, m), 1.88-1.79 (2H, m), 2.16-2.14 (3H, m), 2.81- |
| | 2.75 (2H, m), 3.43 (2H, t, J = 6.4 Hz), 7.94 (1H, d, J = 2.3 Hz), 8.10-8.07 (1H, m), 8.32 |
| | (1H, dd, J = 2.3, 9.2 Hz), 8.39 (1H, d, J = 2.3 Hz), 8.75-8.69 (2H, m), 8.87-8.86 (1H, m), |
| | 8.93 (1H, d, J = 1.9 Hz), 11.10 (1H, s). |
| 33.09 | 1.66-1.62 (4H, m), 1.79-1.77 (1H, m), 2.60-2.58 (3H, m), 2.69-2.64 (2H, m), 3.30- |
| | 3.25 (2H, m), 3.64-3.60 (2H, m), 3.97-3.96 (3H, m), 4.40-4.36 (2H, m), 7.37-7.33 |
| | (2H, m), 7.69 (1H, d, J = 2.4 Hz), 8.20-8.16 (2H, m), 8.78-8.67 (2H, m). |
| 33.10 | 1.43-1.32 (2H, m), 1.75-1.64 (3H, m), 2.02-1.95 (2H, m), 2.22-2.19 (3H, m), 2.62- |
| | 2.59 (3H, m), 2.80-2.73 (2H, m), 3.50-3.47 (2H, m), 7.60-7.56 (1H, m), 7.69-7.63 |
| | (1H, m), 7.87-7.84 (1H, m), 8.17-8.11 (2H, m), 8.31 (1H, d, J = 2.3 Hz), 8.57-8.47 (1H, |
| | m), 10.29 (1H, s). |
| 33.11 | 1.68-1.60 (2H, m), 2.03-1.93 (3H, m), 2.31-2.23 (2H, m), 2.79 (3H, s), 3.09-3.06 (2H, |
| | m), 3.76 (2H, s), 8.14-8.09 (6H, m), 8.59-8.59 (1H, m), 8.80-8.72 (1H, m), 10.58- |
| | 10.49 (1H, m), 11.24 (1H, s). |
| 33.12 | 1.31-1.19 (2H, m), 1.62-1.51 (1H, m), 1.66 (2H, d, J = 12.9 Hz), 1.84-1.77 (2H, m), |
| | 2.15-2.13 (3H, m), 2.76 (2H, d, J = 11.4 Hz), 3.39 (2H, t, J = 6.0 Hz), 6.92-6.87 (3H, m), |
| | 7.51 (1H, t, J = 8.0 Hz), 7.87 (1H, d, J = 6.0 Hz), 8.00-7.93 (2H, m), 8.14-8.09 (1H, m), |
| | 8.39 (1H, d, J = 2.3 Hz), 8.73-8.68 (1H, m), 10.65 (1H, s). |
| 33.13 | 1.31-1.20 (2H, m), 1.57-1.53 (1H, m), 1.66 (2H, d, J = 12.4 Hz), 1.81 (2H, t, J = 11.5 Hz), |
| | 2.11-2.01 (1H, m), 2.15 (3H, d, J = 3.1 Hz), 2.45-2.34 (1H, m), 2.62-2.54 (1H, m), 2.84- |
| | 2.76 (3H, m), 3.34 (2H, d, J = 2.4 Hz), 5.46-5.39 (1H, m), 5.82-5.80 (2H, m), 6.44- |
| | 6.40 (1H, m), 7.76 (2H, d, J = 2.9 Hz), 8.28-8.24 (1H, m), 8.83 (1H, s), 8.92 (1H, d, J = 6.4 |
| | Hz). |
| 33.14 | 1.36-1.24 (2H, m), 1.72-1.61 (3H, m), 2.01 (2H, t, J = 11.4 Hz), 2.24 (3H, s), 2.91-2.84 |
| | (2H, m), 3.41 (2H, t, J = 6.1 Hz), 7.70 (1H, d, J = 2.6 Hz), 7.91 (1H, d, J = 2.4 Hz), 8.22 (1H, |
| | s), 8.36 (1H, d, J = 2.4 Hz), 8.47 (1H, d, J = 2.4 Hz), 8.64 (1H, d, J = 2.3 Hz), 8.76 (1H, t, |
| | J = 5.8 Hz), 10.82 (1H, s), 11.98-11.96 (1H, m). |
| 33.15 | 1.28-1.24 (2H, m), 1.63-1.53 (1H, m), 1.69 (2H, d, J = 13.0 Hz), 1.87-1.79 (2H, m), |
| | 2.16-2.15 (3H, m), 2.77 (2H, d, J = 11.3 Hz), 3.42 (2H, t, J = 6.2 Hz), 7.29-7.16 (2H, m), |
| | 7.57 (1H, s), 7.91 (1H, d, J = 2.2 Hz), 7.98 (1H, dd, J = 0.5, 7.5 Hz), 8.39-8.37 (1H, m), |
| | 8.77 (1H, t, J = 5.8 Hz), 11.15-11.13 (1H, m), 11.62-11.58 (1H, m). |
| 33.16 | 1.29-1.17 (2H, m), 1.68-1.46 (3H, m), 1.84-1.75 (2H, m), 2.14-2.13 (3H, m), 2.75 |
| | (2H, dd, J = 2.9, 8.3 Hz), 3.34 (2H, s), 4.63-4.59 (2H, m), 6.72 (2H, s), 6.88 (1H, d, J = 5.5 |
| | Hz), 7.68-7.58 (2H, m), 7.81-7.76 (2H, m), 8.11 (1H, s), 8.28-8.27 (1H, m), 8.87-8.81 |
| | (1H, m), 9.41 (1H, t, J = 6.3 Hz). |
| 33.17 | 1.28-1.12 (3H, m), 1.65-1.49 (3H, m), 1.85-1.77 (2H, m), 2.15-2.14 (3H, m), 2.76 |
| | (2H, d, J = 11.4 Hz), 3.93-3.92 (4H, m), 4.44-4.42 (2H, m), 7.37-7.30 (2H, m), 7.74- |
| | 7.72 (1H, m), 8.27-8.24 (2H, m), 8.69-8.64 (1H, m), 8.82-8.77 (1H, m), 8.88 (1H, s). |
| 33.18 | 1.31-1.16 (3H, m), 1.39 (1H, s), 1.67-1.52 (4H, m), 1.93-1.86 (2H, m), 2.18 (3H, s), |
| | 2.38-2.34 (1H, m), 2.80 (2H, d, J = 11.5 Hz), 3.87 (1H, s), 4.65-4.61 (2H, m), 6.72-6.68 |
| | (2H, m), 7.04 (1H, d, J = 6.1 Hz), 7.40-7.36 (1H, m), 7.84-7.79 (2H, m), 8.02-7.98 (1H, |
| | m), 8.29-8.24 (3H, m), 8.80 (1H, t, J = 5.8 Hz), 9.35 (1H, t, J = 6.1 Hz). |
| 33.19 | 1.28-1.16 (2H, m), 1.65-1.49 (3H, m), 1.83-1.75 (2H, m), 2.13-2.12 (3H, m), 2.74 |
| | (2H, d, J = 11.5 Hz), 4.58-4.54 (2H, m), 6.62-6.57 (2H, m), 6.94-6.90 (1H, m), 7.23- |
| | 7.18 (1H, m), 7.41-7.40 (1H, m), 7.83-7.81 (2H, m), 8.30-8.28 (1H, m), 8.78 (1H, t, |
| | J = 5.8 Hz), 9.52 (1H, t, J = 6.3 Hz). |
| 33.20 | 1.28-1.16 (2H, m), 1.56-1.47 (1H, m), 1.63 (2H, d, J = 12.3 Hz), 1.84-1.76 (2H, m), |
| | 2.13-2.12 (3H, m), 2.77-2.71 (2H, m), 3.97 (3H, s), 4.56 (2H, d, J = 6.3 Hz), 6.62-6.59 |
| | (2H, m), 6.82 (1H, d, J = 5.6 Hz), 7.41 (1H, s), 7.59 (1H, s), 7.66 (1H, d, J = 5.6 Hz), 7.85- |
| | 7.83 (1H, m), 8.31-8.30 (1H, m), 8.80 (1H, t, J = 5.6 Hz), 9.27 (1H, t, J = 6.3 Hz). |
| 33.21 | 1.30-1.24 (1H, m), 1.40 (2H, q, J = 12.9 Hz), 1.91-1.84 (3H, m), 2.75 (3H, s), 2.95-2.87 |
| | (2H, m), 3.44-3.40 (2H, m), 4.74-4.69 (2H, m), 7.42 (1H, d, J = 6.7 Hz), 7.80-7.71 (2H, |
| | m), 7.87-7.84 (1H, m), 8.11-8.09 (2H, m), 8.16-8.14 (2H, m), 8.32-8.30 (1H, m), 8.81- |
| | 8.76 (1H, m), 9.11-9.02 (1H, m), 9.71-9.65 (1H, m). |
| 33.22 | 1.15-1.26 (2H, m), 1.44-1.53 (1H, m), 1.58-1.65 (2H, m), 1.75-1.84 (2H, m), 2.13 |
| | (3H, s), 2.74 (2H, d, J = 11.0 Hz), 3.26-3.32 (2H, m), 3.94 (3H, s), 4.40 (2H, d, J = 5.7 Hz), |
| | 7.29 (1H, t, J = 54.2 Hz), 7.32-7.41 (2H, m), 7.70 (1H, d, J = 2.4 Hz), 8.23 (1H, d, J = 2.4 |
| | Hz), 8.57-8.63 (1H, m), 8.72-8.78 (1H, m), 8.88-8.93 (1H, m) |
| 33.23 | 1.44-1.53 (2H, m), 1.81-1.87 (3H, m), 2.40 (6H, s), 2.70 (3H, d, J = 4.8 Hz), 2.83-2.93 |
| | (2H, m), 3.35-3.41 (4H, m), 3.88-3.94 (2H, m), 4.50 (2H, d, J = 5.6 Hz), 7.13 (2H, s), |
| | 7.76 (1H, d, J = 2.4 Hz), 8.24 (3H, s), 8.25 (1H, d, J = 2.4 Hz), 8.59-8.65 (1H, m), 8.73- |
| | 8.79 (1H, m), 9.89 (1H, s). |

### Biological Methods

### Determination of the % Inhibition for FXIIa

Factor XIIa inhibitory activity in vitro was determined using standard published methods (see e.g. Shori et al., Biochem. Pharmacol., 1992, 43, 1209; Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC50 value for the test compound was determined.

Data acquired from this assay are shown in Table 13 using the following scale:

| Category | IC₅₀ (nM) |
|---|---|
| A | <300 |
| B | 300-1,000 |
| C | 1,000-3,000 |
| D | 3,000-10,000 |
| E | 10,000-40,000 |

TABLE 13
Human FXIIa data, molecular weight and LCMS data

| Example | Human FXIIa | Molecular | LCMS Mass |
|---|---|---|---|
| number | IC50 (nM) | weight | Ion |
| 1.01 | E | 395.2 | 396.2 |
| 1.02 | E | 417.1 | 418.1 |
| 1.03 | E | 425.2 | 426.1 |
| 1.04 | E | 403.1 | 404.4 |
| 1.05 | E | 447.1 | 448.4 |
| 1.06 | D | 495.1 | 496.4 |
| 1.07 | E | 460.2 | 461.5 |
| 1.08 | D | 385.1 | 386.4 |
| 1.09 | C | 398.2 | 399.5 |
| 1.10 | C | 438.2 | 439.5 |
| 1.11 | D | 440.2 | 441.4 |
| 1.12 | C | 453.2 | 454.5 |
| 1.13 | B | 438.2 | 439.5 |
| 1.14 | D | 447.1 | 448.4 |
| 1.15 | E | 461.2 | 462.5 |
| 1.16 | D | 421.1 | 422.5 |
| 1.17 | D | 488.1 | 489.6 |
| 1.18 | E | 397.2 | 398.5 |
| 1.19 | D | 412.1 | 413.4 |
| 1.20 | D | 426.2 | 427.4 |
| 1.21 | E | 461.1 | 462.5 |
| 1.22 | D | 486.2 | 487.0 |
| 1.23 | D | 418.1 | 419.4 |
| 1.24 | D | 418.1 | 419.7 |
| 1.25 | B | 454.2 | 455.6 |
| 1.26 | C | 495.3 | 496.7 |
| 1.27 | E | 500.2 | 501.3 |
| 1.28 | B | 501.2 | 502.4 |
| 1.29 | D | 438.2 | 439.4 |
| 1.30 | D | 515.2 | 516.7 |
| 1.31 | C | 514.2 | 515.6 |
| 1.32 | E | 514.2 | 515.1 |
| 1.33 | E | 500.2 | 501.5 |
| 1.34 | E | 514.2 | 515.1 |
| 1.35 | E | 438.2 | 439.5 |
| 1.36 | C | 467.2 | 468.1 |
| 1.37 | E | 433.1 | 434.4 |
| 1.38 | E | 433.1 | 434.5 |
| 1.39 | E | 446.2 | 447.5 |
| 1.40 | E | 446.2 | 447.5 |
| 1.41 | E | 446.2 | 447.5 |
| 1.42 | E | 496.1 | 497.5 |
| 1.43 | D | 418.1 | 419.5 |
| 1.44 | E | 488.2 | 489.6 |
| 1.45 | E | 454.2 | 455.5 |
| 1.46 | E | 452.2 | 453.5 |
| 1.47 | C | 480.2 | 481.6 |
| 1.48 | C | 516.2 | 517.7 |
| 1.49 | B | 466.2 | 467.6 |
| 1.50 | B | 464.2 | 465.6 |
| 1.51 | A | 452.2 | 453.5 |
| 1.52 | B | 450.2 | 451.5 |
| 1.53 | B | 492.2 | 493.6 |
| 1.54 | B | 482.2 | 483.6 |
| 1.55 | B | 480.2 | 481.6 |
| 1.56 | B | 478.2 | 479.5 |
| 1.57 | D | 506.2 | 507.5 |
| 1.58 | C | 508.2 | 509.6 |
| 1.59 | B | 461.2 | 462.5 |
| 1.60 | C | 424.2 | 425.5 |
| 1.61 | D | 466.2 | 467.5 |
| 1.62 | D | 502.2 | 503.5 |
| 1.63 | B | 515.2 | 516.2 |
| 1.64 | B | 466.2 | 467.3 |
| 1.65 | D | 521.2 | 522.2 |
| 1.66 | D | 521.2 | 522.2 |
| 1.67 | C | 518.2 | 519.2 |
| 1.68 | B | 518.2 | 519.2 |
| 1.69 | D | 480.2 | 481.1 |
| 1.70 | E | 424.1 | 425.3 |
| 1.71 | B | 452.2 | 453.4 |
| 1.72 | E | 424.1 | 425.3 |
| 4.01 | C | 404.2 | 405.3 |
| 4.02 | B | 418.2 | 419.3 |
| 4.03 | B | 405.2 | 406.3 |
| 4.04 | C | 405.2 | 406.4 |
| 4.05 | D | 405.2 | 406.3 |
| 4.06 | C | 434.2 | 435.3 |
| 4.07 | B | 438.2 | 439.3 |
| 4.08 | E | 438.2 | 439.4 |
| 4.09 | A | 482.1 | 483.3 |
| 4.10 | B | 444.3 | 445.4 |
| 4.11 | B | 481.3 | 482.0 |
| 4.12 | C | 434.2 | 435.3 |
| 4.13 | B | 418.2 | 419.3 |
| 4.14 | E | 418.2 | 419.4 |
| 4.15 | C | 480.3 | 481.4 |
| 4.16 | B | 481.3 | 482.4 |
| 4.17 | C | 444.3 | 445.4 |
| 4.18 | B | 481.3 | 482.4 |
| 4.19 | A | 472.2 | 473.3 |
| 4.20 | B | 485.3 | 486.5 |
| 4.21 | B | 432.3 | 433.5 |
| 4.22 | E | 454.2 | 455.4 |
| 4.23 | B | 439.2 | 440.3 |
| 4.24 | B | 439.2 | 440.3 |
| 4.25 | B | 435.2 | 436.1 |
| 4.26 | C | 404.2 | 405.2 |
| 32.05 | D | 479.2 | 480.4 |
| 8.01 | E | 424.2 | 425.5 |
| 8.02 | E | 461.2 | 462.5 |
| 8.03 | E | 438.2 | 439.4 |
| 8.04 | E | 383.1 | 384.3 |
| 8.05 | E | 532.2 | 533.3 |
| 8.06 | C | 438.2 | 439.4 |
| 8.07 | D | 425.2 | 426.5 |
| 8.08 | E | 473.2 | 474.6 |
| 8.09 | C | 515.2 | 515.8 |
| 11.01 | E | 395.2 | 396.23 |
| 11.02 | D | 460.2 | 461.5 |
| 11.03 | D | 425.2 | 426.5 |
| 11.04 | E | 403.1 | 404.1 |
| 11.05 | C | 412.2 | 413.1 |
| 11.06 | D | 504.2 | 505.3 |
| 11.07 | C | 518.2 | 519.6 |
| 11.08 | E | 515.2 | 516.3 |
| 11.09 | E | 518.2 | 519.3 |
| 11.10 | D | 514.2 | 515.5 |
| 11.11 | E | 447.1 | 448.5 |
| 11.12 | D | 495.1 | 496.3 |
| 11.13 | E | 411.1 | 412.4 |
| 11.14 | D | 438.2 | 439.5 |
| 11.15 | E | 453.2 | 454.4 |
| 11.16 | D | 438.2 | 439.5 |
| 11.17 | E | 421.1 | 422.4 |
| 11.18 | E | 418.1 | 419.4 |
| 11.19 | E | 418.1 | 419.4 |
| 11.20 | C | 454.2 | 455.5 |
| 11.21 | D | 412.2 | 413.4 |
| 11.22 | D | 495.3 | 496.4 |
| 11.23 | D | 504.2 | 505.4 |
| 11.24 | D | 467.2 | 468.5 |
| 11.25 | E | 433.1 | 434.5 |
| 11.26 | E | 446.2 | 447.5 |
| 11.27 | E | 488.2 | 489.5 |
| 11.28 | E | 442.1 | 443.5 |
| 11.29 | E | 385.1 | 386.4 |
| 11.30 | E | 398.2 | 399.5 |
| 11.31 | E | 422.1 | 423.5 |
| 11.32 | E | 480.2 | 481.6 |
| 11.33 | E | 424.2 | 425.5 |
| 11.34 | E | 410.2 | 411.5 |
| 11.35 | E | 438.2 | 439.5 |
| 11.36 | D | 452.2 | 453.5 |
| 11.37 | E | 466.2 | 467.6 |
| 11.38 | E | 488.1 | 489.5 |
| 11.39 | E | 452.2 | 453.5 |
| 11.40 | E | 493.1 | 494.5 |
| 11.41 | E | 468.2 | 469.5 |
| 11.42 | E | 450.2 | 451.5 |
| 13.01 | E | 438.1 | 439.3 |
| 13.02 | E | 434.1 | 435.4 |
| 13.03 | E | 489.2 | 490.4 |
| 13.04 | E | 419.1 | 420.4 |
| 13.05 | E | 404.1 | 405.4 |
| 13.06 | E | 438.1 | 439.4 |
| 13.07 | E | 429.1 | 430.4 |
| 13.08 | E | 429.1 | 430.4 |
| 13.09 | D | 429.1 | 430.4 |
| 13.10 | E | 447.1 | 448.4 |
| 13.11 | E | 434.1 | 435.4 |
| 13.12 | E | 434.1 | 435.4 |
| 13.13 | D | 458.1 | 459.4 |
| 13.14 | E | 487.1 | 488.5 |
| 13.15 | E | 482.1 | 483.4 |
| 13.16 | D | 518.2 | 519.3 |
| 13.17 | E | 439.2 | 440.4 |
| 13.18 | E | 425.2 | 426.4 |
| 13.19 | E | 399.1 | 400.4 |
| 13.20 | E | 419.1 | 420.3 |
| 13.21 | E | 419.1 | 420.3 |
| 13.22 | E | 441.2 | 442.4 |
| 13.23 | E | 413.2 | 414.4 |
| 13.24 | C | 488.2 | 489.5 |
| 13.25 | D | 488.2 | 489.5 |
| 13.26 | D | 439.2 | 440.5 |
| 13.27 | D | 453.2 | 454.5 |
| 16.01 | E | 424.2 | 425.1 |
| 16.02 | E | 383.1 | 384.3 |
| 16.03 | E | 473.2 | 474.4 |
| 16.04 | E | 474.2 | 475.6 |
| 16.05 | D | 481.2 | 482.4 |
| 19.01 | E | 384.2 | 385.2 |
| 19.02 | E | 356.0 | 357.1 |
| 19.03 | E | 317.1 | 317.89 |
| 19.04 | E | 312.1 | 312.78 |
| 19.05 | E | 311.1 | 311.77 |
| 19.06 | D | 346.0 | 347.2 |
| 22.01 | D | 487.1 | 488.4 |
| 22.02 | E | 458.1 | 459.4 |
| 22.03 | E | 447.1 | 448.4 |
| 22.04 | E | 517.2 | 518.5 |
| 22.05 | E | 487.1 | 488.1 |
| 22.06 | E | 439.2 | 440.4 |
| 22.07 | E | 439.2 | 440.5 |
| 22.08 | E | 425.2 | 426.4 |
| 22.09 | E | 399.1 | 400.3 |
| 22.10 | E | 441.2 | 442.4 |
| 22.11 | E | 453.2 | 454.4 |
| 22.12 | D | 488.2 | 489.4 |
| 22.13 | E | 488.2 | 489.2 |
| 28.01 | E | 431.2 | 432.4 |
| 28.02 | E | 515.2 | 516.3 |
| 28.03 | D | 532.2 | 533.3 |
| 28.04 | E | 425.2 | 426.4 |
| 28.05 | E | 417.1 | 418.5 |
| 28.06 | E | 451.1 | 452.5 |
| 29.01 | E | 354.1 | 355.2 |
| 29.03 | D | 434.2 | 435.1 |
| 29.04 | E | 376.2 | 377.1 |
| 29.07 | E | 486.2 | 487.3 |
| 29.08 | E | 487.2 | 488.3 |
| 29.09 | D | 457.3 | 458.0 |
| 29.10 | D | 423.2 | 424.0 |
| 29.11 | D | 491.2 | 492.0 |
| 33.01 | D | 474.1 | 475.26 |
| 33.02 | E | 380.2 | 381.4 |
| 33.03 | E | 393.2 | 394.4 |
| 33.04 | E | 391.2 | 392.3 |
| 33.05 | D | 526.2 | 527.2 |
| 33.06 | E | 343.2 | 344.4 |
| 33.07 | E | 391.2 | 392.4 |
| 33.08 | E | 377.2 | 378.3 |
| 33.09 | E | 498.2 | 499.5 |
| 33.10 | E | 380.2 | 381.3 |
| 33.11 | E | 408.2 | 409.3 |
| 33.12 | C | 391.2 | 392.4 |
| 33.13 | D | 381.2 | 382.4 |
| 33.14 | E | 399.2 | 400.4 |
| 33.15 | E | 398.2 | 399.2 |
| 33.16 | D | 405.2 | 406.3 |
| 33.17 | E | 454.2 | 455.4 |
| 33.18 | B | 419.2 | 420.3 |
| 33.19 | E | 423.2 | 424.3 |
| 33.20 | C | 435.2 | 436.3 |
| 33.21 | D | 411.2 | 412.4 |
| 33.22 | E | 504.2 | 505.3 |
| 33.23 | E | 396.3 | 397.5 |

### Determination of the % Inhibition for FXIa

FXIa inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human FXIa (Enzyme Research Laboratories) was incubated at 25° C. with the fluorogenic substrate Z-Gly-Pro-Arg-AFC and various concentrations of the test compound. Residual enzyme activity (initial rate of reaction) was determined by measuring the change in fluorescence at 410 nm and the IC50 value for the test compound was determined.

TABLE 13
Selectivity; FXIa data

| Ex. No. | Human FXIa IC50 (nM) |
|---|---|
| 1.43 | >40,000 |
| 1.44 | >40,000 |
| 1.45 | >40,000 |
| 1.46 | >40,000 |
| 1.47 | >40,000 |
| 1.48 | >40,000 |
| 1.49 | >40,000 |
| 1.50 | >40,000 |
| 1.51 | >40,000 |
| 1.52 | >40,000 |
| 1.53 | >40,000 |
| 1.54 | >40,000 |
| 1.55 | >40,000 |
| 1.56 | >40,000 |
| 1.57 | >40,000 |
| 1.58 | >40,000 |
| 1.59 | >40,000 |
| 1.60 | >40,000 |
| 1.61 | >40,000 |
| 1.62 | >40,000 |
| 1.63 | >40,000 |
| 1.64 | >40,000 |
| 1.65 | >40,000 |
| 1.66 | >40,000 |
| 1.67 | >40,000 |
| 1.68 | >40,000 |
| 1.69 | >40,000 |
| 1.70 | >40,000 |
| 1.71 | >40,000 |
| 1.72 | 3,160 |
| 4.01 | >40,000 |
| 4.02 | >40,000 |
| 4.03 | >40,000 |
| 4.04 | >40,000 |
| 4.05 | >40,000 |
| 4.06 | >40,000 |
| 4.07 | 23,600 |
| 4.08 | >40,000 |
| 4.09 | >40,000 |
| 4.10 | >40,000 |
| 4.11 | >40,000 |
| 4.12 | >40,000 |
| 4.13 | >40,000 |
| 4.14 | >40,000 |
| 4.15 | >40,000 |
| 4.16 | >40,000 |
| 4.17 | >40,000 |
| 4.18 | 23,300 |
| 4.19 | >40,000 |
| 4.20 | 12,700 |
| 4.21 | >40,000 |
| 4.22 | >40,000 |
| 4.23 | >40,000 |
| 4.24 | >40,000 |
| 4.25 | >40,000 |
| 8.08 | >40,000 |
| 8.09 | >40,000 |
| 11.28 | >40,000 |
| 11.29 | >40,000 |
| 11.30 | >40,000 |
| 11.31 | >40,000 |
| 11.32 | >40,000 |
| 11.33 | >40,000 |
| 11.34 | >40,000 |
| 11.35 | >40,000 |
| 11.36 | >40,000 |
| 11.37 | >40,000 |
| 11.38 | >40,000 |
| 11.39 | >40,000 |
| 11.40 | >40,000 |
| 11.41 | >40,000 |
| 11.42 | >40,000 |
| 13.26 | >40,000 |
| 13.27 | >40,000 |
| 16.04 | >40,000 |
| 16.05 | >40,000 |
| 29.07 | 21,000 |
| 29.08 | >40,000 |
| 33.01 | >40,000 |
| 33.02 | 20,000 |
| 33.03 | 20,000 |
| 33.04 | >40,000 |
| 33.05 | 33,500 |
| 33.06 | 20,000 |
| 33.07 | >40,000 |
| 33.08 | >40,000 |
| 33.09 | 33,500 |
| 33.10 | 20,000 |
| 33.11 | >40,000 |
| 33.12 | >40,000 |
| 33.13 | 39,800 |
| 33.14 | >40,000 |
| 33.15 | >40,000 |
| 33.16 | >40,000 |
| 33.17 | >40,000 |
| 33.18 | >40,000 |
| 33.19 | >40,000 |
| 33.20 | 13,500 |
| 33.21 | >40,000 |

### NUMBERED EMBODIMENTS

1. A compound of formula (I),

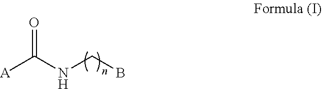

wherein:
n is 0, 1, or 2;
A is a 6-membered heteroaryl of formula (II),

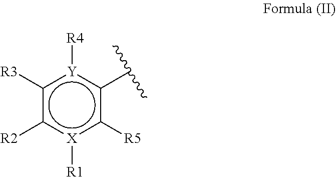

wherein X and Y are independently selected from C and N, wherein at least one of X or Y is N;
wherein R5 is selected from —NR12(CH₂)₀₋₃(heterocyclyl), —NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁₋₄NR13R14, and —NR12(CH₂)₀₋₃O(aryl);
wherein R2 and R3 are independently selected from H, halo, alkoxy, alkyl, cycloalkyl, aryl, and heteroaryl;
wherein R1 and R4 are independently absent, or independently selected from H, halo, alkoxy, alkyl, cycloalkyl, aryl, and heteroaryl; or
wherein X and Y are independently selected from C and N, wherein at least one of X or Y is N;
wherein R1, R4, and R5 are independently absent or independently selected from H, halo and alkyl;
wherein one of R2 or R3 is

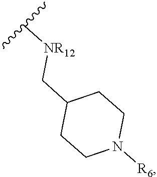

and the other of R2 or R3 is selected from H, halo or alkyl; wherein R6 is H, alkyl, or heteroaryl^{b}; or
wherein X and Y are independently selected from C and N, wherein at least one of X or Y is N;
wherein R1 and R4 are independently absent or independently selected from H, halo and alkyl;
wherein R3 is halo;
wherein R2 is —(CH₂)₀₋₃NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁₋₄NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) and —O—(CH₂)₀₋₃(heteroaryl), and
wherein R5 is H, alkyl and halo; or
wherein X and Y are C;
wherein R4 is H, halo, alkyl;
wherein R5 is H or alkyl;
wherein R3 is H or halo;
wherein one of R1 and R2 is —(CH₂)(heterocyclyl) or —N(R12)CO(CH₂)₀₋₃(heterocyclyl), and the other of R1 and R2 is selected from H and alkyl;
wherein X is C or N, and Y is C;
R1 is absent, H or alkyl;
R4 is H or alkyl;
R5 is H or alkyl;
wherein either: (a) R2 and R3 together with the carbon atoms to which they are bonded form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}, or (b) R2 and R3 are independently selected from H and halo, wherein at least one of R2 or R3 is halo, or (c) R2 and R3 are independently selected from H, aryl^{b} and heteroaryl^{b}, wherein at least one of R2 or R3 is aryl^{b}, or heteroaryl^{b}; B is one of:
(i) a fused 6,5- or 6,6-heteroaromatic bicyclic ring, containing N and, optionally, one or two additional heteroatoms independently selected from N, O and S;
   wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF3 and —NR13R14;
   wherein the 6,5-heteroaromatic bicyclic ring may be attached via the 6- or 5-membered ring;
(ii) phenyl, which may be optionally substituted with 1, 2 or 3 substituents independently selected from, alkyl, heteroaryl, alkoxy, heterocyclyl, OH, halo, CN, CF₃, and a carbon-containing 4-, 5-, 6- or 7-membered heterocylic ring containing 1, 2 or 3 heteroatoms independently selected from N and N12, which may be saturated or unsaturated with 1 or 2 double bonds and which may be optionally mono- or di-substituted with substituents independently selected from oxo, alkyl, alkoxy, OH, halo, and CF₃; or
(iii) phenyl, wherein two adjacent carbon atoms on the phenyl are either linked together by —N═C—N(R8)-C(═O)— to form a quinazolinone or linked together by —CH₂—N(R8)-C(═O)— to form an isoindolinone; or
(iv) heteroaryl; or
(v) a fused 6,5- or 6,6-bicyclic ring containing an aromatic ring fused to a non-aromatic ring and containing N and, optionally, one or two additional heteroatoms independently selected from N, O and S;
   wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14;
   wherein the 6,5-bicyclic ring may be attached via the 6- or 5-membered ring;
      alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro;
      alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, and heterocyclyl^{b};
      alkyl^{b} is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂, —NHCOCH₃, CF₃, halo, oxo, heterocyclyl^{b}, and cyclopropane;
      alkylene is a bivalent linear saturated hydrocarbon having 1 to 5 carbon atoms (C₁-C₅); alkylene may optionally be substituted with 1 or 2 substituents independently selected from alkyl, (C₁-C₅)alkoxy, OH, CN, CF₃, and halo;
      aryl is phenyl, biphenyl or naphthyl; aryl may be optionally substituted with 1, 2 or 3 substituents independently selected from alkyl, alkoxy, OH, —SO₂CH₃, halo, CN, —(CH₂)₀₋₃—O-heteroaryl^{b}, aryl^{b}, —O-aryl^{b}, —(CH₂)₀₋₃-heterocyclyl^{b}, —(CH₂)₁₃-aryl^{b}, —(CH₂)₀₋₃-heteroaryl^{b}, —COOR13, —CONR13R14, —(CH₂)₀₋₃—NR13R14, OCF₃ and CF₃; or two adjacent carbon ring atoms on the aryl may be optionally linked by a heteroalkylene to form a non-aromatic ring containing 5, 6, or 7 ring members; or optionally wherein two adjacent ring atoms on aryl are linked to form a 5- or 6-membered aromatic ring containing 1 or 2 heteroatoms that are selected from N, NR8, S, and O, which may be optionally substituted as for heteroaryl^{b};
      aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R12)₂, halo, CN, and CF₃; or two adjacent carbon ring atoms on the aryl may be optionally linked by a heteroalkylene to form a non-aromatic ring containing 5, 6, or 7 ring members;
      cycloalkyl is a monocyclic saturated hydrocarbon ring of between 3 and 6 carbon atoms (C₃-C₆); cycloalkyl may optionally be substituted with 1 or 2 substituents independently selected from alkyl^{b}, (C₁-C₅)alkoxy, OH, CN, CF₃, and halo;
      halo is F, Cl, Br, or I;
      heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₆)alkoxy, OH, CN, CF₃, and halo;
      heteroaryl is a 5- or 6-membered carbon-containing aromatic ring containing 1, 2, 3, or 4 ring members that are selected from N, NR8, S, and O; heteroaryl may be optionally substituted with 1, 2 or 3 substituents independently selected from alkyl, alkoxy, aryl^{b}, OH, OCF₃, halo, heterocyclyl^{b}, CN, and CF₃;
      heteroaryl^{b} is a 5- or 6-membered carbon-containing aromatic ring containing one, two or three ring members that are selected from N, NR8, S, and O; heteroaryl^{b} may be optionally substituted with 1, 2 or 3 substituents independently selected from methyl, ethyl, propyl, isopropyl, alkoxy, CH₂aryl^{b}, OH, OCF₃, halo, CN, and CF₃;
      heterocyclyl is a 4-, 5-, 6-, or 7-membered carbon-containing non-aromatic ring containing one, two or three ring members that are selected from N, NR8, S, SO, SO₂ and O; heterocyclyl may be optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl;
      heterocyclyl^{b} is a 4-, 5-, 6-, or 7-membered carbon-containing non-aromatic ring containing one, two or three ring members that are selected from N, NR12, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃;
      R13 and R14 are independently selected from H, —SO₂CH₃, alkyl^{b}, heteroaryl^{b}, and cycloalkyl; or R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R13 and R14 together with the nitrogen atom to which they are attached form a carbon-containing 5- or 6-membered heterocylic ring, which is fused to an aryl^{b} or a heteroaryl^{b};
      R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, and —(CH₂)₀₋₃heterocyclyl^{b}; or R8 is a carbon-containing 4-, 5-, 6- or 7-membered heterocylic ring containing 1, 2 or 3 heteroatoms independently selected from N, N12, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃;
      R12 is independently selected from H, —SO₂CH₃, methyl, ethyl, propyl, isopropyl, and cycloalkyl;
      and tautomers, isomers, stereoisomers (including enantiomers, diastereoisomers and racemic and scalemic mixtures thereof), deuterated isotopes, and pharmaceutically acceptable salts and/or solvates thereof.

2. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 0.

3. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 1.

4. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 2.

5. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6-membered heteroaryl of formula (II),

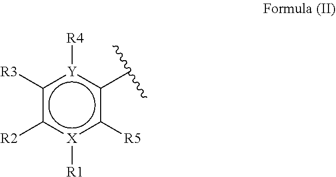

wherein X and Y are independently selected from C and N, wherein at least one of X or Y is N;
wherein R5 is selected from —NR12(CH₂)₀₋₃(heterocyclyl), —NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁₋₄NR13R14, and —NR12(CH₂)₀₋₃O(aryl);
wherein R2, R3 and R4 are independently selected from H, halo, alkoxy, alkyl, cycloalkyl, aryl, and heteroaryl.

6. A compound of formula (I) according to numbered embodiment 5, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N, and R1 is absent.

7. A compound of formula (I) according to numbered embodiment 5, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C.

8. A compound of formula (I) according to any of numbered embodiments 5 to 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N, and R4 is absent.

9. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NR12(CH₂)₀₋₃(heterocyclyl).

10. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —O(CH₂)₀₋₃(heterocyclyl).

11. A compound of formula (I) according to any of numbered embodiments 9 or 10, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein two adjacent ring atoms on heterocyclyl are linked to form a 5- or 6-membered aromatic ring containing 1 or 2 heteroatoms that are selected from N, NR8, S, and O.

12. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is piperidinyl.

13. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is substituted with methyl or ethyl.

14. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is substituted with cyclopropyl.

15. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is substituted with —CH₂CH₂OCH₃.

16. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein two adjacent ring atoms on heterocyclyl are linked to form imidazole.

17. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is piperazinyl.

18. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is piperidinyl, optionally substituted with oxo.

19. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is tetrahydropyranyl.

20. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is pyrrolidine, optionally substituted with oxo.

21. A compound of formula (I) according to any of numbered embodiments 9 to 11, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R5 is morpholinyl.

22. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NR12(CH₂)₀₋₃(heteroaryl).

23. A compound of formula (I) according to numbered embodiment 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heteroaryl on R5 is pyridinyl.

24. A compound of formula (I) according to numbered embodiment 22, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heteroaryl on R5 is imidazole.

25. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NR12(CH₂)₀₋₃(aryl).

26. A compound of formula (I) according to numbered embodiment 25, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NH(aryl).

27. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —O(CH₂)₀₋₃(aryl).

28. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NR12(CH₂)₀₋₃O(aryl).

29. A compound of formula (I) according to any of numbered embodiments 25 to 28, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the aryl on R5 is phenyl.

30. A compound of formula (I) according to any of numbered embodiments 25 to 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein two adjacent carbon ring atoms on the aryl may be optionally linked by a heteroalkylene to form a non-aromatic ring containing 5, 6, or 7 ring members.

31. A compound of formula (I) according to numbered embodiment 30, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein two adjacent carbon ring atoms on the aryl may be optionally linked by a heteroalkylene to form piperidine.

32. A compound of formula (I) according to any of numbered embodiments 25 to 27, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the aryl on R5 is phenyl, wherein two adjacent carbon ring atoms on the aryl may be optionally linked by a heteroalkylene to form a non-aromatic ring containing 5, 6, or 7 ring members.

33. A compound of formula (I) according to any of numbered embodiments 25 to 27, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the aryl on R5 is phenyl, wherein two adjacent ring atoms on aryl are linked to form a 5- or 6-membered aromatic ring containing 1 or 2 heteroatoms that are selected from N, NR8, S, and O, which may be optionally substituted as for heteroaryl^{b}.

34. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —NR13R14.

35. A compound of formula (I) according to any of numbered embodiments 5 to 8, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —O—(CH₂)₁₋₄NR13R14.

36. A compound of formula (I) according to any of numbered embodiments 34 to 35, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, on the R5, the R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃.

37. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

38. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is halo.

39. A compound of formula (I) according to numbered embodiment 38, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is chloro.

40. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkoxy.

41. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkyl.

42. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is cycloalkyl.

43. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is aryl.

44. A compound of formula (I) according to any of numbered embodiments 5 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is heteroaryl.

45. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

46. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

47. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is chloro.

48. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkoxy.

49. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

50. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is cycloalkyl.

51. A compound of formula (I) according to any of numbered embodiments 5 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is aryl.

52. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is heteroaryl.

53. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

54. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

55. A compound of formula (I) according to numbered embodiment 54, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is chloro.

56. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkoxy.

57. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

58. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is cycloalkyl.

59. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is aryl.

60. A compound of formula (I) according to any of numbered embodiments 5 to 51, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is heteroaryl.

61. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

62. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.

63. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is chloro.

64. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkoxy.

65. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

66. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is cycloalkyl.

67. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is aryl.

68. A compound of formula (I) according to any of numbered embodiments 5 and 7 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is heteroaryl.

69. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

70. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

71. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is chloro.

72. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkoxy.

73. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

74. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is cycloalkyl.

75. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is aryl.

76. A compound of formula (I) according to any of numbered embodiments 5 to 7 and 9 to 36, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is heteroaryl.

77. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6-membered heteroaryl of formula (II),

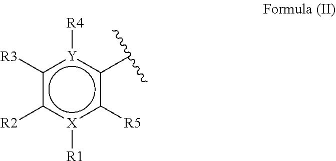

wherein X and Y are independently selected from C and N, wherein at least one of X or Y is N;
wherein R1, R4, and R5 are independently absent or independently selected from H, halo and alkyl;
wherein one of R2 or R3 is

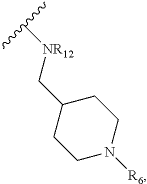

and the other of R2 or R3 is selected from H, halo or alkyl; and wherein R6 is H, alkyl, or heteroaryl^{b}.

78. A compound of formula (I) according to numbered embodiment 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

79. A compound of formula (I) according to numbered embodiment 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C.

80. A compound of formula (I) according to any of numbered embodiments 77 to 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N.

81. A compound of formula (I) according to any of numbered embodiments 77 to 80, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is

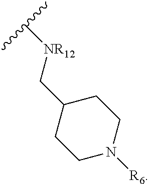

82. A compound of formula (I) according to any of numbered embodiments 77 to 81, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

83. A compound of formula (I) according to any of numbered embodiments 77 to 81, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

84. A compound of formula (I) according to numbered embodiment 83, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is chloro.

85. A compound of formula (I) according to any of numbered embodiments 77 to 81, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

86. A compound of formula (I) according to any of numbered embodiments 77 to 80, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is

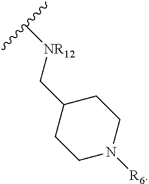

87. A compound of formula (I) according to numbered embodiment 86, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

88. A compound of formula (I) according to numbered embodiment 86, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

89. A compound of formula (I) according to numbered embodiments 88, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is chloro.

90. A compound of formula (I) according to numbered embodiment 86, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

91. A compound of formula (I) according to any of numbered embodiments 77 to 90, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R12 is H.

92. A compound of formula (I) according to any of numbered embodiments 77 to 90, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R12 is alkyl.

93. A compound of formula (I) according to numbered embodiment 92, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R12 is methyl.

94. A compound of formula (I) according to any of numbered embodiments 77 to 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is H.

95. A compound of formula (I) according to any of numbered embodiments 77 to 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is alkyl.

96. A compound of formula (I) according to numbered embodiment 95, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is methyl.

97. A compound of formula (I) according to any of numbered embodiments 77 to 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is hereroaryl^{b}.

98. A compound of formula (I) according to numbered embodiment 97, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is pyridyl.

99. A compound of formula (I) according to any of numbered embodiments 77 to 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

100. A compound of formula (I) according to any of numbered embodiments 77 to 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

101. A compound of formula (I) according to any of numbered embodiments 77 to 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.

102. A compound of formula (I) according to numbered embodiment 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is chloro.

103. A compound of formula (I) according to any of numbered embodiments 77 to 102, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

104. A compound of formula (I) according to any of numbered embodiments 77 to 102, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

105. A compound of formula (I) according to any of numbered embodiments 77 to 102, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

106. A compound of formula (I) according to numbered embodiment 105, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is chloro.

107. A compound of formula (I) according to any of numbered embodiments 77 to 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

108. A compound of formula (I) according to any of numbered embodiments 77 to 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

109. A compound of formula (I) according to any of numbered embodiments 77 to 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is halo.

110. A compound of formula (I) according to numbered embodiment 109, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is chloro.

111. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6-membered heteroaryl of formula (II),

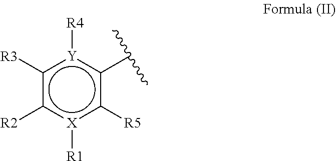

wherein X and Y are independently selected from C and N, wherein at least one of X or Y is N;
wherein R1 and R4 are independently absent or independently selected from H, halo and alkyl;
wherein R3 is halo;
wherein R2 is —(CH₂)₀₋₃NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁₋₄NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) and —O—(CH₂)₀₋₃(heteroaryl), and
wherein R5 is H, alkyl and halo.

112. A compound of formula (I) according to numbered embodiment 111, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

113. A compound of formula (I) according to any of numbered embodiments 111 to 112, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C.

114. A compound of formula (I) according to any of numbered embodiments 111 to 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N.

115. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR13R14.

116. A compound of formula (I) according to numbered embodiment 115, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)NR13R14, wherein R13 and R14, together with the nitrogen atom to which they are attached form piperazine, which may be optionally substituted in the same manner as R13 and R14, as defined in claim 1.

117. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the piperazine on R2 has an NR8 group, wherein R8 is pyridinyl.

118. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —NR12(CH₂)₀₋₃NR13R14.

119. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —O—(CH₂)₁₋₄NR13R14.

120. A compound of formula (I) according to any of numbered embodiments 115 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein on R2, R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃.

121. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —NR12(CH₂)₀₋₃(aryl).

122. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃O(CH₂)₀₋₃(aryl).

123. A compound of formula (I) according to any of numbered embodiments 121 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein aryl on R2 is phenyl.

124. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(heteroaryl).

125. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —O—(CH₂)₀₋₃(heteroaryl).

126. A compound of formula (I) according to any of numbered embodiments 124 to 125, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heteroaryl on R2 is pyridinyl.

127. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)NR12(CH₂)₀₋₃(heterocyclyl).

128. A compound of formula (I) according to any of numbered embodiments 111 to 114, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —O—(CH₂)₀₋₃(heterocyclyl).

129. A compound of formula (I) according to any of numbered embodiments 127 to 128, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R2 is piperidinyl.

130. A compound of formula (I) according to any of numbered embodiments 111 to 129, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is chloro.

131. A compound of formula (I) according to any of numbered embodiments 111 to 130, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

132. A compound of formula (I) according to any of numbered embodiments 111 to 130, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.

133. A compound of formula (I) according to numbered embodiment 132, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is chloro.

134. A compound of formula (I) according to any of numbered embodiments 111 to 130, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

135. A compound of formula (I) according to any of numbered embodiments 111 to 134, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

136. A compound of formula (I) according to any of numbered embodiments 111 to 134, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

137. A compound of formula (I) according to numbered embodiment 136, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is chloro.

138. A compound of formula (I) according to any of numbered embodiments 111 to 134, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

139. A compound of formula (I) according to any of numbered embodiments 111 to 138, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

140. A compound of formula (I) according to any of numbered embodiments 111 to 138, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

141. A compound of formula (I) according to any of numbered embodiments 111 to 138, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is halo.

142. A compound of formula (I) according to numbered embodiment 141, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is chloro.

143. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6-membered heteroaryl of formula (II),

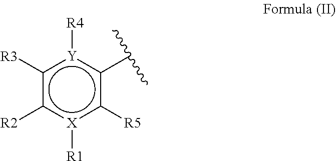

wherein X and Y are C;
wherein R4 is H, halo, alkyl;
wherein R5 is H or alkyl;
wherein R3 is H or halo;
wherein one of R1 and R2 is —(CH₂)(heterocyclyl) or —N(R12)CO(CH₂)₀₋₃(heterocyclyl), and the other of R1 and R2 is selected from H and alkyl;

144. A compound of formula (I) according to numbered embodiment 143, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)(heterocyclyl).

145. A compound of formula (I) according to numbered embodiment 144, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

146. A compound of formula (I) according to numbered embodiment 144, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkyl.

147. A compound of formula (I) according to numbered embodiment 143, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)(heterocyclyl).

148. A compound of formula (I) according to numbered embodiment 147, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

149. A compound of formula (I) according to numbered embodiment 147, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

150. A compound of formula (I) according to any of numbered embodiments 143 to 149, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl is piperazinyl.

151. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the piperazinyl is substituted with pyridinyl.

152. A compound of formula (I) according to any of numbered embodiments 143 to 149, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R2 is piperidinyl.

153. A compound of formula (I) according to any of numbered embodiments 143 to 152, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

154. A compound of formula (I) according to any of numbered embodiments 143 to 152, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

155. A compound of formula (I) according to numbered embodiment 157, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is chloro.

156. A compound of formula (I) according to any of numbered embodiments 143 to 156, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

157. A compound of formula (I) according to any of numbered embodiments 143 to 156, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

158. A compound of formula (I) according to numbered embodiment 157, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is chloro.

159. A compound of formula (I) according to any of numbered embodiments 143 to 158, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

160. A compound of formula (I) according to any of numbered embodiments 143 to 159, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

161. A compound of formula (I) according to any of numbered embodiments 143 to 159, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

162. A compound of formula (I) according to any of numbered embodiments 143 to 159, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

163. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6-membered heteroaryl of formula (II),

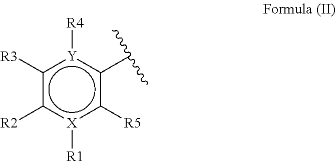

wherein X is C or N, and Y is C;
R1 is absent, H or alkyl;
R4 is H or alkyl;
R5 is H or alkyl;
wherein either: (a) R2 and R3 together with the carbon atoms to which they are bonded form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}, or (b) R2 and R3 are independently selected from H and halo, wherein at least one of R2 or R3 is halo, or (c) R2 and R3 are independently selected from H, aryl^{b} and heteroaryl^{b}, wherein at least one of R2 or R3 is aryl^{b}, or heteroaryl^{b}

164. A compound of formula (I) according to numbered embodiment 163, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C.

165. A compound of formula (I) according to numbered embodiment 163, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

166. A compound of formula (I) according to any of numbered embodiments 163 to 165, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 together with the carbon atoms to which they are bonded form phenyl or a 5- or 6-membered nitrogen-containing heteroaryl, wherein phenyl may be optionally substituted as for aryl^{b}, and wherein the 5- or 6-membered nitrogen-containing heteroaryl may be optionally substituted as for heteroaryl^{b}.

167. A compound of formula (I) according to numbered embodiment 166, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 together with the carbon atoms to which they are bonded form phenyl, wherein phenyl may be optionally substituted as for aryl^{b}.

168. A compound of formula (I) according to any of numbered embodiments 163 to 165, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 together with the carbon atoms to which they are bonded form a 5- or 6-membered nitrogen-containing heteroaryl, wherein the 5- or 6-membered nitrogen-containing heteroaryl may be optionally substituted as for heteroaryl^{b}.

169. A compound of formula (I) according to numbered embodiment 163 to 165, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 are independently selected from H and halo, wherein at least one of R2 or R3 is halo.

170. A compound of formula (I) according to numbered embodiment 163 to 165, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 are independently selected from H, aryl^{b} and heteroaryl^{b}, wherein at least one of R2 or R3 is aryl^{b}, or heteroaryl^{b}.

171. A compound of formula (I) according to numbered embodiment 163 to 170, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

172. A compound of formula (I) according to numbered embodiment 163 to 170, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

173. A compound of formula (I) according to numbered embodiment 163 to 172, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

174. A compound of formula (I) according to numbered embodiment 163 to 172, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

175. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a fused 6,5- or 6,6-heteroaromatic bicyclic ring, containing N and, optionally, one or two additional heteroatoms independently selected from N, O and S;
wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14;
wherein the 6,5-heteroaromatic bicyclic ring may be attached via the 6- or 5-membered ring.

176. A compound of formula (I) according to any of numbered embodiments 1 to 174, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a fused 6,5- or 6,6-bicyclic ring containing an aromatic ring fused to a non-aromatic ring and containing N and, optionally, one or two additional heteroatoms independently selected from N, O and S;
wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14;
wherein the 6,5-bicyclic ring may be attached via the 6- or 5-membered ring.

177. A compound of formula (I) according to any of numbered embodiments 1 to 175, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is quinolinyl.

178. A compound of formula (I) according to any of numbered embodiments 1 to 175, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is quinoxaline.

179. A compound of formula (I) according to any of numbered embodiments 1 to 175, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is benzoxazole.

180. A compound of formula (I) according to any of numbered embodiments 1 to 175, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is azaindole.

181. A compound of formula (I) according to any of numbered embodiments 176, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a fused 6,5-bicyclic ring attached via the 5-membered ring, the 5-membered ring is cyclopropane, and the 6-membered ring is pyridine.

182. A compound of formula (I) according to any of numbered embodiments 1 to 180, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with halo.

183. A compound of formula (I) according to numbered embodiment 182, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with chloro.

184. A compound of formula (I) according to numbered embodiment 182, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with fluoro.

185. A compound of formula (I) according to any of numbered embodiments 1 to 180, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with alkoxy.

186. A compound of formula (I) according to numbered embodiment 185, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with methoxy.

187. A compound of formula (I) according to any of numbered embodiments 1 to 180, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with —NR13R14.

188. A compound of formula (I) according to numbered embodiment 187, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein —NR13R14 is —NH₂.

189. A compound of formula (I) according to any of numbered embodiments 1 to 180, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with alkyl.

190. A compound of formula (I) according to numbered embodiment 189, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with methyl.

191. A compound of formula (I) according to any of numbered embodiments 1 to 174, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl, which may be optionally substituted with 1, 2 or 3 substituents independently selected from, alkyl, heteroaryl, alkoxy, heterocyclyl, OH, halo, CN, CF₃, and a carbon-containing 4-, 5-, 6- or 7-membered heterocylic ring containing 1, 2 or 3 heteroatoms independently selected from N and N12, which may be saturated or unsaturated with 1 or 2 double bonds and which may be optionally mono- or di-substituted with substituents independently selected from oxo, alkyl, alkoxy, OH, halo, and CF₃.

192. A compound of formula (I) according to numbered embodiment 191, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with alkoxy.

193. A compound of formula (I) according to numbered embodiment 192, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with methoxy.

194. A compound of formula (I) according to numbered embodiment 191, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with halo.

195. A compound of formula (I) according to numbered embodiment 194, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with chloro.

196. A compound of formula (I) according to numbered embodiment 194, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with fluoro.

197. A compound of formula (I) according to any of numbered embodiments 191 to 196, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with heteroaryl.

198. A compound of formula (I) according to numbered embodiment 197, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with tetrazolyl.

199. A compound of formula (I) according to numbered embodiment 197, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with triazole.

200. A compound of formula (I) according to numbered embodiment 191, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with alkyl.

201. A compound of formula (I) according to any of numbered embodiments 191 or 200, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with —CH₂NH₂.

202. A compound of formula (I) according to numbered embodiment 197, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with a carbon-containing 4-, 5-, 6- or 7-membered heterocylic ring containing 1, 2 or 3 heteroatoms independently selected from N and N12, which may be saturated or unsaturated with 1 or 2 double bonds and which may be optionally mono- or di-substituted with substituents independently selected from oxo, alkyl, alkoxy, OH, halo, and CF₃.

203. A compound of formula (I) according to any of numbered embodiments 1 to 174, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl, wherein two adjacent carbon atoms on the phenyl are either linked together by —N═C—N(R8)-C(═O)— to form a quinazolinone.

204. A compound of formula (I) according to any of numbered embodiments 1 to 174, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl, wherein two adjacent carbon atoms on the phenyl are either linked together by —CH₂—N(R8)-C(═O)— to form an isoindolinone.

205. A compound of formula (I) according to any of numbered embodiments 1 to 174, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is heteroaryl.

206. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heteroaryl ring contains only carbon and nitrogen.

207. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is imidazolyl.

208. A compound selected from any of Tables 1 to 11, or a pharmaceutically acceptable salt, solvate, or solvate of a salt thereof.

209. A compounds according to any preceding claim, selected from examples: 1.51, 4.09, 4.19, 1.13, 1.25, 1.28, 1.49, 1.5, 1.52, 1.53, 1.54, 1.55, 1.56, 1.59, 1.63, 1.64, 1.68, 1.71, 4.02, 4.03, 4.07, 4.1, 4.11, 4.13, 4.16, 4.18, 4.2, 4.21, 4.23, 4.24, 4.25, 33.18; and pharmaceutically acceptable salts and/or solvates thereof.

210. A compounds according to any preceding claim, selected from examples: 1.51, 4.09, 4.19; and pharmaceutically acceptable salts and/or solvates thereof.

211. A compound according to any preceding numbered embodiment.

212. A pharmaceutically acceptable salt according to any of numbered embodiments 1 to 210.

213. A pharmaceutically acceptable solvate according to any of numbered embodiments 1 to 210.

214. A pharmaceutically acceptable solvate of a salt according to any of numbered embodiments 1 to 210.

215. A pharmaceutical composition comprising:
(i) a compound according to numbered embodiment 211, the pharmaceutically acceptable salt according to numbered embodiment 212, the pharmaceutically acceptable solvate according to numbered embodiment 213, or the pharmaceutically acceptable solvate of a salt according to numbered embodiment 214; and
(ii) at least one pharmaceutically acceptable excipient.

216. A compound as defined in numbered embodiment 211, a pharmaceutically acceptable salt according to numbered embodiment 212, a pharmaceutically acceptable solvate according to numbered embodiment 213, a pharmaceutically acceptable solvate of a salt according to numbered embodiment 214, or a pharmaceutical composition as defined in numbered embodiment 215, for use in medicine.

217. The use of a compound as defined in numbered embodiment 211, a pharmaceutically acceptable salt according to numbered embodiment 212, a pharmaceutically acceptable solvate according to numbered embodiment 213, a pharmaceutically acceptable solvate of a salt according to numbered embodiment 214, or a pharmaceutical composition as defined in numbered embodiment 215, in the manufacture of a medicament for the treatment or prevention of a disease or condition in which Factor XIIa activity is implicated.

218. A method of treatment of a disease or condition in which Factor XIIa activity is implicated comprising administration to a subject in need thereof a therapeutically effective amount of a compound as defined in numbered embodiment 211, a pharmaceutically acceptable salt according to numbered embodiment 212, a pharmaceutically acceptable solvate according to numbered embodiment 213, a pharmaceutically acceptable solvate of a salt according to numbered embodiment 214 or a pharmaceutical composition as defined in numbered embodiment 215.

219. A compound as defined in numbered embodiment 211, a pharmaceutically acceptable salt according to numbered embodiment 212, a pharmaceutically acceptable solvate according to numbered embodiment 213, a pharmaceutically acceptable solvate of a salt according to numbered embodiment 214, or a pharmaceutical composition as defined in numbered embodiment 215, for use in a method of treatment of a disease or condition in which Factor XIIa activity is implicated.

220. The use of numbered embodiment 217, the method of numbered embodiment 218, or a compound, a pharmaceutically acceptable salt, a pharmaceutically acceptable solvate, a pharmaceutically acceptable solvate of a salt, or a pharmaceutical composition for use as defined in numbered embodiment 219, wherein, the disease or condition in which Factor XIIa activity is implicated is a bradykinin-mediated angioedema.

221. The use of numbered embodiment 220, the method of numbered embodiment 220, or a compound, a pharmaceutically acceptable salt, a pharmaceutically acceptable solvate, a pharmaceutically acceptable solvate of a salt, or a pharmaceutical composition for use as defined in numbered embodiment 220, wherein the bradykinin-mediated angioedema is hereditary angioedema.

222. The use of numbered embodiment 220, the method of numbered embodiment 220, or a compound, a pharmaceutically acceptable salt, a pharmaceutically acceptable solvate, a pharmaceutically acceptable solvate of a salt, or a pharmaceutical composition for use as defined in numbered embodiment 220, wherein the bradykinin-mediated angioedema is non hereditary.

223. The use of numbered embodiment 217, the method of numbered embodiment 218, or a compound, a pharmaceutically acceptable salt, a pharmaceutically acceptable solvate, a pharmaceutically acceptable solvate of a salt, or a pharmaceutical composition for use as defined in numbered embodiment 219, wherein the disease or condition in which Factor XIIa activity is implicated is selected from vascular hyperpermeability, stroke including ischemic stroke and haemorrhagic accidents; retinal edema; diabetic retinopathy; DME; retinal vein occlusion; and AMD.

224. The use of numbered embodiment 217, the method of numbered embodiment 218, or a compound, a pharmaceutically acceptable salt, a pharmaceutically acceptable solvate, a pharmaceutically acceptable solvate of a salt, or a pharmaceutical composition for use as defined in numbered embodiment 219, wherein, the disease or condition in which Factor XIIa activity is implicated is a thrombotic disorder.

225. The use of numbered embodiment 224, the method of numbered embodiment 224, or a compound, a pharmaceutically acceptable salt, a pharmaceutically acceptable solvate, a pharmaceutically acceptable solvate of a salt, or a pharmaceutical composition for use as defined in numbered embodiment 224, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.

226. The use of numbered embodiment 217, the method of numbered embodiment 218, or a compound, a pharmaceutically acceptable salt, a pharmaceutically acceptable solvate, a pharmaceutically acceptable solvate of a salt, or a pharmaceutical composition for use as defined in numbered embodiment 219, wherein, the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

227. The use of any of numbered embodiments 217 or 220 to 226, the method of any of numbered embodiments 218 or 220 to 226, or a compound, a pharmaceutically acceptable salt, a pharmaceutically acceptable solvate, a pharmaceutically acceptable solvate of a salt, or a pharmaceutical composition for use as defined in any of numbered embodiments 219 or 220 to 226, wherein the compound targets FXIIa.

1. A compound of formula (I): 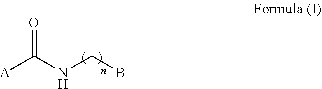
wherein:
n is 0, 1, or 2;
A is a 6-membered heteroaryl of formula (II): 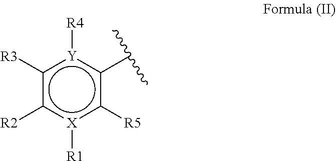
wherein:
(i) X and Y are independently selected from C or N, wherein at least one of X or Y is N;
R5 is selected from —NR12(CH₂)₀₋₃(heterocyclyl), —NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁₋₄NR13R14, or —NR12(CH₂)₀₋₃O(aryl);
R2 and R3 are independently selected from H, halo, alkoxy, alkyl, cycloalkyl, aryl, or heteroaryl;
R1 and R4 are independently absent, halo, alkoxy, alkyl, cycloalkyl, aryl, or heteroaryl; or
(ii) X and Y are independently selected from C or N, wherein at least one of X or Y is N;
R1, R4, and R5 are independently absent, H, halo or alkyl;
one of R2 or R3 is 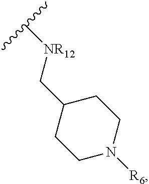
and the other of R2 or R3 is selected from H, halo or alkyl;
R6 is H, alkyl, or heteroaryl^{b}; or
(iii) X and Y are independently selected from C or N, wherein at least one of X or Y is N;
R1 and R4 are independently absent, halo or alkyl;
R3 is halo;
R2 is —NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁-4NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) or —O—(CH₂)₀₋₃(heteroaryl), and
R5 is H, alkyl and halo; or
(iv) X and Y are C;
R4 is H, halo, alkyl;
R5 is H or alkyl;
R3 is H or halo;
one of R1 and R2 is —(CH₂)(heterocyclyl) or —N(R12)CO(CH₂)₀₋₃(heterocyclyl), and the other of R1 and R2 is selected from H or alkyl; or
(v) X is C or N, and Y is C;
R1 is absent, H or alkyl;
R4 is H or alkyl; R5 is H or alkyl;
wherein: (a) R2 and R3 together with the carbon atoms to which they are bonded form phenyl or a 5- or 6-membered nitrogen-containing heteroaryl, wherein phenyl is optionally substituted as for aryl^{b}, and wherein the 5- or 6-membered nitrogen-containing heteroaryl is optionally substituted as for heteroaryl^{b}, or (b) R2 and R3 are independently selected from H or halo, wherein at least one of R2 or R3 is halo, or (c) R2 and R3 are independently selected from H, aryl^{b} or heteroaryl^{b}, wherein at least one of R2 or R3 is aryl^{b}, or heteroaryl^{b};
B is:
(i) a fused 6,5- or 6,6-heteroaromatic bicyclic ring, containing N and, optionally, one or two additional heteroatoms independently selected from N, O or S; wherein the fused 6,5- or 6,6-heteroaromatic bicyclic ring is optionally substituted with 1, 2, or 3 substituents selected from alkyl, alkoxy, OH, halo, CN, —COOR13, —CONR13R14, CF3 or —NR13R14; wherein the 6,5-heteroaromatic bicyclic ring optionally is attached via the 6- or 5-membered ring; or
(ii) phenyl, which is optionally substituted with 1, 2 or 3 substituents independently selected from, alkyl, heteroaryl, alkoxy, heterocyclyl, OH, halo, CN, CF₃, or a carbon-containing 4-, 5-, 6- or 7-membered ring containing 1, 2 or 3 heteroatoms independently selected from N or NR12, which is saturated or unsaturated with 1 or 2 double bonds and optionally mono- or di-substituted with substituents independently selected from oxo, alkyl, alkoxy, OH, halo, or CF₃; or
(iii) phenyl, wherein two adjacent carbon atoms on the phenyl are either linked together by —N═C—N(R8)-C(═O)— to form a quinazolinone or linked together by —CH₂—N(R8)-C(═O)— to form an isoindolinone; or
(iv) heteroaryl; or
(v) a fused 6,5- or 6,6-bicyclic ring containing an aromatic ring fused to a non-aromatic ring and containing N and, optionally, one or two additional heteroatoms independently selected from N, O or S;
wherein the fused 6,5- or 6,6-bicyclic ring is optionally substituted with 1, 2, or 3 substituents selected from alkyl, alkoxy, OH, halo, CN, —COOR13, —CONR13R14, CF₃ or —NR13R14;
wherein the 6,5-bicyclic ring optionally is attached via the 6- or 5-membered ring;
alkoxy is a linear O-linked hydrocarbon of between 1 and 6 carbon atoms (C₁-C₆) or a branched O-linked hydrocarbon of between 3 and 6 carbon atoms (C3-C6); alkoxy is optionally substituted with 1 or 2 substituents independently selected from OH, CN, CF₃, —N(R12)₂ or fluoro;
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, or heterocyclyl^{b};
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1 or 2 substituents independently selected from (C₁-C₆)alkoxy, OH, —N(R12)₂, —NHCOCH₃, CF₃, halo, oxo, heterocyclyl^{b}, or cyclopropane;
alkylene is a bivalent linear saturated hydrocarbon having 1 to 5 carbon atoms (C₁-C₅); alkylene is optionally substituted with 1 or 2 substituents independently selected from alkyl, (C₁-C₆)alkoxy, OH, CN, CF₃, or halo;
aryl is phenyl, biphenyl or naphthyl; aryl is optionally substituted with 1, 2 or 3 substituents independently selected from alkyl, alkoxy, OH, —SO₂CH₃, halo, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR13, —CONR13R14, —(CH₂)₀₋₃—NR13R14, OCF₃ or CF₃; or two adjacent carbon ring atoms on the aryl are optionally linked by a heteroalkylene to form a non-aromatic ring containing 5, 6, or 7 ring members; or wherein two adjacent ring atoms on the aryl are linked to form a 5- or 6-membered aromatic ring containing 1 or 2 heteroatoms that are selected from N, NR8, S, or O, which is optionally substituted as for heteroaryl^{b};
aryl^{b} is phenyl, biphenyl or naphthyl, which is optionally substituted with 1, 2 or 3 substituents independently selected from methyl, ethyl, propyl, isopropyl, alkoxy, OH, —SO₂CH₃, N(R12)₂, halo, CN, or CF₃; or two adjacent carbon ring atoms on the aryl are optionally linked by a heteroalkylene to form a non-aromatic ring containing 5, 6, or 7 ring members;
cycloalkyl is a monocyclic saturated hydrocarbon ring of between 3 and 6 carbon atoms (C₃-C₆); cycloalkyl is optionally substituted with 1 or 2 substituents independently selected from alkyl^{b}, (C₁-C₆)alkoxy, OH, CN, CF₃, or halo;
halo is F, Cl, Br, or I;
heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene is optionally substituted with 1 or 2 substituents independently selected from alkyl (C₁-C₆)alkoxy, OH, CN, CF₃, or halo;
heteroaryl is a 5- or 6-membered carbon-containing aromatic ring containing 1, 2, 3, or 4 ring members that are selected from N, NR8, S, or O; heteroaryl is optionally substituted with 1, 2 or 3 substituents independently selected from alkyl, alkoxy, aryl^{b}, OH, OCF₃, halo, heterocyclyl^{b}, CN, or CF₃;
heteroaryl^{b} is a 5- or 6-membered carbon-containing aromatic ring containing one, two or three ring members that are selected from N, NR8, S, or O; heteroaryl^{b} is optionally substituted with 1, 2 or 3 substituents independently selected from methyl, ethyl, propyl, isopropyl, alkoxy, CH₂aryl^{b}, OH, OCF₃, halo, CN, or CF₃;
heterocyclyl is a 4-, 5-, 6-, or 7-membered carbon-containing non-aromatic ring containing one, two or three ring members that are selected from N, NR8, S, SO, SO₂ or O; heterocyclyl is optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) r CF₃; optionally wherein two ring atoms on heterocyclyl are linked with an alkylene to form a non-aromatic ring containing 5, 6, or 7 ring members; or optionally two adjacent ring atoms on heterocyclyl are linked to form a 5- or 6-membered aromatic ring containing 1 or 2 heteroatoms that are selected from N, NR8, S, or O; or optionally a carbon ring atom on heterocyclyl is substituted with a heteroalkylene such that the carbon ring atom on heterocyclyl together with the heteroalkylene forms a heterocyclyl^{b} that is spiro to ring heterocyclyl;
heterocyclyl^{b} is a 4-, 5-, 6-, or 7-membered carbon-containing non-aromatic ring containing one, two or three ring members that are selected from N, NR12, S, SO, SO₂ or O; heterocyclyl^{b} is optionally substituted with 1, 2, 3, or 4 substituents independently selected from methyl, ethyl, propyl, isopropyl, alkoxy, OH, OCF₃, halo, oxo, CN, or CF₃;
R13 and R14 are independently selected from H, —SO₂CH₃, alkyl^{b}, heteroaryl^{b}, or cycloalkyl; or R13 and R14 together with the nitrogen atom to which they are attached form a carbon-containing 4-, 5-, 6- or 7-membered heterocyclic ring, optionally containing an additional heteroatom selected from N, NR8, S, SO, SO₂, or O, which is saturated or unsaturated with 1 or 2 double bonds and optionally mono- or di-substituted with substituents independently selected from oxo, alkyl^{b}, alkoxy, OH, halo, —SO₂CH₃, or CF₃; or R13 and R14 together with the nitrogen atom to which they are attached form a carbon-containing 5- or 6-membered heterocyclic ring, which is fused to an aryl^{b} or a heteroaryl^{b};
R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, or —(CH₂)₀₋₃heterocyclyl^{b}; or R8 is a carbon-containing 4-, 5-, 6- or 7-membered heterocyclic ring containing 1, 2 or 3 heteroatoms independently selected from N, NR12, S, SO, SO₂, or O, which is saturated or unsaturated with 1 or 2 double bonds and optionally mono- or di-substituted with substituents independently selected from oxo, alkyl^{b}, alkoxy, OH, halo, —SO₂CH₃, or CF₃;
R12 is independently selected from H, —SO₂CH₃, methyl, ethyl, propyl, isopropyl, or cycloalkyl;
or a tautomer, isomer, stereoisomer deuterated isotope, pharmaceutically acceptable salt and/or solvate thereof.

2. The compound of formula (I) according to claim 1, or a tautomer, isomer, stereoisomer a deuterated isotope, a pharmaceutically acceptable salt and/or solvate thereof, wherein A is a 6-membered heteroaryl of formula (II): 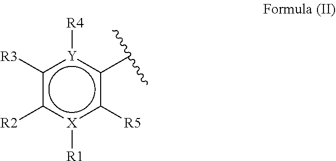
wherein:
X and Y are independently selected from C or N, wherein at least one of X or Y is N;
R5 is selected from —NR12(CH₂)₀₋₃(heterocyclyl), —NR12(CH₂)₀₋₃(heteroaryl), —NR12(CH₂)₀₋₃(aryl), —NR13R14, —O(CH₂)₀₋₃(aryl), —O(CH₂)₀₋₃(heterocyclyl), —O—(CH₂)₁-4NR13R14, or —NR12(CH₂)₀₋₃O(aryl);
R2, R3 and R4 are independently selected from H, halo, alkoxy, alkyl, cycloalkyl, aryl, or heteroaryl.

3. The compound of formula (I) according to claim 2, or a tautomer, isomer, stereoisomer a deuterated isotope, a pharmaceutically acceptable salt and/or solvate thereof, wherein X is N.

4. The compound of formula (I) according to claim 2, or a tautomer, isomer, stereoisomer a deuterated isotope, a pharmaceutically acceptable salt and/or solvate thereof, wherein R3 is halo.

5. The compound of formula (I) according to claim 2, or a tautomer, isomer, stereoisomer a deuterated isotope, a pharmaceutically acceptable salt and/or solvate thereof, wherein R5 is —NR12(CH₂)(heterocyclyl), wherein “heterocyclyl” is optionally substituted.

6. compound of formula (I) according to claim 2, or a tautomer, isomer, stereoisomer a deuterated isotope, a pharmaceutically acceptable salt and/or solvate thereof, wherein R5 is —NR12(CH₂)(piperidinyl), wherein “piperidinyl” is optionally substituted as defined for “heterocyclyl”.

7. The compound of formula (I) according to claim 1, or a tautomer, isomer, stereoisomer a deuterated isotope, a pharmaceutically acceptable salt and/or solvate thereof, wherein A is a 6-membered heteroaryl of formula (II): 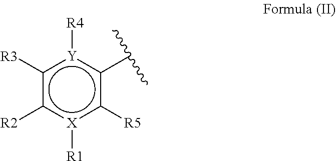
wherein:
X and Y are independently selected from C or N, wherein at least one of X or Y is N;
R1, R4, and R5 are independently absent, H, halo or alkyl;
one of R2 or R3 is 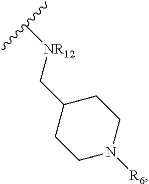
and the other of R2 or R3 is selected from H, halo or alkyl; and
R6 is H, alkyl, or heteroaryl^{b}.

8. The compound of formula (I) according to claim 7, or a tautomer, isomer, stereoisomer a deuterated isotope, a pharmaceutically acceptable salt and/or solvate thereof, wherein X is N.

9. The compound of formula (I) according to claim 7, or a tautomer, isomer, stereoisomer a deuterated isotope, a pharmaceutically acceptable salt and/or solvate thereof, wherein Y is N.

10. The compound of formula (I) according to claim 7, or a tautomer, isomer, stereoisomer a deuterated isotope, a pharmaceutically acceptable salt and/or solvate thereof, wherein R6 is alkyl.

11. The compound of formula (I) according to claim 1, or a tautomer, isomer, stereoisomer a deuterated isotope, a pharmaceutically acceptable salt and/or solvate thereof, wherein A is a 6-membered heteroaryl of formula (II): 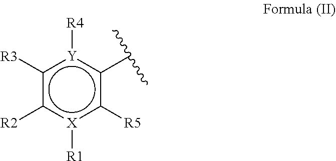
wherein:
X and Y are independently selected from C or N, wherein at least one of X or Y is N;
R1 and R4 are independently absent, e H, halo or alkyl;
R3 is halo;
R2 is —(CH₂)₀₋₃NR13R14, —NR12(CH₂)₀₋₃(aryl), —NR12(CH₂)₀₋₃NR13R14, —(CH₂)NR12(CH₂)₀₋₃(heterocyclyl), —O—(CH₂)₁-4NR13R14, —(CH₂)₀₋₃NR12(CH₂)₀₋₃(heteroaryl), —(CH₂)₀₋₃O(CH₂)₀₋₃(aryl), —O—(CH₂)₀₋₃(heterocyclyl) or —O—(CH₂)₀₋₃(heteroaryl); and
R5 is H, alkyl or halo.

12. The compound of formula (I) according to claim 11, or a tautomer, isomer, stereoisomer a deuterated isotope, a pharmaceutically acceptable salt and/or solvate thereof, wherein X is N.

13. The compound of formula (I) according to claim 11, or a tautomer, isomer, stereoisomer a deuterated isotope, a pharmaceutically acceptable salt and/or solvate thereof, wherein R3 is halo.

14. The compound of formula (I) according to claim 11, or a tautomer, isomer, stereoisomer a deuterated isotope, a pharmaceutically acceptable salt and/or solvate thereof, wherein R2 is —(CH₂)₀₋₃NR13R14.

15. The compound of formula (I) according to claim 14, or a tautomer, isomer, stereoisomer a deuterated isotope, a pharmaceutically acceptable salt and/or solvate thereof, wherein R2 is —(CH₂)NR13R14, wherein R13 and R14, together with the nitrogen atom to which they are attached form piperazine, which is optionally substituted.

16. The compound of formula (I) according to claim 15, or a tautomer, isomer, stereoisomer a deuterated isotope, a pharmaceutically acceptable salt and/or solvate thereof,
wherein the piperazine on R2 has an NR8 group, wherein R8 is pyridinyl.

17. The compound of formula (I) according to claim 1, or a tautomer, isomer, stereoisomer a deuterated isotope, a pharmaceutically acceptable salt and/or solvate thereof, wherein A is a 6-membered heteroaryl of formula (II): 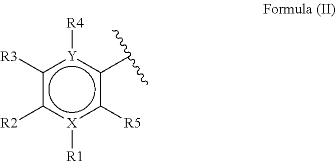
wherein:
X and Y are C;
R4 is H, halo, or alkyl;
R5 is H or alkyl;
R3 is H or halo; and
one of R1 and R2 is —(CH₂)(heterocyclyl) or —N(R12)CO(CH₂)₀₋₃(heterocyclyl), and the other of R1 and R2 is selected from H or alkyl.

18. The compound of formula (I) according to claim 17, or a tautomer, isomer, stereoisomer a deuterated isotope, a pharmaceutically acceptable salt and/or solvate thereof, wherein R3 is halo.

19. The compound of formula (I) according to claim 17, or a tautomer, isomer, stereoisomer a deuterated isotope, a pharmaceutically acceptable salt and/or solvate thereof, wherein R2 is —(CH₂)(heterocyclyl).

20. The compound of formula (I) according to claim 19, or a tautomer, isomer, stereoisomer a deuterated isotope, a pharmaceutically acceptable salt and/or solvate thereof, wherein R2 is —(CH₂)(piperazinyl).

21. The compound of formula (I) according to claim 19, or a tautomer, isomer, stereoisomer a deuterated isotope, a pharmaceutically acceptable salt and/or solvate thereof, wherein the heterocyclyl on R2 has an NR8 group, and R8 is pyridinyl.

22. The compound of formula (I) according to claim 1, or a tautomer, isomer, stereoisomer a deuterated isotope, a pharmaceutically acceptable salt and/or solvate thereof, wherein A is a 6-membered heteroaryl of formula (II): 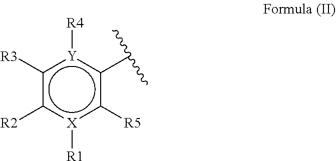
wherein:
X is C or N, and Y is C;
R1 is absent, H or alkyl;
R4 is H or alkyl;
R5 is H or alkyl;
wherein: (a) R2 and R3 together with the carbon atoms to which they are bonded form phenyl or a 5- or 6-membered nitrogen-containing heteroaryl, wherein phenyl is optionally substituted as for aryl^{b}, and wherein the 5- or 6-membered nitrogen-containing heteroaryl is optionally substituted as for heteroaryl^{b}, or (b) R2 and R3 are independently selected from H or halo, wherein at least one of R2 or R3 is halo, or (c) R2 and R3 are independently selected from H, aryl^{b} or heteroaryl^{b}, wherein at least one of R2 or R3 is aryl^{b}, or heteroaryl^{b}.

23. The compound of formula (I) according to claim 22, or a tautomer, isomer, stereoisomer a deuterated isotope, a pharmaceutically acceptable salt and/or solvate thereof, wherein R2 and R3 together with the carbon atoms to which they are bonded form phenyl or a 5- or 6-membered nitrogen-containing heteroaryl, wherein phenyl is optionally substituted as for aryl^{b}, and wherein the 5- or 6-membered nitrogen-containing heteroaryl is optionally substituted as for heteroaryl^{b}.

24. The compound of formula (I) according to claim 22, or a tautomer, isomer, stereoisomer a deuterated isotope, a pharmaceutically acceptable salt and/or solvate thereof, wherein R2 and R3 are independently selected from H or halo, wherein at least one of R2 or R3 is halo.

25. The compound of formula (I) according to claim 22, or a tautomer, isomer, stereoisomer a deuterated isotope, a pharmaceutically acceptable salt and/or solvate thereof, wherein R2 and R3 are independently selected from H, aryl^{b} or heteroaryl^{b}, wherein at least one of R2 or R3 is aryl^{b}, or heteroaryl^{b}.

26. A compound that is: 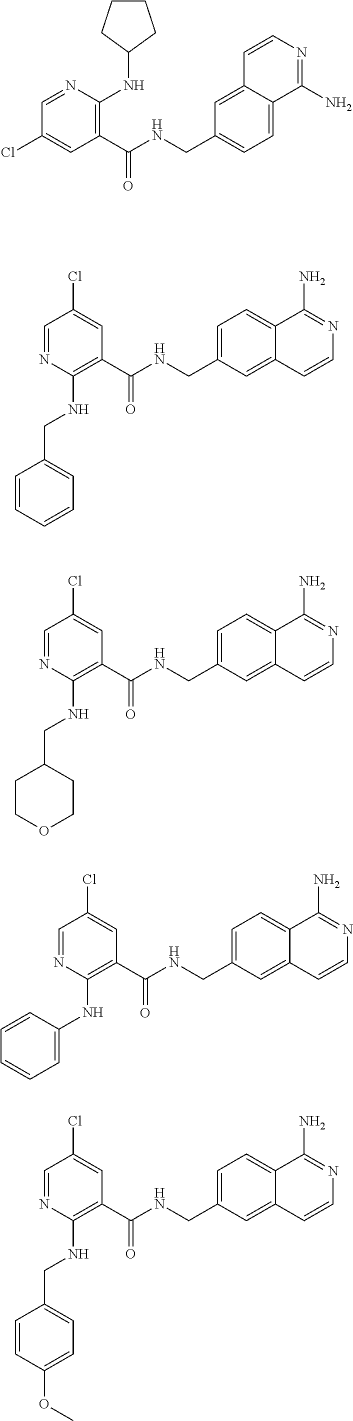 [SMILES: Nc1nccc2cc(CNC(=O)c3cc(Cl)cnc3NC3CCCC3)ccc12 ; Nc1nccc2cc(CNC(=O)c3cc(Cl)cnc3NCc3ccccc3)ccc12 ; Nc1nccc2cc(CNC(=O)c3cc(Cl)cnc3NCC3CCOCC3)ccc12 ; Nc1nccc2cc(CNC(=O)c3cc(Cl)cnc3Nc3ccccc3)ccc12 ; COc1ccc(CNc2ncc(Cl)cc2C(=O)NCc2ccc3c(N)nccc3c2)cc1] 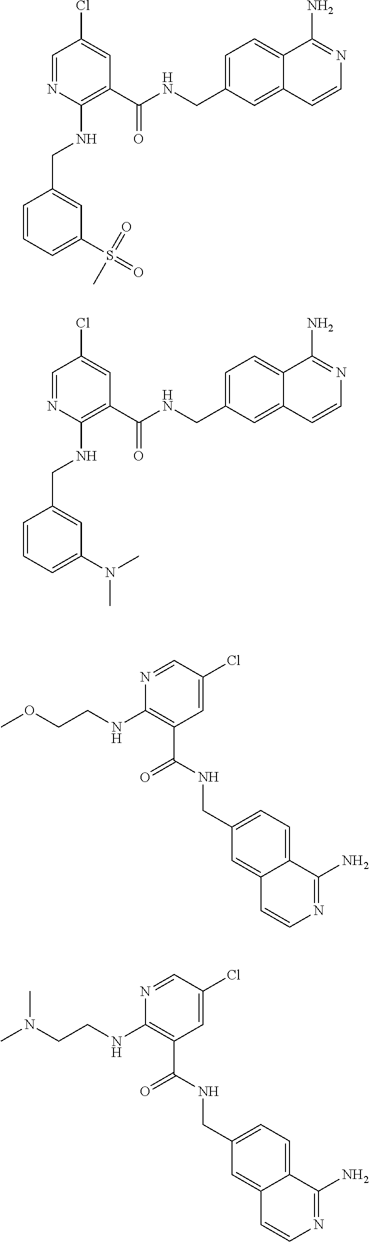 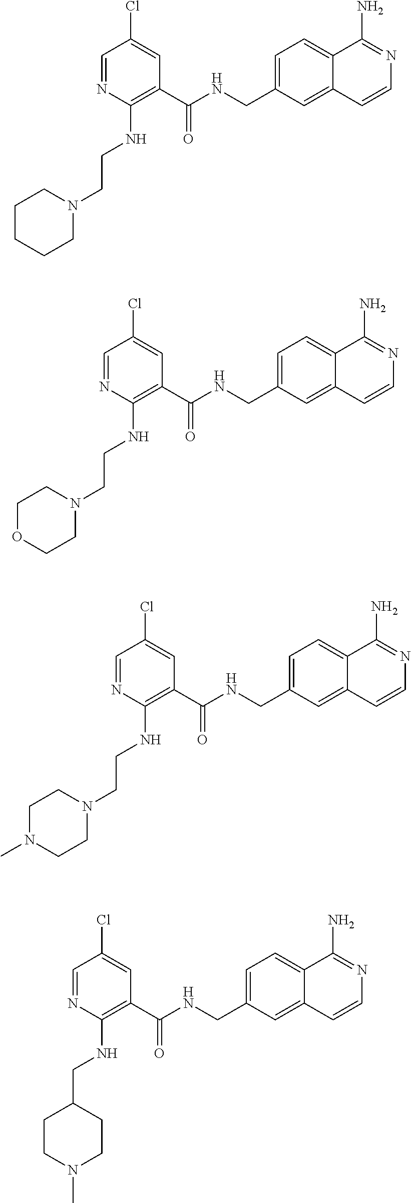 [SMILES: Nc1nccc2cc(CNC(=O)c3cc(Cl)cnc3NCCN3CCCCC3)ccc12 ; Nc1nccc2cc(CNC(=O)c3cc(Cl)cnc3NCCN3CCOCC3)ccc12 ; CN1CCN(CCNc2ncc(Cl)cc2C(=O)NCc2ccc3c(N)nccc3c2)CC1 ; CN1CCC(CNc2ncc(Cl)cc2C(=O)NCc2ccc3c(N)nccc3c2)CC1] 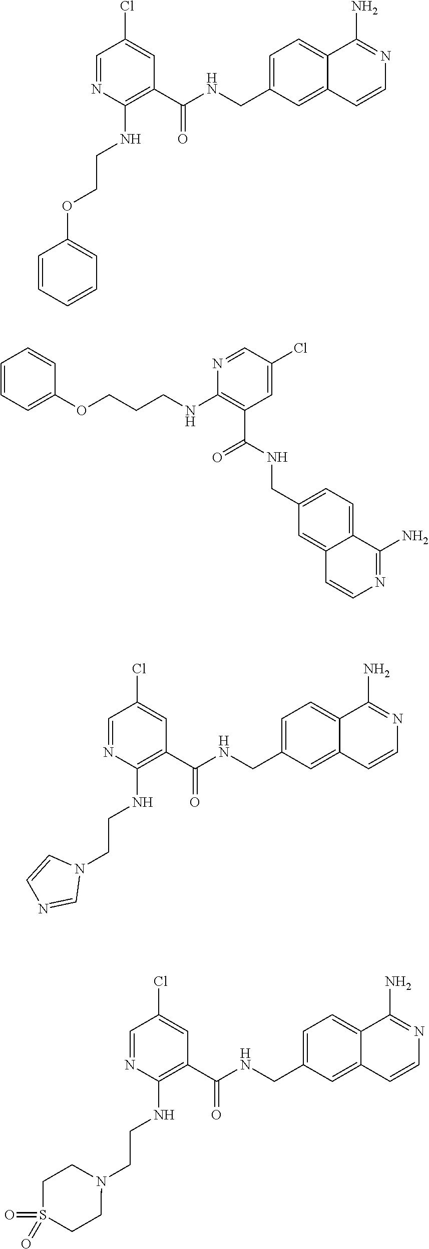 [SMILES: Nc1nccc2cc(CNC(=O)c3cc(Cl)cnc3NCCOc3ccccc3)ccc12 ; Nc1nccc2cc(CNC(=O)c3cc(Cl)cnc3NCCCOc3ccccc3)ccc12 ; Nc1nccc2cc(CNC(=O)c3cc(Cl)cnc3NCCn3ccnc3)ccc12 ; Nc1nccc2cc(CNC(=O)c3cc(Cl)cnc3NCCN3CCS(=O)(=O)CC3)ccc12] 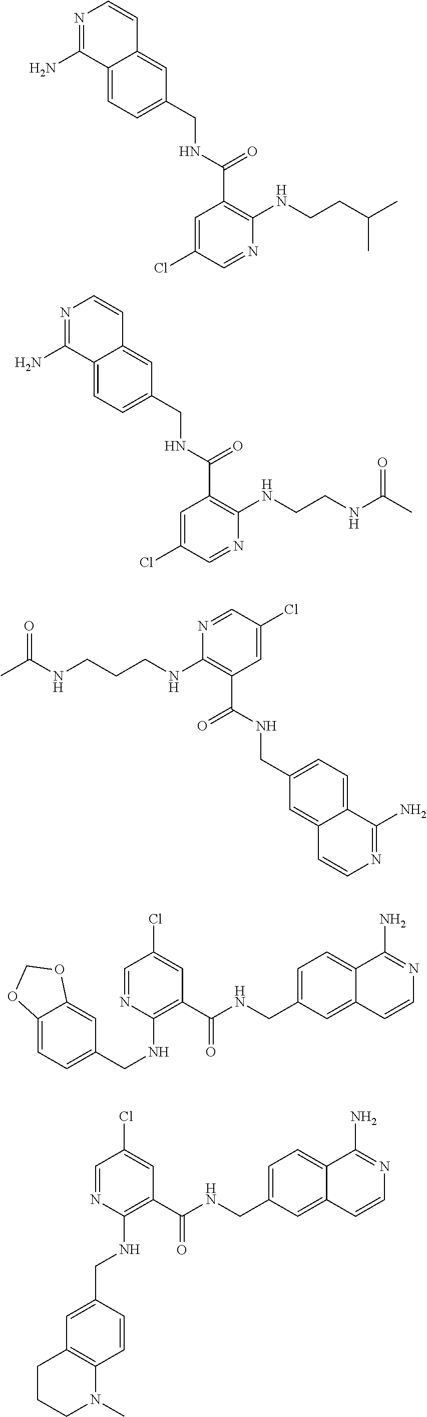 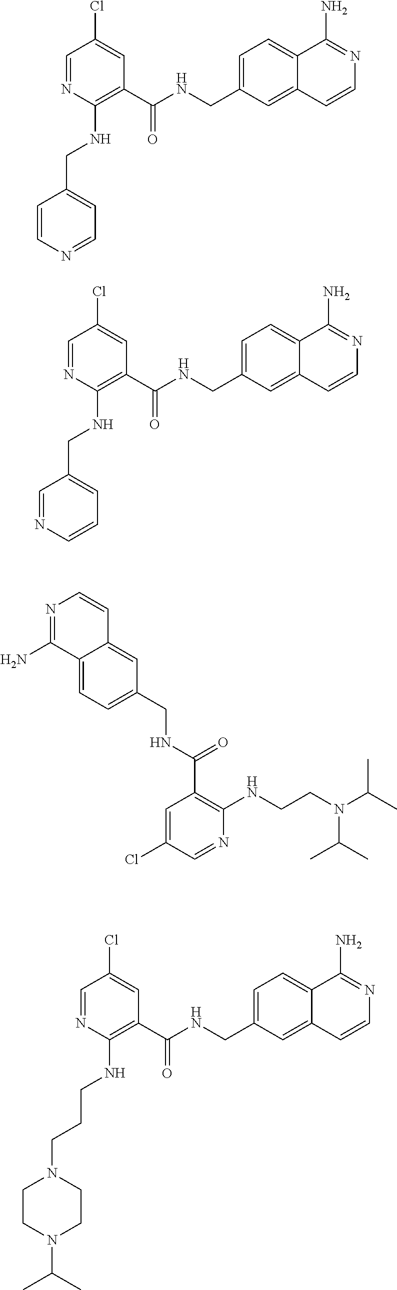 [SMILES: Nc1nccc2cc(CNC(=O)c3cc(Cl)cnc3NCc3ccncc3)ccc12 ; Nc1nccc2cc(CNC(=O)c3cc(Cl)cnc3NCc3cccnc3)ccc12 ; CC(C)N(CCNc1ncc(Cl)cc1C(=O)NCc1ccc2c(N)nccc2c1)C(C)C ; CC(C)N1CCN(CCCNc2ncc(Cl)cc2C(=O)NCc2ccc3c(N)nccc3c2)CC1] 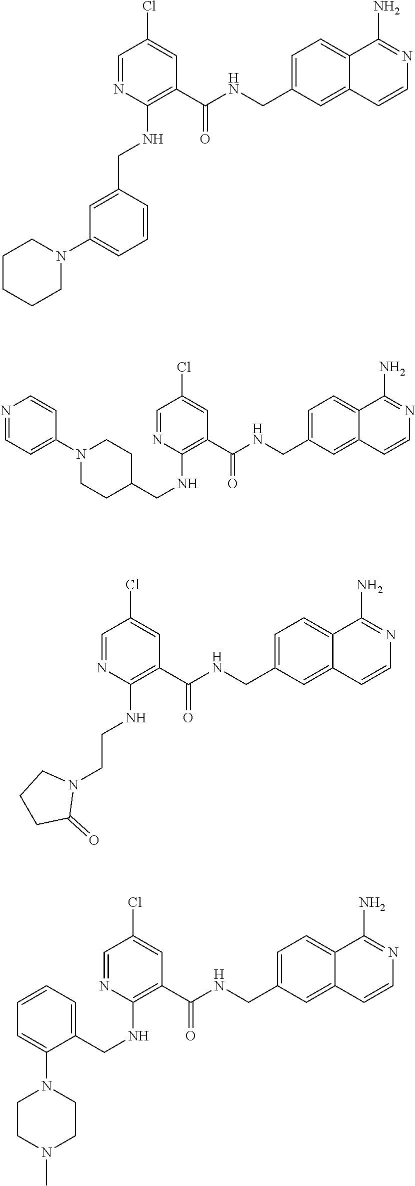 [SMILES: Nc1nccc2cc(CNC(=O)c3cc(Cl)cnc3NCc3cccc(N4CCCCC4)c3)ccc12 ; Nc1nccc2cc(CNC(=O)c3cc(Cl)cnc3NCC3CCN(c4ccncc4)CC3)ccc12 ; Nc1nccc2cc(CNC(=O)c3cc(Cl)cnc3NCCN3CCCC3=O)ccc12 ; CN1CCN(c2ccccc2CNc2ncc(Cl)cc2C(=O)NCc2ccc3c(N)nccc3c2)CC1] 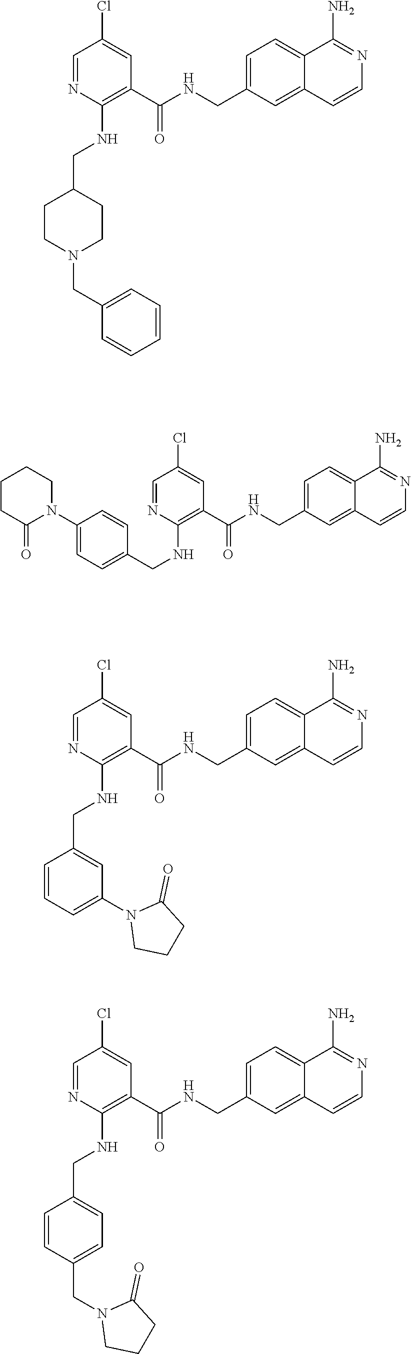 [SMILES: Nc1nccc2cc(CNC(=O)c3cc(Cl)cnc3NCC3CCN(Cc4ccccc4)CC3)ccc12 ; Nc1nccc2cc(CNC(=O)c3cc(Cl)cnc3NCc3ccc(N4CCCCC4=O)cc3)ccc12 ; Nc1nccc2cc(CNC(=O)c3cc(Cl)cnc3NCc3cccc(N4CCCC4=O)c3)ccc12 ; Nc1nccc2cc(CNC(=O)c3cc(Cl)cnc3NCc3ccc(CN4CCCC4=O)cc3)ccc12] 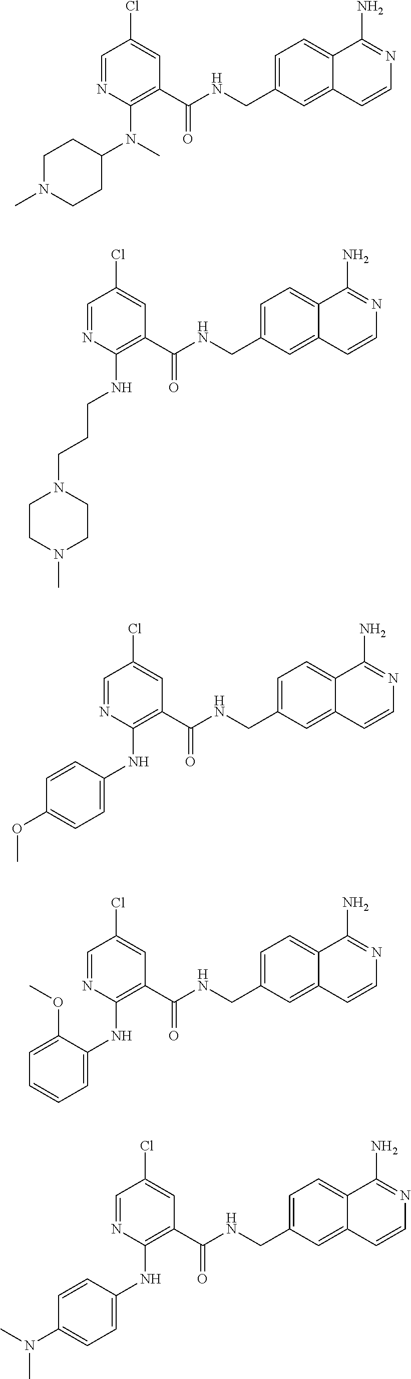 [SMILES: CN1CCC(N(C)c2ncc(Cl)cc2C(=O)NCc2ccc3c(N)nccc3c2)CC1 ; CN1CCN(CCCNc2ncc(Cl)cc2C(=O)NCc2ccc3c(N)nccc3c2)CC1 ; COc1ccc(Nc2ncc(Cl)cc2C(=O)NCc2ccc3c(N)nccc3c2)cc1 ; COc1ccccc1Nc1ncc(Cl)cc1C(=O)NCc1ccc2c(N)nccc2c1 ; CN(C)c1ccc(Nc2ncc(Cl)cc2C(=O)NCc2ccc3c(N)nccc3c2)cc1] 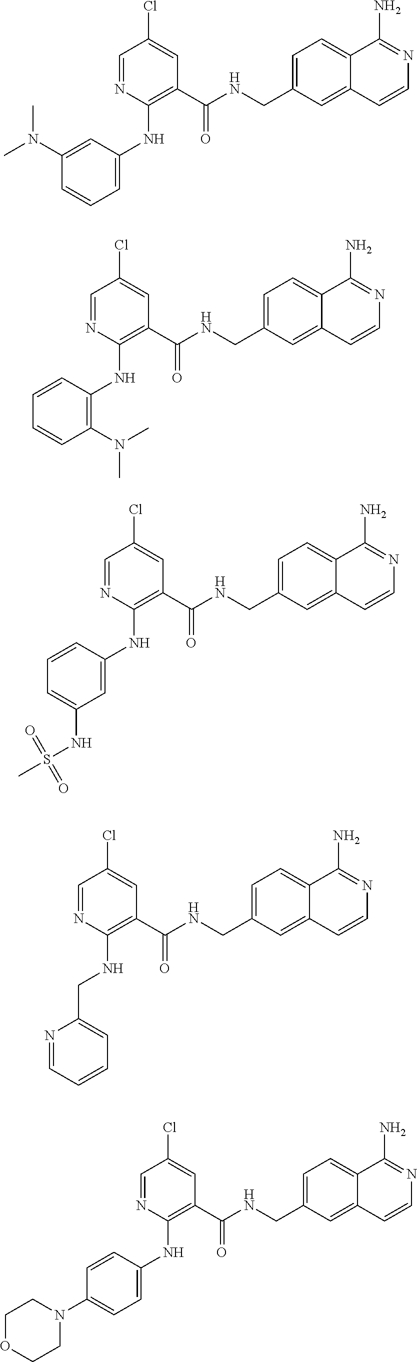 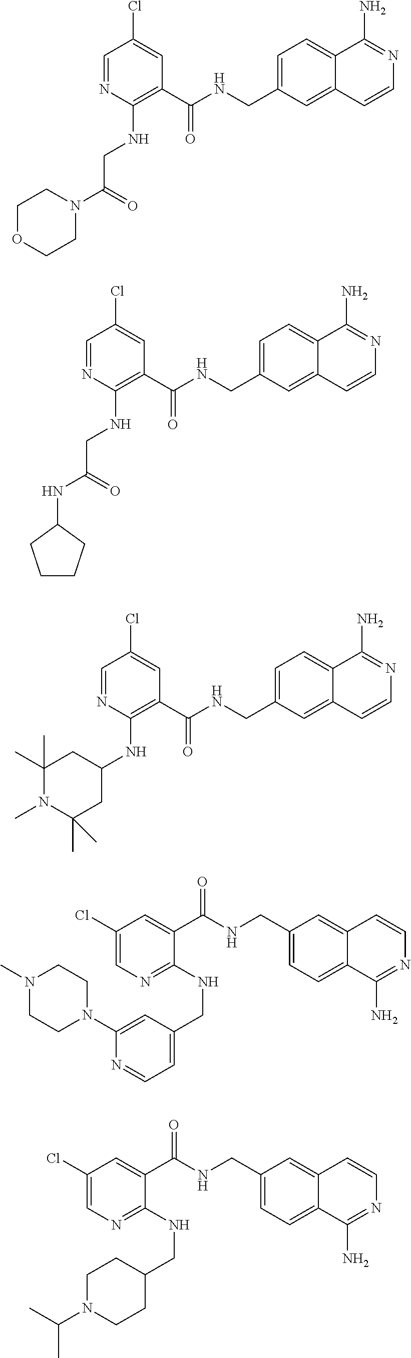 [SMILES: Nc1nccc2cc(CNC(=O)c3cc(Cl)cnc3NCC(=O)N3CCOCC3)ccc12 ; Nc1nccc2cc(CNC(=O)c3cc(Cl)cnc3NCC(=O)NC3CCCC3)ccc12 ; CN1C(C)(C)CC(Nc2ncc(Cl)cc2C(=O)NCc2ccc3c(N)nccc3c2)CC1(C)C ; CN1CCN(c2cc(CNc3ncc(Cl)cc3C(=O)NCc3ccc4c(N)nccc4c3)ccn2)CC1 ; CC(C)N1CCC(CNc2ncc(Cl)cc2C(=O)NCc2ccc3c(N)nccc3c2)CC1] 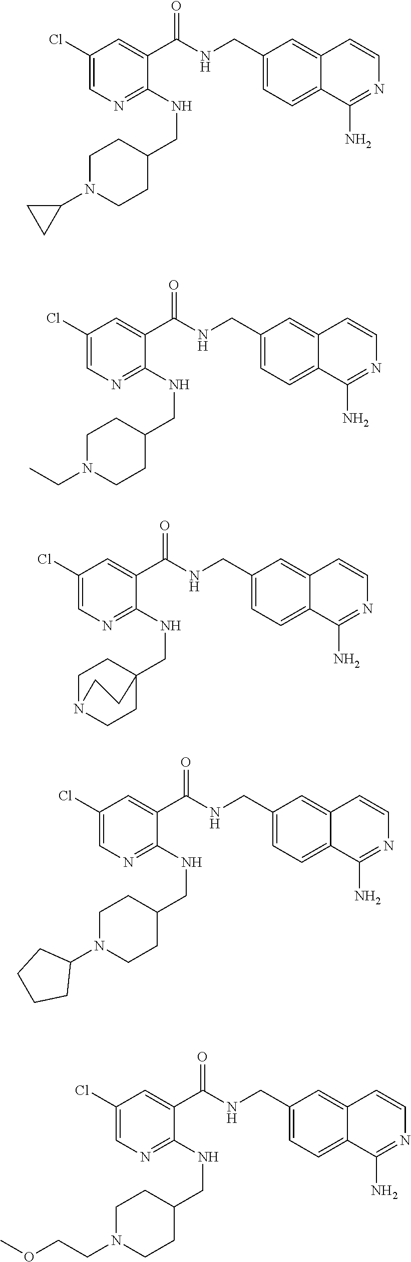 [SMILES: Nc1nccc2cc(CNC(=O)c3cc(Cl)cnc3NCC3CCN(C4CC4)CC3)ccc12 ; CCN1CCC(CNc2ncc(Cl)cc2C(=O)NCc2ccc3c(N)nccc3c2)CC1 ; Nc1nccc2cc(CNC(=O)c3cc(Cl)cnc3NCC34CCN(CC3)CC4)ccc12 ; Nc1nccc2cc(CNC(=O)c3cc(Cl)cnc3NCC3CCN(C4CCCC4)CC3)ccc12 ; COCCN1CCC(CNc2ncc(Cl)cc2C(=O)NCc2ccc3c(N)nccc3c2)CC1] 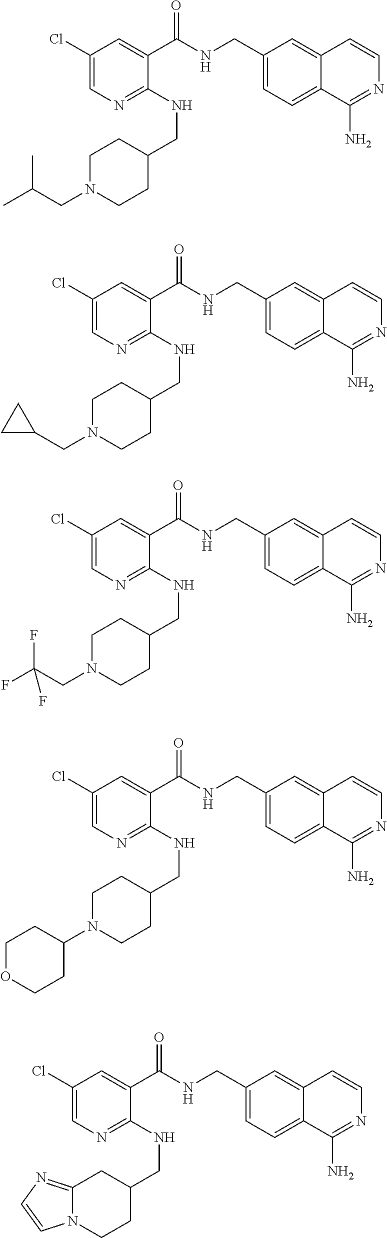 [SMILES: CC(C)CN1CCC(CNc2ncc(Cl)cc2C(=O)NCc2ccc3c(N)nccc3c2)CC1 ; Nc1nccc2cc(CNC(=O)c3cc(Cl)cnc3NCC3CCN(CC4CC4)CC3)ccc12 ; Nc1nccc2cc(CNC(=O)c3cc(Cl)cnc3NCC3CCN(CC(F)(F)F)CC3)ccc12 ; Nc1nccc2cc(CNC(=O)c3cc(Cl)cnc3NCC3CCN(C4CCOCC4)CC3)ccc12 ; Nc1nccc2cc(CNC(=O)c3cc(Cl)cnc3NCC3CCn4ccnc4C3)ccc12] 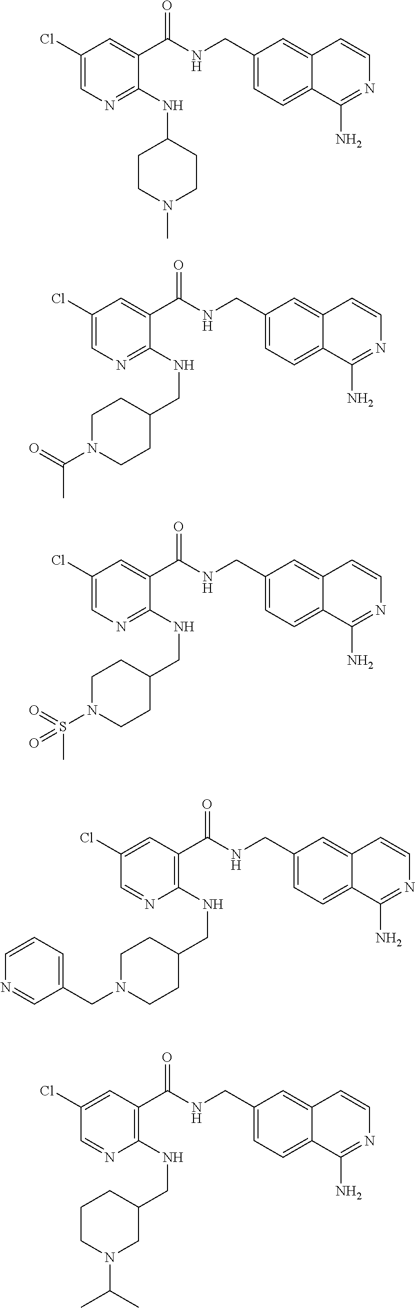 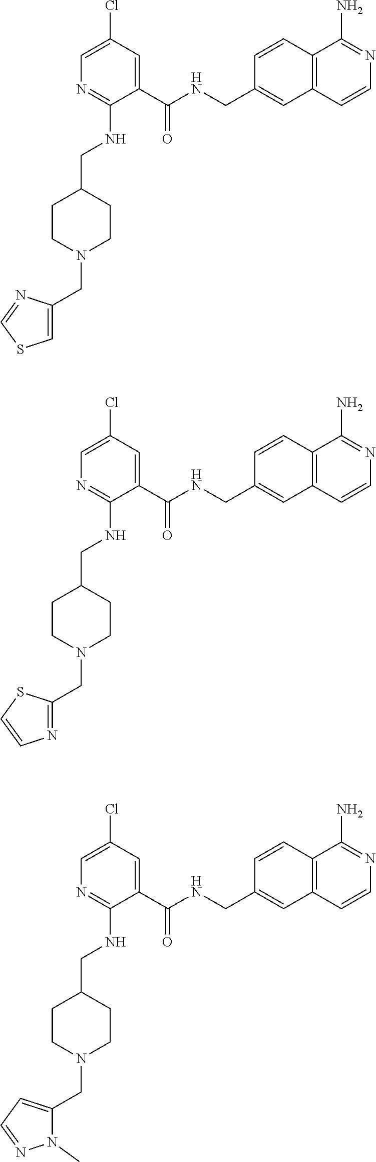 [SMILES: Nc1nccc2cc(CNC(=O)c3cc(Cl)cnc3NCC3CCN(Cc4cscn4)CC3)ccc12 ; Nc1nccc2cc(CNC(=O)c3cc(Cl)cnc3NCC3CCN(Cc4nccs4)CC3)ccc12 ; Cn1nccc1CN1CCC(CNc2ncc(Cl)cc2C(=O)NCc2ccc3c(N)nccc3c2)CC1] 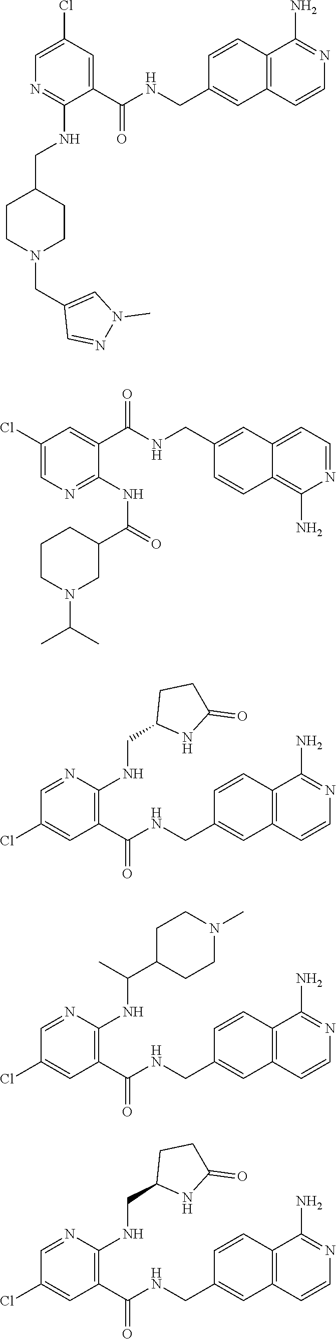 [SMILES: Cn1cc(CN2CCC(CNc3ncc(Cl)cc3C(=O)NCc3ccc4c(N)nccc4c3)CC2)cn1 ; CC(C)N1CCCC(C(=O)Nc2ncc(Cl)cc2C(=O)NCc2ccc3c(N)nccc3c2)C1 ; Nc1nccc2cc(CNC(=O)c3cc(Cl)cnc3NC[C@@H]3CCC(=O)N3)ccc12 ; CC(Nc1ncc(Cl)cc1C(=O)NCc1ccc2c(N)nccc2c1)C1CCN(C)CC1 ; Nc1nccc2cc(CNC(=O)c3cc(Cl)cnc3NC[C@H]3CCC(=O)N3)ccc12] 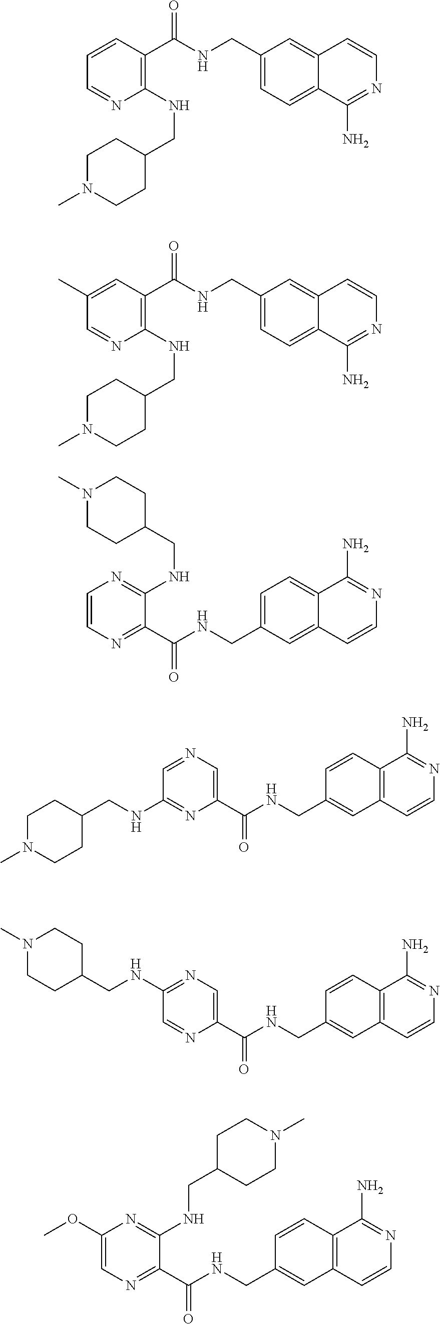 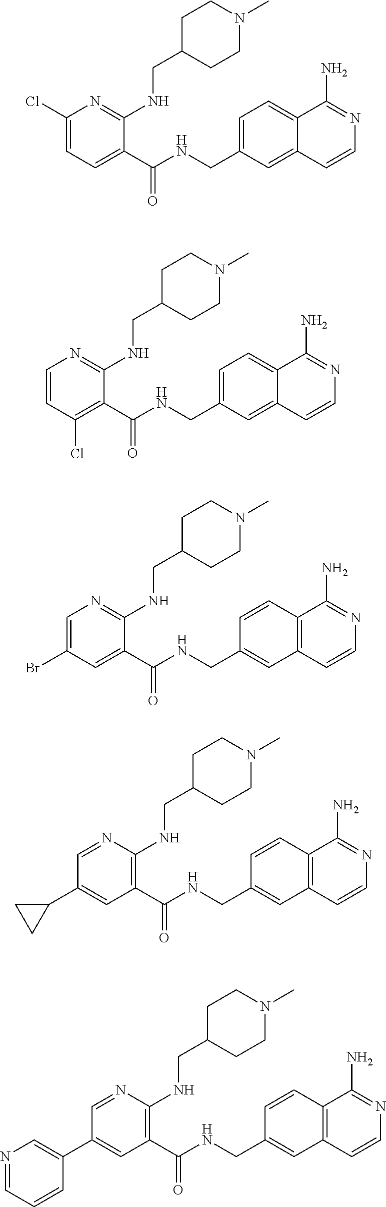 [SMILES: CN1CCC(CNc2nc(Cl)ccc2C(=O)NCc2ccc3c(N)nccc3c2)CC1 ; CN1CCC(CNc2nccc(Cl)c2C(=O)NCc2ccc3c(N)nccc3c2)CC1 ; CN1CCC(CNc2ncc(Br)cc2C(=O)NCc2ccc3c(N)nccc3c2)CC1 ; CN1CCC(CNc2ncc(C3CC3)cc2C(=O)NCc2ccc3c(N)nccc3c2)CC1 ; CN1CCC(CNc2ncc(-c3cccnc3)cc2C(=O)NCc2ccc3c(N)nccc3c2)CC1] 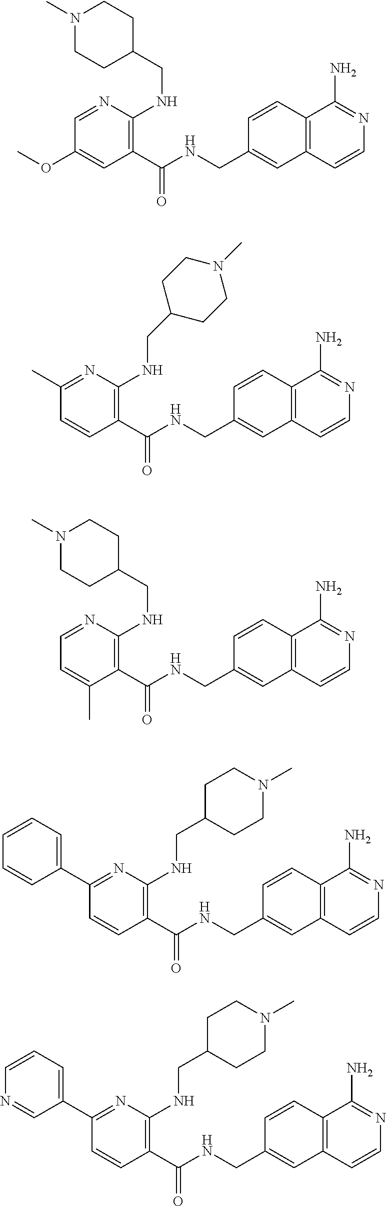 [SMILES: COc1cnc(NCC2CCN(C)CC2)c(C(=O)NCc2ccc3c(N)nccc3c2)c1 ; Cc1ccc(C(=O)NCc2ccc3c(N)nccc3c2)c(NCC2CCN(C)CC2)n1 ; Cc1ccnc(NCC2CCN(C)CC2)c1C(=O)NCc1ccc2c(N)nccc2c1 ; CN1CCC(CNc2nc(-c3ccccc3)ccc2C(=O)NCc2ccc3c(N)nccc3c2)CC1 ; CN1CCC(CNc2nc(-c3cccnc3)ccc2C(=O)NCc2ccc3c(N)nccc3c2)CC1] 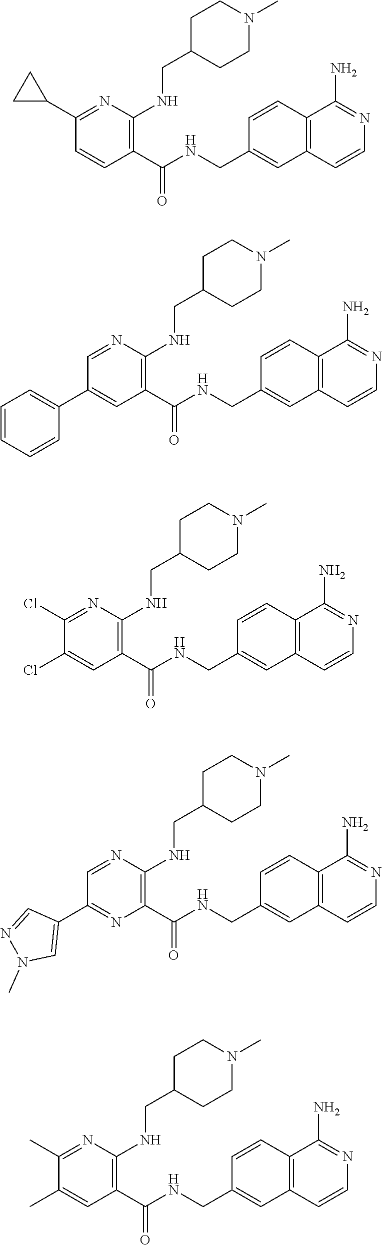 [SMILES: CN1CCC(CNc2nc(C3CC3)ccc2C(=O)NCc2ccc3c(N)nccc3c2)CC1 ; CN1CCC(CNc2ncc(-c3ccccc3)cc2C(=O)NCc2ccc3c(N)nccc3c2)CC1 ; CN1CCC(CNc2nc(Cl)c(Cl)cc2C(=O)NCc2ccc3c(N)nccc3c2)CC1 ; CN1CCC(CNc2ncc(-c3cnn(C)c3)nc2C(=O)NCc2ccc3c(N)nccc3c2)CC1 ; Cc1cc(C(=O)NCc2ccc3c(N)nccc3c2)c(NCC2CCN(C)CC2)nc1C] 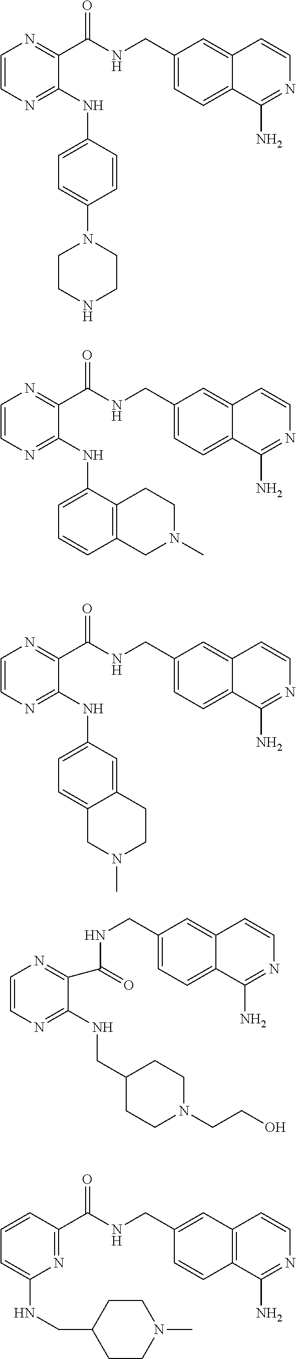 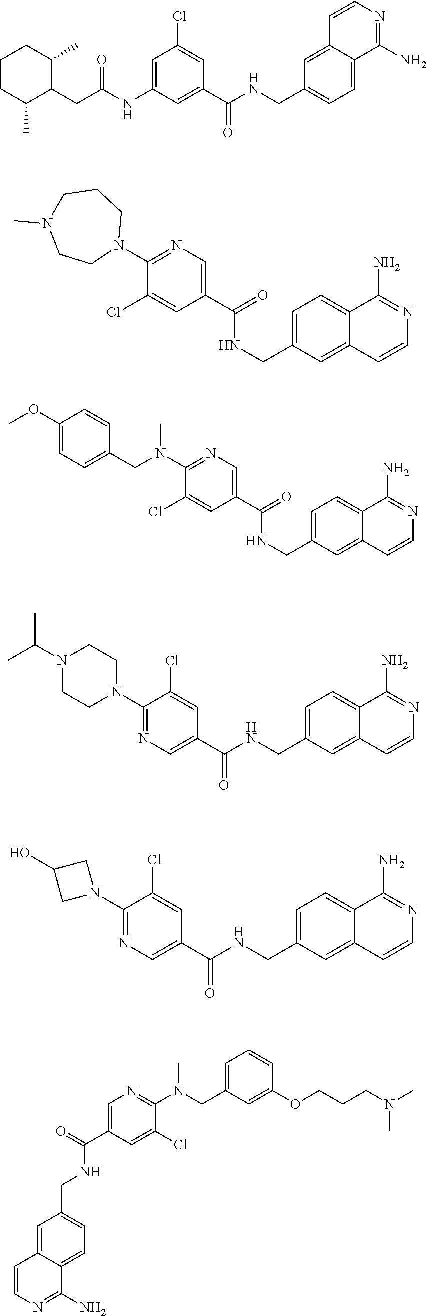 [SMILES: C[C@@H]1CCC[C@H](C)C1CC(=O)Nc1cc(Cl)cc(C(=O)NCc2ccc3c(N)nccc3c2)c1 ; CN1CCCN(c2ncc(C(=O)NCc3ccc4c(N)nccc4c3)cc2Cl)CC1 ; COc1ccc(CN(C)c2ncc(C(=O)NCc3ccc4c(N)nccc4c3)cc2Cl)cc1 ; CC(C)N1CCN(c2ncc(C(=O)NCc3ccc4c(N)nccc4c3)cc2Cl)CC1 ; Nc1nccc2cc(CNC(=O)c3cnc(N4CC(O)C4)c(Cl)c3)ccc12 ; CN(C)CCCOc1cccc(CN(C)c2ncc(C(=O)NCc3ccc4c(N)nccc4c3)cc2Cl)c1] 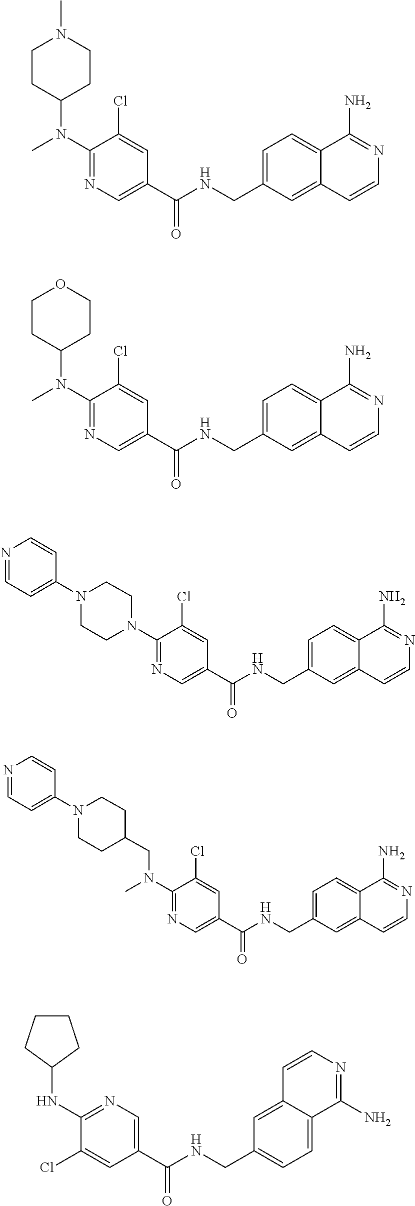 [SMILES: CN1CCC(N(C)c2ncc(C(=O)NCc3ccc4c(N)nccc4c3)cc2Cl)CC1 ; CN(c1ncc(C(=O)NCc2ccc3c(N)nccc3c2)cc1Cl)C1CCOCC1 ; Nc1nccc2cc(CNC(=O)c3cnc(N4CCN(c5ccncc5)CC4)c(Cl)c3)ccc12 ; CN(CC1CCN(c2ccncc2)CC1)c1ncc(C(=O)NCc2ccc3c(N)nccc3c2)cc1Cl ; Nc1nccc2cc(CNC(=O)c3cnc(NC4CCCC4)c(Cl)c3)ccc12] 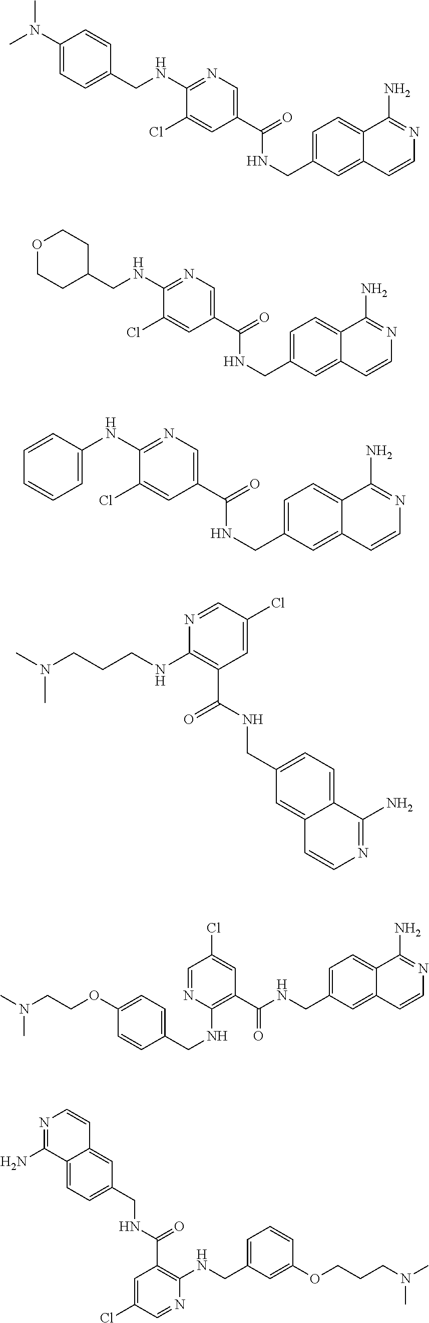 [SMILES: CN(C)c1ccc(CNc2ncc(C(=O)NCc3ccc4c(N)nccc4c3)cc2Cl)cc1 ; Nc1nccc2cc(CNC(=O)c3cnc(NCC4CCOCC4)c(Cl)c3)ccc12 ; Nc1nccc2cc(CNC(=O)c3cnc(Nc4ccccc4)c(Cl)c3)ccc12 ; CN(C)CCCNc1ncc(Cl)cc1C(=O)NCc1ccc2c(N)nccc2c1 ; CN(C)CCOc1ccc(CNc2ncc(Cl)cc2C(=O)NCc2ccc3c(N)nccc3c2)cc1 ; CN(C)CCCOc1cccc(CNc2ncc(Cl)cc2C(=O)NCc2ccc3c(N)nccc3c2)c1] 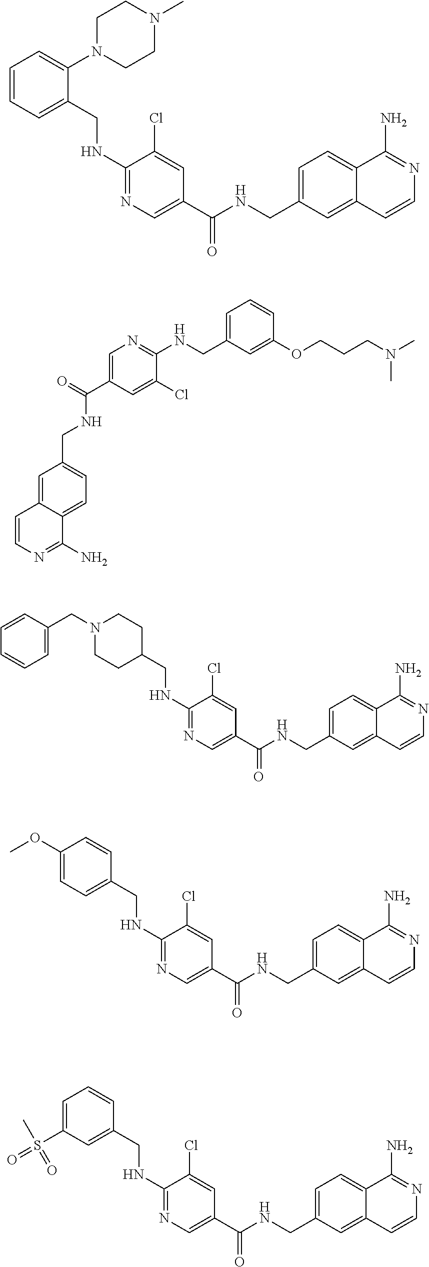 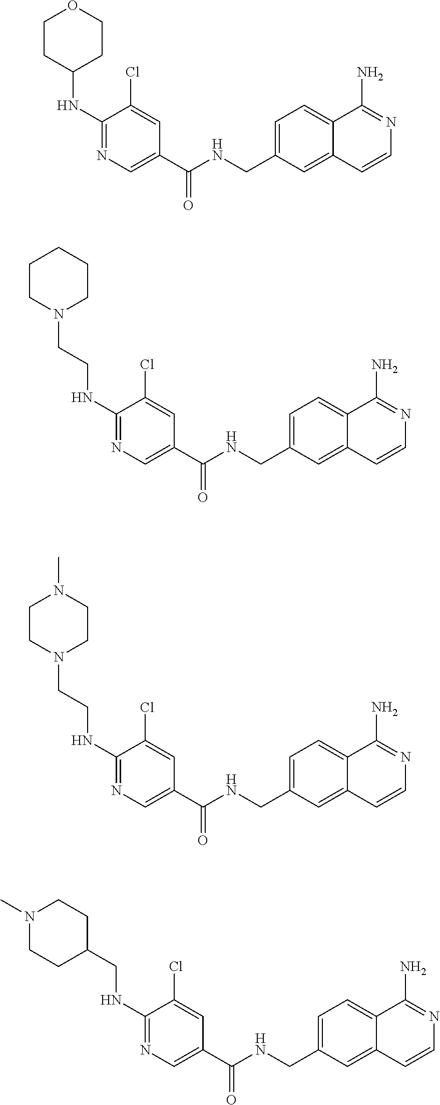 [SMILES: Nc1nccc2cc(CNC(=O)c3cnc(NC4CCOCC4)c(Cl)c3)ccc12 ; Nc1nccc2cc(CNC(=O)c3cnc(NCCN4CCCCC4)c(Cl)c3)ccc12 ; CN1CCN(CCNc2ncc(C(=O)NCc3ccc4c(N)nccc4c3)cc2Cl)CC1 ; CN1CCC(CNc2ncc(C(=O)NCc3ccc4c(N)nccc4c3)cc2Cl)CC1] 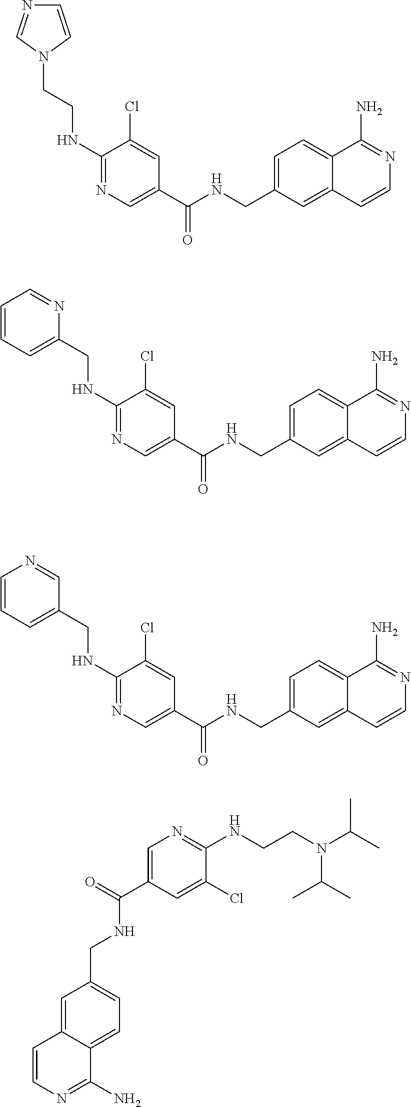 [SMILES: Nc1nccc2cc(CNC(=O)c3cnc(NCCn4ccnc4)c(Cl)c3)ccc12 ; Nc1nccc2cc(CNC(=O)c3cnc(NCc4ccccn4)c(Cl)c3)ccc12 ; Nc1nccc2cc(CNC(=O)c3cnc(NCc4cccnc4)c(Cl)c3)ccc12 ; CC(C)N(CCNc1ncc(C(=O)NCc2ccc3c(N)nccc3c2)cc1Cl)C(C)C] 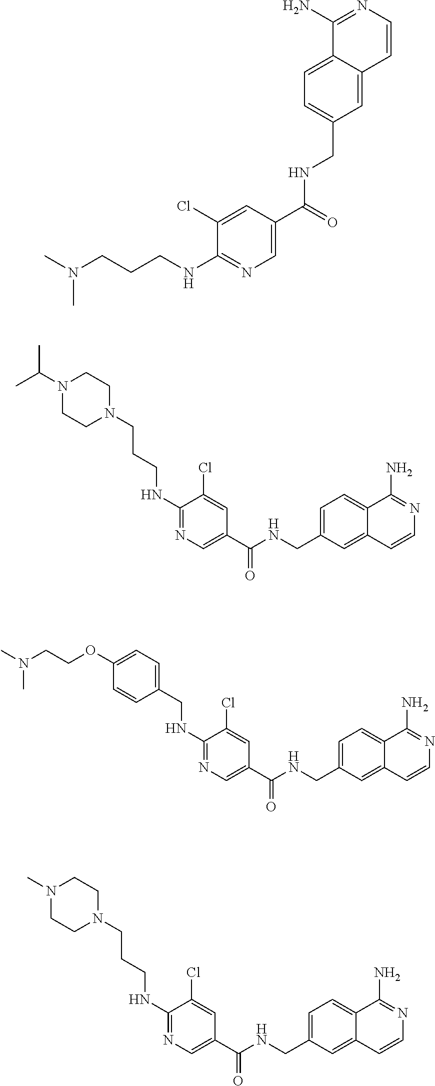 [SMILES: CN(C)CCCNc1ncc(C(=O)NCc2ccc3c(N)nccc3c2)cc1Cl ; CC(C)N1CCN(CCCNc2ncc(C(=O)NCc3ccc4c(N)nccc4c3)cc2Cl)CC1 ; CN(C)CCOc1ccc(CNc2ncc(C(=O)NCc3ccc4c(N)nccc4c3)cc2Cl)cc1 ; CN1CCN(CCCNc2ncc(C(=O)NCc3ccc4c(N)nccc4c3)cc2Cl)CC1] 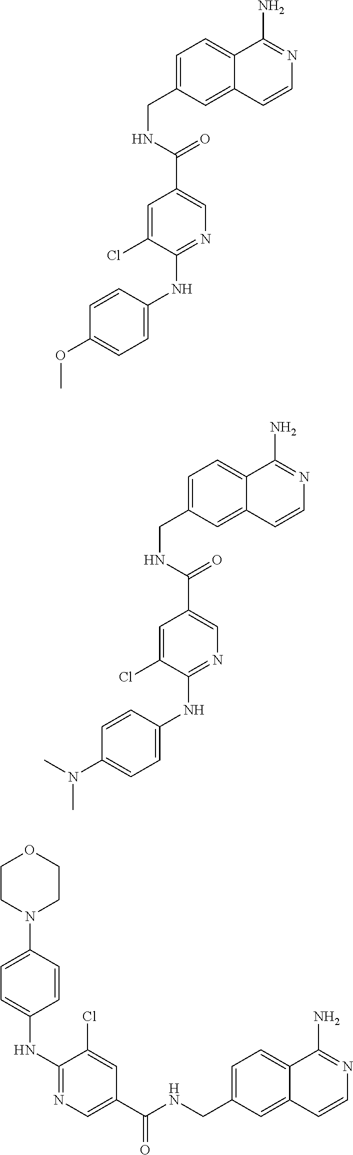 [SMILES: COc1ccc(Nc2ncc(C(=O)NCc3ccc4c(N)nccc4c3)cc2Cl)cc1 ; CN(C)c1ccc(Nc2ncc(C(=O)NCc3ccc4c(N)nccc4c3)cc2Cl)cc1 ; Nc1nccc2cc(CNC(=O)c3cnc(Nc4ccc(N5CCOCC5)cc4)c(Cl)c3)ccc12] 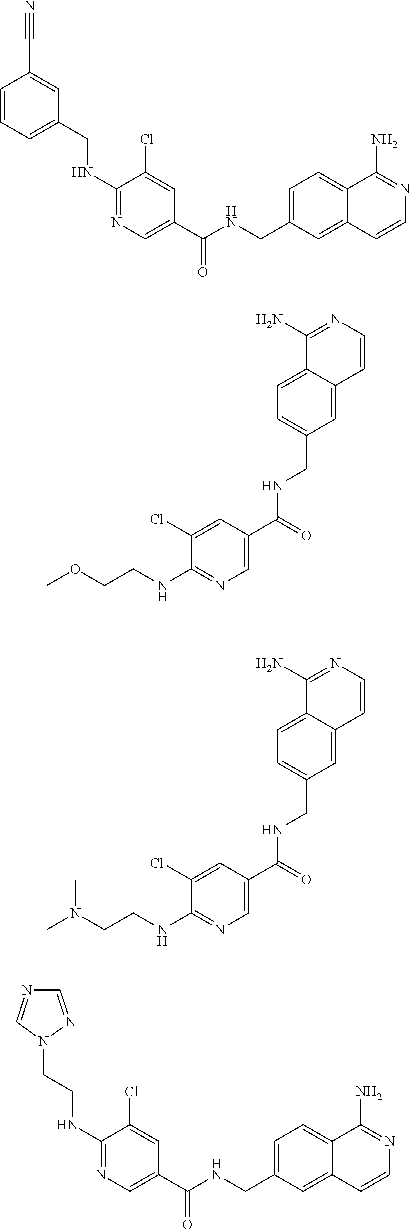 [SMILES: N#Cc1cccc(CNc2ncc(C(=O)NCc3ccc4c(N)nccc4c3)cc2Cl)c1 ; COCCNc1ncc(C(=O)NCc2ccc3c(N)nccc3c2)cc1Cl ; CN(C)CCNc1ncc(C(=O)NCc2ccc3c(N)nccc3c2)cc1Cl ; Nc1nccc2cc(CNC(=O)c3cnc(NCCn4cncn4)c(Cl)c3)ccc12] 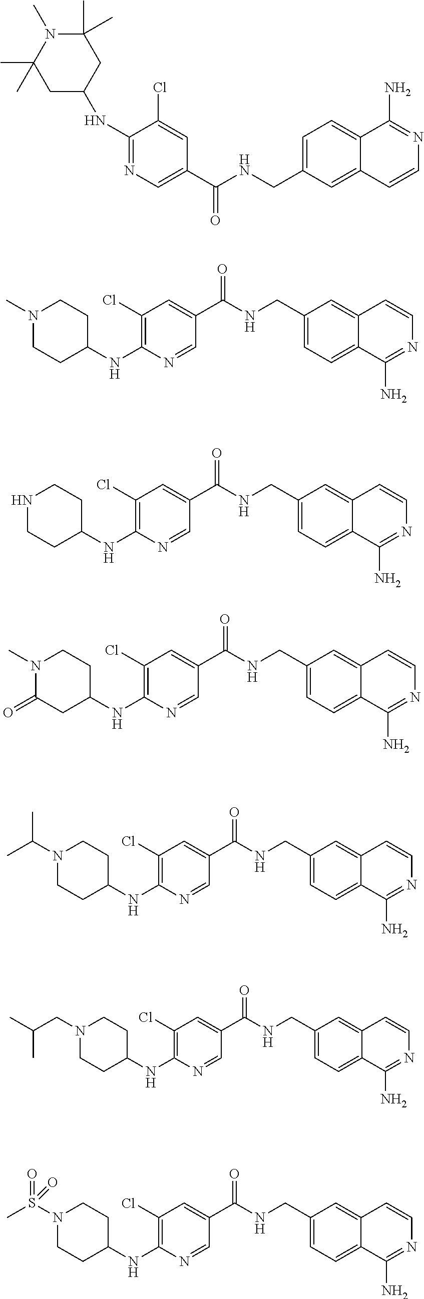 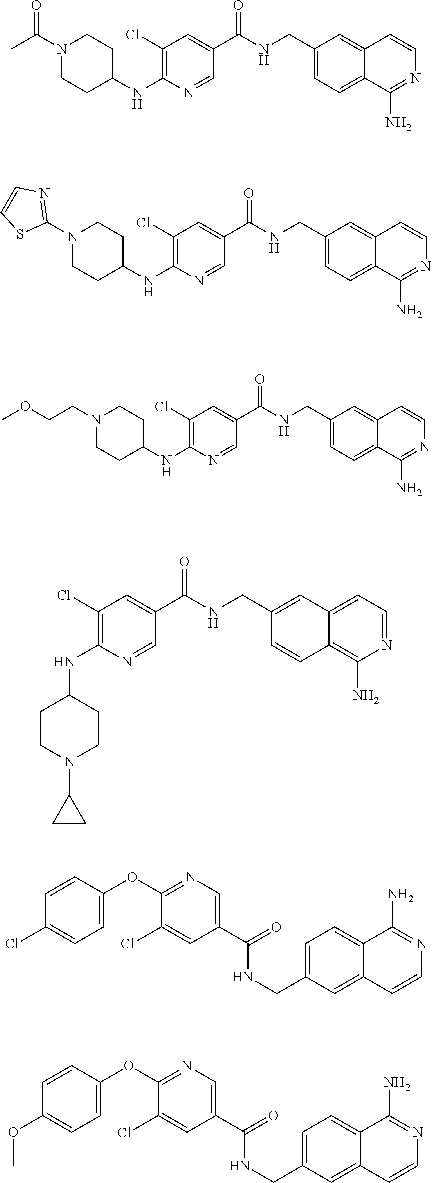 [SMILES: CC(=O)N1CCC(Nc2ncc(C(=O)NCc3ccc4c(N)nccc4c3)cc2Cl)CC1 ; Nc1nccc2cc(CNC(=O)c3cnc(NC4CCN(c5nccs5)CC4)c(Cl)c3)ccc12 ; COCCN1CCC(Nc2ncc(C(=O)NCc3ccc4c(N)nccc4c3)cc2Cl)CC1 ; Nc1nccc2cc(CNC(=O)c3cnc(NC4CCN(C5CC5)CC4)c(Cl)c3)ccc12 ; Nc1nccc2cc(CNC(=O)c3cnc(Oc4ccc(Cl)cc4)c(Cl)c3)ccc12 ; COc1ccc(Oc2ncc(C(=O)NCc3ccc4c(N)nccc4c3)cc2Cl)cc1] 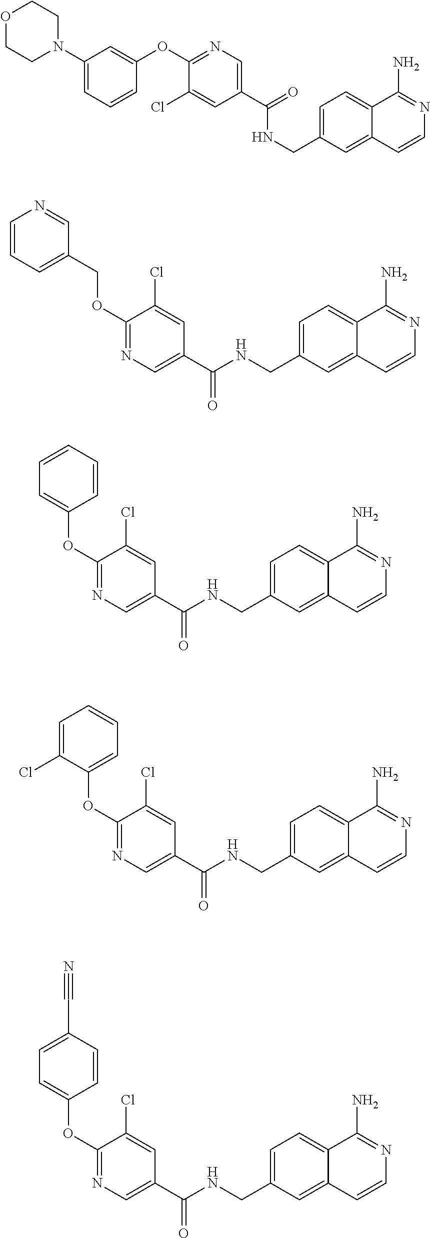 [SMILES: Nc1nccc2cc(CNC(=O)c3cnc(Oc4cccc(N5CCOCC5)c4)c(Cl)c3)ccc12 ; Nc1nccc2cc(CNC(=O)c3cnc(OCc4cccnc4)c(Cl)c3)ccc12 ; Nc1nccc2cc(CNC(=O)c3cnc(Oc4ccccc4)c(Cl)c3)ccc12 ; Nc1nccc2cc(CNC(=O)c3cnc(Oc4ccccc4Cl)c(Cl)c3)ccc12 ; N#Cc1ccc(Oc2ncc(C(=O)NCc3ccc4c(N)nccc4c3)cc2Cl)cc1] 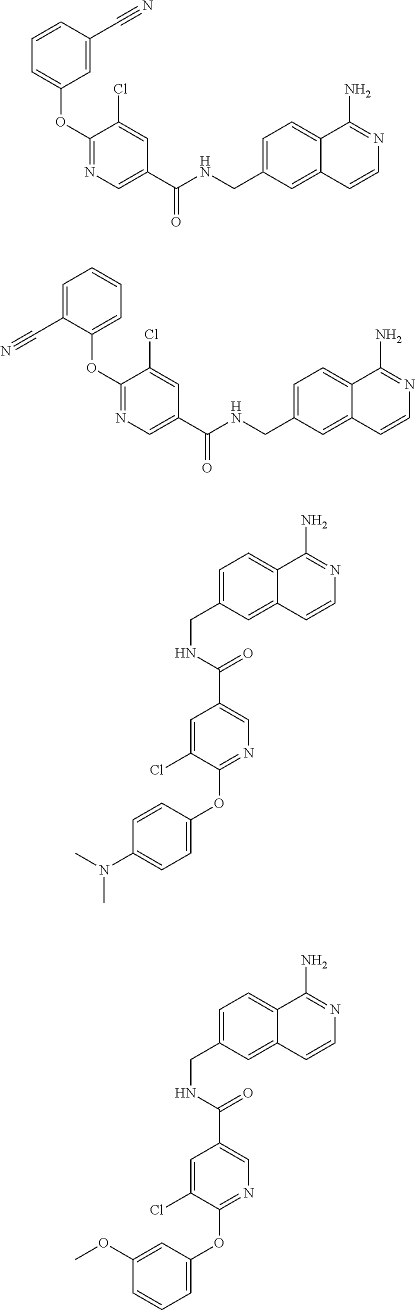 [SMILES: N#Cc1cccc(Oc2ncc(C(=O)NCc3ccc4c(N)nccc4c3)cc2Cl)c1 ; N#Cc1ccccc1Oc1ncc(C(=O)NCc2ccc3c(N)nccc3c2)cc1Cl ; CN(C)c1ccc(Oc2ncc(C(=O)NCc3ccc4c(N)nccc4c3)cc2Cl)cc1 ; COc1cccc(Oc2ncc(C(=O)NCc3ccc4c(N)nccc4c3)cc2Cl)c1] 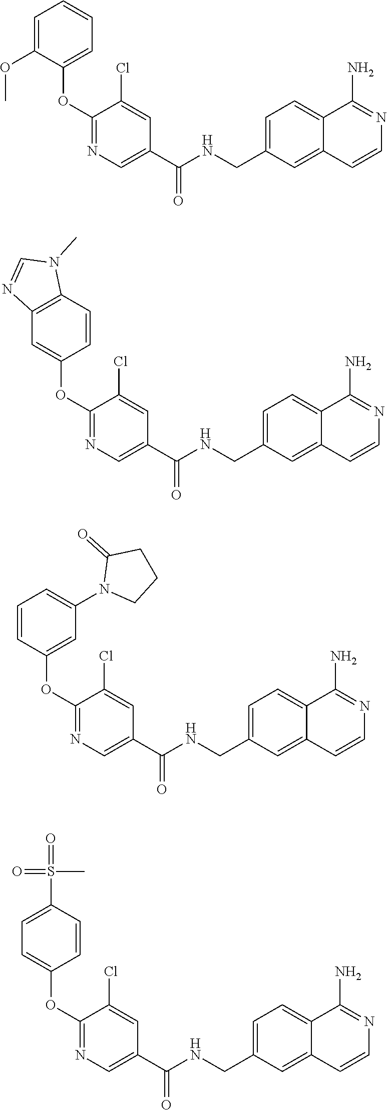 [SMILES: COc1ccccc1Oc1ncc(C(=O)NCc2ccc3c(N)nccc3c2)cc1Cl ; Cn1cnc2cc(Oc3ncc(C(=O)NCc4ccc5c(N)nccc5c4)cc3Cl)ccc21 ; Nc1nccc2cc(CNC(=O)c3cnc(Oc4cccc(N5CCCC5=O)c4)c(Cl)c3)ccc12 ; CS(=O)(=O)c1ccc(Oc2ncc(C(=O)NCc3ccc4c(N)nccc4c3)cc2Cl)cc1] 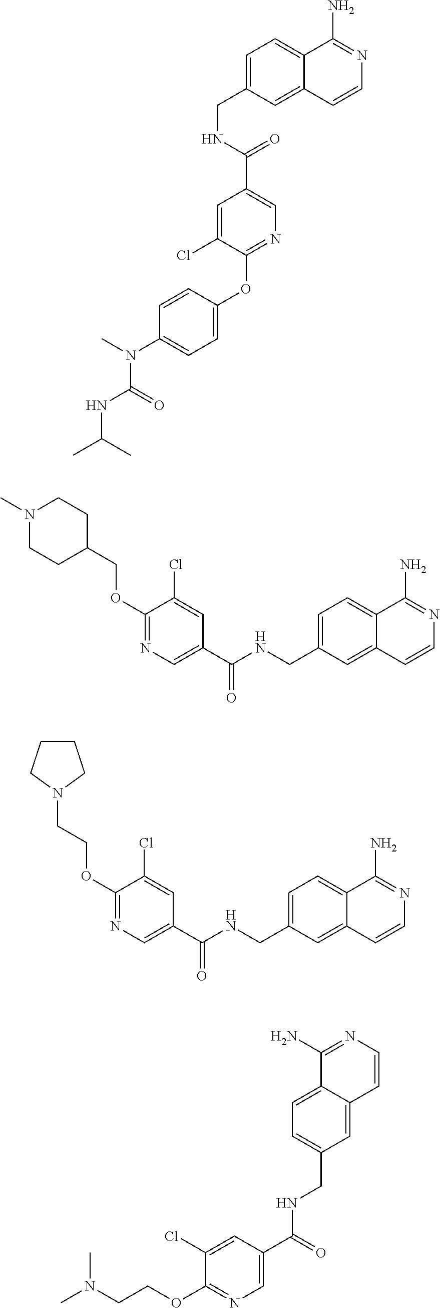 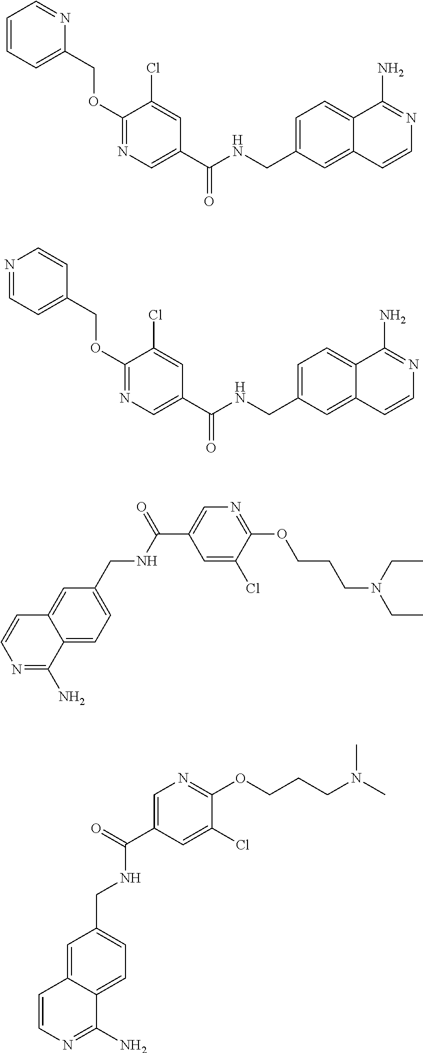 [SMILES: Nc1nccc2cc(CNC(=O)c3cnc(OCc4ccccn4)c(Cl)c3)ccc12 ; Nc1nccc2cc(CNC(=O)c3cnc(OCc4ccncc4)c(Cl)c3)ccc12 ; CCN(CC)CCCOc1ncc(C(=O)NCc2ccc3c(N)nccc3c2)cc1Cl ; CN(C)CCCOc1ncc(C(=O)NCc2ccc3c(N)nccc3c2)cc1Cl] 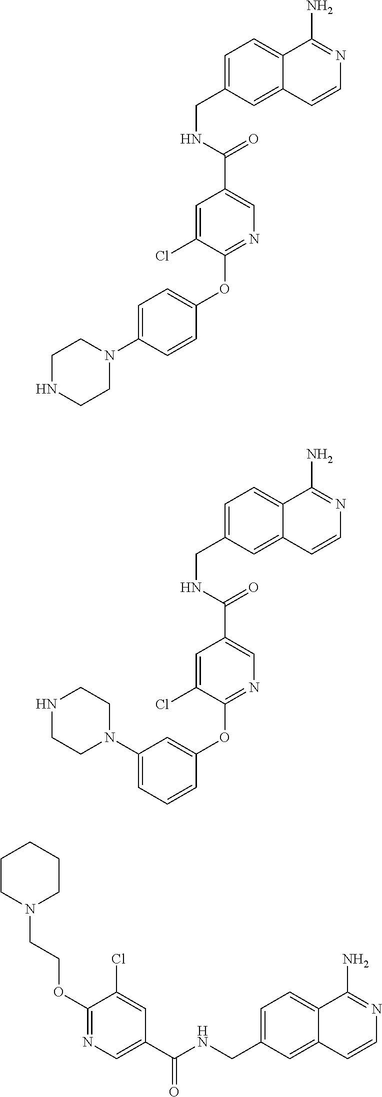 [SMILES: Nc1nccc2cc(CNC(=O)c3cnc(Oc4ccc(N5CCNCC5)cc4)c(Cl)c3)ccc12 ; Nc1nccc2cc(CNC(=O)c3cnc(Oc4cccc(N5CCNCC5)c4)c(Cl)c3)ccc12 ; Nc1nccc2cc(CNC(=O)c3cnc(OCCN4CCCCC4)c(Cl)c3)ccc12] 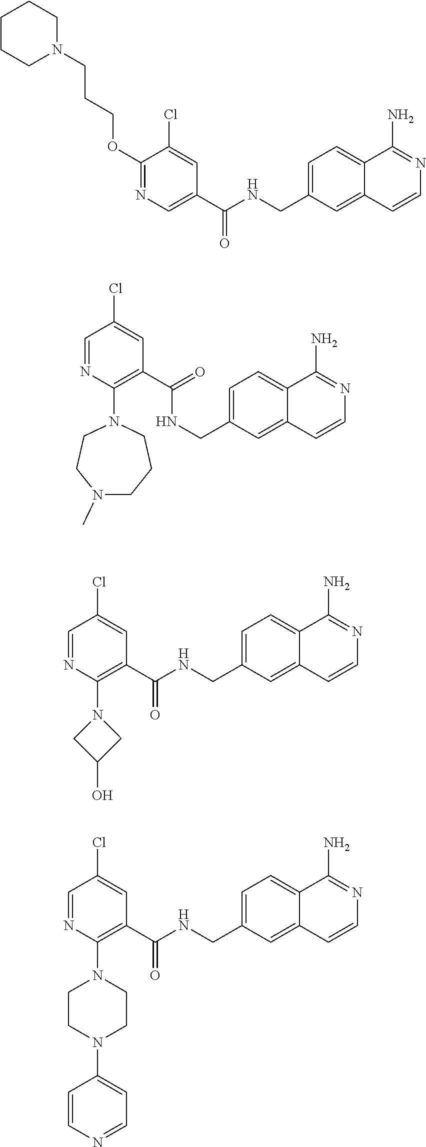 [SMILES: Nc1nccc2cc(CNC(=O)c3cnc(OCCCN4CCCCC4)c(Cl)c3)ccc12 ; CN1CCCN(c2ncc(Cl)cc2C(=O)NCc2ccc3c(N)nccc3c2)CC1 ; Nc1nccc2cc(CNC(=O)c3cc(Cl)cnc3N3CC(O)C3)ccc12 ; Nc1nccc2cc(CNC(=O)c3cc(Cl)cnc3N3CCN(c4ccncc4)CC3)ccc12] 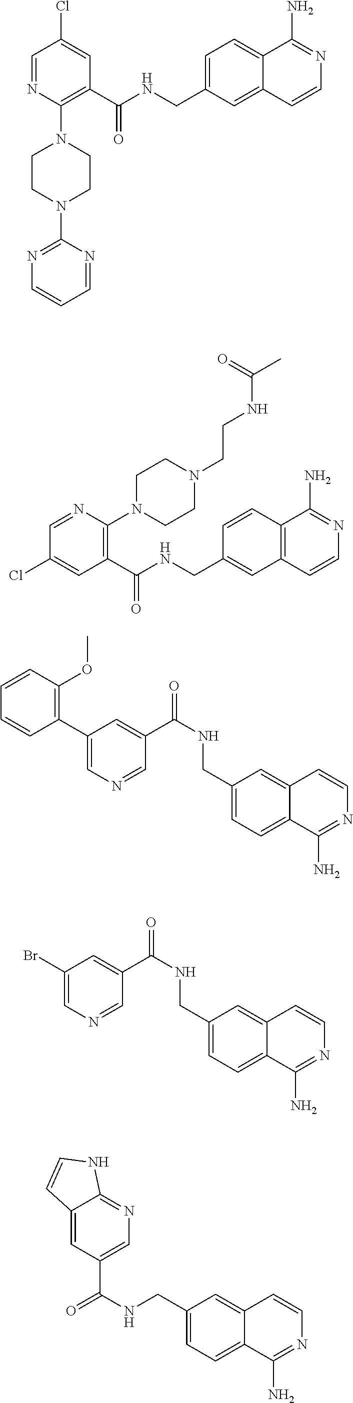 [SMILES: Nc1nccc2cc(CNC(=O)c3cc(Cl)cnc3N3CCN(c4ncccn4)CC3)ccc12 ; CC(=O)NCCN1CCN(c2ncc(Cl)cc2C(=O)NCc2ccc3c(N)nccc3c2)CC1 ; COc1ccccc1-c1cncc(C(=O)NCc2ccc3c(N)nccc3c2)c1 ; Nc1nccc2cc(CNC(=O)c3cncc(Br)c3)ccc12 ; Nc1nccc2cc(CNC(=O)c3cnc4[nH]ccc4c3)ccc12] 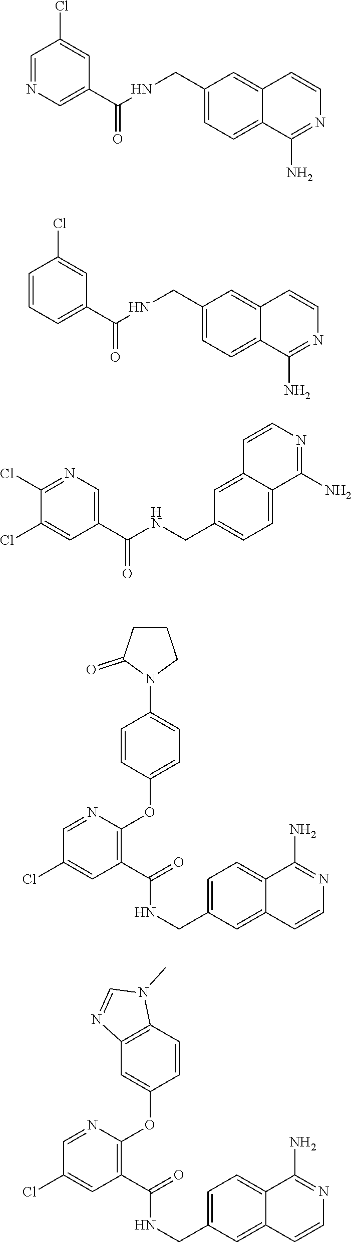 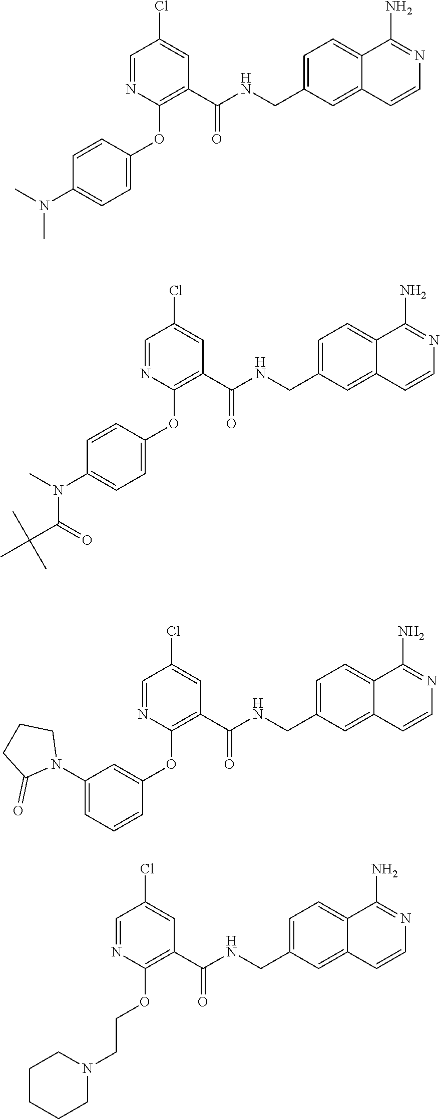 [SMILES: CN(C)c1ccc(Oc2ncc(Cl)cc2C(=O)NCc2ccc3c(N)nccc3c2)cc1 ; CN(C(=O)C(C)(C)C)c1ccc(Oc2ncc(Cl)cc2C(=O)NCc2ccc3c(N)nccc3c2)cc1 ; Nc1nccc2cc(CNC(=O)c3cc(Cl)cnc3Oc3cccc(N4CCCC4=O)c3)ccc12 ; Nc1nccc2cc(CNC(=O)c3cc(Cl)cnc3OCCN3CCCCC3)ccc12] 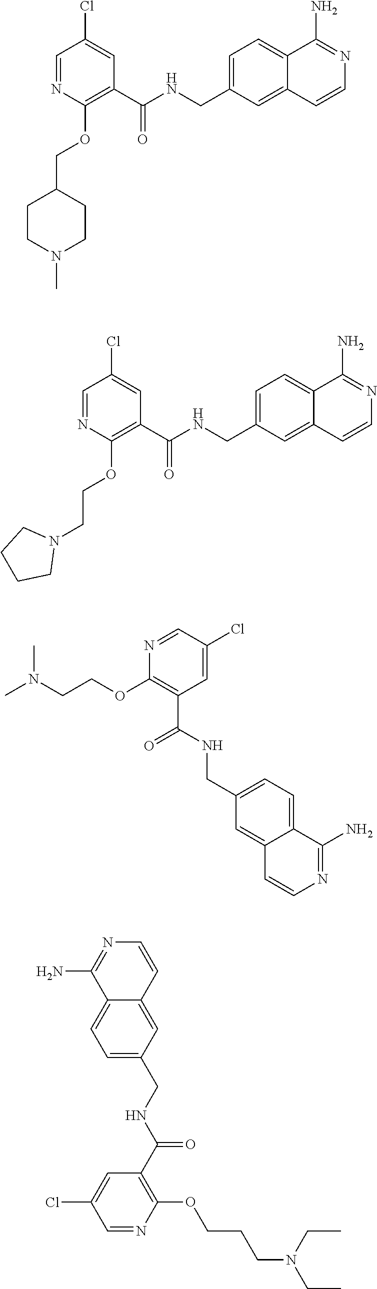 [SMILES: CN1CCC(COc2ncc(Cl)cc2C(=O)NCc2ccc3c(N)nccc3c2)CC1 ; Nc1nccc2cc(CNC(=O)c3cc(Cl)cnc3OCCN3CCCC3)ccc12 ; CN(C)CCOc1ncc(Cl)cc1C(=O)NCc1ccc2c(N)nccc2c1 ; CCN(CC)CCCOc1ncc(Cl)cc1C(=O)NCc1ccc2c(N)nccc2c1] 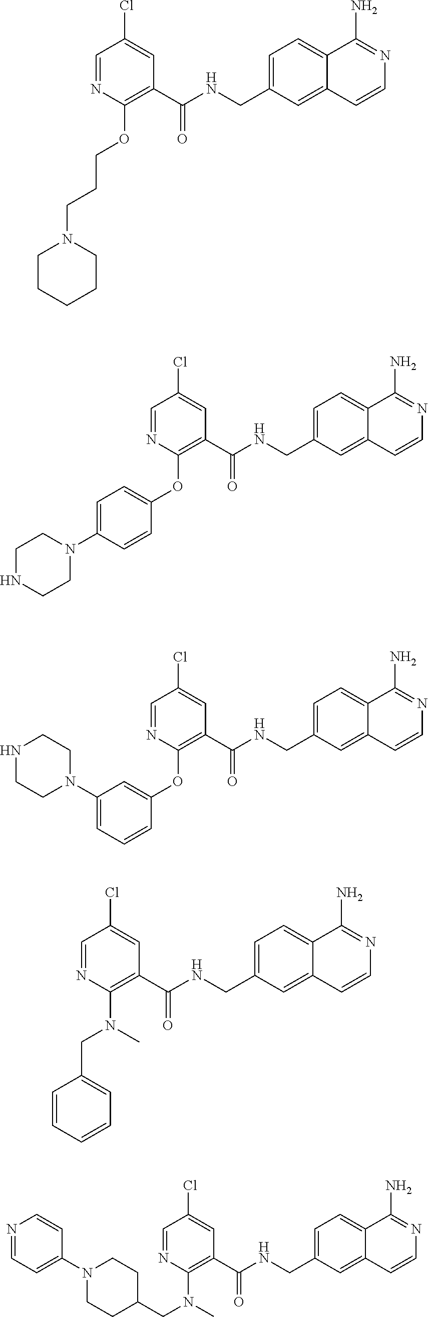 [SMILES: Nc1nccc2cc(CNC(=O)c3cc(Cl)cnc3OCCCN3CCCCC3)ccc12 ; Nc1nccc2cc(CNC(=O)c3cc(Cl)cnc3Oc3ccc(N4CCNCC4)cc3)ccc12 ; Nc1nccc2cc(CNC(=O)c3cc(Cl)cnc3Oc3cccc(N4CCNCC4)c3)ccc12 ; CN(Cc1ccccc1)c1ncc(Cl)cc1C(=O)NCc1ccc2c(N)nccc2c1 ; CN(CC1CCN(c2ccncc2)CC1)c1ncc(Cl)cc1C(=O)NCc1ccc2c(N)nccc2c1] 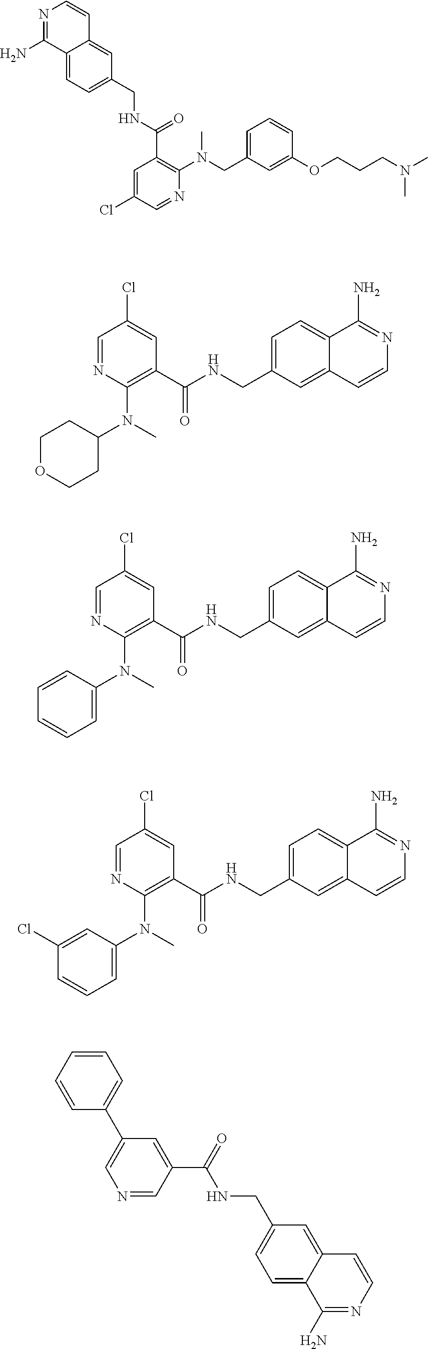 [SMILES: CN(C)CCCOc1cccc(CN(C)c2ncc(Cl)cc2C(=O)NCc2ccc3c(N)nccc3c2)c1 ; CN(c1ncc(Cl)cc1C(=O)NCc1ccc2c(N)nccc2c1)C1CCOCC1 ; CN(c1ccccc1)c1ncc(Cl)cc1C(=O)NCc1ccc2c(N)nccc2c1 ; CN(c1cccc(Cl)c1)c1ncc(Cl)cc1C(=O)NCc1ccc2c(N)nccc2c1 ; Nc1nccc2cc(CNC(=O)c3cncc(-c4ccccc4)c3)ccc12] 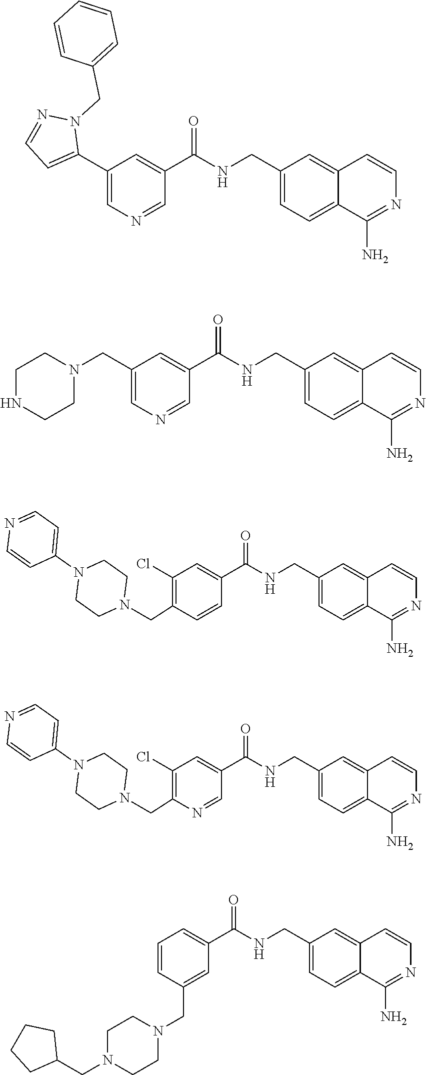 [SMILES: Nc1nccc2cc(CNC(=O)c3cncc(-c4ccnn4Cc4ccccc4)c3)ccc12 ; Nc1nccc2cc(CNC(=O)c3cncc(CN4CCNCC4)c3)ccc12 ; Nc1nccc2cc(CNC(=O)c3ccc(CN4CCN(c5ccncc5)CC4)c(Cl)c3)ccc12 ; Nc1nccc2cc(CNC(=O)c3cnc(CN4CCN(c5ccncc5)CC4)c(Cl)c3)ccc12 ; Nc1nccc2cc(CNC(=O)c3cccc(CN4CCN(CC5CCCC5)CC4)c3)ccc12] 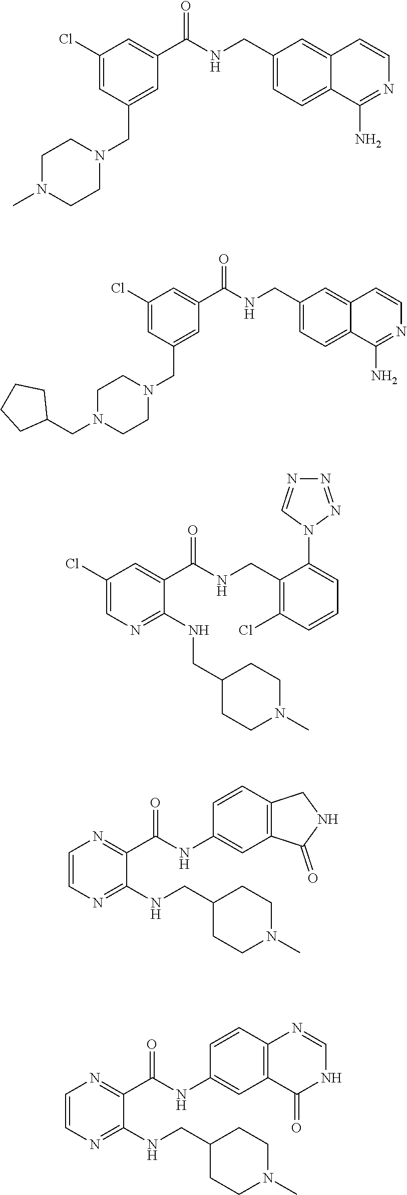 [SMILES: CN1CCN(Cc2cc(Cl)cc(C(=O)NCc3ccc4c(N)nccc4c3)c2)CC1 ; Nc1nccc2cc(CNC(=O)c3cc(Cl)cc(CN4CCN(CC5CCCC5)CC4)c3)ccc12 ; CN1CCC(CNc2ncc(Cl)cc2C(=O)NCc2c(Cl)cccc2-n2cnnn2)CC1 ; CN1CCC(CNc2nccnc2C(=O)Nc2ccc3c(c2)C(=O)NC3)CC1 ; CN1CCC(CNc2nccnc2C(=O)Nc2ccc3nc[nH]c(=O)c3c2)CC1] 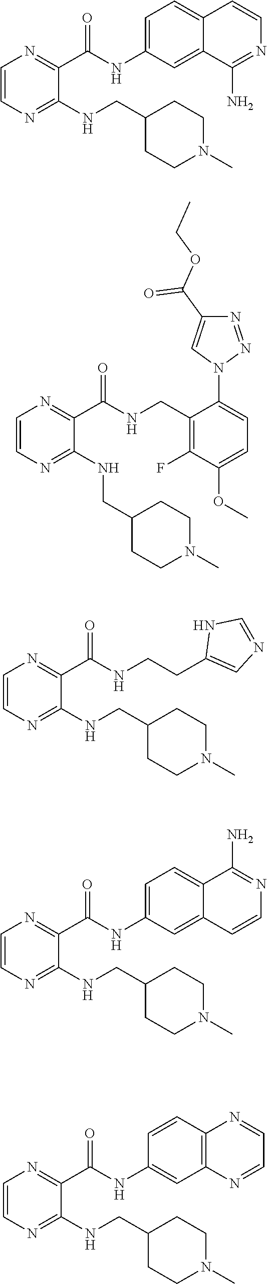 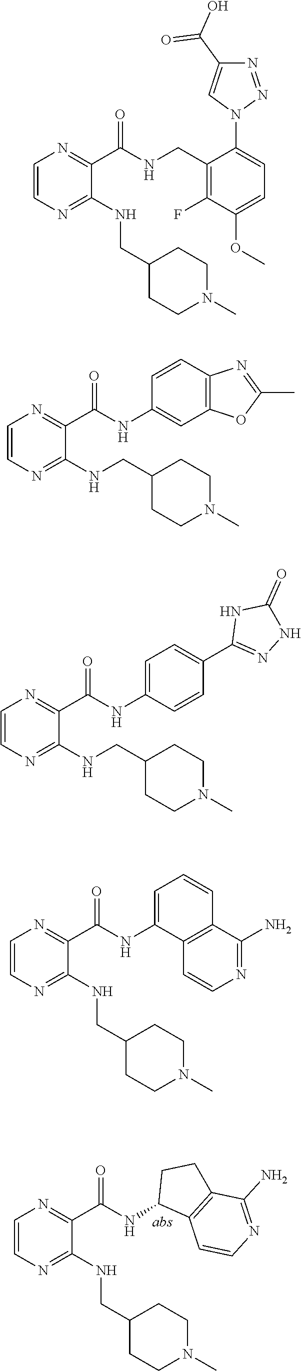 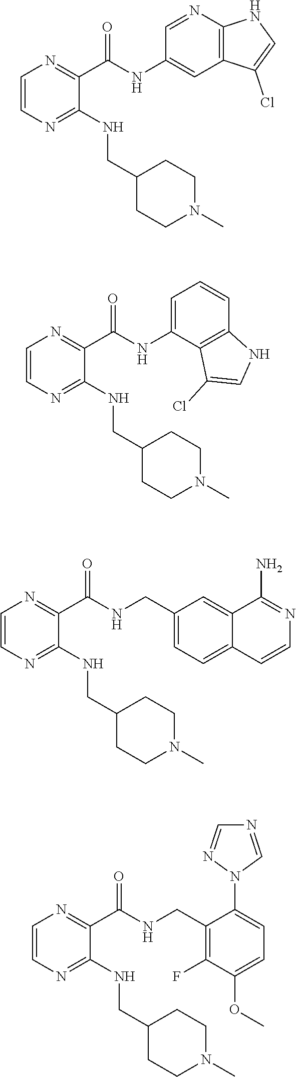 [SMILES: CN1CCC(CNc2nccnc2C(=O)Nc2cnc3[nH]cc(Cl)c3c2)CC1 ; CN1CCC(CNc2nccnc2C(=O)Nc2cccc3[nH]cc(Cl)c23)CC1 ; CN1CCC(CNc2nccnc2C(=O)NCc2ccc3ccnc(N)c3c2)CC1 ; COc1ccc(-n2cncn2)c(CNC(=O)c2nccnc2NCC2CCN(C)CC2)c1F] 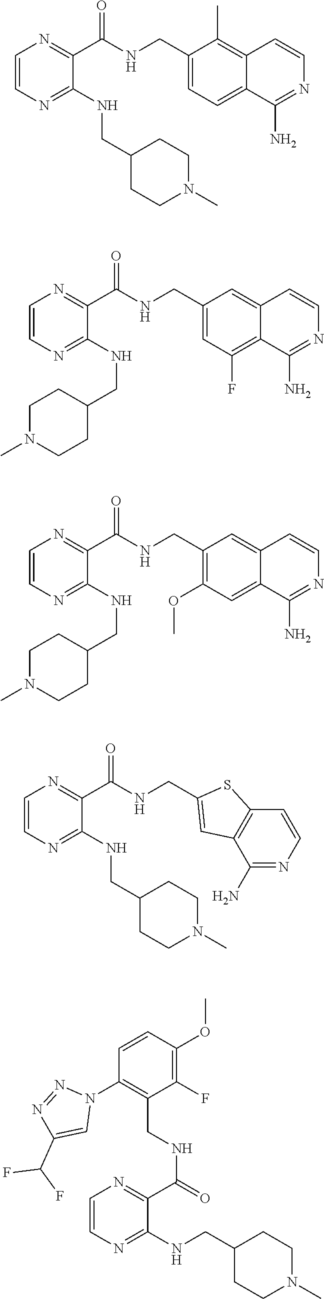 [SMILES: Cc1c(CNC(=O)c2nccnc2NCC2CCN(C)CC2)ccc2c(N)nccc12 ; CN1CCC(CNc2nccnc2C(=O)NCc2cc(F)c3c(N)nccc3c2)CC1 ; COc1cc2c(N)nccc2cc1CNC(=O)c1nccnc1NCC1CCN(C)CC1 ; CN1CCC(CNc2nccnc2C(=O)NCc2cc3c(N)nccc3s2)CC1 ; COc1ccc(-n2cc(C(F)F)nn2)c(CNC(=O)c2nccnc2NCC2CCN(C)CC2)c1F] 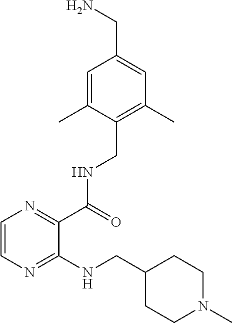 [SMILES: Cc1cc(CN)cc(C)c1CNC(=O)c1nccnc1NCC1CCN(C)CC1]
or a pharmaceutically acceptable salt and/or solvate thereof.

27. A pharmaceutical composition comprising: the compound, or a pharmaceutically acceptable salt and/or solvate thereof, according to claim 1, and at least one pharmaceutically acceptable excipient.

28. (canceled)

29. (canceled)

30. A method of treating a disease or condition in which Factor XIIa activity is implicated, comprising administering to a subject in need thereof a therapeutically effective amount of the compound, or a pharmaceutically acceptable salt and/or solvate thereof, of claim 1.

31. (canceled)

32. The method of claim 30, wherein the disease or condition in which Factor XIIa activity is implicated is a bradykinin-mediated angioedema.

33. The method of claim 32, wherein the bradykinin-mediated angioedema is hereditary angioedema.

34. The method of claim 32, wherein the bradykinin-mediated angioedema is non hereditary.

35. The method of claim 30, wherein the disease or condition in which Factor XIIa activity is implicated is selected from vascular hyperpermeability; stroke including ischemic stroke and haemorrhagic accidents; retinal edema; diabetic retinopathy; DME; retinal vein occlusion; or AMD.

36. The method of claim 30, wherein the disease or condition in which Factor XIIa activity is implicated is a thrombotic disorder.

37. The method of claim 36, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.

38. The method of claim 30, wherein the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy or migraine; sepsis; bacterial sepsis; inflammation; vascular hyperpermeability; or anaphylaxis.

39. The method of claim 30 wherein the compound targets FXIIa.

40. The compound of claim 1, wherein the stereoisomer is an enantiomer, diastereoisomer, racemic mixture, or scalemic mixture.
